(19) United States
(12) Patent Application Publication
(10) Pub. No.: US 2026/0185069 A1
(43) Pub. Date: Jul. 2, 2026

(54) DNA RECOMBINASE FUSIONS

(71) Applicants: Arc Research Institute, Palo Alto, CA (US); The Regents of the University of California, Oakland, CA (US)

(72) Inventors: Alison FANTON, San Francisco, CA (US); Patrick HSU, Palo Alto, CA (US)

(21) Appl. No.: 19/126,084

(22) PCT Filed: Nov. 1, 2023
(86) PCT No.: PCT/US23/78337

§ 371 Date: Apr. 30, 2025

Related U.S. Application Data
(60) Provisional application No. 63/421,480, filed on Nov. 1, 2022.
(60) Provisional application No. 63/516,424, filed on Jul. 28, 2023.

(51) Int. Cl. C12N 9/22; C12N 9/00; C12N 15/113; C12N 15/52; C12N 15/62; C12N 15/85

(52) U.S. Cl. CPC C12N 9/226; C12N 9/93; C12N 15/113; C12N 15/52; C12N 15/625; C12N 15/85; C12Y 605/01; C12N 2310/20

(57) ABSTRACT

Disclosed herein are fusion polypeptides and nucleic acid encoding the same, wherein the fusion polypeptide comprises a large serine recombinase (LSR) portion and a DNA binding domain (DBD) portion.

The International Patent application claims the benefit of and priority to U.S. Application No. 63/421,480, filed Nov. 1, 2022, entitled “RECOMBINASES FOR INTEGRATING DNA,” and U.S. Application No. 63/516,424, filed Jul. 28, 2023, entitled “DNA RECOMBINASE FUSIONS,” the contents of which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in XML format and is hereby incorporated by reference in its entirety. Said XML copy, created on Oct. 30, 2023, is named 2220476_00123WO1_SL.xml and is 620,017 bytes in size.

### BACKGROUND OF THE INVENTION

Large serine recombinases (LSRs) are bacteriophage-encoded DNA integrases that can facilitate the site-specific and unidirectional insertion of phage DNA into bacterial genomes via recombination of two attachment sites, termed attP (phage) and attB (bacteria). These enzymes have been shown to site-specifically integrate DNA payloads containing a donor attachment site (attD, which could correspond to the native attP or attB) in mammalian cells, both at pre-installed integration sites or at endogenous genomic pseudosites with high sequence similarity to their cognate acceptor attachment sites (attA). If the attA sequence is found in the human genome, it is termed an attH sequence. But, despite their sequence specificity, LSRs may integrate into numerous sites in the human genome due to the presence of multiple loci with sufficient integration site sequences.

Manipulation of eukaryotic genomes, particularly the integration of multi-kilobase DNA sequences, remains challenging and limits the rapidly growing fields of synthetic biology and cell engineering. Challenges include low insertion efficiency, high indel rates, and cargo size limitations, with limited success for cargoes larger than 1 kilobase (kb). In particular, LSRs can be limited by a low integration efficiency. Thus, there remains a need for improved genetic engineering systems and methods.

### SUMMARY OF THE INVENTION

It is understood that any of the embodiments described below can be combined in any desired way, and that any embodiment or combination of embodiments can be applied to each of the aspects described below, unless the context indicates otherwise.

In certain aspects, described herein is a nucleic acid comprising a sequence encoding a fusion polypeptide, wherein the fusion polypeptide comprises a large serine recombinase (LSR) portion and a DNA binding domain (DBD) portion. In some embodiments, the nucleic acid sequence encodes a fusion polypeptide wherein the LSR portion is fused N-terminal to the DBD portion. In some embodiments, the nucleic acid sequence encoding the fusion polypeptide further comprises a nucleic acid sequence encoding a peptide linker positioned between a nucleic acid sequence encoding the LSR portion and a nucleic acid sequence encoding the DBD portion. In some embodiments, the nucleic acid sequence encodes a fusion polypeptide wherein the LSR portion is fused N-terminal to the DBD portion by the peptide linker.

In some embodiments, the peptide linker encoded by the nucleic acid comprises at least one amino acid. In some embodiments, the peptide linker encoded by the nucleic acid comprises 2 to 100 amino acids. In some embodiments, the peptide linker encoded by the nucleic acid comprises 15 to 70 amino acids. In some embodiments, the peptide linker encoded by the nucleic acid comprises glycine and serine residues. In some embodiments, the peptide linker encoded by the nucleic acid comprises GGS, GGSS (SEQ ID NO: 584), GGGS (SEQ ID NO: 572), or GGGGS (SEQ ID NO: 596) repeats. In some embodiments, the peptide linker encoded by the nucleic acid comprises one or more XTEN16 repeats. In some embodiments, the polypeptide linker encoded by the nucleic acid comprises one XTEN16 repeat, two XTEN16 repeats, or three XTEN16 repeats. In some embodiments, the polypeptide linker encoded by the nucleic acid comprises the amino acid sequence of SEQ ID NOs:11-15. In some embodiments, the nucleic acid sequence encoding the polypeptide linker comprises SEQ ID NOs:20-24.

In some embodiments, the LSR portion encoded by the nucleic acid comprises an amino acid sequence at least 90% identical to SEQ ID NOs: 1-5, 432-443, 445-446, 448-467, 469-476, 478-492, 494-501, 276, 279, 282, 285, 288, or 291. In some embodiments, the LSR portion encoded by the nucleic acid comprises an amino acid sequence of SEQ ID NOs: 1-5, 432-443, 445-446, 448-467, 469-476, 478-492, 494-501, 276, 279, 282, 285, 288, or 291. In some embodiments, the LSR portion encoded by the nucleic acid comprises Dn29 (SEQ ID NO:1), Pf80 (SEQ ID NO:2), Cp36 (SEQ ID NO:3), Nm60 (SEQ ID NO:4), or Si74 (SEQ ID NO:5). In some embodiments, the nucleic acid sequence encoding the LSR portion comprises a nucleic acid sequence at least 90% identical to SEQ ID NOs:6-10. In some embodiments, the nucleic acid sequence encoding the LSR portion comprises a nucleic acid sequence of SEQ ID NOs:6-10.

In some embodiments, the fusion polypeptide encoded by the nucleic acid further comprises one or more nuclear localization signals (NLSs). In some embodiments, the DBD portion encoded by the nucleic acid comprises Cas9, Cpf1, Cas12b, Cas12c, Cas12d, Cas12e, Cas12f, Cas12h, Cas12i, or Cas12g. In some embodiments, the Cas9, Cpf1, Cas12b, Cas12c, Cas12d, Cas12e, Cas12f, Cas12h, Cas12i, or Cas12g lack nuclease and/or nickase activity. In some embodiments, the DBD portion encoded by the nucleic acid comprises dCas9. In some embodiments, the DBD portion encoded by the nucleic acid comprises an amino acid sequence at least 90% identical to dCas9 (SEQ ID NO:29), dCas9-HF1 (SEQ ID NO:30), dCas9-SpG (SEQ ID NO:31), or dCas9-SpG-HF1 (SEQ ID NO:32). In some embodiments, the DBD portion encoded by the nucleic acid comprises an amino acid sequence of dCas9 (SEQ ID NO:29), dCas9-HF1 (SEQ ID NO:30), dCas9-SpG (SEQ ID NO:31), or dCas9-SpG-HF1 (SEQ ID NO:32).

In some embodiments, the nucleic acid sequence encoding the DBD portion comprises a nucleic acid sequence at least 90% identical SEQ ID NOs:33-36. In some embodiments, the nucleic acid sequence encoding the DBD portion comprises a nucleic acid sequence of SEQ ID NOs:33-36.

In some embodiments, the fusion polypeptide encoded by the nucleic acid comprises Dn29 (SEQ ID NO: 1) and dCas9 (SEQ ID NO: 29), Pf80 (SEQ ID NO:2) and dCas9 (SEQ ID NO: 29), Cp36 (SEQ ID NO:3) and dCas9 (SEQ ID NO: 29), Nm60 (SEQ ID NO:4) and dCas9 (SEQ ID NO: 29), or Si74 (SEQ ID NO:5) and dCas9 (SEQ ID NO: 29). In some embodiments, the fusion polypeptide encoded by the nucleic acid further comprises a peptide linker positioned between the nucleic acid sequence encoding the LSR portion and nucleic acid sequence encoding the DBD portion wherein the LSR portion is fused N-terminal to the DBD portion by the peptide linker and the peptide linker encoded by the nucleic acid comprises (GGS)₈ (SEQ ID NO: 11), (GGGGS)₆ (SEQ ID NO: 598), S(GGGGS)₆S (SEQ ID NO: 12), XTEN16 (SEQ ID NO: 13), XTEN32-(GGSS)₂ (SEQ ID NO: 14), or XTEN48-(GGSS)₂ (SEQ ID NO: 15). In some embodiments, the fusion polypeptide encoded by the nucleic acid comprises an amino acid sequence at least 90% identical to SEQ ID NOs: 37-42. In some embodiments, the fusion polypeptide encoded by the nucleic acid comprises an amino acid sequence of SEQ ID NOs: 37-42. In some embodiments, the DBD portion of the fusion polypeptide encoded by the nucleic acid binds to a guide RNA (gRNA).

In certain aspects, described herein is a vector comprising any of the nucleic acids of the invention. In certain aspects, described herein is a host cell comprising the vector of the invention.

In certain aspects, described herein is a nucleic acid editing system comprising a first nucleic acid encoding an LSR-DBD as described herein and a second nucleic acid encoding a gRNA. In some embodiments, the gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides upstream or downstream of a dinucleotide core of an attachment site of the LSR portion of the fusion polypeptide on a DNA of interest. In some embodiments, the spacer sequence portion is 16 to 20 nucleotides long. In some embodiments, the gRNA encoded by the nucleic acid is an sgRNA. In some embodiments, immediately 3′ to the target nucleic acid sequence on the DNA of interest is a PAM sequence.

In some embodiments, the target nucleic acid sequence is within 80 nucleotides upstream or downstream of a dinucleotide core of an attA site of the LSR portion of the fusion polypeptide on a target DNA of interest. In some embodiments, the attA site is a pseudosite in a mammalian target DNA of interest. In some embodiments, the attA site is a pseudosite in the human genome (attH). In some embodiments, the fusion polypeptide encoded by the nucleic acid comprises Dn29 (SEQ ID NO: 1) and dCas9 (SEQ ID NO: 29) and the attH site is chr10:21130404-21130406:−, chr11:77367459-77367461:−, chr1:230490334-230490336:+, chr2:14280297-14280299:+, chr9:116464427-116464429:+, chr20:38982599-38982601:+, chr5:3553012-3553014:−, chr7:134676315-134676317:−, chr10:58514255-58514257:+, or chr4:92338934-92338936:+. In some embodiments, the fusion polypeptide encoded by the nucleic acid comprises Pf80 (SEQ ID NO:2) and dCas9 (SEQ ID NO: 29) and the attH site is chr11:64243293-64243295.

In some embodiments, the tracr RNA portion comprises SEQ ID NO: 153. In some embodiments, the target nucleic acid sequence is within 80 nucleotides upstream of a dinucleotide core of an attachment site of the LSR portion of the fusion polypeptide on a DNA of interest. In some embodiments, the target nucleic acid sequence is within 80 nucleotides downstream of a dinucleotide core of an attachment site of the LSR portion of the fusion polypeptide on a DNA of interest.

In some embodiments, the nucleic acid editing system further comprises a third nucleic acid encoding a second gRNA. In some embodiments, the second gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides downstream of a dinucleotide core of an attachment site of the LSR portion of the fusion polypeptide on a DNA of interest. In some embodiments, the spacer sequence portion of the second gRNA is 16 to 20 nucleotides long. In some embodiments, the second gRNA encoded by the nucleic acid is an sgRNA. In some embodiments, immediately 3′ to the target nucleic acid sequence on the DNA of interest is a PAM sequence.

In some embodiments, the nucleic acid editing system further comprises a third nucleic acid comprising a donor DNA sequence which comprises an attD attachment site of the LSR portion of the fusion polypeptide and a nucleic acid sequence for insertion into the target DNA of interest. In some embodiments, the third nucleic acid further comprises a portion that has the same target nucleic acid sequence for the gRNA as the target DNA of interest.

In some embodiments, the fusion polypeptide encoded by the nucleic acid comprises: (a) Dn29 (SEQ ID NO: 1) and dCas9 (SEQ ID NO: 29), the attH site on the target DNA of interest is chromosomal locus chr10:21130404-21130406:−, chr11:77367459-77367461:−, chr1:230490334-230490336:+, chr2:14280297-14280299:+, chr9:116464427-116464429:+, chr20:38982599-38982601:+, chr5:3553012-3553014:−, chr7:134676315-134676317:−, chr10:58514255-58514257:+, or chr4:92338934-92338936:+ or comprises the attH sequence found at said chromosomal locus, and the attD attachment site of the donor DNA sequence comprises SEQ ID NO: 154, or a sequence 90% identical to SEQ ID NO: 154; (b) Pf80 (SEQ ID NO: 2) and dCas9 (SEQ ID NO: 29), the attH site on the target DNA of interest is chromosomal locus chr11:64243293-64243295:+, chr1:162878224-162878226:+, chr11:92763120-92763122:−, chr9:103309977-103309979:−, chr13:91145766-91145768:+, chr2:102467361-102467363:+, chr13:99865454-99865456:+, chr9:113640780-113640782:−, chr9:123986548-123986550:−, chr15:53565450-53565452:−, or comprises the attH sequence found at said chromosomal locus, and the attD attachment site of the donor DNA sequence comprises SEQ ID NO: 265, or a sequence 90% identical to SEQ ID NO: 265; (c) Cp36 (SEQ ID NO: 3) and dCas9 (SEQ ID NO: 29), the attH site on the target DNA of interest is chromosomal locus chr16:2789124-2789126:+, chr22:43958465-43958467:−, chr10:117762740-117762742:+, chr7:157294532-157294534:−, chr13:20558930-20558932:−, chr6:151120348-151120350:−, chr10:101429887-101429889:+, chr1:20686551-20686553:+, chr19:50987430-50987432:+, chr4:183226741-183226743:−, or comprises the attH sequence found at said chromosomal locus, and the attD attachment site of the donor DNA sequence comprises SEQ ID NO: 267, or a sequence 90% identical to SEQ ID NO: 267; (d) Nm60 (SEQ ID NO: 4) and dCas9 (SEQ ID NO: 29), the attH site on the target DNA of interest is chromosomal locus chr9:83308042-83308044:−, chr13:79497139-79497141:−, chr9:131409759-131409761:+, chr4:55980785-55980787:+, chr5:96968267-96968269:+, chr6:37700280-37700282:−, chr19:17495840-17495842:−, chr5:126546219-126546221:+, chr10:15703649-15703651:−, chr10:395348-395350:+, or comprises the attH sequence found at said chromosomal locus, and the attD attachment site of the donor DNA sequence comprises SEQ ID NO: 234, or a sequence 90% identical to SEQ ID NO: 234; or (e) Si74 (SEQ ID NO: 5) and dCas9 (SEQ ID NO: 29), the attH site on the target DNA of interest is chromosomal locus chr7:155557356-155557358:+, chr9:77155112-77155114:−, or comprises the attH sequence found at said chromosomal locus, and the attD attachment site of the donor DNA sequence comprises SEQ ID NO: 266, or a sequence 90% identical to SEQ ID NO: 266.

In some embodiments, the third nucleic acid is a plasmid. In some embodiments, the third nucleic acid is a linear amplicon.

In certain aspects, described herein is a vector comprising any of the nucleic acids of the invention. In certain aspects, described herein is a host cell comprising any of the vector(s) of the invention. In some embodiments, the nucleic acid encoding the fusion polypeptide, the nucleic acid encoding the gRNA, or both, and/or, where present, the third nucleic acid encoding the second gRNA are expressed from an inducible promoter.

In certain aspects, described herein is a method of integrating a donor DNA sequence into a target DNA of interest of a cell, the method comprising introducing into the cell: a nucleic acid editing system of the invention. In some embodiments, the cell is a mammalian cell. In some embodiments, the cell is a human cell. In some embodiments, the cell is a human embryonic stem cell. In some embodiments, the cell is a hepatocellular carcinoma cell. In some embodiments, the cell is a HEK cell. In some embodiments, the target DNA of interest of the cell was engineered before introduction of the nucleic acid editing system to contain an attA attachment site. In some embodiments, the donor DNA comprises an LSR attD attachment site which is integrated into the target DNA of interest. In some embodiments, the target DNA of interest of the cell is the genome of the cell. In some embodiments, the target DNA of interest of the cell is a plasmid.

In certain aspects, described herein is a method of inverting a DNA sequence of a target DNA of interest, the method comprising introducing into a cell: a nucleic acid editing system of the invention, wherein attD and attA attachment sites of the LSR portion of the fusion polypeptide are present on the same DNA target molecule of interest in reverse orientation. In some embodiments, the target DNA of interest of the cell was engineered before introduction of the nucleic acid editing system to contain an attA attachment site. In some embodiments, the target DNA of interest of the cell was engineered before introduction of the nucleic acid editing system to contain an attD attachment site. In some embodiments, the target DNA of interest of the cell is the genome of the cell.

In certain aspects, described herein is a method of excising a DNA sequence of a target DNA of interest, the method comprising introducing into a cell: a nucleic acid editing system of the invention, wherein attD and attA attachment sites of the LSR portion of the fusion polypeptide are present on the same DNA target molecule of interest in the same orientation. In some embodiments, the target DNA of interest of the cell was engineered before introduction of the nucleic acid editing system to contain an attA attachment site. In some embodiments, the target DNA of interest of the cell was engineered before introduction of the nucleic acid editing system to contain an attD attachment site. In some embodiments, the target DNA of interest of the cell is the genome of the cell.

In certain aspects, described herein is a method of translocating DNA sequences between two linear target DNA molecules of interest, the method comprising introducing into a cell: a nucleic acid editing system of the invention, wherein an attD attachment site of the LSR portion of the fusion polypeptide is present on a first linear target DNA molecule and an attA attachment site of the LSR portion of the fusion polypeptide is present on a second linear target DNA molecule. In some embodiments, the first target DNA molecules of interest of the cell was engineered before introduction of the nucleic acid editing system to contain an attA attachment site. In some embodiments, the second target DNA molecules of interest of the cell was engineered before introduction of the nucleic acid editing system to contain an attD attachment site. In some embodiments, the linear target DNA molecules of interest of the cell are chromosomes of the cell.

Other embodiments of the invention are further described in the following sections of the application, including the Drawings, Detailed Description, Examples, and Claims. Still other objects and advantages of the invention will become apparent by those of skill in the art from the disclosure herein, which are simply illustrative and not restrictive. Thus, other embodiments will be recognized by the ordinarily skilled artisan without departing from the spirit and scope of the invention.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIG. 1A shows a schematic of LSR mediated irreversible, kilobase-scale, and site-specific genomic insertions between two DNA attachment sequences, attP and attB.

FIG. 1B shows that LSRs can mediate integration into pre-installed landing pads or endogenous pseudosites. Pseudosites can be empirically identified by expressing an LSR and delivering a DNA cargo (such as a cargo comprising a reporter gene) carrying an attachment site into a cell. If the DNA cargo integrates into the genome, this genomic locus is determined to contain a pseudosite. The genomic locus can be sequenced according to methods known in the art. For example, sequencing primers can be designed to target the sequence of the integrated DNA cargo such that sequence information of the genomic locus in the vicinity of the cargo can be obtained and analyzed for similarity to the attachment site sequence of the DNA cargo construct that mediated its integration.

FIG. 2 shows an RNA-guided DNA binding domain co-localizes an integrase to a genomic pseudosite (attH), resulting in targeted integration of the donor DNA via integrase-mediated recombination.

FIGS. 3A-B show LSR “Dn29” is a genome targeting LSR with favorable efficiency and specificity. 62% of integrations occur at the top 5 sites. FIG. 3B is taken from Supplementary FIG. 4E of Durrant, M. G., Fanton, A., Tycko, J. et al. Systematic discovery of recombinases for efficient integration of large DNA sequences into the human genome, Nat Biotechnol 41, 488-499 (2023).

FIG. 4 shows LSRs bind attP and attB in a tetrameric complex. Figure taken from Rutherford et al. Curr Opin Struct Biol. 2014.

FIG. 5 shows that LSR N-terminus is critical for tetrameric complex formation and subunit rotation. Figure modified from Rutherford et al. Curr Opin Struct Biol. 2014.

FIG. 6 shows exemplary designs of Dn29-dCas9 fusion constructs (see FIGS. 33-36 for sequences) and pseudosite integration efficiency at attH1 measured with non-targeting guide qPCR. The data shows fusions with Dn29 at the N-terminus and dCas9 at the C-terminus have improved integration efficiencies over wild-type Dn29 and fusions with dCas9 at the N-terminus.

FIG. 7 shows that the construct architecture is generalizable to another LSR “Cp36”. FIG. 7 shows pseudosite integration efficiency at attH1 with a non-target guide, measured with qPCR. The data shows fusions with Cp36 at the N-terminus and dCas9 at the C-terminus have improved integration efficiencies over wild-type Cp36 and fusions with dCas9 at the N-terminus.

FIG. 8 shows a model of a LSR-dCas9 fusion construct in a tetrameric complex, targeting a genomic pseudosite with a single guide RNA. The guide RNA (shown as a line within the four outermost lobes) has complementarity to a genomic region proximal to the integration site, resulting in a single dCas9 monomer being bound to the genomic DNA (bottom left outer lobe showing the gRNA hybridizing to a sequence upstream of the integration site), and the other three monomers being unbound.

FIG. 9 shows Dn29-dCas9 targeting to attH1. Top shows the position of the spacer of the gRNAs and the sequences it targets relative to attH1. Bottom shows pseudosite integration efficiency at attH1 measured by qPCR as a fold change in comparison to two non-targeting guide (NTG) controls.

FIG. 10 shows Dn29-dCas9 mediated cargo integration, targeted to attH1, validated with orthogonal readout methods. Top shows integration at attH1 measured with ddPCR. Bottom shows the total integration efficiency (at any genomic locus) via integration of an mCherry expressing plasmid and flow readout of stable mCherry expression.

FIG. 11 shows Dn29-dCas9 targeting to attH3. Top shows qPCR readout, displayed as fold change compared to two non-targeting guide controls. Bottom shows absolute efficiency measured by ddPCR.

FIG. 12 shows another LSR ortholog (Pf80) can be targeted to pseudosites via dCas9 fusions. Top left shows the relative integration efficiency of Pf80 into its human genomic pseudosites, with the top site (attH1) at locus chr11:64.243,293. Top right shows the integration efficiency at attH1 using Pf80-dCas9 fusion vs Pf80 and various gRNAs proximal to, overlapping with, or within attH1. Bottom shows SEQ ID NO: 534 with the location of the spacer sequences of each gRNA relative to the attH1 pseudosite. Generally, gRNA spacers can be designed to target sequences within 200, 175, 150, 125, 100, 90, 80, 70, 60, 50, 40, 30, 20, 15, 10, or 5 nucleotides from a dinucleotide core sequence of a target attachment site.

FIG. 13 shows another LSR ortholog (Nm60) can be targeted to pseudosites via dCas9 fusions. Top shows the integration efficiency of Nm60-dCas9 into its top pseudosite at chr9:83308042 with various gRNAs. Bottom shows SEQ ID NOs: 535-536 and the location of the spacer sequences of each gRNA relative to the attH1 pseudosite.

FIG. 14 shows dCas9 fusions increase integration efficiency up to 30% at attH1, 8% at attH3 (left). Fold change over a non-targeting guide ranges from 3-11 (right).

FIG. 15 shows a schematic of a non-limiting embodiment of the plasmids that can be used to effectuate DNA insertion (top). The bottom panel shows the percentage integration and different molar ratios of the three plasmids.

FIG. 16 shows a schematic of delivering a mixed population of targeted LSR-dCas9 fusions and unfused LSR monomers, that can assemble into a tetrameric complex.

FIG. 17 shows partial or complete separation of LSR and dCas9 reduces integration efficiency.

FIG. 18 shows integration efficiency as a factor of distance from the core. Distance is measured from the center of the dinucleotide core to the position between the spacer and the PAM (NGG). Data is cumulative over 5 experiments: Dn29-XTEN32-(GGSS)₂-dCas9 to 3 pseudosites, Si74-XTEN32-(GGSS)₂-dCas9 to a landing pad attB at AAVS1, Pf80-XTEN32-(GGSS)₂-dCas9 to attH1. “(GGSS)₂” is disclosed as SEQ ID NO: 585.

FIG. 19 shows a schematic of an embodiment of a design modification to optimize integration efficiency. Shown here is targeting two dCas9s with two guide RNAs, one on either side of the pseudosite, which will facilitate in LSR recruitment and dimer formation on the genomic attachment site.

FIG. 20 shows percentage integration using single and multiplexed guides as indicated for Dn29-dCas9 targeting attH3, measured by ddPCR. The final column in each plot is a hypothetical integration efficiency if combining single guides was additive. SEQ ID NO: 537 and the location of the spacer sequences of each gRNAs relative to the attH3 pseudosite is shown in the schematic.

FIG. 21 shows single and multiplexed guides as indicated for Dn29-dCas9 targeting attH1, measured by qPCR. The location of the binding site for the gRNAs is shown in the schematic.

FIG. 22 shows a schematic of an embodiment of a design modification to optimize integration efficiency. Shown here is guide RNAs targeting the donor plasmid to facilitate recruitment of donor plasmid into the nucleus. In some embodiments, multiple guide RNAs can be used, where the guide RNAs include one or more different gRNAs that target sequences proximal or (proximal and overlapping) to the pseudosite as shown in FIG. 19 and one or more gRNAs that target the donor plasmid.

FIG. 23 shows the integration efficiency when delivering two guide RNAs, one targeting the pseudosite and the second targeting the donor plasmid, shown as fold change compared to a non-targeting guide. The only donor-targeting gRNA with a significant effect is guide 8. SEQ ID NOs: 538-539 and the location of the spacer sequences of each donor targeting gRNAs relative to attD is shown in the schematic.

FIG. 24 shows the specificity of Dn29 vs Dn29-dCas9 fusions. On the left is a plot of all detected integration sites for Dn29, ranked by the number of UMIs sequenced at each locus. The top site, chr10:21, 130, 404, is attH1. When using Dn29-dCas9 and guide 3 targeting attH1, the percent of all integrations that occur at attH1 increases to ˜78% (right).

FIG. 25 shows the specificity of Dn29-(GGGGS)₆-dCas9 and Dn29-XTEN32-(GGSS)₂-dCas9 targeting attH3, given as the percent of unique integrations (UMIs) that occur at that locus. “(GGGGS)₆” and “(GGSS)₂” are disclosed as SEQ ID NOS 598 and 585, respectively.

FIG. 26 shows the correlation between specificity and efficiency, across multiple guides, for Dn29-dCas9 targeting. On the left, 6 guides targeting attH3 are measured for efficiency by ddPCR and specificity by the percent of UMIs that occur at the targeted pseudosite (attH3). Shown are two different fusion construct designs, Dn29-(GGGGS)₆-dCas9 and Dn29-XTEN32-(GGSS)₂-dCas9. “(GGGGS)₆” and “(GGSS)₂” are disclosed as SEQ ID NOS 598 and 585, respectively. On the right, 2 targeting guides for attH1 and a non-targeting guide are measured for efficiency by ddPCR and specificity by the percent of UMIs that occur at attH1.

FIG. 27 shows a schematic of a productive recombination reaction between attP and attB when the dinucleotide cores are matching between the two sequences (top) compared to a non-productive recombination reaction between mis-matched dinucleotide cores (bottom). In a non-productive reaction, ligation between the half sites cannot occur, so the attachment sites will return to a second subunit rotation step and ligate the original attP and attB back together. For the recombination to be directional, the central dinucleotide needs to be non-palindromic.

FIG. 28 shows a schematic of the attachment site orientations resulting in integration, inversion, deletion, chromosomal translocation, and linear donor integration. In some embodiments, LSR fusions, including LSR-dCas9 fusions, can be used to integrate an attachment site near an endogenous attachment site (including pseudosites) to effectuate inversion or excision. For inversion, an attachment site would be integrated in the reverse orientation relative to the attachment site in the target nucleic acid. For excision, an attachment site would be integrated in the same orientation relative to the attachment site in the target nucleic acid. In some embodiments, LSR fusions, including LSR-dCas9 fusions, can be used to integrate an attachment site on a different chromosome to an endogenous attachment site (including pseudosites) to effectuate chromosomal translocation. In other embodiments, an exogenous piece of DNA, either circular or linear, can be delivered with the LSR fusion to effectuate integration or linear donor integration. In the case of linear donor integration, the double stranded break that occurs after recombination with a linear amplicon is repaired by endogenous DNA repair pathways, such as non-homologous end joining.

FIG. 29 shows the integration efficiency at attH1 when fusing a PAM flexible dCas9 variant, dCas9-SpG, to Dn29. Shown are guides targeting various NGG PAMs, which should be targetable by both dCas9 and dCas9-SpG, and NGN PAMs, which should be only targetable by dCas9-SpG. Data shown is qPCR, normalized to dCas9 with a non-targeting guide. For each guide the data is provided for “Dn29-dCas” followed by “Dn29-dCas9-SpG.” The “mismatched LSR control” is the 3^{rd} data point for guide “NTG” and the “Dn29” is the 4^{th} data point for guide “NTG”.

FIG. 30 shows the same dataset as FIG. 29 but with fold change normalized to the Dn29-dCas9 fusion construct with each guide to highlight the SpG-specific effects. For each guide the data is provided for “Dn29-dCas” followed by “Dn29-dCas9-SpG.” The “mismatched LSR control” is the 3^{rd} data point for guide “NTG” and the “Dn29” is the 4^{th} data point for guide “NTG”.

FIG. 31 shows a schematic (top) and results (bottom) of a single guide dual targeting design, where the genomic protospacer (DNA sequence targeted by the gRNA spacer) is included on the donor DNA molecule adjacent to the attD such that a single guide can be used to target both the genome and the donor attachment sites. Data shown is qPCR, normalized to the attD donor without a protospacer.

FIG. 32 shows examples of attachment site sequence logos for Nm60 attB, Fm04 attB, Bt24 attB, and Dn29 attB. These motifs are generated by alignment of the top 100 or 300 genomic integration sites of the cognate attP sequence. The height of the letter at each position indicates the level of enrichment for that nucleotide at that position. Additional attB sequence motifs for LSRs Cp36, Enc9, Pc01, Bt24, Dn29, Pf80, Sp36, and Enc3 are provided and described in Supplemental FIG. 6C of Durrant, M. G., Fanton, A., Tycko, J. et al. Systematic discovery of recombinases for efficient integration of large DNA sequences into the human genome, Nat Biotechnol 41, 488-499 (2023), the content of which is hereby incorporated by reference in its entirety.

FIGS. 33-40 disclose various sequences described herein.

FIG. 41 shows Dn29-dCas9 mediated integration of a plasmid donor at attH1 in H1 human embryonic stem cells.

FIG. 42 shows Dn29-dCas9 mediated integration of a plasmid donor at attH1 in HepG2 hepatocellular carcinoma cell line.

### DETAILED DESCRIPTION

The present invention relates to a fusion of a large serine recombinase (LSR) to a DNA binding domain (DBD). The LSR recognizes two DNA sequences, also known as attachment sites, one of which is the target site and the other is a DNA sequence often found on a separate DNA molecule. The LSR performs site-specific recombination, integrating the DNA found on the separate DNA molecule into the target site. And in cases where the attachment sites are on the same molecule, depending on their relative orientation, LSR can perform excision or inversion recombination reactions. Further, translocation may occur when the attachment sites are on different molecules in a particular relative orientation. The DNA binding domain is targeted, via direct protein-DNA binding or RNA-guided targeting, to a site proximal to, overlapping with, or within the LSR target site, directing the LSR to a single, specific DNA attachment site, such as a pseudosite in a mammalian genome. This design increases on-target integration efficiency up to 30-fold compared to an LSR without the fusion to DNA binding domain, and greatly increases the ratio of on-target to off-target integrations.

The term “cellular DNA” refers to, without limitation, genomic or non-genomic DNA that exists within a cell or the isolated form of such DNA. Genomic or non-genomic DNA includes without limitation, chromosomal or non-chromosomal DNA such as episomal, viral, plasmid, mitochondrial, or chloroplast DNA.

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, guide RNA (gRNA), messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The practice of aspects of the present invention can employ, unless otherwise indicated, conventional techniques of cell biology, cell culture, molecular biology, transgenic biology, microbiology, recombinant DNA, and biochemistry, which are within the skill of the art. Such techniques are explained fully in the literature. See, e.g., Molecular Cloning A Laboratory Manual, 3rd Ed., ed. By Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986) and subsequent versions thereof, the contents of each of which are hereby incorporated by reference in their entireties.

One skilled in the art can obtain a protein in several ways, which include, but are not limited to, isolating the protein via biochemical means or expressing a nucleotide sequence encoding the protein of interest by genetic engineering methods, including, but not limited to, cell-based methods and cell-free methods.

A protein is encoded by a nucleic acid (including, for example, genomic DNA, messenger RNA (mRNA), complementary DNA (cDNA), synthetic DNA, as well as any form of corresponding RNA). Nucleic acids encoding a protein can be produced via recombinant DNA technology and such recombinant nucleic acids can be prepared by conventional techniques, including chemical synthesis, genetic engineering, enzymatic techniques, or a combination thereof.

### LSR-DBD Fusions

The present invention relates to a fusion of a large serine recombinase (LSR) to a DNA binding domain (DBD), which are also referred to herein as “LSR-DBD” fusions. In some embodiments, the LSR portion is fused directly to the DBD portion. In some embodiments, the LSR-DBD fusion comprises a linker between the LSR and DBD portions of the fusion protein. The use of “LSR-DBD” is intended to encompass both embodiments unless specified otherwise (i.e., “-” in “LSR-DBD” indicates both a direct bond or a linker between the LSR and DBD portions of the LSR-DBD fusion protein). Without being bound by theory, the inventive fusions direct an LSR to a specific target site via DNA binding domain fusions to increase efficiency and specificity of the LSR. Without being bound by theory these fusions will increase the local concentration of LSR monomers at target DNA attachment sites, cause longer duration of LSR residence at target DNA attachment sites, provide for improved target DNA scanning efficiency or kinetics and/or provide increased chromatin accessibility by dual protein-mediated binding to two sites.

The LSR, DBD, and where used, linker portions, for use in LSR-DBD fusions are described below.

### Large Serine Recombinases (LSRs)

Recombinases (which may also be referred to as integrases) are a family of enzymes that mediate site-specific recombination between specific DNA sequences recognized by the enzyme. The natural purpose of recombinases is to insert DNA, such as, e.g., viral genomes or non-viral mobile genetic elements, into a host cell to establish the transition between the lytic and lysogenic cycles. Recombinases can be classified into two groups, the tyrosine recombinases and the serine recombinases, based on the active amino acid (tyrosine or serine) involved in the catalytic domain of the enzyme. Serine recombinases create double strand breaks in DNA by forming covalent 5′-phosphoserine bonds with the DNA, followed by strand exchange and ligation. On the other hand, tyrosine recombinases work by cleaving single DNA strands to form covalent 3′-phosphotyrosine bonds with the DNA, followed by a Holliday junction-like intermediate state.

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Examples of serine recombinases include, without limitation, large and small serine recombinases such as, but not limited to Dn29, Pf80, Cp36, Nm60, Si74, Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, Sa11 (SEQ ID NOs: 1-5, 432-443, 445-446, 448-467, 469-476, 478-492, 494-501, 276, 279, 282, 285, 288, 291, respectively), Hin, Gin, Tn3, β-six, CinH, ParA, γδ, φC31, TP901, TG1, φBT1, R4, φRV1, φFC1, MR11, A118, U153, and gp29. Recombinases have numerous applications, including the creation of gene knockouts/knock-ins and gene therapy applications.

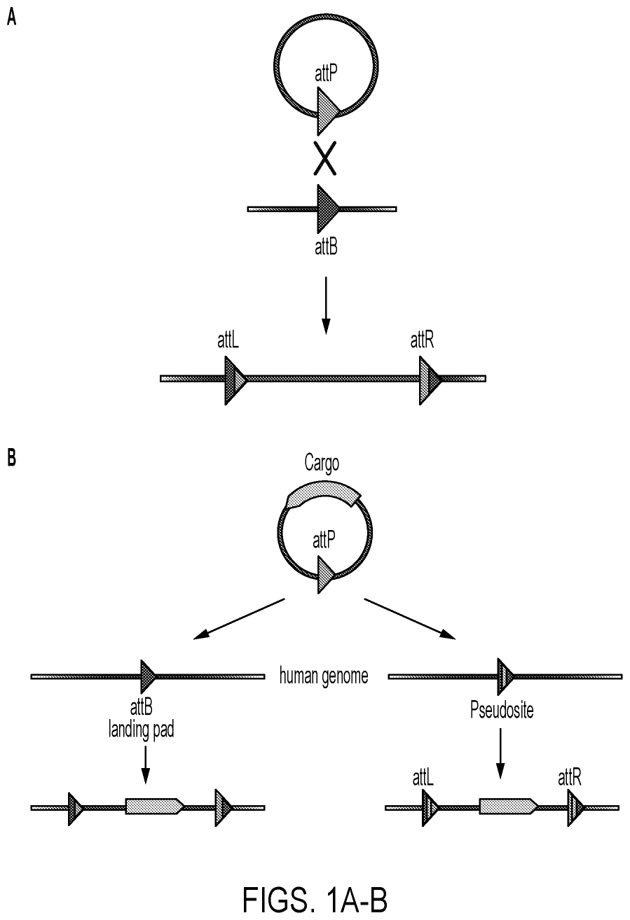

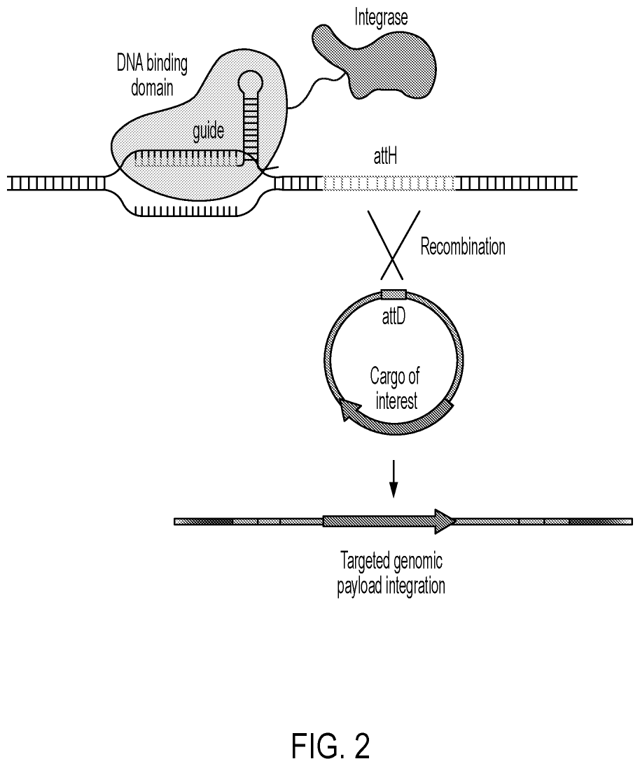

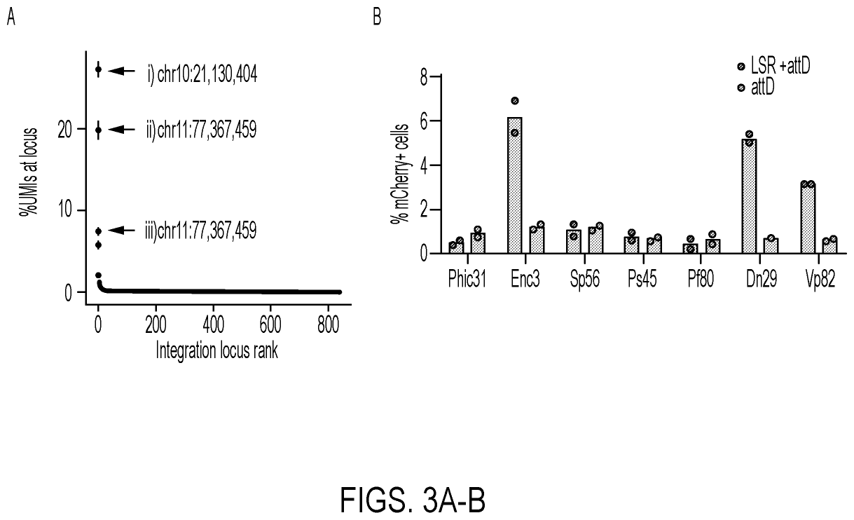

Large serine recombinases are efficient, directional, and specific recombinases for DNA integration in mammalian cells. For example, see FIG. 1A. Examples of large serine recombinases provided herein or useful in the nucleic acids, polypeptides, compositions, systems, and methods disclosed herein include, but are not limited to, KSSJEB, PattyP, Doom, Scowl, Lockley, Switzer, Bob3, Trouble, Abrogate, Anglerfish, Sarfire, SkiPole, ConceptII, Museum, Severus, Rey, Bongo, Airmid, Benedict, Theia, Hinder, Icleared, Sheen, Mundrea, Veracruz, and Rebeuca, from the recently sequenced Mycobacteriophage, and the previously characterized Peaches, PhiC31, BxZ2, as well as Dn29, Pf80, Cp36, Nm60, Si74, Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 1-5, 432-443, 445-446, 448-467, 469-476, 478-492, 494-501, 276, 279, 282, 285, 288, 291, respectively). In particular, LSRs Dn29, Pf80, Cp36, Nm60, or Si74 (SEQ ID NOs: 1-5) are useful in the nucleic acids, polypeptides, compositions, systems, and methods disclosed herein.

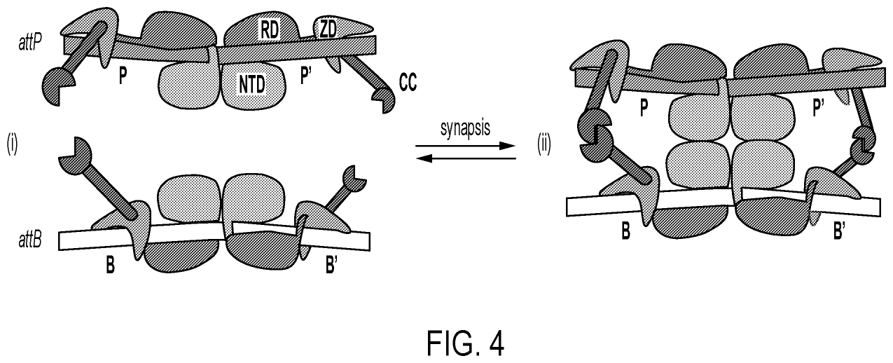

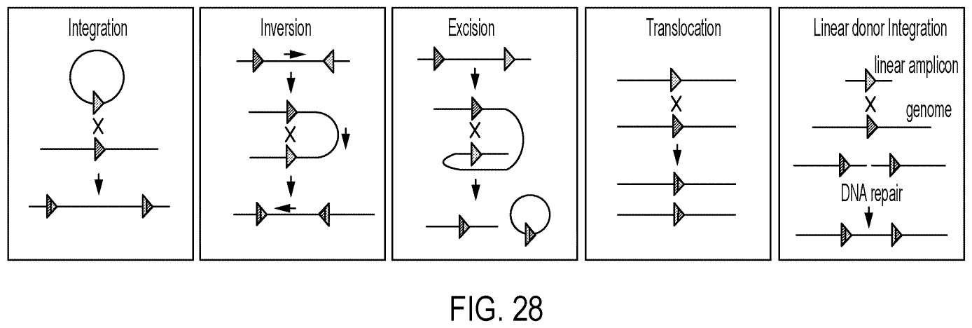

The LSR recognizes two DNA sequences, also known as attachment sites, one of which is the target site and the other is a DNA sequence found on a separate DNA molecule (for integration embodiments). See FIG. 28. LSRs perform a site-specific recombination between the two attachment sites as shown in FIG. 1A. The native attachment sites targeted by LSRs are termed “attP” (phage) and “attB” (bacteria) sites wherein each of the attP and attB sites comprises two half-sites joined at a central sequence. The central sequence consists of a central dinucleotide sequence, described further herein. In general, the recombination reaction is performed by a tetramer of the recombinase, in which each subunit is bound to a half-site of the attP or attB site as shown in FIG. 4. During the recombination reaction, each of the attP and attB sites is cut into two half-sites, in which each half-site has an overhang region comprising the central sequence (e.g. the central dinucleotide). When applied for genomic integration of donor cargos into a genome, the terms attD (donor) and attA (acceptor) may be used to refer to the two attachment sites. Either an attP or an attB can be the attD or attA, depending on which sequence is chosen to be present on the donor molecule (e.g., if attP is attD, then attB is attA; if attB is attD, then attP is attA). In another embodiment, the attD integrates directly into an endogenous pseudosite natively found in the target genome. As described in the Examples and in Durrant et al., NBT 2022, pseudosites can be experimentally determined by analyzing the sequences adjacent to successful integration of a donor molecule with an attD site—where the pseudosites will be adjacent to the attD half-sites. In the case of mammalian genome integration(s), for example, human genome integration endogenous pseudosite(s) is (are) termed attH; and therefore an attH site is a type of attA.

In some aspects, a LSR is used for site-specific recombination, wherein DNA strand exchange takes place between DNA sequences possessing attB and attP sites (or attD and attA sites), and wherein the recombinase rearranges DNA segments by recognizing and binding to the attB and attP sites, at which they cleave the DNA backbone, exchange the two DNA helices involved and rejoin the DNA strands.

LSRs can also site-specifically integrate DNA sequences of interest containing an attD into a DNA target of mammalian cells, both at pre-installed integration sites (e.g., a pre-installed attA) or at endogenous genomic pseudosites (e.g., attH). For example, as shown in FIG. 1B a donor DNA sequence of interest containing a native attP site can be integrated into a DNA target with the corresponding native attB acceptor attachment site (also referred to as a “landing pad”). A donor DNA sequence of interest containing a native attB site can be integrated into a DNA target with the corresponding attP acceptor attachment site (also referred to as a “landing pad”). Mammalian DNA may also contain endogenous genomic pseudosites which have high sequence similarity to an attA site, and can functionally recombine with an attD. If the attA sequence is found in a mammalian genome, for example the human genome, it is termed an attH sequence. For example, as shown in FIG. 1B a donor DNA sequence of interest containing a native attP site can be integrated into a DNA target with an attH pseudosite with high sequence similarity to the corresponding native attB acceptor attachment site. A donor DNA sequence of interest containing a native attB site can be integrated into a DNA target with an attH pseudosite with high sequence similarity to the corresponding native attP acceptor attachment site. Thus, LSRs can be used to integrate a DNA sequence of interest into a target DNA, such as a cellular DNA. Despite their sequence specificity, LSRs may integrate into numerous sites in a mammalian genome, such as the human genome, due to the presence of multiple loci with sufficient “attH” integration site sequences.

The systematic discovery of recombinases for integrating DNA into the human genome is described in WO2023/081762 and Durrant M G, Fanton A, Tycko J, Hinks M, Chandrasekaran S S, Perry N T, Schaepe J, Du P P, Lotfy P, Bassik M C, Bintu L, Bhatt A S, Hsu PD. Systematic discovery of recombinases for efficient integration of large DNA sequences into the human genome. Nat Biotechnol., 41 (4): 488-499, Epub Oct. 10, 2022, the entire contents of both of which are hereby incorporated by reference in their entireties.

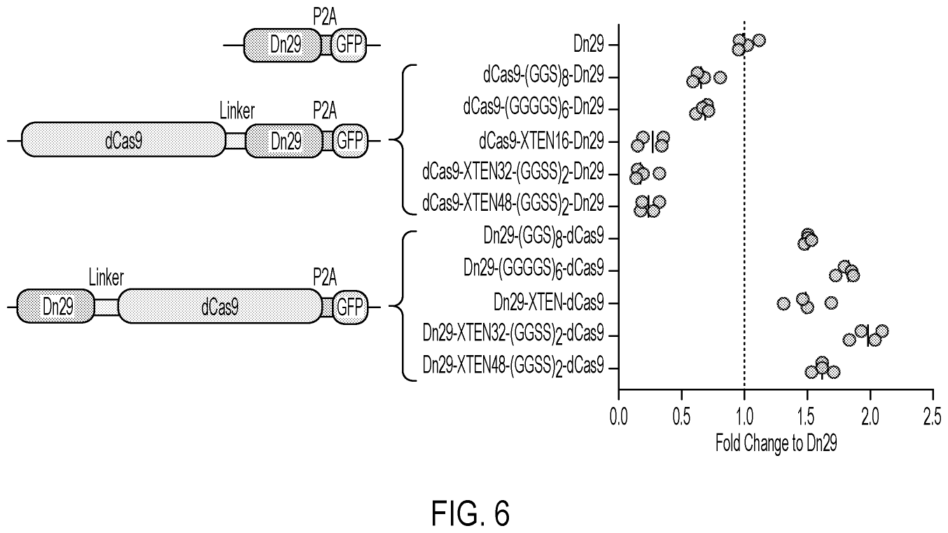

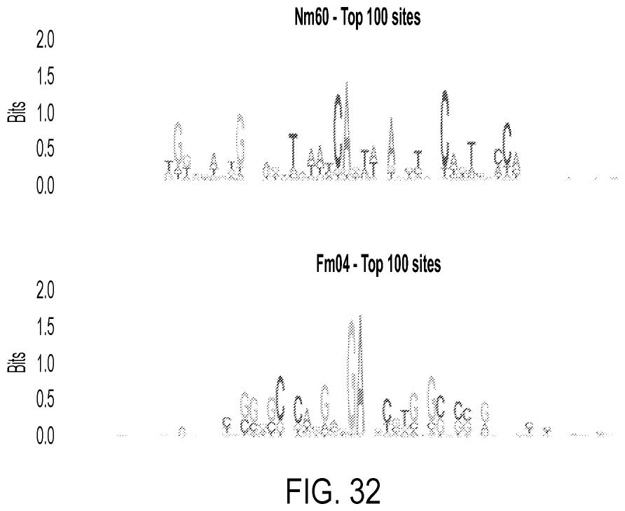

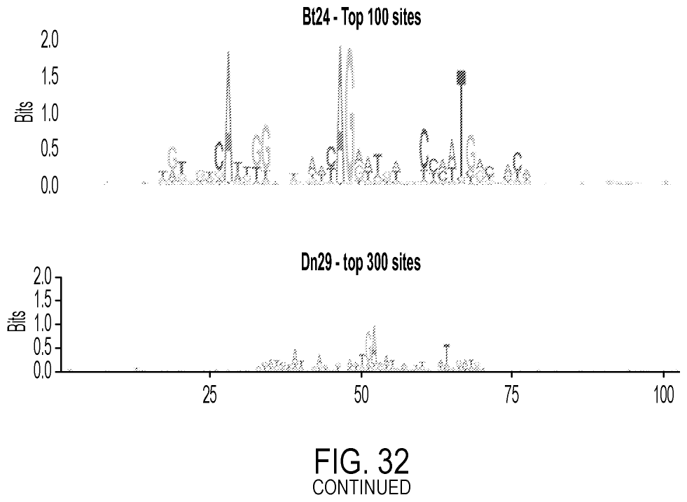

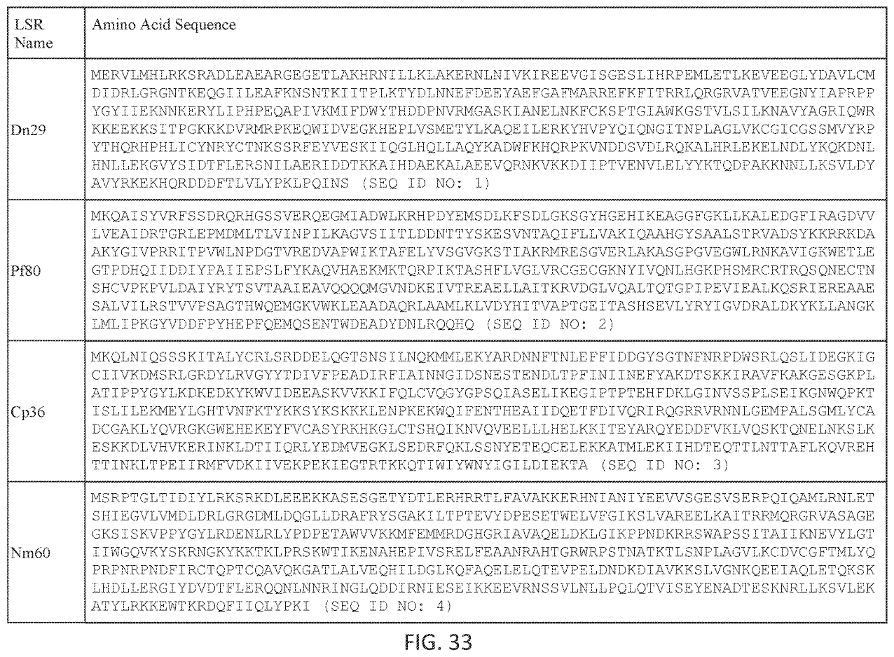

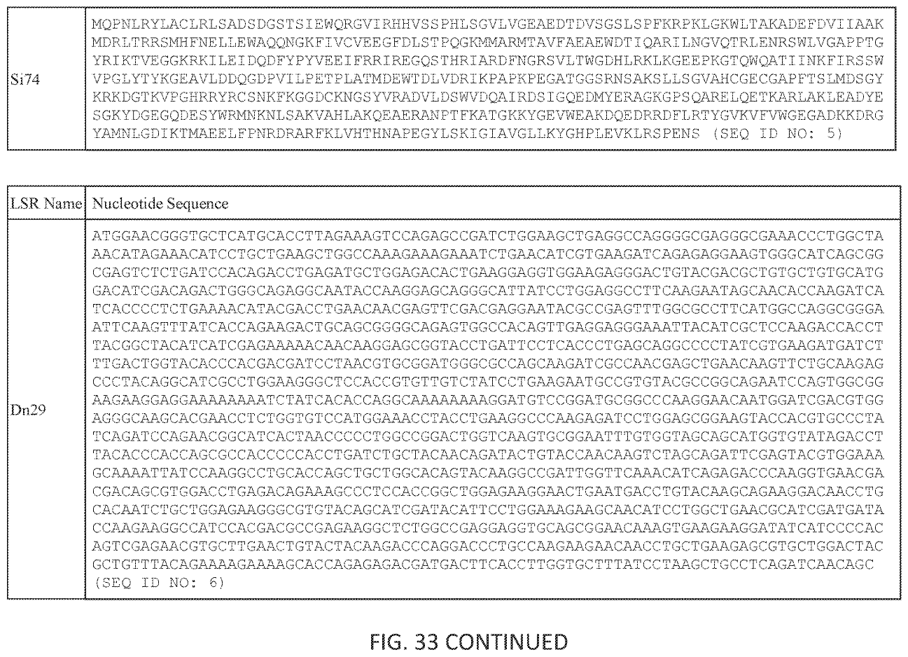

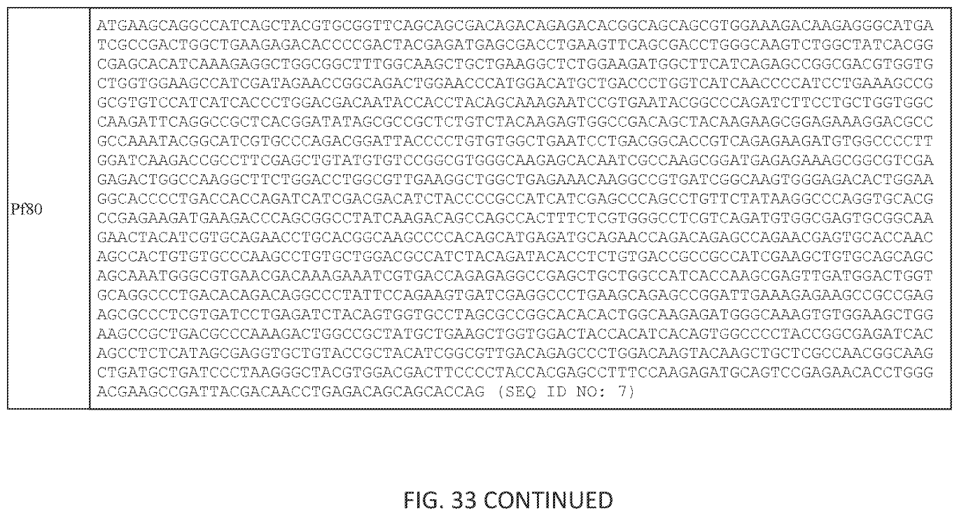

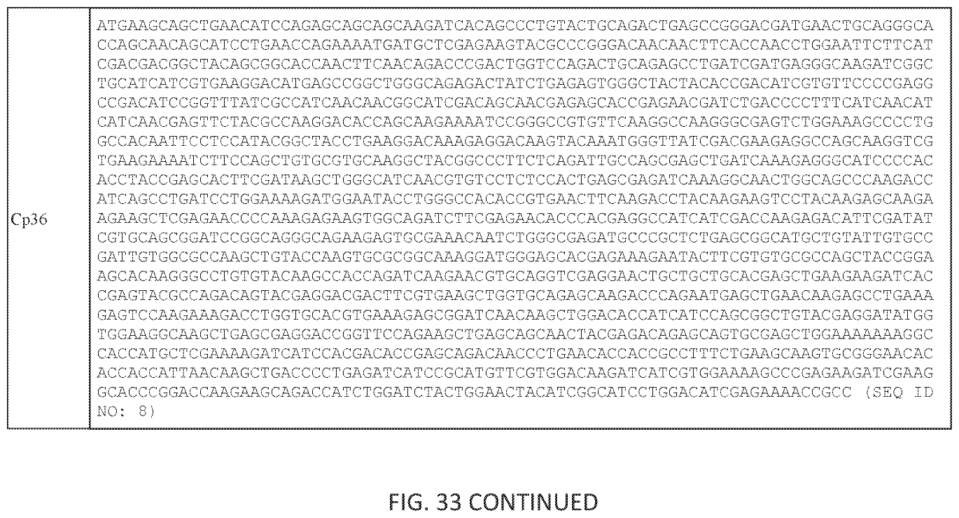

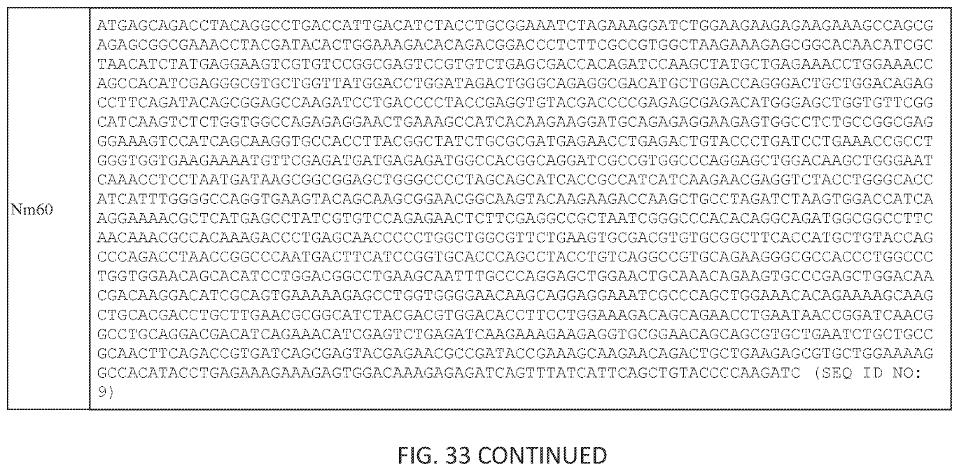

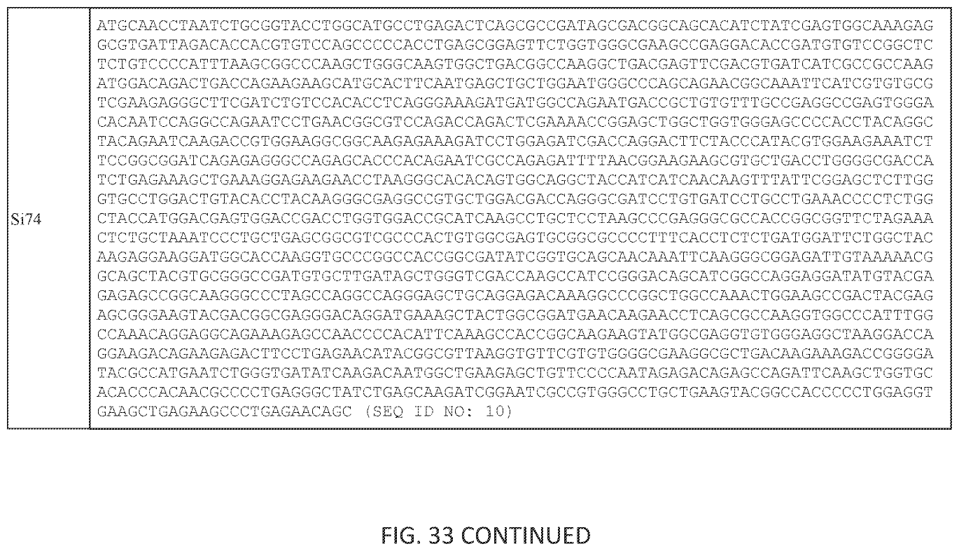

Exemplary LSRs that may be used in the LSR-DBD fusions described herein include, without limitation, the LSRs in FIG. 33 (Dn29, Pf80, Cp36, Nm60, or Si74 (SEQ ID NOs: 1-5, respectively)). The native attP and attB sequences for the LSRs in FIG. 33 are provided as SEQ ID NOs: 304 (attP Cp36), 307 (attP Dn29), 328 (attP Nm60), 337 (attP Pf80), 353 (attP Si74), 374 (attB Cp36), 377 (attB Dn29), 398 (attB Nm60), 407 (attB Pf80), and 423 (attB Si74). In some embodiments, the attachment site for the LSR portion of an LSR-DBD fusion comprises a sequence that follows the consensus sequence logo motifs for the corresponding LSR provided in FIG. 32 or in Supplemental FIG. 6C of Durrant, M. G., Fanton, A., Tycko, J. et al. Systematic discovery of recombinases for efficient integration of large DNA sequences into the human genome, Nat Biotechnol 41, 488-499 (2023), the content of which is hereby incorporated by reference in its entirety.

In certain aspects, described herein is an LSR-DBD fusion comprising the amino acid sequence of Dn29, Pf80, Cp36, Nm60, or Si74 (SEQ ID NOs: 1-5, respectively). In certain aspects, described herein is a nucleic acid encoding an LSR-DBD fusion comprising the amino acid sequence of Dn29, Pf80, Cp36, Nm60, or Si74 (SEQ ID NOs: 1-5, respectively). In some embodiments, the nucleic acid sequence encoding the LSR portion comprises SEQ ID NOs 6-10.

In certain aspects, described herein is an LSR-DBD fusion comprising an amino acid sequence having 70% identity to Dn29, Pf80, Cp36, Nm60, or Si74 (SEQ ID NOs: 1-5, respectively). In some embodiments, the amino acid sequence has 75%, 80%, 85%, 90%, 91%, 92%, 93%, 94%, 95%, 96%, 97%, 98%, or 99% identity to Dn29, Pf80, Cp36, Nm60, or Si74 (SEQ ID NOs: 1-5, respectively). In certain aspects, described herein is a nucleic acid encoding an LSR-DBD fusion comprising an amino acid sequence having 70% identity to Dn29, Pf80, Cp36, Nm60, or Si74 (SEQ ID NOs: 1-5, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Dn29, Pf80, Cp36, Nm60, or Si74 (SEQ ID NOs: 1-5, respectively).

In certain aspects, described herein is an LSR-DBD fusion, wherein the LSR portion consists of the amino acid sequence of Dn29, Pf80, Cp36, Nm60, or Si74, (SEQ ID NOs: 1-5, respectively). In certain aspects, described herein is a nucleic acid encoding an LSR-DBD fusion, wherein the LSR portion consists of the amino acid sequence of Dn29, Pf80, Cp36, Nm60, or Si74, (SEQ ID NOs: 1-5, respectively). In some embodiments, the nucleic acid sequence encoding the LSR portion consists of SEQ ID NOs 6-10.

Additional exemplary LSRs that may be used in the LSR-DBD fusions described herein include, without limitation, the LSRs Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82.

In certain aspects, described herein is an LSR-DBD fusion comprising the amino acid sequence of Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In certain aspects, described herein is a nucleic acid encoding an LSR-DBD fusion comprising the amino acid sequence of Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In some embodiments, the nucleic acid sequence encoding the LSR portion comprises SEQ ID NOs: 6-10, or 515-533.

In certain aspects, described herein is an LSR-DBD fusion comprising an amino acid sequence having 70% identity to Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In some embodiments, the amino acid sequence has 75%, 80%, 85%, 90%, 91%, 92%, 93%, 94%, 95%, 96%, 97%, 98%, or 99% identity to Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In certain aspects, described herein is a nucleic acid encoding an LSR-DBD fusion comprising an amino acid sequence having 70% identity to Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 438, 445, 448, 457, 459, 462, 467, 469, 471, 482, 495, 498, 499, 500, 501, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively).

In certain aspects, described herein is an LSR-DBD fusion, wherein the LSR portion consists of the amino acid sequence of Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In certain aspects, described herein is a nucleic acid encoding an LSR-DBD fusion, wherein the LSR portion consists of the amino acid sequence of Dn29, Pf80, Cp36, Nm60, Si74, Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82 (SEQ ID NOs: 1-5, 433, 435, 437, 438, 445, 448, 457, 459, 462, 467, 469, 471, 479, 482, 495, 498, 499, 500, 501, respectively). In some embodiments, the nucleic acid sequence encoding the LSR portion consists of SEQ ID NOs: 6-10, or 515-533.

Additional exemplary LSRs that may be used in the LSR-DBD fusions described herein include, without limitation, the LSRs Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively).

In certain aspects, described herein is an LSR-DBD fusion comprising the amino acid sequence of Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively). In certain aspects, described herein is a nucleic acid encoding an LSR-DBD fusion comprising the amino acid sequence of Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively).

In certain aspects, described herein is an LSR-DBD fusion comprising an amino acid sequence having 70% identity to Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively). In some embodiments, the amino acid sequence has 75%, 80%, 85%, 90%, 91%, 92%, 93%, 94%, 95%, 96%, 97%, 98%, or 99% identity to Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively). In certain aspects, described herein is a nucleic acid encoding an LSR-DBD fusion comprising an amino acid sequence having 70% identity to Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively).

In certain aspects, described herein is an LSR-DBD fusion, wherein the LSR portion consists of the amino acid sequence of Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively). In certain aspects, described herein is a nucleic acid encoding an LSR-DBD fusion, wherein the LSR portion consists of the amino acid sequence of Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 432-443, 445-446, 448-467, 469-476, 478-492, 494-501, 276, 279, 282, 285, 288, 291, respectively).

In certain aspects, described herein is an LSR-DBD fusion, wherein the LSR portion comprises LSR means for mediating recombination of DNA between recombinase recognition sequences. In certain aspects, described herein is a nucleic acid encoding an LSR-DBD fusion, wherein the LSR portion comprises LSR means for mediating recombination of DNA between recombinase recognition sequences. In some embodiments, the LSR means for mediating recombination of DNA between recombinase recognition sequences is Dn29, Pf80, Cp36, Nm60, Si74, Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 1-5, 432-443, 445-446, 448-467, 469-476, 478-492, 494-501, 276, 279, 282, 285, 288, 291, respectively).

Serine recombinases typically possess a catalytic domain at the N-terminus of about 150 amino acid residues. Several amino acids in the catalytic domain are highly conserved and are known to contribute to the structure of the active site. Serine recombinases further comprise attachments to the catalytic domain at the C-terminal which can vary in sizes. For LSRs the attachment group can be a complex multidomain region with both regulatory and DNA-binding functions.

In some embodiments, the LSR-DBD fusion comprises a catalytic domain of a large serine recombinase. By “catalytic domain of a large serine recombinase,” it is meant that an LSR-DBD fusion protein includes a domain comprising an amino acid sequence of (e.g., derived from) a large serine recombinase, such that the domain is sufficient to induce recombination when contacted with a target nucleic acid (either alone or with additional factors including other large serine recombinase catalytic domains which may or may not form part of the LSR-DBD fusion protein). In some embodiments, a catalytic domain of a large serine recombinase excludes a DNA binding domain of the large serine recombinase. In some embodiments, the catalytic domain of a large serine recombinase includes part or all of a large serine recombinase, e.g., the catalytic domain may include a large serine recombinase domain and a DNA binding domain, or parts thereof, or the catalytic domain may include a large serine recombinase domain and a DNA binding domain that is mutated or truncated to abolish DNA binding activity. Large serine recombinases and catalytic domains of large serine recombinases are known to those of skill in the art, and include, for example, those described herein. In some embodiments, the catalytic domain is derived from any large serine recombinase.

In some embodiments, the LSR used in the LSR-DBD fusions described herein includes, without limitation, a LSR comprising one or more of the following amino acid motifs, written in the common Prosite format, where x is any amino acid and x(n) represents n number of any amino acid (e.g., x(3) is xxx or 3 consecutive amino acids):

Motif 1:
[AEILSTVY]-[ADEGKQRST]-x(3)-[EG]-x-[ACFLMV]-x-[AFILMTV]-x(2)-
[FHILMNV]-[AGSV]-[ADILSTV]-x-[AGS]-x(3)-[KRSV]-[ADEGKNST]-[AEIKMNQST]-
[FILMST]-x-[DELQSV]-[ENQR]-x(4)-[AFHIKLMNQRSV]-x-[AEGHKLMNQRSV]
Motif 2:
[AGI]-[DEGNPSTV]-[DGNQS]-[AHNQRTVY]-x-[ADEHILPQRTY]-
[ADEQR]-[FIKL]-x-[DEFGNQRSTV]-[AILSTV]-[DEIKLNQRSTV]-[ADEKMNRSTV]-
[AGQRST]-x-[ADEKLQRT]-x-[ALMV]
Motif 3:
[ADFILMNSY]-x(2)-[AIKMSV]-x-[AFGILMV]-x(3)-[QRT]-[AGS]-x-
[DEGNQS]-E-S-x-[AHKNRSTV]-K-x(2)-[LMRY]-[AINQSTV]-[AEFIKLNRTV]-x-
[AFHLNQSTY]-[AILMNRSTVY]
Motif 4:
[EKNTGSLDVARP]-[EHITGSLDVAP]-x-[MITSLVARP]-
[EKNITGSDQVARP]-[EGSDARP]-[ILDAR]-[MHKTLVQDAR]-[EKITGSLDQVA]-
[EKHDQVAR]-[MHISLVQAR]-[QEKNMSLDVAR]-[EKHGSLDQAR]-[EYKNIHLVA]-
x-[EKITGSLDQAR]-[EKHTGDQAR]-x-[QEKNTGSDVAR]-[QEKNTGSVDAR]-
[ISWLVFAR]-[QEMTGSLVDA]-[EKNITGSDARP]-[EMILDQA]-[EYILVFAR]-
[EMTGSLDVAR]-[EKNGSLDQAR]-[QEGVDARP]
Motif 5:
[ADEHKNQRS]-[ADEFGHKMNQRSWY]-[EFY]-[FHLWY]-x-
[ADEFIKLMNQRSTY]-[FIQSTV]-[AGKLNRSTV]-[ADEHKNQRTY]-[INQR]-
[FILMQS]-x(2)-[AGKNS]-[KMQRSTV]-x(2)-[AEGKMNSTY]
Motif 6:
W-[AEHNRSTV]-x-[AGNST]-[FGLMNQSTV]-[ILPV]-x(2)-[ILTV]-x(4)-
[ACGMQRST]-x-[ILVY]-G-[DEHNQS]-x-[EHILMQRT]-[AEFHLNPY]-
[CFHKMNQRTY]-[DEFIKLNQRSTV]
Motif 7:
[AGINSTV]-x-[AIS]-x-[FILMY]-E-[IR]-x(2)-[DILT]-x-[AEIKMQS]-R-[ITV]-x-
[ADGRST]-x-[FKLMY]-[AEHIKLMNQRVWY]-x-[AIKLMR]
Motif 8:
[FY]-[DEKQS]-[EKLMQ]-[KLR]-[KLV]-x-[GN]-[DEHKLMR]-[ST]-x-
[FHIQSTVW]
Motif 9:
[ILV]-x(2)-[ADFHILMNQSVY]-x(3)-[AGS]-x-[DEIKNQRS]-[EQ]-S-x(2)-
[AK]-[AQRS]-x-[LMR]-[ILQRSV]-x-[ADEGHIQRS]-[AKNQSTV]-[AHKRWY]-x-
[AGHIKQRST]-x-[CHIKLRV]
Motif 10:
R-[LMQR]-[ANS]-[NPST]-W
Motif 11:
[ILV]-[AV]-x-[AFHILQWY]-[IMV]-x-[ELQT]-[AIV]-F
Motif 12:
R-[DKNRSV]-[ADEFGKPQS]-[AEIKLSTV]-x-[FGILNV]-[AFILQRVY]-
[DEILMNQSTV]-[DEFILMQTVY]-[IKLRV]-[DEKNQR]-[DEFKLNQWY]-[FL]
Motif 13:
[AEFILMNQSTVY]-[AFGILMRSTV]-x(3)-[ADEFGHLMNST]-x(2)-[DMNS]-
[DEQ]-x-[CFHLTVY]-x-[AEKLRY]-x(2)-[ALS]-x-[DEKNQRS]-[GIMQRTV]-
[DHKNQR]-x-[AGILNSTV]-[FHIKLMNQVWY]

Thus, in certain aspects, described herein is an LSR-DBD fusion, wherein the LSR portion comprises the amino acid sequence of one or more motifs selected from Motif 1-Motif 13. In certain aspects, described here is a nucleic acid encoding an LSR-DBD fusion wherein the LSR portion comprises the amino acid sequence of one or more motifs selected from Motif 1-Motif 13.

In certain aspects, described herein is an LSR-DBD fusion, wherein the LSR portion comprises the amino acid sequence of Motif 2 and comprises an amino acid sequence having 70% identity to Si74 (SEQ ID NO: 5). In some embodiments, the amino acid sequence has 75%, 80%, 85%, 90%, 91%, 92%, 93%, 94%, 95%, 96%, 97%, 98%, or 99% identity to Si74 (SEQ ID NO: 5). In certain aspects, described herein is a nucleic acid encoding an LSR-DBD fusion, wherein the LSR portion comprises the amino acid sequence of Motif 2 and comprises an amino acid sequence having 70% identity to Si74 (SEQ ID NO: 5). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Si74 (SEQ ID NO: 5).

In certain aspects, described herein is an LSR-DBD fusion, wherein the LSR portion comprises the amino acid sequence of Motif 3 and comprises an amino acid sequence having 70% identity to Bm99, Cs56, or Vp82 (SEQ ID NOs: 433, 445, 501, respectively). In some embodiments, the amino acid sequence has 75%, 80%, 85%, 90%, 91%, 92%, 93%, 94%, 95%, 96%, 97%, 98%, or 99% identity to Bm99, Cs56, or Vp82 (SEQ ID NOs: 433, 445, 501, respectively). In certain aspects, described herein is a nucleic acid encoding an LSR-DBD fusion, wherein the LSR portion comprises the amino acid sequence of Motif 3 and comprises an amino acid sequence having 70% identity to Bm99, Cs56, or Vp82 (SEQ ID NOs: 433, 445, 501, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Bm99, Cs56, or Vp82 (SEQ ID NOs: 433, 445, 501, respectively).

In certain aspects, described herein is an LSR-DBD fusion, wherein the LSR portion comprises the amino acid sequence of Motif 4 and comprises an amino acid sequence having 70% identity to Me99 (SEQ ID NOs: 467). In some embodiments, the amino acid sequence has 75%, 80%, 85%, 90%, 91%, 92%, 93%, 94%, 95%, 96%, 97%, 98%, or 99% identity to Me99 (SEQ ID NOs: 467). In certain aspects, described herein is a nucleic acid encoding an LSR-DBD fusion, wherein the LSR portion comprises the amino acid sequence of Motif 4 and comprises an amino acid sequence having 70% identity to Me99 (SEQ ID NOs: 467). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Me99 (SEQ ID NOs: 467).

In certain aspects, described herein is an LSR-DBD fusion, wherein the LSR portion comprises the amino acid sequence of Motif 5 and comprises an amino acid sequence having 70% identity to Dn29, Nm60, or Bt24 (SEQ ID NOs: 1, 4, 435, respectively). In some embodiments, the amino acid sequence has 75%, 80%, 85%, 90%, 91%, 92%, 93%, 94%, 95%, 96%, 97%, 98%, or 99% identity to Dn29, Nm60, or Bt24 (SEQ ID NOs: 1, 4, 435, respectively). In certain aspects, described herein is a nucleic acid encoding an LSR-DBD fusion, wherein the LSR portion comprises the amino acid sequence of Motif 5 and comprises an amino acid sequence having 70% identity to Dn29, Nm60, or Bt24 (SEQ ID NOs: 1, 4, 435, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Dn29, Nm60, or Bt24 (SEQ ID NOs: 1, 4, 435, respectively).

In certain aspects, described herein is an LSR-DBD fusion, wherein the LSR portion comprises the amino acid sequence of Motif 6 and comprises an amino acid sequence having 70% identity to Vh19 or Vh73 (SEQ ID NOs: 499, 500 respectively). In some embodiments, the amino acid sequence has 75%, 80%, 85%, 90%, 91%, 92%, 93%, 94%, 95%, 96%, 97%, 98%, or 99% identity to Vh19 or Vh73 (SEQ ID NOs: 499, 500 respectively). In certain aspects, described herein is a nucleic acid encoding an LSR-DBD fusion, wherein the LSR portion comprises the amino acid sequence of Motif 6 and comprises an amino acid sequence having 70% identity to Vh19 or Vh73 (SEQ ID NOs: 499, 500 respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Vh19 or Vh73 (SEQ ID NOs: 499, 500 respectively).

In certain aspects, described herein is an LSR-DBD fusion, wherein the LSR portion comprises the amino acid sequence of Motif 7 and comprises an amino acid sequence having 70% identity to Fm04, uCb4, or Cb16 (SEQ ID NOs: 459, 498, 438, respectively). In some embodiments, the amino acid sequence has 75%, 80%, 85%, 90%, 91%, 92%, 93%, 94%, 95%, 96%, 97%, 98%, or 99% identity to Fm04, uCb4, or Cb16 (SEQ ID NOs: 459, 498, 438, respectively). In certain aspects, described herein is a nucleic acid encoding an LSR-DBD fusion, wherein the LSR portion comprises the amino acid sequence of Motif 7 and comprises an amino acid sequence having 70% identity to Fm04, uCb4, or Cb16 (SEQ ID NOs: 459, 498, 438, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Fm04, uCb4, or Cb16 (SEQ ID NOs: 459, 498, 438, respectively).

In certain aspects, described herein is an LSR-DBD fusion, wherein the LSR portion comprises the amino acid sequence of Motif 8 and comprises an amino acid sequence having 70% identity to Ec03, or Kp03 (SEQ ID NOs: 448, 462, respectively). In some embodiments, the amino acid sequence has 75%, 80%, 85%, 90%, 91%, 92%, 93%, 94%, 95%, 96%, 97%, 98%, or 99% identity to Ec03, or Kp03 (SEQ ID NOs: 448, 462, respectively). In certain aspects, described herein is a nucleic acid encoding an LSR-DBD fusion, wherein the LSR portion comprises the amino acid sequence of Motif 8 and comprises an amino acid sequence having 70% identity to Ec03, or Kp03 (SEQ ID NOs: 448, 462, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Ec03, or Kp03 (SEQ ID NOs: 448, 462, respectively).

In certain aspects, described herein is an LSR-DBD fusion, wherein the LSR portion comprises the amino acid sequence of Motif 9 and comprises an amino acid sequence having 70% identity to Pa03 (SEQ ID NO: 471). In some embodiments, the amino acid sequence has 75%, 80%, 85%, 90%, 91%, 92%, 93%, 94%, 95%, 96%, 97%, 98%, or 99% identity to Pa03 (SEQ ID NO: 471). In certain aspects, described herein is a nucleic acid encoding an LSR-DBD fusion, wherein the LSR portion comprises the amino acid sequence of Motif 9 and comprises an amino acid sequence having 70% identity to Pa03 (SEQ ID NO: 471). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Pa03 (SEQ ID NO: 471).

In certain aspects, described herein is an LSR-DBD fusion, wherein the LSR portion comprises the amino acid sequence of Motif 11 and comprises an amino acid sequence having 70% identity to Pf80, or Ps45 (SEQ ID NOs: 2, 482, respectively). In some embodiments, the amino acid sequence has 75%, 80%, 85%, 90%, 91%, 92%, 93%, 94%, 95%, 96%, 97%, 98%, or 99% identity to Pf80, or Ps45 (SEQ ID NOs: 2, 482, respectively). In certain aspects, described herein is a nucleic acid encoding an LSR-DBD fusion, wherein the LSR portion comprises the amino acid sequence of Motif 11 and comprises an amino acid sequence having 70% identity to Pf80, or Ps45 (SEQ ID NOs: 2, 482, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Pf80, or Ps45 (SEQ ID NOs: 2, 482, respectively).

In certain aspects, described herein is an LSR-DBD fusion, wherein the LSR portion comprises the amino acid sequence of Motif 13 and comprises an amino acid sequence having 70% identity to Cp36 (SEQ ID NO: 3). In some embodiments, the amino acid sequence has 75%, 80%, 85%, 90%, 91%, 92%, 93%, 94%, 95%, 96%, 97%, 98%, or 99% identity to Cp36 (SEQ ID NO: 3). In certain aspects, described herein is a nucleic acid encoding an LSR-DBD fusion, wherein the LSR portion comprises the amino acid sequence of Motif 13 and comprises an amino acid sequence having 70% identity to Cp36 (SEQ ID NO: 3). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to Cp36 (SEQ ID NO: 3).

### DNA Binding Domains (DBDs)

As an RNA-guided nuclease, Cas proteins have been adapted for targeted gene editing and selection in a variety of organisms. Nuclease-null Cas variants that have no substantial nuclease activity are useful to localize proteins and RNA to nearly any set of dsDNA sequences.

In some embodiments, the DNA binding domain of the LSR-DBD fusion described herein comprises a modified form of a Cas protein, for example, without limitation, Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5, which forms a complex with a guide RNA. When the DBD is a Cas protein in complex with a guide RNA, the Cas protein can bind a target DNA via the guide RNA spacer sequence, which base pairs with a complementary target DNA sequence proximal to, overlapping with, or within the recombinase target site. In some instances, the modified form of the Cas protein comprises an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas protein. In preferred embodiments, the modified form of the Cas protein has no substantial nuclease activity. When the DBD of the LSR-DBD fusion is a modified form of a Cas protein that has no substantial nuclease activity, it can be referred to as a “dead Cas” or “dCas”. In some embodiments, a Cas protein may have nickase activity. In some embodiments, the modified form of the Cas protein has no substantial nickase activity. In some embodiments, the modified form of the Cas protein has no substantial nickase activity and no substantial nuclease activity

A person of skill in the art recognizes that Cas proteins can be isolated from different bacterial species. In some embodiments, the DNA binding domain of the LSR-DBD fusion described herein comprises a Cas protein from Streptococcus pyogenes, Staphylococcus aureus, Neisseria meningitidis, Campylobacter jejuni, Streptococcus thermophilus, Lachnospiraceae bacterium, Acidaminococcus sp., Alicyclobacillus acidiphilus, or Bacillus hisashii. In some embodiments, the DNA binding domain of the LSR-DBD fusion described herein comprises Cas9 from Streptococcus pyogenes or a dCas9 form thereof. In some embodiments, the DNA binding domain of the LSR-DBD fusion described herein comprises Cas9 from Staphylococcus aureus or a dCas9 form thereof.

In certain aspects, described herein is an LSR-DBD fusion comprising the amino acid sequence of dCas9, Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5. In certain aspects, described herein is a nucleic acid encoding an LSR-DBD fusion comprising the amino acid sequence of dCas9, Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5. In some embodiments, the DNA binding domain of the LSR-DBD fusion described herein comprises Streptococcus pyogenes dCas9. In some embodiments, the DNA binding domain of the LSR-DBD fusion described herein comprises Staphylococcus aureus dCas9.

In certain aspects, described herein is an LSR-DBD fusion comprising the amino acid sequence of dCas9, dCas9-HF1, dCas9-SpG, dCas9-SpG-HF1 (SEQ ID NOs: 29-32, respectively). In certain aspects, described herein is a nucleic acid encoding an LSR-DBD fusion comprising the amino acid sequence of dCas9, dCas9-HF1, dCas9-SpG, dCas9-SpG-HF1 (SEQ ID NOs: 29-32, respectively). In some embodiments, the nucleic acid sequence encoding the DBD portion comprises SEQ ID NOs: 33-36.

In certain aspects, described herein is an LSR-DBD fusion comprising an amino acid sequence having 70% identity to dCas9, dCas9-HF1, dCas9-SpG, dCas9-SpG-HF1 (SEQ ID NOs: 29-32, respectively). In some embodiments, the amino acid sequence has 75%, 80%, 85%, 90%, 91%, 92%, 93%, 94%, 95%, 96%, 97%, 98%, or 99% identity to dCas9, dCas9-HF1, dCas9-SpG, dCas9-SpG-HF1 (SEQ ID NOs: 29-32, respectively). In certain aspects, described herein is a nucleic acid encoding an LSR-DBD fusion comprising an amino acid sequence having 70% identity to dCas9, dCas9-HF1, dCas9-SpG, dCas9-SpG-HF1 (SEQ ID NOs: 29-32, respectively). In some embodiments, the nucleic acid encoding an LSR-DBD fusion comprises an amino acid sequence having 75%, 80%, 85%, 90%, 91%, 92%, 93%, 94%, 95%, 96%, 97%, 98%, or 99% identity to dCas9, dCas9-HF1, dCas9-SpG, dCas9-SpG-HF1 (SEQ ID NOs: 29-32, respectively).

In certain aspects, described herein is an LSR-DBD fusion, wherein the DBD portion consists of the amino acid sequence of dCas9, dCas9-HF1, dCas9-SpG, dCas9-SpG-HF1 (SEQ ID NOs: 29-32, respectively). In certain aspects, described herein is a nucleic acid encoding an LSR-DBD fusion, wherein the DBD portion consists of the amino acid sequence of dCas9, dCas9-HF1, dCas9-SpG, dCas9-SpG-HF1. In some embodiments, the nucleic acid sequence encoding the DBD portion consists of SEQ ID NOs: 33-36.

In certain aspects, described herein is an LSR-DBD fusion, wherein the DBD portion comprises DBD means for binding a target DNA sequence proximal to, overlapping with, or within the recombinase target site. In certain aspects, described herein is a nucleic acid encoding an LSR-DBD fusion, wherein the DBD portion comprises DBD means for binding a target DNA sequence proximal to, overlapping with, or within the recombinase target site. In some embodiments, the DBD means for binding a target DNA sequence proximal to, overlapping with, or within the recombinase target site is dCas9, dCas9-HF1, dCas9-SpG, dCas9-SpG-HF1 (SEQ ID NOs: 29-32, respectively), Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5.

In other embodiments, other DNA binding domains may be used (e.g., ZFPs or TALEs) that bind to a DNA target site proximal to, overlapping with, or within the recombinase target site. In some embodiments, the DNA binding domain binds to a DNA target nucleic acid sequence within 200 nucleotides upstream or downstream of a dinucleotide core of an attachment site of the LSR portion of the fusion polypeptide on a DNA of interest. In some embodiments, the DNA binding domain binds to a DNA target nucleic acid sequence within 100 nucleotides upstream or downstream of a dinucleotide core of an attachment site of the LSR portion of the fusion polypeptide on a DNA of interest. In some embodiments, the DNA binding domain binds to a DNA target nucleic acid sequence within 80 nucleotides upstream or downstream of a dinucleotide core of an attachment site of the LSR portion of the fusion polypeptide on a DNA of interest. In some embodiments, the DNA binding domain binds to a DNA target nucleic acid sequence within 50 nucleotides upstream or downstream of a dinucleotide core of an attachment site of the LSR portion of the fusion polypeptide on a DNA of interest. In some embodiments, one of the two or more domains is a zinc finger (ZF) or TALE DNA binding domain. A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. Zinc finger and TALE binding domains can be “engineered” to bind to a predetermined nucleotide sequence, for example via engineering (altering one or more amino acids) of the recognition helix region of a naturally occurring zinc finger or TALE protein. Therefore, engineered DNA binding proteins (zinc fingers or TALEs) are proteins that are non-naturally occurring.

### Linkers

In some embodiments, the fusion between the LSR and DBD protein may include a linker. The term “linker,” as used herein, refers to a chemical group or a molecule linking two molecules or moieties, e.g., LSR and Cas protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker may comprise a peptide or a non-peptide moiety. In some embodiments, the linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

Exemplary linkers include, for example, flexible, glycine-serine (GlySer or GS) linkers for use in the LSR-DBD fusions described herein. In some embodiments a “GGS” linker is used, which can be used in various repeats, for example in repeats of 1 (GGS), 2 ((GGS)₂) (SEQ ID NO: 562), 3 ((GGS)₃) (SEQ ID NO: 563), 4 ((GGS)₄) (SEQ ID NO: 564), 5 ((GGS)₅) (SEQ ID NO: 565), 6 ((GGS)₆) (SEQ ID NO: 566), 7 ((GGS)₇) (SEQ ID NO: 567), 8 ((GGS)₈) (SEQ ID NO: 11), 9 ((GGS)₉) (SEQ ID NO: 568), 10 ((GGS)₁₀) (SEQ ID NO: 569), 11 ((GGS)₁₁) (SEQ ID NO: 570), 12 ((GGS)₁₂) (SEQ ID NO: 571), or more, to provide suitable lengths, as required. In some embodiments a “GGGS” linker (SEQ ID NO: 572) is used, which can be used in various repeats, for example in repeats of 1 (GGGS) (SEQ ID NO: 572), 2 ((GGGS)₂) (SEQ ID NO: 573), 3 ((GGGS)₃) (SEQ ID NO: 574), 4 ((GGGS)₄) (SEQ ID NO: 575), 5 ((GGGS)₅) (SEQ ID NO: 576), 6 ((GGGS)₆) (SEQ ID NO: 577), 7 ((GGGS)₇) (SEQ ID NO: 578), 8 ((GGGS)₈) (SEQ ID NO: 579), 9 ((GGGS)₉) (SEQ ID NO: 580), 10 ((GGGS)₁₀) (SEQ ID NO: 581), 11 ((GGGS)₁₁) (SEQ ID NO: 582), 12 ((GGGS)₁₂) (SEQ ID NO: 583), or more, to provide suitable lengths, as required. In some embodiments a “GGSS” linker (SEQ ID NO: 584) is used, which can be used in various repeats, for example in repeats of 1 (GGSS) (SEQ ID NO: 584), 2 ((GGSS)₂) (SEQ ID NO: 585), 3 ((GGSS)₃) (SEQ ID NO: 586), 4 ((GGSS)₄) (SEQ ID NO: 587), 5 ((GGSS)₅) (SEQ ID NO: 588), 6 ((GGSS)₆) (SEQ ID NO: 589), 7 ((GGSS)₇) (SEQ ID NO: 590), 8 ((GGSS)₈) (SEQ ID NO: 591), 9 ((GGSS)₉) (SEQ ID NO: 592), 10 ((GGSS)₁₀) (SEQ ID NO: 593), 11 ((GGSS)₁₁) (SEQ ID NO: 594), 12 ((GGSS)₁₂) (SEQ ID NO: 595), or more, to provide suitable lengths, as required. In some embodiments a “GGGGS” linker (SEQ ID NO: 596) is used, which can be used in various repeats, for example, they can be used in repeats of 3 ((GGGGS)₃) (SEQ ID NO: 597), or 6 ((GGGGS)₆) (SEQ ID NO: 598), 9 ((GGGGS)₉) (SEQ ID NO: 599) or 12 ((GGGGS)₁₂) (SEQ ID NO: 600) or more, to provide suitable lengths, as required. Other alternatives are (GGGGS)₁ (SEQ ID NO: 596), (GGGGS)₂ (SEQ ID NO: 601), (GGGGS)₄, (SEQ ID NO: 602) (GGGGS)₅ (SEQ ID NO: 603), (GGGGS)₇ (SEQ ID NO: 604), (GGGGS)₈ (SEQ ID NO: 605), (GGGGS)₁₀ (SEQ ID NO: 606), or (GGGGS)₁₁ (SEQ ID NO: 607). Additional glycine and/or serine residues can be included at the ends of the linker or between the various repeats, for example, S(GGGGS)₆S (SEQ ID NO: 12).

In some embodiments, XTEN linkers are used in the LSR-DBD fusions described herein. For example, in some embodiments, XTEN16 (SGSETPGTSESATPESS (SEQ ID NO: 13)) is used. In some embodiments, XTEN32, or XTEN48, which have two and three repeats of XTEN16, respectively are used. In some embodiments, additional XTEN16 repeats can be used to provide suitable lengths, as required.

In some embodiments, an alpha-helical linker such as (Ala(GluAlaAlaAlaLys)Ala) (SEQ ID NO: 608) is also contemplated for use in the LSR-DBD fusions described herein.

In some embodiments, rigid linkers are contemplated, such as (EAAAK)₃ (SEQ ID NO: 609), (EAAAK)ₙ(n=1-3) (SEQ ID NO: 610), A(EAAK)₄(ALEA(EAAAK)₄A (SEQ ID NO: 611), PAPAP (SEQ ID NO: 612), AEAAAKEAAAKA (SEQ ID NO: 613), (Ala-Pro)ₙ (n=10-34) (SEQ ID NO: 614) for use in the LSR-DBD fusions described herein. In some embodiments, cleavable linkers are contemplated, such as, disulfide bonds, VSQTSKLTR|AETVFPDV (SEQ ID NO: 615), PLG|LWA (SEQ ID NO: 616), RVL|AEA (SEQ ID NO: 631), EDVVCC|SMSY (SEQ ID NO: 617), GGIER|GS (SEQ ID NO: 618), TRHRQPR|GWE (SEQ ID NO: 619), AGNRVRR|SVG (SEQ ID NO: 620), RRRRRRR|R|R (SEQ ID NO: 621) for use in the LSR-DBD fusions described herein.

In some embodiments, 2A self-cleaving peptides are used in the LSR-DBD fusions described herein. These peptides share a core sequence motif of DXEXNPGP (SEQ ID NO: 622). In some embodiments, T2A linker (GSG)EGRGSLLTCGDVEENPGP(S) (SEQ ID NO: 623) is used. In some embodiments, P2A linker (GSG)ATNFSLLKQAGDVEENPGP(S) (SEQ ID NO: 624) is used. In some embodiments, E2A linker (GSG)QCTNYALLKLAGDVESNPGP(S) (SEQ ID NO: 625) is used. In some embodiments, F2A linker (GSG)VKQTLNFDLLKLAGDVESNPGP(S) (SEQ ID NO: 626) is used. The linkers can comprise optional “GSG” residues at the N-terminus and optional “S” residue at the C-terminus as indicated in parentheses.

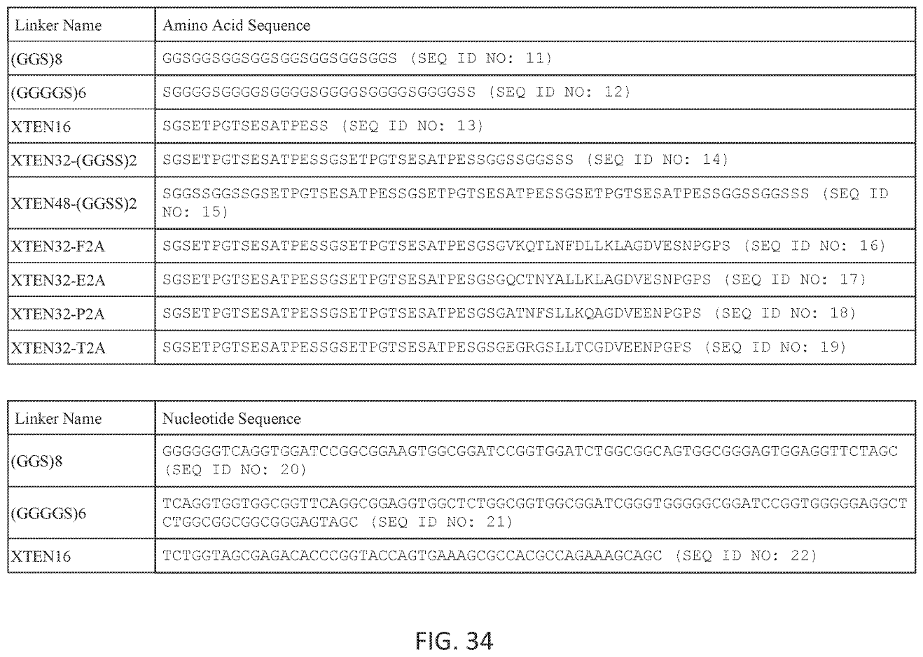

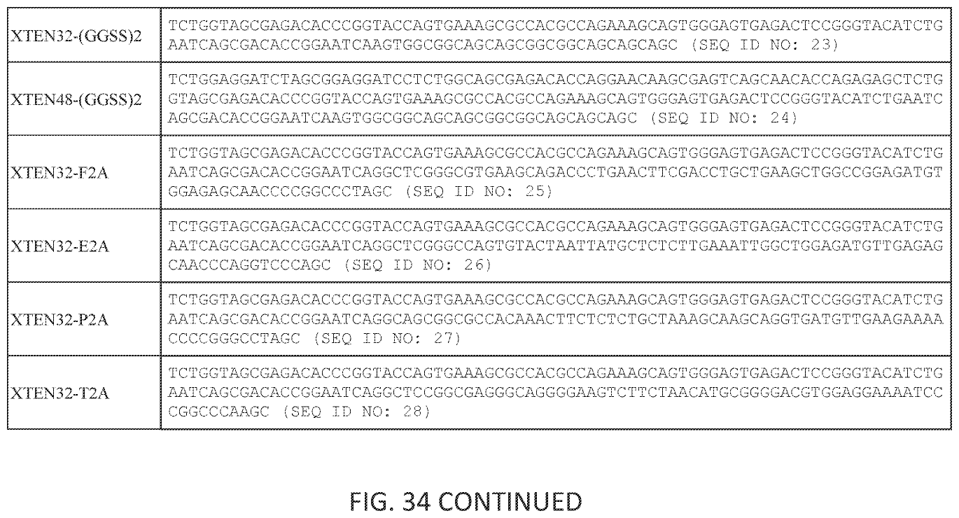

In some embodiments, a linker for use in the LSR-DBD fusions described herein can comprise a combination of one or more of a GlySer linker, an XTEN linker, and/or a 2A self-cleaving peptides described above. Exemplary, non-limiting linkers for use in the LSR-DBD fusions described herein are provided in FIG. 34.

In other embodiments, the linker is at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 30 amino acids, at least 40 amino acids, at least 50 amino acids, at least 60 amino acids, at least 70 amino acids, at least 80 amino acids, at least 90 amino acids, at least 100 amino acids, at least 200 amino acids, at least 300 amino acids, at least 400 amino acids or at least 500 amino acids in length.

In some embodiments, the LSR is fused directly to a DBD by a covalent bond. In certain embodiments, the covalent bond is a carbon-carbon bond, disulfide bond, carbon-heteroatom bond, a carbon-nitrogen bond of an amide linkage, etc. In certain embodiments, the LSR is fused to a DBD by a linker that is a peptide or based on amino acids. In other embodiments, the linker is not peptide-like. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates. In other embodiments, the linker comprises amino acids. In certain embodiments, the linker comprises a peptide.

In certain aspects, described herein is an LSR-DBD fusion wherein the LSR is fused directly to the DBD. In certain aspects, described herein is a nucleic acid encoding an LSR-DBD fusion wherein the LSR is fused directly to the DBD. In certain aspects, described herein is an LSR-DBD fusion comprising an LSR portion, DBD portion, fused together via a peptide linker. In certain aspects, described herein is a nucleic acid encoding an LSR-DBD fusion comprising an LSR portion, DBD portion, fused together via a peptide linker. In some embodiments, the peptide linker is 2 to 100 amino acids long. In some embodiments, the peptide linker is 2 to 50 amino acids long. In some embodiments, the peptide linker is 2 to 30 amino acids long. In some embodiments, the peptide linker comprises glycine and serine residues. In some embodiments, the peptide linker comprises only glycine and serine residues. In some embodiments, the peptide linker is 2 to 30 amino acids long and comprises only glycine and serine residues. In some embodiments, the peptide linker is 24 amino acids long and comprises only glycine and serine residues. In some embodiments, the peptide linker is 30 amino acids long and comprises only glycine and serine residues. In some embodiments, the peptide linker comprises GGS repeats. In some embodiments, the peptide linker comprises 2-12 GGS repeats (SEQ ID NO: 627). In some embodiments, the peptide linker consists of 2-12 GGS repeats (SEQ ID NO: 627). In some embodiments, the peptide linker comprises 8 GGS repeats (SEQ ID NO: 11). In some embodiments, the peptide linker consists of 8 GGS repeats (SEQ ID NO: 11). In some embodiments, the peptide linker comprises GGSS repeats (SEQ ID NO: 584). In some embodiments, the peptide linker comprises 2-12 GGSS repeats (SEQ ID NO: 629). In some embodiments, the peptide linker consists of 2-12 GGSS repeats (SEQ ID NO: 629). In some embodiments, the peptide linker comprises 2 GGSS repeats (SEQ ID NO: 585). In some embodiments, the peptide linker comprises GGGGS repeats (SEQ ID NO: 596). In some embodiments, the peptide linker comprises 2-12 GGGGS repeats (SEQ ID NO: 630). In some embodiments, the peptide linker consists of 2-12 GGGGS repeats (SEQ ID NO: 630). In some embodiments, the peptide linker comprises 6 GGGGS repeats (SEQ ID NO: 598). In some embodiments, the peptide linker consists of 6 GGGGS repeats (SEQ ID NO: 598). In some embodiments, the peptide linker comprises an XTEN16 sequence. In some embodiments, the peptide linker consists of an XTEN16 sequence. In some embodiments, the peptide linker comprises an XTEN32 sequence. In some embodiments, the peptide linker consists of an XTEN32 sequence. In some embodiments, the peptide linker comprises an XTEN48 sequence. In some embodiments, the peptide linker consists of an XTEN48 sequence. In some embodiments, the peptide linker comprises an F2A, E2A, P2A or T2A sequence. In some embodiments, the peptide linker consists of an F2A, E2A, P2A or T2A sequence. In some embodiments, the peptide linker comprises an XTEN16 sequence and one or more glycine or serine residues at the N- or C-terminus of the XTEN16 sequence. In some embodiments, the peptide linker comprises an XTEN32 sequence and one or more glycine or serine residues at the N- or C-terminus of the XTEN32 sequence. In some embodiments, the peptide linker comprises an XTEN48 sequence and one or more glycine or serine residues at the N- or C-terminus of the XTEN48 sequence. In some embodiments, the peptide linker comprises one or more XTEN16 sequences (e.g., XTEN16, XTEN32, XTEN48) and one or more GGSS (SEQ ID NO: 584), GGS, or GGGGS (SEQ ID NO: 596) repeats. In some embodiments, the peptide linker comprises one or more XTEN16 sequences (e.g., XTEN16, XTEN32, XTEN48) and one or more F2A, E2A, P2A or T2A sequence. In some embodiments, the peptide linker comprises one or more GGSS (SEQ ID NO: 584), GGS, or GGGGS (SEQ ID NO: 596) repeats and one or more F2A, E2A, P2A or T2A sequence. In some embodiments, the peptide linker comprises the amino acid sequence of SEQ ID NOs: 11-19. In some embodiments, the nucleic acid sequence encoding the peptide linker portion comprises SEQ ID NOs: 20-28.

In certain aspects, described herein is an LSR-DBD fusion comprising a peptide linker means for fusing together the LSR portion and DBD portion. In certain aspects, described herein is a nucleic acid encoding an LSR-DBD fusion comprising a peptide linker means for fusing together the LSR portion and DBD portion.

In some embodiments, the fusion protein further comprises or consists essentially of or consists of a localization (nuclear import or export) signal as, or as part of, the linker between the DBD (e.g., Cas enzyme) portion and the LSR portion. HA or Flag tags are also within the gambit of the invention as linkers. The linkers allow the user to engineer appropriate amounts of “mechanical flexibility”.

Contemplated herein are fusions oriented in either orientation. Thus, in some embodiments, the LSR is fused to the C-terminus of a DBD. Alternatively, the LSR is fused to the N-terminus of a DBD. In another instance, the LSR is fused to a position other than the C-terminus or the N-terminus of a DBD, e.g., an internal residue of a DBD. Fusions oriented with the LSR at the N-terminus, e.g., LSR-dCas9 or LSR-linker-dCas9, are preferable to fusions oriented with the LSR at the C-terminus, e.g., dCas9-LSR or dCas9-linker-LSR. Thus, in certain aspects, described herein is an LSR-DBD fusion wherein the LSR portion is N-terminal to the DBD portion. In certain aspects, described herein is a nucleic acid encoding an LSR-DBD fusion wherein the LSR portion is N-terminal to the DBD portion.

Longer linkers are preferable as well, for example Dn29-XTEN32-(GGSS)₂-XTEN-dCas9 is preferable to Dn29-XTEN16-dCas9. Dn29-(GGGGS)₆-dCas9 is preferable to Dn29-(GGS)₈-dCas9. “(GGSS)₂”, “(GGGGS)₆” and “(GGS)₈” are disclosed as SEQ ID NOS 585, 598 and 11, respectively. Linker flexibility is also a factor, as more flexible linkers (GGS and GGGGS (SEQ ID NO: 596)) are preferable than more rigid linkers (XTEN16) in the dCas9-linker-Dn29 fusions.

In certain aspects, described herein is an LSR-DBD fusion comprising any of the LSR and DBD portions described herein. In certain aspects, described herein is a nucleic acid encoding an LSR-DBD fusion comprising any of the LSR and DBD portions described herein. In some embodiments, the LSR portion comprises: (a) the amino acid sequence of Dn29, Pf80, Cp36, Nm60, Si74, Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 1-5, 432-443, 445-446, 448-467, 469-476, 478-492, 494-501, 276, 279, 282, 285, 288, 291, respectively), (b) an amino acid sequence having 70%, 75%, 80%, 85%, 90%, 91%, 92%, 93%, 94%, 95%, 96%, 97%, 98%, or 99% identity to the amino acid sequence of (a), (c) an amino acid sequence of Motif 1-Motif 13, (d) an amino acid sequence of Motif 1-Motif 13 and an amino acid sequence having 70%, 75%, 80%, 85%, 90%, 91%, 92%, 93%, 94%, 95%, 96%, 97%, 98%, or 99% identity to the amino acid sequence of (a), or (e) LSR means for mediating recombination of DNA between recombinase recognition sequences; and the DBD portion comprises: (f) an amino acid sequence of Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5, (g) the amino acid sequence of dCas9, dCas9-HF1, dCas9-SpG, dCas9-SpG-HF1 (SEQ ID NOs: 29-32, respectively), (h) an amino acid sequence having 70%, 75%, 80%, 85%, 90%, 91%, 92%, 93%, 94%, 95%, 96%, 97%, 98%, or 99% identity to the amino acid sequence of (f), or (i) DBD means for binding a target DNA sequence proximal to, overlapping with, or within the recombinase target site.

In certain aspects, described herein is an LSR-DBD fusion comprising an LSR portion comprising the amino acid sequence of Dn29, Pf80, Cp36, Nm60, or Si74 (SEQ ID NOs: 1-5, respectively) and a DBD portion comprising the amino acid sequence of dCas9, dCas9-HF1, dCas9-SpG, or dCas9-SpG-HF1 (SEQ ID NOs: 29-32, respectively). In certain aspects, described herein is a nucleic acid encoding an LSR-DBD fusion comprising an LSR portion comprising the amino acid sequence of Dn29, Pf80, Cp36, Nm60, or Si74 (SEQ ID NOs: 1-5, respectively) and a DBD portion comprising the amino acid sequence of dCas9, dCas9-HF1, dCas9-SpG, or dCas9-SpG-HF1 (SEQ ID NOs: 29-32, respectively). In some embodiments, the LSR portion comprises Dn29 (SEQ ID NO: 1) and the DBD portion comprises dCas9 (SEQ ID NO: 29). In some embodiments, the LSR portion comprises Pf80 (SEQ ID NO: 2) and the DBD portion comprises dCas9 (SEQ ID NO: 29). In some embodiments, the LSR portion comprises Cp36 (SEQ ID NO: 3) and the DBD portion comprises dCas9 (SEQ ID NO: 29). In some embodiments, the LSR portion comprises Nm60 (SEQ ID NO: 4) and the DBD portion comprises dCas9 (SEQ ID NO: 29). In some embodiments, the LSR portion comprises Si74 (SEQ ID NO: 5) and the DBD portion comprises dCas9 (SEQ ID NO: 29). In some embodiments, the amino acid sequence of the LSR portion has 70%, 75%, 80%, 85%, 90%, 91%, 92%, 93%, 94%, 95%, 96%, 97%, 98%, or 99% identity to the amino acid sequence of Dn29, Pf80, Cp36, Nm60, or Si74 (SEQ ID NOs: 1-5, respectively). In some embodiments, the amino acid sequence of the DBD portion has 70%, 75%, 80%, 85%, 90%, 91%, 92%, 93%, 94%, 95%, 96%, 97%, 98%, or 99% identity to the amino acid sequence of dCas9, dCas9-HF1, dCas9-SpG, or dCas9-SpG-HF1 (SEQ ID NOs: 29-32, respectively).

In certain aspects, described herein is an LSR-DBD fusion comprising an LSR portion comprising LSR means for mediating recombination of DNA between recombinase recognition sequences and a DBD portion comprising DBD means for binding a target DNA sequence proximal to, overlapping with, or within the recombinase target site. In certain aspects, described herein is a nucleic acid encoding an LSR-DBD fusion comprising an LSR portion comprising LSR means for mediating recombination of DNA between recombinase recognition sequences and a DBD portion comprising DBD means for binding a target DNA sequence proximal to, overlapping with, or within the recombinase target site.

In certain aspects, described herein is an LSR-DBD fusion comprising any of the LSR, DBD, and linker portions described herein. In certain aspects, described herein is a nucleic acid encoding an LSR-DBD fusion comprising any of the LSR, DBD, and linker portions described herein. In some embodiments, the LSR portion comprises a) the amino acid sequence of Dn29, Pf80, Cp36, Nm60, Si74, Bc30, Bm99, Bs46, Bt24, Bu30, Bxb1, Cb16, Cc91, Cd04, Cd15, Cd16, Cd31, Cs56, Ct03, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, No67, Pa01, Pa03, Pc01, Pc64, Pf13, Pf15, Pf48, Ph43, PhiC31, Pp20, Ps40, Ps45, Rb27, Rh64, R109, Sa01, Sa02, Sa10, Sa34, Sa51, Se37, Sh25, Sm18, Sp56, Td01, Td08, uCb4, Vh19, Vh73, Vp82, Cd08, CMp1, E101, Pa19, Pg17, or Sa11 (SEQ ID NOs: 1-5, 432-443, 445-446, 448-467, 469-476, 478-492, 494-501, 276, 279, 282, 285, 288, 291, respectively), b) an amino acid sequence having 70%, 75%, 80%, 85%, 90%, 91%, 92%, 93%, 94%, 95%, 96%, 97%, 98%, or 99% identity to the amino acid sequence of a), c) an amino acid sequence of Motif 1-Motif 13, d) an amino acid sequence of Motif 1-Motif 13 and an amino acid sequence having 70%, 75%, 80%, 85%, 90%, 91%, 92%, 93%, 94%, 95%, 96%, 97%, 98%, or 99% identity to the amino acid sequence of a), or e) LSR means for mediating recombination of DNA between recombinase recognition sequences; the DBD portion comprises f) an amino acid sequence of Cas9, Cpf1, Cas12b, Cas12c, Cas12d, Cas12e, Cas12f, Cas12g, Cas12h, Cas12i, Cas3, Cas8a-c, Cas10, Cse1, Csy1, Csn1, Csn2, Cas4, Csm2, Cm5, Cas1, Cas2, Cas7, C2c3, C2c2, C2c1, or Cas5, g) the amino acid sequence of dCas9, dCas9-HF1, dCas9-SpG, or dCas9-SpG-HF1 (SEQ ID NOs: 29-32, respectively), h) an amino acid sequence having 70%, 75%, 80%, 85%, 90%, 91%, 92%, 93%, 94%, 95%, 96%, 97%, 98%, or 99% identity to the amino acid sequence of f), or i) DBD means for binding a target DNA sequence proximal to, overlapping with, or within the recombinase target site; and the linker portion comprising j) a peptide linker, k) a peptide linker comprising one or more glycine-serine repeats, l) a peptide linker comprising one or more XTEN linkers, m) a peptide linker comprising one or more glycine-serine repeats and one or more XTEN linkers, n) an amino acid comprising SEQ ID NOs: 11-19, o) an amino acid sequence having 70%, 75%, 80%, 85%, 90%, 91%, 92%, 93%, 94%, 95%, 96%, 97%, 98%, or 99% identity to the amino acid sequence of k), or p) peptide linker means for fusing together the LSR portion and DBD portion.

In certain aspects, described herein is an LSR-DBD fusion comprising an LSR portion comprising the amino acid sequence of Dn29, Pf80, Cp36, Nm60, or Si74 (SEQ ID NOs: 1-5, respectively), a DBD portion comprising the amino acid sequence of dCas9, dCas9-HF1, dCas9-SpG, or dCas9-SpG-HF1 (SEQ ID NOs: 29-32, respectively), and a linker portion comprising the amino acid sequence of SEQ ID NOs: 11-19. In certain aspects, described herein is a nucleic acid encoding an LSR-DBD fusion comprising an LSR portion comprising the amino acid sequence of Dn29, Pf80, Cp36, Nm60, or Si74 (SEQ ID NOs: 1-5, respectively) and a DBD portion comprising the amino acid sequence of dCas9, dCas9-HF1, dCas9-SpG, or dCas9-SpG-HF1 (SEQ ID NOs: 29-32, respectively), and a linker portion comprising the amino acid sequence of SEQ ID NOs: 11-19. In some embodiments, the LSR portion comprises Dn29 (SEQ ID NO: 1), the DBD portion comprises dCas9 (SEQ ID NO: 29), and the linker portion comprises the amino acid sequence of SEQ ID NOs: 11-19. In some embodiments, the LSR portion comprises Pf80 (SEQ ID NO: 2), the DBD portion comprises dCas9 (SEQ ID NO: 29), and the linker portion comprises the amino acid sequence of SEQ ID NOs: 11-19. In some embodiments, the LSR portion comprises Cp36 (SEQ ID NO: 3), the DBD portion comprises dCas9 (SEQ ID NO: 29), and the linker portion comprises the amino acid sequence of SEQ ID NOs: 11-19. In some embodiments, the LSR portion comprises Nm60 (SEQ ID NO: 4), the DBD portion comprises dCas9 (SEQ ID NO: 29), and the linker portion comprises the amino acid sequence of SEQ ID NOs: 11-19. In some embodiments, the LSR portion comprises Si74 (SEQ ID NO: 5), the DBD portion comprises dCas9 (SEQ ID NO: 29), and the linker portion comprises the amino acid sequence of SEQ ID NOs: 11-19. In some embodiments, the amino acid sequence of the LSR portion has 70%, 75%, 80%, 85%, 90%, 91%, 92%, 93%, 94%, 95%, 96%, 97%, 98%, or 99% identity to the amino acid sequence of Dn29, Pf80, Cp36, Nm60, or Si74 (SEQ ID NOs: 1-5, respectively). In some embodiments, the amino acid sequence of the DBD portion has 70%, 75%, 80%, 85%, 90%, 91%, 92%, 93%, 94%, 95%, 96%, 97%, 98%, or 99% identity to the amino acid sequence of dCas9, dCas9-HF1, dCas9-SpG, or dCas9-SpG-HF1 (SEQ ID NOs: 29-32, respectively). In some embodiments, the amino acid sequence of the linker portion has 70%, 75%, 80%, 85%, 90%, 91%, 92%, 93%, 94%, 95%, 96%, 97%, 98%, or 99% identity to the amino acid sequence of SEQ ID NOs: 11-19.

In certain aspects, described herein is an LSR-DBD fusion comprising an LSR portion comprising LSR means for mediating recombination of DNA between recombinase recognition sequences, a DBD portion comprising DBD means for binding a target DNA sequence proximal to, overlapping with, or within the recombinase target site, and peptide linker means for fusing together the LSR portion and DBD portion. In certain aspects, described herein is a nucleic acid encoding an LSR-DBD fusion comprising an LSR portion comprising LSR means for mediating recombination of DNA between recombinase recognition sequences, a DBD portion comprising DBD means for binding a target DNA sequence proximal to, overlapping with, or within the recombinase target site, and peptide linker means for fusing together the LSR portion and DBD portion.

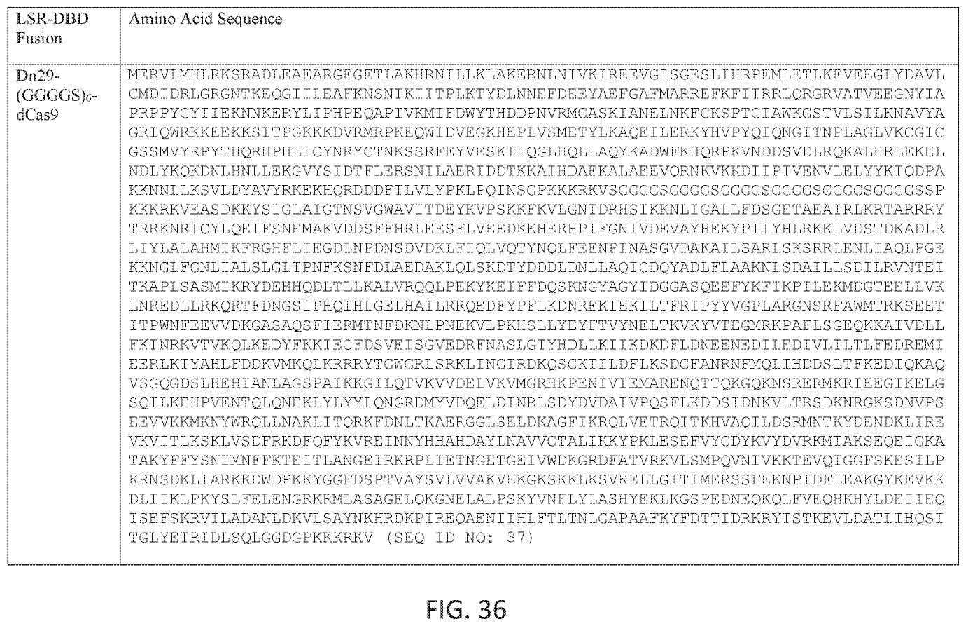

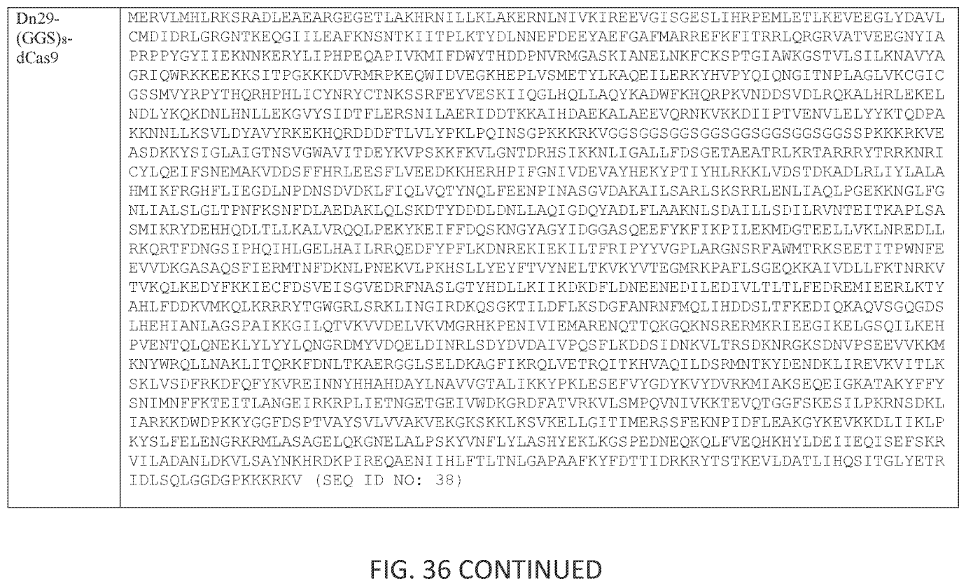

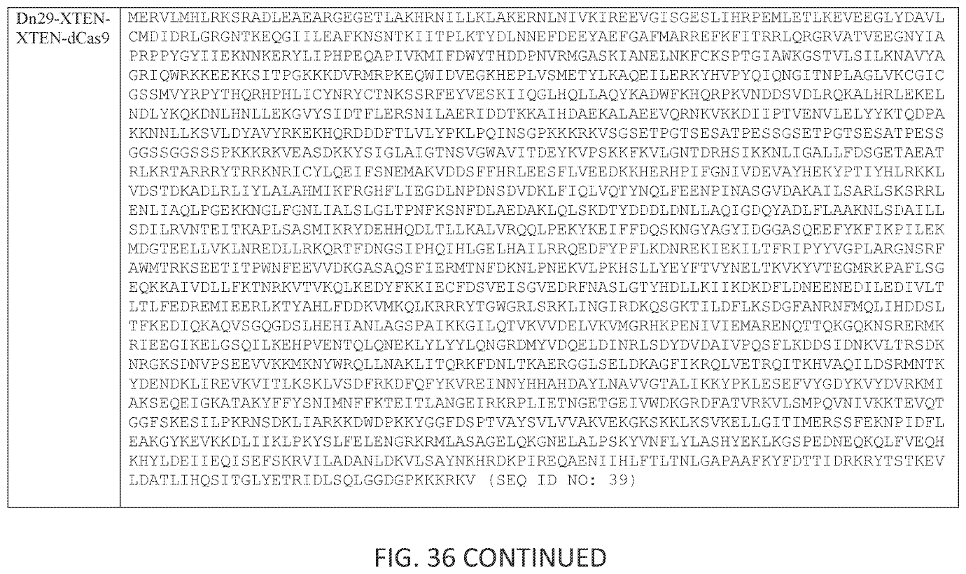

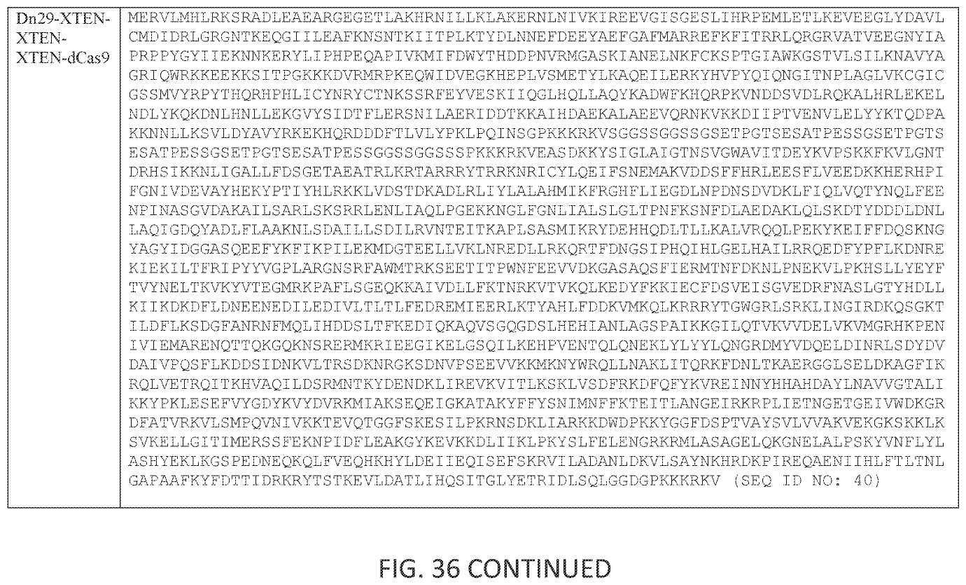

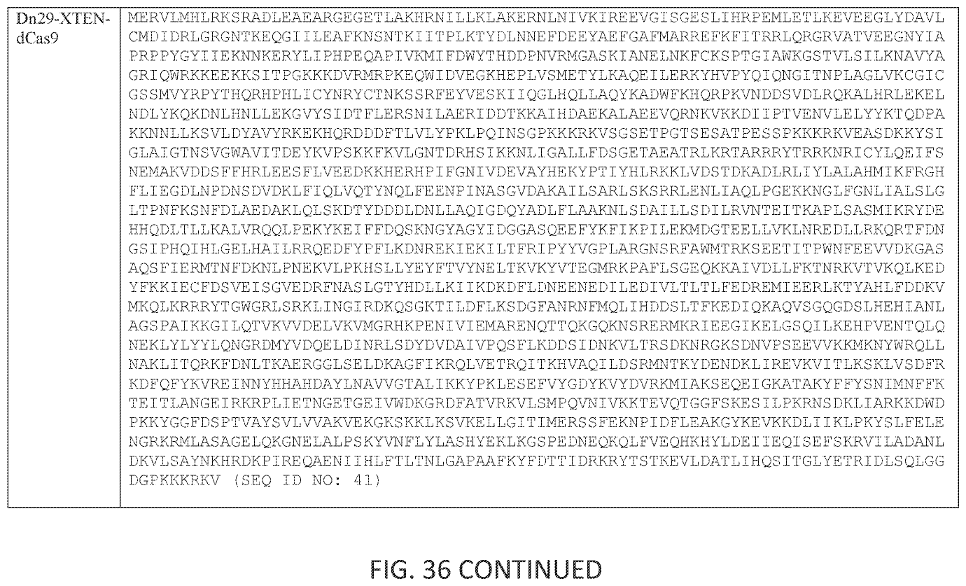

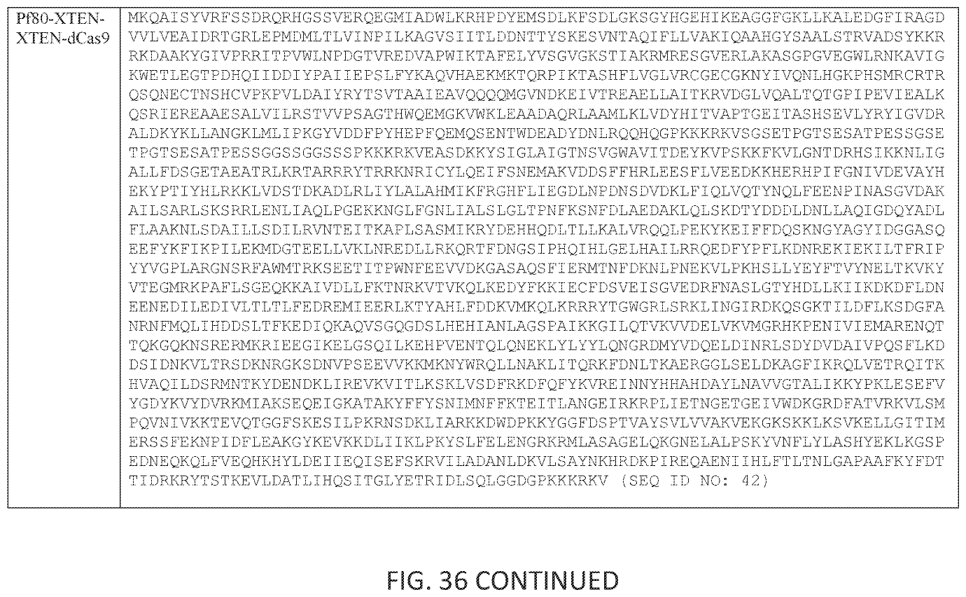

In certain aspects, described herein is an LSR-DBD fusion comprising the amino acid sequences provided in FIG. 36 (SEQ ID NOs: 37-42). In certain aspects, described herein is a nucleic acid encoding an LSR-DBD fusion comprising the amino acid sequences provided in FIG. 36 (SEQ ID NOs: 37-42). In some embodiments, the amino acid sequence has 70%, 75%, 80%, 85%, 90%, 91%, 92%, 93%, 94%, 95%, 96%, 97%, 98%, or 99% identity to the amino acid sequence of SEQ ID NOs: 37-42. In certain aspects, described herein is an LSR-DBD fusion consists of the amino acid sequences provided in FIG. 36 (SEQ ID NOs: 37-42). In certain aspects, described herein is a nucleic acid encoding an LSR-DBD fusion consisting of the amino acid sequences provided in FIG. 36 (SEQ ID NOs: 37-42).

In any of the embodiments described herein, a nucleotide sequence encoding the LSR-DBD fusion polypeptide or the LSR, DBD, and/or linker portions thereof can be codon-optimized. This type of optimization is known in the art and entails the mutation of foreign-derived DNA to mimic the codon preferences of the intended host organism or cell while encoding the same protein. Thus, the codons are changed, but the encoded protein remains unchanged. For example, if the intended target cell was a human cell, a human codon-optimized Cas protein (or variant, e.g., dCas) would be a suitable DBD. Any suitable DBD can be codon optimized. As another non-limiting example, if the intended host cell were a mouse cell, then a mouse codon-optimized Cas protein (or variant, e.g., dCas) would be a suitable DBD. While codon optimization is not required, it is acceptable and may be preferable in certain cases.

Protein-mediated recruitment refers to the fusion of the DBD and LSR to two interacting protein domains that can allow trans expression of each protein and subsequent recruitment to create the fusion. Some example systems include, but are not limited to, SunTag (a protein scaffold containing peptide epitopes fused to the dCas9 protein). The LSR can be fused to single-chain variable fragment (scFV) antibodies, which when delivered in trans, are recruited to the peptide epitopes), SpyTag (a 13 residue peptide called Spytag and a 116 residue complementary domain) are fused to the DBD and LSR respectively, which when delivered in trans, spontaneously assemble creating a covalent isopeptide bond), coiled-coil peptide heterodimers, or SnoopTag and SnoopCatcher can also be used.

Inducible recruitment refers to a DBD and an LSR fused to inducible binding proteins, whereupon stimulus such as small molecules or light, cause dimerization, recruiting the LSR to the DBD (e.g., dCas9). Examples of the system include FK506 binding protein 12 (FKBP) and FKBP rapamycin binding (FRB) domains, that dimerize upon rapamycin induction, pMag and nMag, which dimerize upon exposure to blue light, and DmrA/DmrC, which dimerize in the presence of rapamycin analog known as the A/C heterodimerizer.

### LSR Donor and Acceptor Attachment Sites

Recombination sites for the LSR of the LSR-DBD fusions described herein are typically between 30 and 200 nucleotides in length and comprising two motifs with a partial inverted-repeat symmetry, which flank a central crossover sequence at which the recombination takes place. Recombinases bind to these inverted-repeated sequences, which are specific to each recombinase, and are herein referred to as “recombinase recognition sequences,” “recombinase recognition sites,” “attP sites,” “attB sites,” “attD sites,” “attH sites,” “attA sites,” “attachment sites,” “pseudosites,” “genomic pesudosites,” or “genomic insertion sites”. In some embodiments, an attB site is present in the target DNA sequence (such as cellular DNA) and an attP site is present in the DNA sequence to be integrated into the target DNA sequence. In some embodiments, an attP site is present in the target DNA sequence (such as cellular DNA) and an attB site is present in the DNA sequence to be integrated into the target DNA sequence. As disclosed herein, “attD” refers to a donor attachment site, which could be an attP or an attB site, “attA” refers to the cognate acceptor site and “attH” refers to integration sites found natively in a mammalian genome, for example the human genome. A “landing pad,” is an exogenous DNA sequence that includes an attachment site of a LSR integrated into a location of the target DNA. A landing pad can be integrated into a target DNA using any method known in the art, such as by using a zinc finger nuclease, TALEN, or the CRISPR-Cas system, or by using an LSR-DBD fusion described herein.

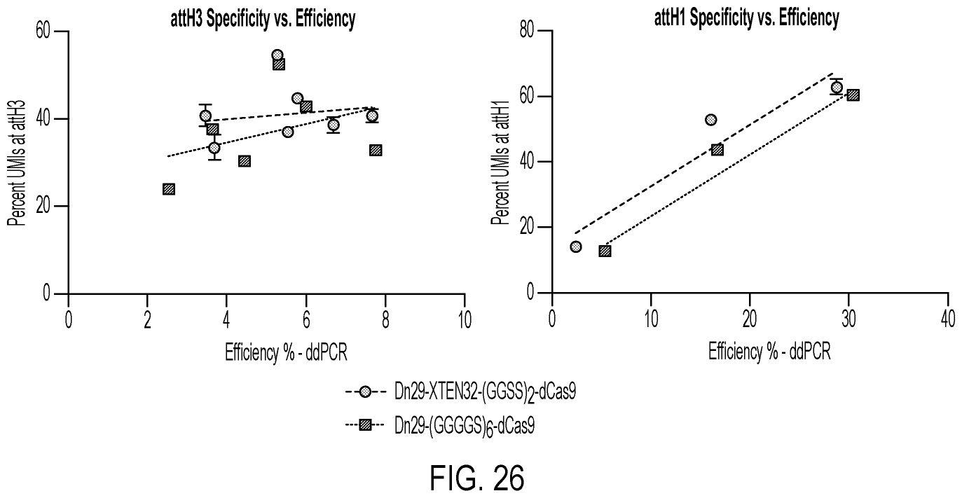

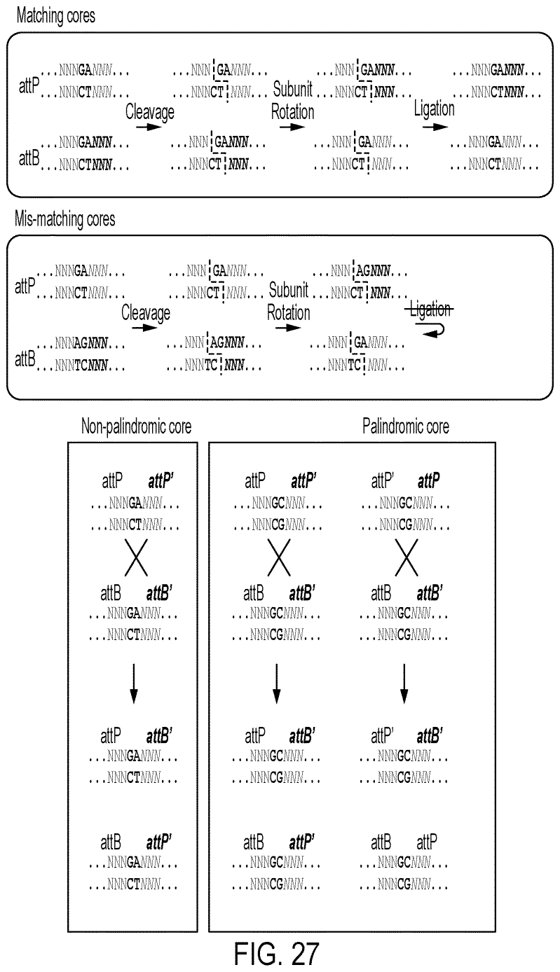

During recombination, crossover occurs at the central dinucleotide of the attB/attP sites. The sequence of the central dinucleotide is the sole determinant of the directionality of the recombination. For the recombination to be directional, the central dinucleotide needs to be non-palindromic. See FIG. 26. For example, the central dinucleotide sequence found in the attB/attP sites for large serine recombinases, which are strictly directional, can be AA, TT, GG, CC, AG, GA, AC, CA, TG, GT, TC, or CT. A schematic is provided in FIG. 27.

The outcome of recombination depends, in part, on the location and orientation of the attachment sites. For example, inversion recombination happens between two inverted attachment sites located on the same DNA molecule. A DNA loop formation brings the two attachment sites together, at which point DNA cleavage, strand exchange, and ligation occur. This reaction is ATP independent. The end result of such an inversion recombination event is that the stretch of DNA between the repeated site inverts (i.e., the stretch of DNA reverses orientation) such that what was the coding strand is now the non-coding strand and vice versa. Such reactions, the DNA is conserved with no net gain or no loss of DNA. Conversely, excisive recombination occurs between two attachment sites that are oriented in the same direction on the same DNA molecule. In this case, the intervening DNA is excised/removed. Integrative recombination can occur between two attachment sites that are located on different DNA molecules, where one of the DNA molecules is circular (for integration of the entire circular molecule). If the other DNA molecule is cellular or genomic DNA, the two molecules are combined into one molecule, with the circular DNA integrated into the cellular or genomic DNA. Finally, translocation occurs upon recombination of two attachment sites found on different, linear DNA molecules. A schematic for insertion/integration, excision, inversion, and translocation is provided in FIG. 28.

LSRs have two attachment sites to which it binds and recombines sequence-specifically. In some embodiments, target DNA, with an introduced attachment site is targeted. In another embodiment, to target sequences endogenously present in a target DNA, a sequence similar to the desired attachment site sequence must be present in the target DNA, such as in a genome or other cellular DNA. In some embodiments, a LSR that has the ability to target endogenous sequences can be used in the LSR-DBD fusion. Another factor that may be relevant is the number of endogenous sites that the LSR can integrate into. Having fewer (but not 0) integration sites may increase efficiency of integration into a single pseudosite, since there will be fewer potential off-target sites which may act as a sink for LSRs thus reducing on-target efficiency. Thus, in some embodiments, a LSR that has the ability to target a single or up to thousands of endogenous sequences can be used in the LSR-DBD fusion.

### Guide Polynucleotides

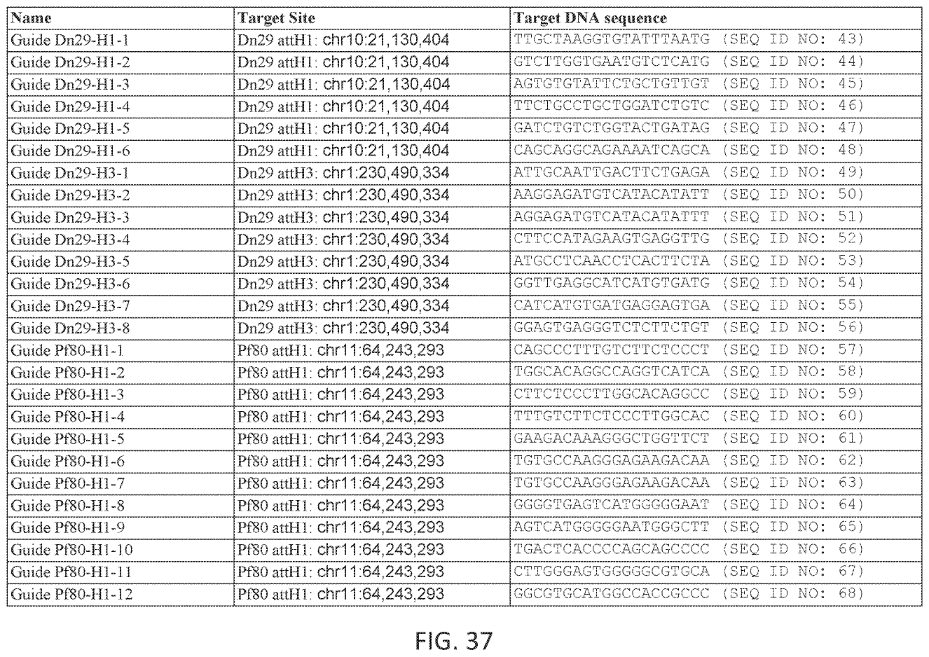

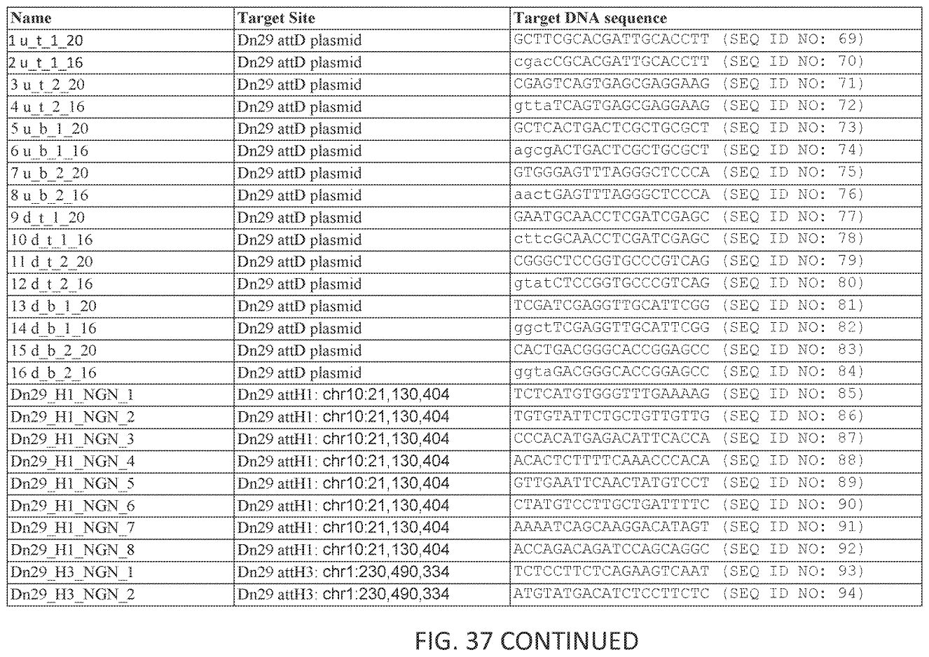

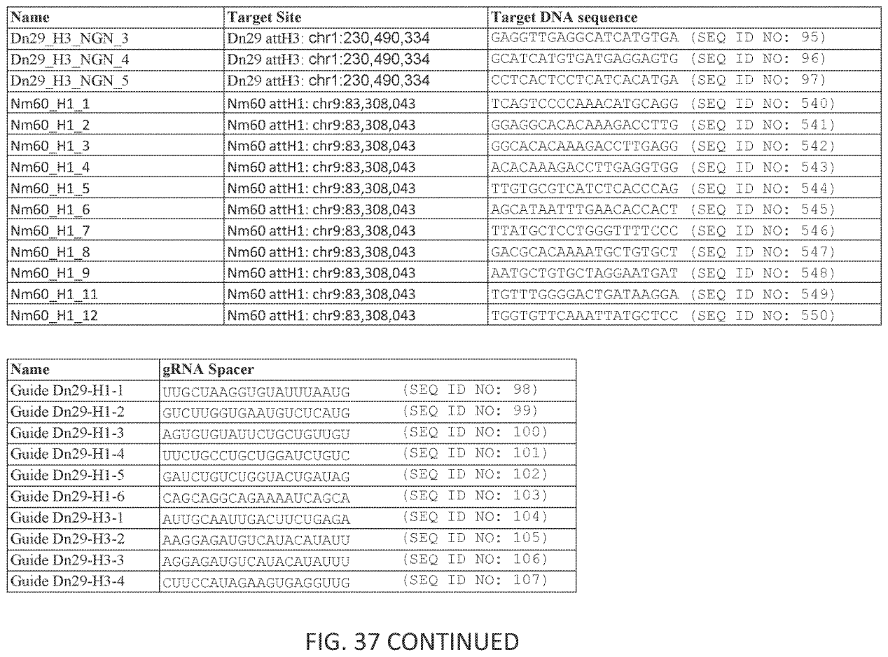

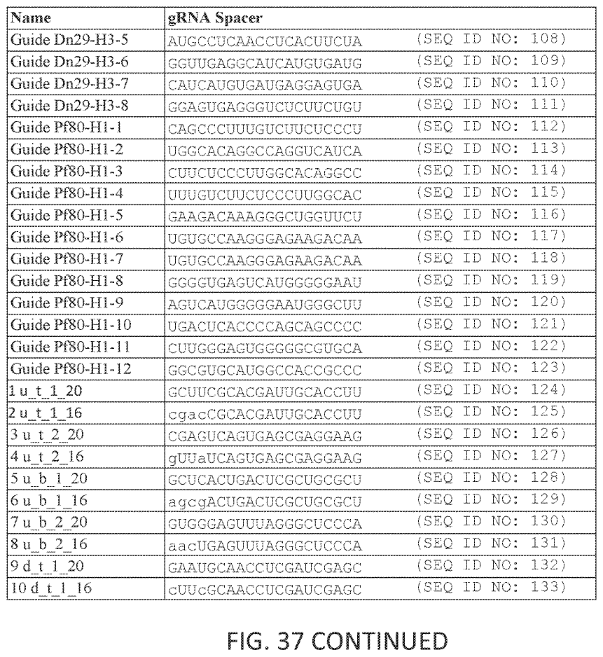

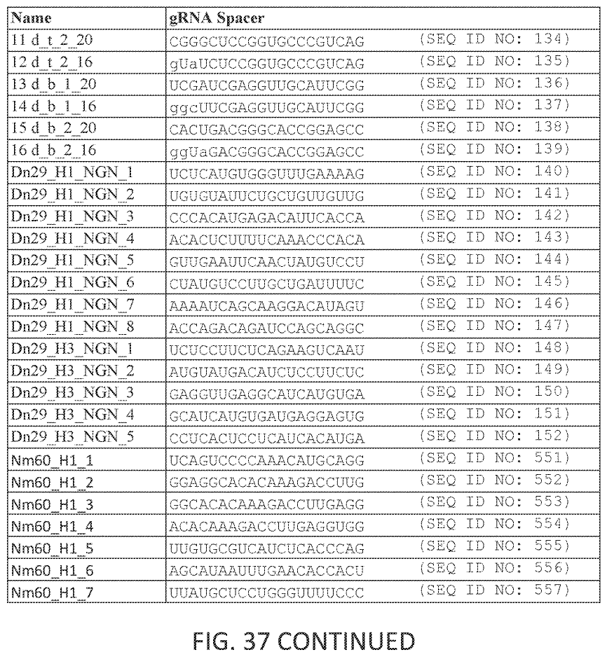

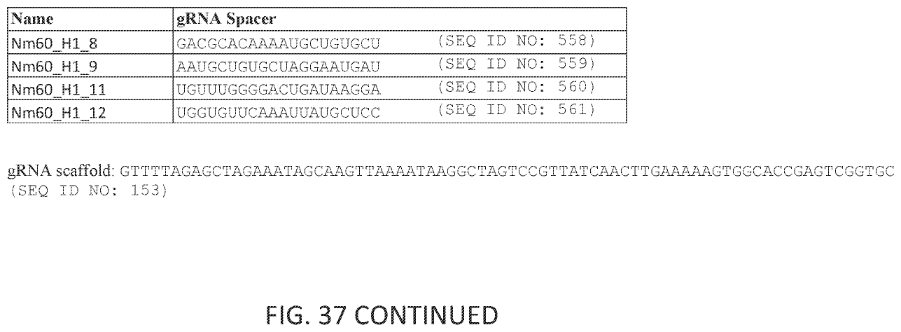

When a Cas protein domain is used as the DBD portion of the LSR-DBD fusion, the Cas portion is capable of binding one or more guide RNAs (gRNAs), in which the spacer sequences are including, but not limited to, those described in FIG. 37, and thereby directs or targets the LSR-DBD fusion to a target nucleic acid of interest. In some embodiments, a guide RNA is used that targets a target sequence present on an acceptor target DNA of interest. In some embodiments, a guide RNA is used that targets a target sequence present on a donor DNA of interest. In some embodiments, the system described herein uses two guide RNAs, one that targets a target sequence present on an acceptor target DNA of interest and a second that targets a target sequence present on a donor DNA of interest. In some embodiments, the system described herein uses two guide RNAs, one that targets a target sequence present on an acceptor target DNA of interest and a second that targets a second target sequence present on the acceptor target DNA of interest. In some embodiments, the first and second target sequences on the acceptor target DNA of interest are on either side of the LSR attachment site in the target DNA of interest. In some embodiments, a guide RNA is used that targets a target sequence present on an acceptor target DNA of interest and a target sequence present on a donor DNA of interest, wherein the target sequences are the same. For example, the target sequence targeted by the guide in the acceptor target DNA of interest is included on the donor DNA molecule proximal to, overlapping with, or within the attD site. In some embodiments, more than two guide RNA sequences are used, for example one or more guide RNA sequences that target(s) one or more target sequences present on a donor DNA molecule of interest and one or more guide RNA sequences that target(s) one or more target sequences present on an acceptor target DNA of interest.

As used herein, the term “guide polynucleotide” or “guide RNA” or “gRNA”, relates to a polynucleotide sequence that can form a complex with a Cas protein and enables the Cas protein to recognize, bind to, and optionally cleave a DNA target site. The guide RNA is a specific RNA sequence that recognizes a target DNA region of interest and directs the Cas protein, and thus the LSR-DBD fusion, to that site. The gRNA is typically made up of two parts: CRISPR RNA (crRNA) (also referred to as a gRNA spacer or spacer sequence), a nucleotide sequence that binds to a complement of a target DNA sequence, and a trans-activating CRISPR RNA (tracr RNA), which serves as a binding scaffold for the Cas protein. In the context of CRISPR, hybridization between the complementary sequence of a target sequence and a gRNA spacer sequence promotes the formation of a CRISPR complex. Full complementarity is not necessarily required, provided there is sufficient complementarity to cause hybridization and promote formation of a CRISPR complex.

While crRNAs and tracrRNAs exist as two separate RNA molecules in nature, a single RNA molecule can contain both the crRNA sequence fused to the scaffold tracrRNA sequence, referred to as a single guide RNA (sgRNA). In some embodiments, the gRNA is a sgRNA. In some embodiments, the gRNA comprises two separate RNA molecules. The guide polynucleotide sequence can be a RNA sequence, a DNA sequence, or a combination thereof (a RNA-DNA combination sequence), such as the Caribou Biosciences system that uses a “chRDNA” system where the guide polynucleotide is a hybrid RNA/DNA system. Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. See also U.S. Patent Application US 2015-0082478 A1, published on Mar. 19, 2015 and US 2015-0059010 A1, published on Feb. 26, 2015, both are hereby incorporated in its entirety by reference.

In one embodiment of the disclosure, the guide polynucleotide is a sgRNA capable of forming a guide RNA/protein RNP complex with the DBD of the LSR-DBD fusions disclosed herein, wherein said RNP complex can recognize and bind to a complement of a target sequence. One or more target sequences may be present in the acceptor target DNA of interest, the donor DNA of interest, or both.

In one embodiment of the disclosure, the guide polynucleotide is a sgRNA capable of forming a guide RNA/protein RNP complex with the DBD of the LSR-DBD fusions disclosed herein, wherein said complex can recognize and bind to a complement of a target sequence, wherein said sgRNA comprises a “crRNA” or “spacer” or “spacer sequence” linked to a “scaffold” or “scaffold sequence” or “tracrRNA.” One or more target sequences may be present in the acceptor target DNA of interest, the donor DNA of interest, or both.

In one embodiment of the disclosure, the guide polynucleotide is a gRNA capable of forming a guide RNA/protein RNP complex with the DBD of the LSR-DBD fusions disclosed herein, wherein said complex can recognize and bind to a complement of a target sequence, wherein said guide RNA is a duplex molecule comprising a spacer and a scaffold, wherein said spacer comprises a sequence capable of hybridizing to a complement of a target DNA sequence. One or more target sequences may be present in the acceptor target DNA of interest, the donor DNA of interest, or both.

The guide polynucleotide can be a double molecule (also referred to as duplex guide polynucleotide) comprising a spacer sequence and a scaffold sequence. The spacer includes a first nucleotide sequence domain that can hybridize to a nucleotide sequence in a target DNA (i.e., to a nucleotide sequence complementary to a target sequence) and a second nucleotide sequence (also referred to as a “tracr mate” sequence) that is part of a Cas protein recognition (CPR) domain. The tracr mate sequence can be hybridized to a scaffold along a region of complementarity and together form a Cas protein recognition domain or CPR domain. The CPR domain is capable of interacting with a Cas protein. The spacer and the scaffold of the duplex guide polynucleotide can be RNA, DNA, and/or RNA-DNA-combination sequences. In some embodiments, the spacer molecule of the duplex guide polynucleotide is referred to as “spacer DNA” or “crDNA” (when composed of a contiguous stretch of DNA nucleotides) or “spacer RNA” or “crRNA” (when composed of a contiguous stretch of RNA nucleotides), or “spacer DNA-RNA” or “crDNA-RNA” (when composed of a combination of DNA and RNA nucleotides). The size of the fragment of the spacer naturally occurring in Bacteria and Archaea that can be present in a spacer disclosed herein can range from, but is not limited to, 2, 3, 4, 5, 6, 7, 8, 9, 10, 11, 12, 13, 14, 15, 16, 17, 18, 19, 20, 21 or more nucleotides. In some embodiments the scaffold is referred to as “scaffold RNA” or “tracrRNA” (when composed of a contiguous stretch of RNA nucleotides) or “scaffold DNA” or “tracrDNA” (when composed of a contiguous stretch of DNA nucleotides) or “scaffold DNA-RNA” or “tracrDNA-RNA” (when composed of a combination of DNA and RNA nucleotides. In one embodiment, the RNA that guides the RNA/Cas9 RNP complex of the LSR-DBD fusion is a duplexed RNA comprising a duplex spacer-scaffold. The scaffold or tracrRNA contains, in the 5′-to-3′ direction, (i) a sequence that anneals with the repeat region of CRISPR type II crRNA and (ii) a stem loop-containing portion (Deltcheva et al., Nature 471:602-607). The duplex guide polynucleotide can form a complex with a Cas protein portion of the LSR-DBD fusion, wherein said guide polynucleotide/Cas RNP complex (also referred to as a guide polynucleotide/Cas RNP system) can direct the DBD of the LSR-DBD fusion proteins described herein to a target site, enabling the DBD protein to recognize and bind to the target site. See also U.S. Patent Application US 2015-0082478 A1, published on Mar. 19, 2015 and US 2015-0059010 A1, published on Feb. 26, 2015, both are hereby incorporated in its entirety by reference. In some embodiments, the spacer sequence is fused to the 5′ end of the scaffold sequence. Alternatively, the spacer sequence is fused to the 3′ end of the scaffold sequence.

The guide polynucleotide can also be a single molecule (also referred to as single guide polynucleotide) comprising a spacer sequence linked to a scaffold sequence. The single guide polynucleotide comprises a first nucleotide sequence domain that can hybridize to a nucleotide sequence in a target DNA (i.e., to a nucleotide sequence complementary to a target sequence) and comprises a Cas protein recognition domain (CPR domain), that interacts with a Cas protein. By “domain” as used in this context it is meant a contiguous stretch of nucleotides that can be RNA, DNA, and/or RNA-DNA-combination sequence. The spacer domain and/or the CPR domain of a single guide polynucleotide can comprise a RNA sequence, a DNA sequence, or a RNA-DNA-combination sequence. The single guide polynucleotide being comprised of sequences from the spacer and the scaffold may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas protein portion of the LSR-DBD fusion, wherein said guide polynucleotide/Cas RNP complex (also referred to as a guide polynucleotide/Cas RNP system) can direct the DBD of the LSR-DBD fusion proteins described herein to a target site, enabling the DBD to recognize and bind to the target site. See also U.S. Patent Application US 2015-0082478 A1, published on Mar. 19, 2015 and US 2015-0059010 A1, published on Feb. 26, 2015, both are hereby incorporated in its entirety by reference.

In some embodiments, the gRNA comprises a sgRNA comprising a spacer RNA sequence portion and a tracr RNA portion, wherein the nucleic acid sequence of the spacer RNA sequence portion is the same as a target sequence on a DNA target of interest, and thus is complementary to, and hybridizes with the complement of the target sequence on the DNA target of interest. One or more target sequences may be present in the acceptor target DNA of interest, the donor DNA of interest, or both.

In some embodiments, immediately 3′ to the target sequence on the DNA target of interest is a protospacer adjacent motif (“PAM”) sequence. The PAM is a short DNA sequence (usually 2-6 base pairs in length) that, in a CRISPR-Cas9 system, follows the DNA region targeted for cleavage by the CRISPR system. In some embodiments, the DBD portion of the LSR-DBD fusion comprises Streptococcus pyogenes dCas9 which recognizes the PAM sequence 5′-NGG-3′ (where “N” can be any nucleotide base). Thus, in some embodiments, the DNA target of interest comprises a nucleotide sequence that is the same as the spacer sequence of the guide polynucleotide immediately followed in the 3′ direction by “NGG”. A person of skill in the art recognizes that there are different Cas endonucleases isolated from different bacterial species, each of which recognizes a different PAM. In some embodiments, the DBD portion of the LSR-DBD fusion comprises Staphylococcus aureus dCas9 which recognizes the PAM sequence 5′-NGRRT-3′ or 5′- or NGRRN-3′ (where “N” can be any nucleotide base). In some embodiments, the DBD portion of the LSR-DBD fusion comprises Neisseria meningitidis dCas9 which recognizes the PAM sequence 5′-NNNNGATT-3′ (where “N” can be any nucleotide base). In some embodiments, the DBD portion of the LSR-DBD fusion comprises Campylobacter jejuni dCas9 which recognizes the PAM sequence 5′-NNNNRYAC-3′ (where “N” can be any nucleotide base). In some embodiments, the DBD portion of the LSR-DBD fusion comprises Streptococcus thermophilus dCas9 which recognizes the PAM sequence 5′-NNAGAAW-3′ (where “N” can be any nucleotide base). Cas9 mutants that have altered specificity, relaxed PAM requirements, or recognize novel PAM sequences can also be used as a DBD portion of the LSR-DBD fusion. In some embodiments, the DBD portion of the LSR-DBD fusion comprises dCas9-SpG which recognizes the PAM sequence 5′-NGN-3′ (where “N” can be any nucleotide base).

In some embodiments, the guide polynucleotide comprises a spacer sequence portion, wherein the nucleic acid sequence of the spacer sequence portion is the same as a target sequence on a target or donor DNA of interest (except in RNA spacer sequences “T” is “U”), wherein the target sequence is proximal to, overlapping with, or within the attachment site (e.g., attA or attD) of the LSR on a target DNA of interest. In some embodiments, the target sequence on a target or donor DNA of interest is within 300 nucleotides upstream or downstream of an attachment site (e.g., attA or attD) of the LSR of the LSR-DBD fusion on a target or donor DNA of interest, wherein distance is measured from the center of the dinucleotide core of the attachment site to the position between the spacer sequence and the PAM. In some embodiments, the target sequence on a target or donor DNA of interest within 200 nucleotides upstream or downstream of an attachment site (e.g., attA or attD) of the LSR of the LSR-DBD fusion on a target or donor DNA of interest. In some embodiments, the target sequence on a target or donor DNA of interest is within 100 nucleotides upstream or downstream of an attachment site (e.g., attA or attD) of the LSR of the LSR-DBD fusion on a target or donor DNA of interest. In some embodiments, the target sequence on a target or donor DNA of interest is within 80 nucleotides upstream or downstream of an attachment site (e.g., attA or attD) of the LSR of the LSR-DBD fusion on a target or donor DNA of interest. In some embodiments, the target sequence on a target or donor DNA of interest is within 50 nucleotides upstream or downstream of an attachment site (e.g., attA or attD) of the LSR of the LSR-DBD fusion on a target or donor DNA of interest. A target sequence can be on either strand of target or donor DNA of interest. In some embodiments, the guide polynucleotide is a sgRNA. Generally, spacers that are directly proximal to the target integration attachment site, e.g., attH, have the highest integration rates, the spacers farther away have reduced integration, and spacers that overlap with the dinucleotide core of an attachment site greatly reduce or fully ablate integration.

In certain aspects, described herein is a nucleic acid encoding a guide polynucleotide for use with the LSR-DBD fusions described herein. The guide polynucleotide may be encoded on the same nucleic acid molecule as the LSR-DBD fusion and/or as a donor polynucleotide, or may be encoded on a separate nucleic acid molecule. In some embodiments, the guide polynucleotide is a gRNA comprising a spacer sequence portion and a tracr RNA portion. In some embodiments, the guide polynucleotide is a sgRNA comprising a spacer sequence portion and a tracr RNA portion. In some embodiments, the spacer sequence portion is about 20 nucleotides in length. In some embodiments, the spacer sequence portion is 16 nucleotides in length. In some embodiments, the spacer sequence portion is 20 nucleotides in length. In some embodiments, the spacer sequence portion comprises the same nucleotide sequence as a target sequence on a target or donor DNA of interest, wherein the target sequence is proximal to, overlapping with, or within the attachment site (e.g., attA or attD) of the LSR of the LSR-DBD fusion on a target or donor DNA of interest. In some embodiments, the spacer sequence portion comprises the same nucleotide sequence as a target sequence on a target or donor DNA of interest, wherein the target sequence is within 300 nucleotides of the attachment site (e.g., attA or attD) of the LSR of the LSR-DBD fusion on a target or donor DNA of interest. In some embodiments, the spacer sequence portion comprises the same nucleotide sequence as a target sequence on a target or donor DNA of interest, wherein the target sequence is within 200 nucleotides of the attachment site (e.g., attA or attD) of the LSR of the LSR-DBD fusion on a target or donor DNA of interest. In some embodiments, the spacer sequence portion comprises the same nucleotide sequence as a target sequence on a target or donor DNA of interest, wherein the target sequence is within 100 nucleotides of the attachment site (e.g., attA or attD) of the LSR of the LSR-DBD fusion on a target or donor DNA of interest. In some embodiments, the spacer sequence portion comprises the same nucleotide sequence as a target sequence on a target or donor DNA of interest, wherein the target sequence is within 80 nucleotides of the attachment site (e.g., attA or attD) of the LSR of the LSR-DBD fusion on a target or donor DNA of interest. In some embodiments, the DNA sequence immediately 3′ to the target sequence on a target or donor DNA of interest comprises a PAM sequence. In some embodiments, the spacer sequence portion comprises the same nucleotide sequence as a target sequence on a target or donor DNA of interest, wherein the target sequence is within 50 nucleotides of the attachment site (e.g., attA or attD) of the LSR of the LSR-DBD fusion on a target or donor DNA of interest. In some embodiments, the DNA sequence immediately 3′ to the target sequence on a target or donor DNA of interest comprises a PAM sequence. In some embodiments, the DNA sequence immediately 3′ to the target sequence on a target or donor DNA of interest comprises a PAM sequence NGG. In some embodiments, the spacer sequence portion comprises the same nucleotide sequence as a target sequence on a target DNA of interest (e.g., proximal to, overlapping with, or within an attA site). In some embodiments, the spacer sequence portion comprises the same nucleotide sequence as a target sequence on a donor DNA of interest (e.g., proximal to, overlapping with, or within an attD site). In some embodiments, the spacer sequence portion of the gRNA or sgRNA comprises a nucleotide sequence selected from FIG. 37 (SEQ ID NOs: 98-152, 551-561). In some embodiments, the spacer sequence portion of the gRNA or sgRNA comprises a nucleotide sequence selected from FIG. 37 (SEQ ID NOs: 98-152, 551-561) with an additional “G” nucleotide present on the 5′ end. In some embodiments, the spacer sequence portion of the gRNA or sgRNA consists of a nucleotide sequence selected from FIG. 37 (SEQ ID NOs: 98-152, 551-561). In some embodiments, the spacer sequence portion of the gRNA or sgRNA consists of a nucleotide sequence selected from FIG. 37 (SEQ ID NOs: 98-152, 551-561) with an additional “G” nucleotide present on the 5′ end. In some embodiments, the tracr RNA portion of the gRNA or sgRNA comprises SEQ ID NO: 153. In some embodiments, the tracr RNA portion of the gRNA or sgRNA consists of SEQ ID NO: 153. In some embodiments, the spacer sequence portion of the gRNA or sgRNA comprises a nucleotide sequence selected from FIG. 37 (SEQ ID NOs: 98-152, 551-561) and the tracr RNA portion of the gRNA or sgRNA comprises SEQ ID NO: 153. In some embodiments, the spacer sequence portion of the gRNA or sgRNA comprises a nucleotide sequence selected from FIG. 37 (SEQ ID NOs: 98-152, 551-561) with an additional “G” nucleotide present on the 5′ end and the tracr RNA portion of the gRNA or sgRNA comprises SEQ ID NO: 153. In some embodiments, the spacer sequence portion of the gRNA or sgRNA consists of a nucleotide sequence selected from FIG. 37 (SEQ ID NOs: 98-152, 551-561) and the tracr RNA portion of the gRNA or sgRNA consists of SEQ ID NO: 153. In some embodiments, the spacer sequence portion of the gRNA or sgRNA consists of a nucleotide sequence selected from FIG. 37 (SEQ ID NOs: 98-152, 551-561) with an additional “G” nucleotide present on the 5′ end and the tracr RNA portion of the gRNA or sgRNA consists of SEQ ID NO: 153. In some embodiments the gRNA or sgRNA comprises SEQ ID NOs: 98-152, 551-561 immediately followed by SEQ ID NO: 153. In some embodiments the gRNA or sgRNA comprises SEQ ID NOs: 98-152, 551-561 with an additional “G” nucleotide present on the 5′ end immediately followed by SEQ ID NO: 153. In some embodiments the gRNA or sgRNA consists of SEQ ID NOs: 98-152, 551-561 immediately followed by SEQ ID NO: 153. In some embodiments the gRNA or sgRNA consists of SEQ ID NOs: 98-152, 551-561 with an additional “G” nucleotide present on the 5′ end immediately followed by SEQ ID NO: 153.

### Donor DNAs

Certain aspects of the present application are directed to a nucleic acid for use in site-specific insertion of an exogenous nucleic acid, e.g., a gene of interest (GOI), into a target DNA, e.g., a genome. In some embodiments, the exogenous nucleic acid for insertion (e.g., the GOI) can be up to about 10, 15, 20, 25, 30, 35, 40, 45, 50, 55, 60, 65, 70, 75, 80, 85, 90, 95, 100, 105, 110, 115, 120, 125, 130, 140, 150, 160, 170, 180, 190, 200, or 250 kilobases or higher in length. The GOI can include non-coding sequences, including cis regulatory regions and introns.

The donor DNA can contain from 15 bases (b) or base pairs (bp) to about 250 kilobases (kb) or kilobase pairs (kbp) in length (e.g., from about 50, 75, or 100 b or bp to about 110, 120, 125, 150, 200, 225, 250, 275, 300, 325, 350, 375, 400, 425, 450, 475, 500, 525, 550, 575, 600, 650, 700, 750, 800, 850, 900, 950, 1000, 1250, 1500, 1750, 2000, 2250, 2500, 2750, 3000, 3250, 3500, 3750, 4000, 4250, 4500, 4750, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 16,000, 17,000, 18,000, 19,000, 20,000, 21,000, 22,000, 23,000, 24,000, 25,000, 26,000, 27,000, 28,000, 29,000, 30,000 (and increasing by 1,000 increments) up to 250,000 b or bp in length. Longer donor DNA molecules can be provided in the form of a circular or linearized plasmid or as a component of a vector (e.g., as a component of a viral vector), or an amplification or polymerization product thereof. Shorter donor DNA molecules can be provided as double stranded oligonucleotides. Exemplary double-stranded template oligonucleotides are, or are least about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 115, 120, 125, 150, 175, 200, 225, or 250 b or bp in length. The donor DNA can be provided in the reaction mixture for introduction into the cell at a concentration of from about 1 μM to about 200 μM, from about 2 μM to about 190 μM, from about 2 μM to about 180 μM, from about 5 μM to about 180 μM, from about 9 μM to about 180 μM, from about 10 μM to about 150 μM, from about 20 μM to about 140 μM, from about 30 μM to about 130 μM, from about 40 μM to about 120 μM, or from about 45 or 50 μM to about 90 or 100 μM. In some cases, the donor DNA can be provided in the reaction mixture for introduction into the cell at a concentration of, or of about, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 35 μM, 40 μM, 45 μM, 50 μM, 55 μM, 60 μM, 70 μM, 80 μM, 90 μM, 100 μM, 110 μM, 115 μM, 120 μM, 130 μM, 140 μM, 150 μM, 160 μM, 170 μM, 180 μM, 190 μM, 200 μM, or more.

In some embodiments, the donor DNA comprises a target sequence which is the same nucleotide sequence as the spacer sequence portion of a guide polynucleotide (e.g., gRNA, sgRNA). In some embodiments, the donor DNA comprises a target sequence which is the same as the target sequence of the target DNA of interest so that the same guide polynucleotide sequence can be used to target the LSR-DBD fusion to the donor and target DNA of interest.

The donor DNA can contain a wide variety of different sequences. In some cases, the donor DNA encodes a stop codon, or frame shift, as compared to the target genomic region prior to cleavage and recombination. Such a donor DNA can be useful for knocking out or inactivating a gene or portion thereof. In some cases, the donor DNA encodes one or more missense mutations or in-frame insertions or deletions as compared to the target genomic region. Such a donor DNA can be useful for altering the expression level or activity (e.g., ligand specificity) of a target gene or portion thereof.

As another example, the donor DNA can encode a wild-type sequence for rescuing the expression level or activity of a target endogenous gene or protein. For instance, T cells containing a mutation in the FoxP3 gene, or a promoter region thereof, can be rescued to treat X-linked IPEX or systemic lupus erythematous. Alternatively, the donor DNA can encode a sequence that results in lower expression or activity of a target gene. For example, an increased immunotherapeutic response can be achieved by deleting or reducing the expression or activity of FoxP3 in T cells prepared for immunotherapy against a cancer or infectious disease target.

As another example, the donor DNA can encode a mutation that alters the function of a target gene. For instance, the donor DNA can encode a mutation of a cell surface protein necessary for viral recognition or entry. The mutation can reduce the ability of the virus to recognize or infect the target cell. For example, mutations of CCR5 or CXCR4 can confer increased resistance to HIV infection in CD4+ T cells.

In some cases, the donor DNA encodes a sequence that, although adjacent to, is entirely orthogonal to the endogenous sequence. For example, the donor DNA can encode an inducible promoter or repressor element unrelated to the endogenous promoter of a target gene. The inducible promoter or repressor element can be inserted into the promoter region of a target gene to provide temporal and/or spatial control of the target gene expression or activity.

In some instances, the donor DNA sequence includes an attD attachment site, such as an attB or an attP site, of a LSR, a constitutive promoter operably linked to a nucleotide sequence encoding a detectable marker, followed by a nucleotide sequence encoding a first selectable marker.

### Target DNAs

Target DNA can be any type of DNA molecule, in vitro or in vivo, including but not limited to genomic DNA, mitochondrial DNA, eukaryotic DNA, prokaryotic DNA, cDNA, and synthesized DNA. The key requirement for the target DNA is that it contains an LSR attachment site, including but not limited to an attB site, an attP site, an attH site, or a pseudosite.

The target DNA (or target genome) can contain multiple LSR attachment sites. Through the use of LSR-DBD fusions, the DNA-binding domain of the fusion can direct the LSR domain to a single attachment site thereby substantially mitigating off-target recombination.

In some instances, the target DNA sequence includes an attA attachment site, such as an attB or an attP site, of a LSR, a constitutive promoter operably linked to a nucleotide sequence encoding a detectable marker, followed by a nucleotide sequence encoding a first selectable marker. In certain types of landing pads, the attachment site is between the promoter and the nucleotide sequence encoding the detectable protein. When there are more than one landing pads used in a given cell, it is preferred that an attachment site of one landing pad is orthogonal to an attachment site of the same large serine recombinase in any other landing pad. The landing pad is used for further genetic engineering and integration of a nucleic acid molecule of interest via site-specific recombination.

### Nucleic Acid Editing System

In certain aspects, described herein is a nucleic acid editing system comprising a first nucleic acid encoding an LSR-DBD as described herein and a second nucleic acid encoding a gRNA. In some embodiments, the gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides upstream or downstream of a dinucleotide core of an attachment site of the LSR portion of the fusion polypeptide on a DNA of interest. In some embodiments, the spacer sequence portion is 16 to 20 nucleotides long. In some embodiments, the gRNA encoded by the nucleic acid is an sgRNA. In some embodiments, immediately 3′ to the target nucleic acid sequence on the DNA of interest is a PAM sequence. In some embodiments, the first and second nucleic acid are present on the same molecule, for example, but not limited to the same plasmid or vector. In some embodiments, the first and second nucleic acid are present on different molecules, for example, but not limited to different plasmids or vectors.

In some embodiments, the target nucleic acid sequence is within 80 nucleotides upstream or downstream of a dinucleotide core of an attA site of the LSR portion of the fusion polypeptide on a target DNA of interest. In some embodiments, the attA site is a pseudosite in a mammalian target DNA of interest. In some embodiments, the attA site is a pseudosite in the human genome (attH). In some embodiments, the fusion polypeptide encoded by the nucleic acid comprises Dn29 (SEQ ID NO: 1) and dCas9 (SEQ ID NO: 29) and the attH site is chr10:21130404-21130406:−, chr11:77367459-77367461:−, chr1:230490334-230490336:+, chr2:14280297-14280299:+, chr9:116464427-116464429:+, chr20:38982599-38982601:+, chr5:3553012-3553014:−, chr7:134676315-134676317:−, chr10:58514255-58514257:+, or chr4:92338934-92338936:+. In some embodiments, the fusion polypeptide encoded by the nucleic acid comprises Pf80 (SEQ ID NO:2) and dCas9 (SEQ ID NO: 29) and the attH site is chr11:64243293-64243295.

In some embodiments, the tracr RNA portion comprises SEQ ID NO: 153. In some embodiments, the target nucleic acid sequence is within 80 nucleotides upstream of a dinucleotide core of an attachment site of the LSR portion of the fusion polypeptide on a DNA of interest. In some embodiments, the target nucleic acid sequence is within 80 nucleotides downstream of a dinucleotide core of an attachment site of the LSR portion of the fusion polypeptide on a DNA of interest.

In some embodiments, the nucleic acid editing system further comprises a third nucleic acid encoding a second gRNA. In some embodiments, the second gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides downstream of a dinucleotide core of an attachment site of the LSR portion of the fusion polypeptide on a DNA of interest. In some embodiments, the spacer sequence portion of the second gRNA is 16 to 20 nucleotides long. In some embodiments, the second gRNA encoded by the nucleic acid is an sgRNA. In some embodiments, immediately 3′ to the target nucleic acid sequence on the DNA of interest is a PAM sequence. In some embodiments, the first, second and third nucleic acids are present on the same molecule, for example, but not limited to the same plasmid or vector. In some embodiments, the first and second nucleic acid are present on the same molecule, for example, but not limited to the same plasmid or vector and the third nucleic acid is present on a different molecule for example, but not limited to different plasmids or vectors. In some embodiments, the second and third nucleic acid are present on the same molecule, for example, but not limited to the same plasmid or vector and the first nucleic acid is present on a different molecule for example, but not limited to different plasmids or vectors. In some embodiments, the first, second, and third nucleic acid are present on different molecules, for example, but not limited to different plasmids or vectors.

In some embodiments, the nucleic acid editing system further comprises a third nucleic acid comprising a donor DNA sequence which comprises an attD attachment site of the LSR portion of the fusion polypeptide and a nucleic acid sequence for insertion into the target DNA of interest. In some embodiments, the third nucleic acid further comprises a portion that has the same target nucleic acid sequence for the gRNA as the target DNA of interest. In some embodiments, the first, second and third nucleic acids are present on the same molecule, for example, but not limited to the same plasmid or vector. In some embodiments, the first and second nucleic acid are present on the same molecule, for example, but not limited to the same plasmid or vector and the third nucleic acid is present on a different molecule for example, but not limited to different plasmids or vectors. In some embodiments, the second and third nucleic acid are present on the same molecule, for example, but not limited to the same plasmid or vector and the first nucleic acid is present on a different molecule for example, but not limited to different plasmids or vectors. In some embodiments, the first, second, and third nucleic acid are present on different molecules, for example, but not limited to different plasmids or vectors.

In some embodiments, the fusion polypeptide encoded by the nucleic acid comprises: (a) Dn29 (SEQ ID NO: 1) and dCas9 (SEQ ID NO: 29), the attH site on the target DNA of interest is chromosomal locus chr10:21130404-21130406:−, chr11:77367459-77367461:−, chr1:230490334-230490336:+, chr2:14280297-14280299:+, chr9:116464427-116464429:+, chr20:38982599-38982601:+, chr5:3553012-3553014:−, chr7:134676315-134676317:−, chr10:58514255-58514257:+, or chr4:92338934-92338936:+ or comprises the attH sequence found at said chromosomal locus, and the attD attachment site of the donor DNA sequence comprises SEQ ID NO: 154, or a sequence 90% identical to SEQ ID NO: 154; (b) Pf80 (SEQ ID NO: 2) and dCas9 (SEQ ID NO: 29), the attH site on the target DNA of interest is chromosomal locus chr11:64243293-64243295:+, chr1:162878224-162878226:+, chr11:92763120-92763122:−, chr9:103309977-103309979:−, chr13:91145766-91145768:+, chr2:102467361-102467363:+, chr13:99865454-99865456:+, chr9:113640780-113640782:−, chr9:123986548-123986550:−, chr15:53565450-53565452:−, or comprises the attH sequence found at said chromosomal locus, and the attD attachment site of the donor DNA sequence comprises SEQ ID NO: 265, or a sequence 90% identical to SEQ ID NO: 265; (c) Cp36 (SEQ ID NO: 3) and dCas9 (SEQ ID NO: 29), the attH site on the target DNA of interest is chromosomal locus chr16:2789124-2789126:+, chr22:43958465-43958467:−, chr10:117762740-117762742:+, chr7:157294532-157294534:−, chr13:20558930-20558932:−, chr6:151120348-151120350:−, chr10:101429887-101429889:+, chr1:20686551-20686553:+, chr19:50987430-50987432:+, chr4:183226741-183226743:−, or comprises the attH sequence found at said chromosomal locus, and the attD attachment site of the donor DNA sequence comprises SEQ ID NO: 267, or a sequence 90% identical to SEQ ID NO: 267; (d) Nm60 (SEQ ID NO: 4) and dCas9 (SEQ ID NO: 29), the attH site on the target DNA of interest is chromosomal locus chr9:83308042-83308044:−, chr13:79497139-79497141:−, chr9:131409759-131409761:+, chr4:55980785-55980787:+, chr5:96968267-96968269:+, chr6:37700280-37700282:−, chr19:17495840-17495842:−, chr5:126546219-126546221:+, chr10:15703649-15703651:−, chr10:395348-395350:+, or comprises the attH sequence found at said chromosomal locus, and the attD attachment site of the donor DNA sequence comprises SEQ ID NO: 234, or a sequence 90% identical to SEQ ID NO: 234; or (e) Si74 (SEQ ID NO: 5) and dCas9 (SEQ ID NO: 29), the attH site on the target DNA of interest is chromosomal locus chr7:155557356-155557358:+, chr9:77155112-77155114:−, or comprises the attH sequence found at said chromosomal locus, and the attD attachment site of the donor DNA sequence comprises SEQ ID NO: 266, or a sequence 90% identical to SEQ ID NO: 266.

In some embodiments, the third nucleic acid is a plasmid. In some embodiments, the third nucleic acid is a linear amplicon.

In some embodiments, a ratio of donor DNA to target DNA is controlled within the nucleic acid editing system and in methods described herein using the nucleic acid editing system. In some embodiments, the ratio of donor DNA to target DNA is 5:1. In some embodiments, the ratio of donor DNA to target DNA is 4:1. In some embodiments, the ratio of donor DNA to target DNA is 3:1. In some embodiments, the ratio of donor DNA to target DNA is 2:1. In some embodiments, the ratio of donor DNA to target DNA is 1:1. In some embodiments, the ratio of donor DNA to target DNA is 1:2.

### Vectors and Cell Lines

Several aspects of the invention relate to vector systems comprising one or more vectors, or vectors as such comprising nucleic acid sequences encoding the LSR-DBD fusions described herein, encoding guide polynucleotides described herein, and/or comprising donor or target DNA sequences. Vectors can be designed for expression of transcripts (e.g. nucleic acid transcripts, proteins, or enzymes) in prokaryotic or eukaryotic cells. For example, transcripts can be expressed in bacterial cells such as Escherichia coli, insect cells (using baculovirus expression vectors), yeast cells, or mammalian cells. Suitable host cells are discussed further in Goeddel, Gene Expression Technology: Methods In Enzymology 185, Academic Press, San Diego, Calif. (1990), the contents of which is hereby incorporated by reference in its entirety. Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of nucleic acid constructs or one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein (in this case LSR-DBD fusions). Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein, the content of each of which are hereby incorporated by reference in their entireties.

Minicircles are small circular plasmids or DNA vectors that are episomal and are produced as a circular expression cassette devoid of any bacterial plasmid backbone. They can be generated from a parental bacterial plasmid that contains a heterologous nucleic acid and two recombinase target sites by intramolecular (cis-) recombination using a site-specific recombinase, such as PhiC31 integrase. Recombination between the two sites generates a minicircle and a leftover miniplasmid. The minicircle can be recovered via separation from the miniplasmid.

Examples of suitable inducible non-fusion E. coli expression vectors include pTrc (Amrann et al., (1988) Gene 69:301-315) and pET lid (Studier et al., Gene Expression Technology: Methods In Enzymology 185, Academic Press, San Diego, Calif. (1990) 60-89), the contents of each of which are hereby incorporated by reference in their entireties.

In some embodiments, a vector is a yeast expression vector. Examples of vectors for expression in yeast Saccharomyces cerivisae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.), the contents of each of which are hereby incorporated by reference in their entireties.

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39), the contents of each of which are hereby incorporated by reference in their entireties.

In some embodiments, a vector is capable of driving expression of one or more sequences in mammalian cells (e.g., but not limited to, human embryonic stem cells, HEK cells, hepatocellular carcinoma cells) using a mammalian expression vector. Examples of mammalian expression vectors include pCDM8 (Seed, 1987. Nature 329:840) and pMT2PC (Kaufman, et al., 1987. EMBO J. 6:187-195), the contents of each of which are hereby incorporated by reference in their entireties. When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. In some embodiments, the promoter is an Ef1a promoter. In some embodiments, the promoter is a U6 promoter. For other suitable expression systems for both prokaryotic and eukaryotic cells see, e.g., Chapters 16 and 17 of Sambrook, et al., Molecular Cloning: A Laboratory Manual. 2^{nd} ed., Cold Spring Harbor Laboratory, Cold Spring Harbor Laboratory Press, Cold Spring Harbor, N.Y., 1989, the contents of each of which are hereby incorporated by reference in their entireties.

In some embodiments, a vector is capable of driving expression of one or more sequences in plant cells using a plant cell expression vector.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264, 166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546), the contents of each of which are hereby incorporated by reference in their entireties.

In some embodiments, methods for introducing LSR-DBD fusion-gRNA ribonucleoprotein complex into a cell (e.g., a hematopoietic cell or hematopoietic stem cell, including, e.g., such cells from humans) include forming a reaction mixture containing the protein or ribonucleoprotein complex and introducing transient holes in the extracellular membrane of the cell. Such transient holes can be introduced by a variety of methods, including, but not limited to, electroporation, cell squeezing, or contacting with nanowires or nanotubes. Generally, the transient holes are introduced in the presence of the protein or ribonucleoprotein complex and the protein or ribonucleoprotein complex is allowed to diffuse into the cell.

Methods, compositions, and devices for electroporating cells to introduce a protein or ribonucleoprotein complex can include those described in WO/2006/001614 or Kim, J. A. et al. Biosens. Bioelectron. 23, 1353-1360 (2008), the contents of each of which are hereby incorporated by reference in their entireties. Additional or alternative methods, compositions, and devices for electroporating cells to introduce a protein or ribonucleoprotein complex can include those described in U.S. Patent Appl. Pub. Nos. 2006/0094095; 2005/0064596; or 2006/0087522, the contents of each of which are hereby incorporated by reference in their entireties. Additional or alternative methods, compositions, and devices for electroporating cells to introduce a protein or ribonucleoprotein complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842 and Geng, T. et al. J. Control Release 144, 91-100 (2010); and Wang, J., et al. Lab. Chip 10, 2057-2061 (2010), the contents of each of which are hereby incorporated by reference in their entireties.

In some cases, the methods or compositions described in the patents or publications cited herein are modified for protein or ribonucleoprotein delivery. Such modification can include increasing or decreasing voltage, pulse length, and/or the number of pulses. Such modification can further include modification of buffers, media, electrolytic solutions, or components thereof. Electroporation can be performed using devices known in the art, such as a Bio-Rad Gene Pulser Electroporation device, an Invitrogen Neon transfection system, a MaxCyte transfection system, a Lonza Nucleofection device, a NEPA Gene NEPA21 transfection device, a flow through electroporation system containing a pump and a constant voltage supply, or other electroporation devices or systems known in the art.

Methods, compositions, and devices for squeezing or deforming a cell to introduce a protein or ribonucleoprotein complex can include those described herein. Additional or alternative methods, compositions, and devices can include those described in Nano Lett. 2012 Dec. 12; 12(12):6322-7; Proc Natl Acad Sci USA. 2013 Feb. 5; 110(6):2082-7; J Vis Exp. 2013 Nov. 7; (81):e50980; and Integr Biol (Camb). 2014 April; 6(4):470-5, the contents of each of which are hereby incorporated by reference in their entireties. Additional or alternative methods, compositions, and devices can include those described in U.S. Patent Appl. Publ. No. 2014/0287509, the content of which is hereby incorporated by reference in its entirety. Generally, the protein or ribonucleoprotein complex is provided in a reaction mixture containing the cell and the reaction mixture is forced through a cell deforming orifice or constriction. In some cases, the constriction is smaller than the diameter of the cell. In some cases, the constriction contains cell-deforming components such as regions of strong electrostatic charge, regions of hydrophobicity, or regions containing nanowires or nanotubes. The forcing can introduce transient pores into a cell membrane of the cell allowing the protein or ribonucleoprotein complex to enter the cell through the transient pores. In some cases, squeezing or deforming a cell to introduce the protein or ribonucleoprotein can be effective even when the cell is in a non-dividing state.

Methods for introducing a protein or ribonucleoprotein complex into a cell include forming a reaction mixture containing the protein or ribonucleoprotein complex and contacting the cell with the protein or ribonucleoprotein complex to induce receptor-mediated internalization. Compositions and methods for receptor mediated internalization are described, e.g., in Wu et al., J. Biol. Chem. 262, 4429-4432 (1987); and Wagner et al., Proc. Natl. Acad. Sci. USA 87, 3410-3414 (1990), the contents of each of which are hereby incorporated by reference in their entireties. Generally, the receptor-mediated internalization is mediated by interaction between a cell surface receptor and a ligand fused to the protein or fused to the ribonucleoprotein complex (e.g., covalently attached or fused to an RNA in the ribonucleoprotein complex). The ligand can be any protein, small molecule, polymer, or fragment thereof that binds to, or is recognized by, a receptor on the surface of the cell. An exemplary ligand is an antibody or an antibody fragment (e.g., scFv).

In some embodiments, the reaction mixture for introducing the protein or ribonucleoprotein complex into the cell can contain a nucleic acid for directing binding to the target genomic region.

In some embodiments, delivery is via a nucleic acid (e.g., plasmid(s)) transfected into a cell. The transfected nucleic acids (e.g., plasmid(s)) can comprise an expression vector for an LSR-DBD fusion, a nucleic acid (e.g., plasmid) comprising a donor molecule for integration into the cell's genome, and an expression vector for guide polynucleotides (e.g., gRNA or sgRNA).

The nucleic acids may be delivered using adeno associated virus (AAV), lentivirus, adenovirus or other viral vector types, or combinations thereof. The nucleic acids can be packaged into virions using appropriate packaging cells lines as known in the art. In some embodiments, the LSR-DBD fusion protein and one or more exogenous nucleic acids are delivered to a cell using a lentivirus particle.

In some cases, expression of the LSR-DBD fusions described herein and/or the guide polynucleotides are under the control of an inducible promoter or repressor element. The inducible promoter or repressor element can be inserted into the promoter region of a nucleic acid sequence encoding the LSR-DBD fusions described herein and/or the guide polynucleotides to provide temporal and/or spatial control of the expression or activity.

Upon delivery of a nucleic acid encoding an LSR-DBD fusion to a cell, the nucleic acid can be transcribed and translated into an LSR-DBD protein. The LSR-DBD protein can form a tetrameric complex inside the cell. In some embodiments, the nucleic acid encoding an LSR-DBD fusion can be delivered to the cell along with a nucleic acid encoding the LSR. In some embodiments, the LSR and LSR-DBD form a tetrameric complex which can comprise one, two, or three LSR-DBD fusion proteins.

### Applications

Described herein are several applications of the LSR-DBD fusion system described herein including, but not limited to a method for amplicon library installation at genomic landing pads, delivery of cargos without a landing pad with sufficient efficiency to integrate multiple constructs in the same cell simultaneously, and direct targeting of specific sites in a mammalian genome with significantly higher efficiency than PhiC31 (which has ˜1% genome-targeted LSR integration efficiency).

Site-specific nucleases and site-specific recombinases are powerful tools for targeted genome modification in vitro and in vivo. It has been reported that nuclease cleavage in living cells triggers a DNA repair mechanism that frequently results in a modification of the cleaved and repaired genomic sequence, for example, via homologous recombination. Accordingly, the targeted cleavage of a specific unique sequence within a genome using the LSR-DBD fusions described herein opens up new avenues for gene targeting and gene modification in living cells, including cells that are hard to manipulate with conventional gene targeting methods, such as many human somatic or embryonic stem cells. Site-specific recombinases possess all the functionality required to bring about efficient, precise integration, deletion, inversion, or translocation of specified DNA segments without exposed DNA double-stranded breaks.

In some cases, the efficiency of genome-targeted integration using the LSR-DBD fusion proteins described herein can be at least about, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65% 70%, 75%, 80%, 85%, 90%, 95%, 99%, or higher. In some cases, the efficiency of incorporation of the sequence of the donor DNA can be at least, or at least about, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65% 70%, 75%, 80%, 85%, 90%, 95%, 99%, or higher.

In some embodiments, the one or more nucleic acids encoding an LSR-DBD fusion and guide polynucleotide(s) described herein are used to produce a non-human transgenic animal or transgenic plant or transgenic organoid. In some embodiments, the transgenic animal is a mammal, such as a mouse, rat, or rabbit. In certain embodiments, the organism or subject is a plant. In certain embodiments, the organism or subject or plant is algae or crops. In certain embodiments, the subject is an organoid. Methods for producing transgenic plants, organoids, and animals are known in the art, and generally begin with a method of cell transfection, such as described herein. Transgenic animals are also provided, as are transgenic plants, especially crops and algae. The transgenic animal or plant may be useful in applications outside of providing a disease model. These may include food or feed production through expression of, for instance, higher protein, carbohydrate, nutrient or vitamins levels than would normally be seen in the wildtype. In this regard, transgenic plants, especially pulses and tubers, and animals, especially mammals such as livestock (cows, sheep, goats and pigs), but also poultry and edible insects, are preferred.

Transgenic algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries.

In plants, pathogens are often host-specific. For example, Fusarium oxysporum f. sp. Lycopersici causes tomato wilt but attacks only tomato, and F. oxysporum f. dianthii Puccinia graminis f. sp. Tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a gene-for-gene level, plants and pathogens evolve together, and the genetic changes in one balance changes in other. Accordingly, using natural variability, breeders combine most useful genes for yield, quality, uniformity, hardiness, resistance. The sources of resistance genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can analyze the genome of sources of resistance genes, and in varieties having desired characteristics or traits employ the present invention to induce the rise of resistance genes, with more precision than previous mutagenic agents and hence accelerate and improve plant breeding programs.

The invention comprehends the use of the nucleic acids, polypeptides, compositions, systems, and methods disclosed herein to establish and utilize transgenic cells/animals/organoids. Disclosed herein is a non-naturally occurring or engineered composition, or one or more polynucleotides encoding components of said composition, or vector or delivery systems comprising one or more polynucleotides encoding components of said composition for use in a modifying a target cell in vivo, ex vivo or in vitro and, may be conducted in a manner alters the cell such that once modified the progeny or cell line of the modified cell retains the altered phenotype. The modified cells and progeny may be part of a multicellular organism such as a plant or animal with ex vivo or in vivo application of the LSR-DBD fusion system to desired cell types.

The invention may be a therapeutic method of treatment. The therapeutic method of treatment may comprise gene or genome editing, or gene therapy. A method of the invention may be used to create a plant, an animal or cell that may be used to model and/or study genetic or epigenetic conditions of interest, such as through a model of mutations of interest or as a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism, or cell can be a non-human subject, patient, organism or cell. Thus, the invention provides a plant, animal or cell, produced by the present methods, or a progeny thereof. The progeny may be a clone of the produced plant or animal, or may result from sexual reproduction by crossing with other individuals of the same species to introgress further desirable traits into their offspring. The cell may be in vivo or ex vivo in the cases of multicellular organisms, particularly animals or plants. In the instance where the cell is cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

To further increase effectiveness, a gene therapy vehicle can comprise one or more immunosuppressant agents. “Immunosuppressant agent” in this context encompasses any compound which suppresses an immune response. Particularly preferred immunosuppressing drugs are cyclosporine, cyclophosphamide, anti-lymphocyte antibodies (e.g. anti CD20) or anti-cytokine antibodies (e.g. anti-TNF-alpha).

In a preferred embodiment, the gene therapy vehicle according to the invention can also be used in conjunction with another therapeutic reagent. An effective amount of a pharmaceutical composition according to the invention is administered, optionally in combination with another therapeutic treatment or agent, such as an immunosuppressing drug.

In a further embodiment, the present invention provides an ex vivo method for transfecting the LSR-DBD system described herein in relevant host cells (e.g. stem cells). In one embodiment, suitable cells are isolated from the mammal, eventually differentiated in vitro and incubated with an effective amount of a pharmaceutical composition of the present invention. Thereafter, the treated (transfected) cells are re-introduced into the organism.

The gene therapy composition of the invention comprises, in addition to adequate salts (alkali metal as counter ion and dications in formulation) and eventually other therapeutic or immunosuppressive agents, a pharmaceutically acceptable carrier and/or a pharmaceutically acceptable vehicle and/or pharmaceutically acceptable diluent. Appropriate routes for suitable formulation and preparation of the gene therapy vehicle according to the invention are disclosed in Remington: “The Science and Practice of Pharmacy,” 20^{th} Edn., A. R. Gennaro, Editor, Mack Publishing Co., Easton, Pa. (2003), the content of which is hereby incorporated by reference in its entirety. Possible carrier substances for parenteral administration are e.g. sterile water, Ringer, Ringer lactate, sterile sodium chloride solution, polyalkylene glycols, hydrogenated naphthalenes and, in particular, biocompatible lactide polymers, lactide/glycolide copolymers or polyoxyethylene/polyoxy-propylene copolymers. The particular embodiments of the gene therapy formulation are chosen according to the physical properties, for example in respect of solubility, stability, bioavailability or degradability.

Controlled or constant release of the active drug (-like) components according to the invention includes formulations based on lipophilic depots (e.g. fatty acids, waxes or oils). In the context of the present invention, coatings of vaccine substances according to the invention, namely coatings with polymers, are also disclosed (e.g. polyoxamers or polyoxamines). The gene therapy substances or compositions according to the invention can furthermore have protective coatings, e.g. protease inhibitors or permeability intensifiers. Preferred carriers are typically aqueous carrier materials, water for injection (WFI) or water buffered with phosphate, citrate, HEPES or acetate, or Ringer or Ringer Lactate etc. being used, and the pH is typically adjusted to 5.0 to 8.0, preferably 6.5 to 7.5. The carrier or the vehicle will additionally preferably comprise salt constituents, e.g. sodium chloride, potassium chloride or other components which render the solution e.g. isotonic. Furthermore, the carrier or the vehicle can contain, in addition to the abovementioned constituents, additional components, such as human serum albumin (HSA), polysorbate 80, sugars or amino acids.

The mode and method of administration and the dosage of the gene therapy according to the invention depend on the nature of the disease to be treated, where appropriate the stage thereof, and also the body weight, the age and the sex of the patient.

The gene therapy of the present invention may preferably be administered to the patient parenterally, e.g. intravenously, intraarterially, subcutaneously, intradermally, intra-lymph node or intramuscularly. It is also possible to administer the gene therapy topically or orally or intra-nasal. A further injection possibility is into a tumor tissue or tumor cavity (after the tumor is removed by surgery, e.g. in the case of brain tumors).

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of the LSR-DBD fusion system of the present invention; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the LSR-DBD fusion of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

A transgenic cell in which one or more nucleic acids encoding one or more of the components of the present invention are provided or introduced can be operably connected in the cell with a regulatory element comprising a promoter of one or more gene of interest. As used herein, the term “LSR-DBD fusion transgenic cell” refers to a cell, such as a eukaryotic cell, in which an LSR-DBD fusion has been genomically integrated. The nature, type, or origin of the cell are not particularly limiting according to the present invention. Also the way in which the LSR-DBD fusion transgene is introduced in the cell may vary and can be any method as is known in the art. In certain embodiments, the LSR-DBD fusion transgenic cell is obtained by introducing the LSR-DBD fusion transgene in an isolated cell. In certain other embodiments, the LSR-DBD fusion transgenic cell is obtained by isolating cells from an LSR-DBD fusion transgenic organism. By means of example, and without limitation, the LSR-DBD fusion transgenic cell as referred to herein may be derived from an LSR-DBD fusion transgenic eukaryote, such as an LSR-DBD fusion knock-in eukaryote. Reference is made to WO 2014/093622 (PCT/US 13/74667), the contents of which is hereby incorporated by reference in its entirety. Methods of U.S. Pat. Nos. 8,771,985 and 9,567,573 assigned to Sangamo Biosciences, Inc. (and the contents of each of which are hereby incorporated by reference in their entireties) directed to targeting the Rosa locus may be modified to utilize the LSR-DBD fusion system of the present invention. Methods of US Patent Publication No. 20130236946 assigned to Cellectis (the contents of which is hereby incorporated by reference in its entirety) directed to targeting the Rosa locus may also be modified to utilize the LSR-DBD fusion system of the present invention. The LSR-DBD fusion transgene can further comprise a Lox-Stop-poly A-Lox (LSL) cassette thereby rendering LSR-DBD fusion expression inducible by Cre recombinase. Alternatively, the LSR-DBD fusion transgenic cell may be obtained by introducing the LSR-DBD fusion transgene in an isolated cell. Delivery systems for transgenes are well known in the art. By means of example, the LSR-DBD fusion protein transgene may be delivered in for instance eukaryotic cell by means of vector (e.g., AAV, adenovirus, lentivirus) and/or particle and/or nanoparticle delivery, as also described herein elsewhere.

In certain aspects, described herein is a cell comprising a nucleic acid encoding any of the LSR-DBD fusions disclosed herein. In some embodiments, the genome of the cell comprises an attachment site for the LSR portion of the LSR-DBD fusion. Such a cell line can be used in a method wherein a nucleic acid comprising a donor attachment site and a nucleic acid for insertion is introduced into the cell to generate an engineered cell line comprising the nucleic acid of interest inserted into the LSR attachment site. In some embodiments, described herein is a kit comprising a cell, the cell comprising a nucleic acid encoding any of the LSR-DBD fusions disclosed herein. In some embodiments, the genome of the cell of the kit comprises an attachment site for the LSR portion of the LSR-DBD fusion. In some embodiments, the kit further comprises a nucleic acid vector (e.g. plasmid) comprising a donor attachment site. In some embodiments, the nucleic acid vector (e.g. plasmid) of the kit further comprises a multicloning site for insertion of a nucleic acid of interest. In some embodiments the cell is a human cell. In some embodiments, the cell is a human embryonic stem cell. In some embodiments, the cells is a H1 human embryonic stem cell. In some embodiments, the cell is a human cancer cell. In some embodiment, the cell is a human cancer cell line. In some embodiments, the cell is a human liver cancer cell line. In some embodiments, the cell is a hepatocellular carcinoma cell line. In some embodiments, the cell line is HepG2 hepatocellular carcinoma cell line. In some embodiments, the cell is a HEK cell.

Several further aspects of the invention relate to modeling defects associated with a wide range of genetic diseases in plants or animals, which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

In some embodiments, the condition may be neoplasia. In some embodiments, the condition may be Age-related Macular Degeneration. In some embodiments, the condition may be a Schizophrenic Disorder. In some embodiments, the condition may be a Trinucleotide Repeat Disorder. In some embodiments, the condition may be Fragile X Syndrome. In some embodiments, the condition may be a Secretase Related Disorder. In some embodiments, the condition may be a Prion-related disorder. In some embodiments, the condition may be ALS. In some embodiments, the condition may be a drug addiction. In some embodiments, the condition may be Autism. In some embodiments, the condition may be Alzheimer's Disease. In some embodiments, the condition may be inflammation. In some embodiments, the condition may be Parkinson's Disease.

Examples of proteins associated with Parkinson's disease include but are not limited to α-synuclein, DJ-1, LRRK2, PINK1, Parkin, UCHL1, Synphilin-1, and NURR1.

Examples of addiction-related proteins may include ABAT.

Examples of inflammation-related proteins may include the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, or the Fc epsilon R1g (FCER1g) protein encoded by the Fcerlg gene.

Examples of cardiovascular diseases associated proteins may include IL1B (interleukin 1, beta), XDH (xanthine dehydrogenase), TP53 (tumor protein p53), PTGIS (prostaglandin I2 (prostacyclin) synthase), MB (myoglobin), IL4 (interleukin 4), ANGPT1 (angiopoietin 1), ABCG8 (ATP-binding cassette, sub-family G (WHITE), member 8), or CTSK (cathepsin K), for example.

Examples of Alzheimer's disease associated proteins may include the very low density lipoprotein receptor protein (VLDLR) encoded by the VLDLR gene, the ubiquitin-like modifier activating enzyme 1 (UBA1) encoded by the UBA1 gene, or the NEDD8-activating enzyme E1 catalytic subunit protein (UBE1C) encoded by the UBA3 gene.

Examples of proteins associated Autism Spectrum Disorder may include the benzodiazapine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, or the fragile X mental retardation autosomal homolog 2 protein (FXR2) encoded by the FXR2 gene.

Examples of proteins associated Macular Degeneration may include the ATP-binding cassette, sub-family A (ABC1) member 4 protein (ABCA4) encoded by the ABCR gene, the apolipoprotein E protein (APOE) encoded by the APOE gene, or the chemokine (C-C motif) Ligand 2 protein (CCL2) encoded by the CCL2 gene.

Examples of proteins associated Schizophrenia may include NRG1, ErbB4, CPLX1, TPH1, TPH2, NRXN1, GSK3A, BDNF, DISC1, GSK3B, and combinations thereof.

Examples of proteins involved in tumor suppression may include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4.

Examples of proteins associated with a secretase disorder may include PSENEN (presenilin enhancer 2 homolog (C. elegans)), CTSB (cathepsin B), PSEN1 (presenilin 1), APP (amyloid beta (A4) precursor protein), APHIB (anterior pharynx defective 1 homolog B (C. elegans)), PSEN2 (presenilin 2 (Alzheimer disease 4)), or BACE1 (beta-site APP-cleaving enzyme 1).

Examples of proteins associated with Amyotrophic Lateral Sclerosis may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins associated with prion diseases may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins related to neurodegenerative conditions in prion disorders may include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), or ADRAID (Alpha-1D adrenergic receptor for Alpha-1D adrenoreceptor).

Examples of proteins associated with Immunodeficiency may include A2M [alpha-2-macroglobulin]; AANAT [arylalkylamine N-acetyltransferase]; ABCA1 [ATP-binding cassette, sub-family A (ABC1), member 1]; ABCA2 [ATP-binding cassette, sub-family A (ABC1), member 2]; or ABCA3 [ATP-binding cassette, sub-family A (ABC1), member 3]; for example.

Examples of proteins associated with Trinucleotide Repeat Disorders include AR (androgen receptor), FMR1 (fragile X mental retardation 1), HTT (huntingtin), or DMPK (dystrophia myotonica-protein kinase), FXN (frataxin), ATXN2 (ataxin 2).

Examples of proteins associated with Neurotransmission Disorders include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), or HTR2c (5-hydroxytryptamine (serotonin) receptor 2C).

Examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], or ABCA13 [ATP-binding cassette, sub-family A (ABC1), member 13].

Further examples of preferred conditions treatable with the present system may be selected from: Aicardi-Goutières Syndrome; Alexander Disease; Allan-Herndon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease—Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum. As will be apparent, it is envisaged that the present system can be used to target any polynucleotide sequence of interest.

The nucleic acids, polypeptides, compositions, systems, and methods disclosed herein can be used to introduce nucleic acid sequences encoding chimeric antigen receptors into cells. Chimeric antigen receptor molecules are recombinant and are distinguished by their ability to both bind antigen and transduce activation signals via immunoreceptor activation motifs (ITAM's) present in their cytoplasmic tails. Receptor constructs utilizing an antigen-binding moiety (for example, generated from single chain antibodies (scFv)) afford the additional advantage of being “universal” in that they bind native antigen on the target cell surface in an HLA-independent fashion. In some embodiments, the chimeric antigen receptor comprises: a) an intracellular signaling domain, b) a transmembrane domain, and c) an extracellular domain comprising an antigen binding region.

In specific embodiments, intracellular receptor signaling domains in the CAR include those of the T cell antigen receptor complex, such as the zeta chain of CD3, also Fcy RIII costimulatory signaling domains, CD28, CD27, DAP10, CD137, OX40, CD2, alone or in a series with CD3zeta, for example. In specific embodiments, the intracellular domain (which may be referred to as the cytoplasmic domain) comprises part or all of one or more of TCR zeta chain, CD28, CD27, OX40/CD134, 4-1BB/CD137, FcsRIy, ICOS/CD278, IL-2Rbeta/CD122, IL-2Ralpha/CD 132, DAP 10, DAP 12, and CD40. In some embodiments, one employs any part of the endogenous T cell receptor complex in the intracellular domain. One or multiple cytoplasmic domains may be employed, as so-called third generation CARs have at least two or three signaling domains fused together for additive or synergistic effect, for example.

For example, the donor DNA can be used to replace one or more complementary determining regions, or portions thereof, of a T cell receptor chain or antibody gene. Such a donor DNA can thus alter the antigen specificity of a target cell. For instance, the target cell can be altered to recognize, and thereby elicit an immune response against, a tumor antigen or an infectious disease antigen.

In certain embodiments of the invention, the CAR cells are delivered to an individual in need thereof, such as an individual that has cancer or an infection. The cells then enhance the individual's immune system to attack the respective cancer or pathogenic cells. In some cases, the individual is provided with one or more doses of the antigen-specific CAR T-cells. In cases where the individual is provided with two or more doses of the antigen-specific CAR T-cells, the duration between the administrations should be sufficient to allow time for propagation in the individual, and in specific embodiments the duration between doses is 1, 2, 3, 4, 5, 6, 7, or more days.

The source of the allogeneic T cells that are modified to both include a chimeric antigen receptor and that lack functional TCR may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

A composition of the present invention can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present invention, alone or in combination with other active agents, calculated in an amount sufficient to produce the desired effect, in association with a pharmaceutically acceptable diluent, carrier, or vehicle, where appropriate. The specifications for the novel unit dosage forms of the present invention depend on the particular pharmacodynamics associated with the pharmaceutical composition in the particular subject.

Accordingly, the amount of transduced T cells administered should take into account the route of administration and should be such that a sufficient number of the transduced T cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount can be utilized either above, e.g., greater than 5×10⁸ cells, or below, e.g., less than 1×10⁷ cells. The dosing schedule can be based on well-established cell-based therapies (see, e.g., Topalian and Rosenberg, 1987; U.S. Pat. No. 4,690,915, the contents of each of which are hereby incorporated by reference in their entireties), or an alternate continuous infusion strategy can be employed.

These values provide general guidance of the range of transduced T cells to be utilized by the practitioner upon optimizing the method of the present invention for practice of the invention. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

In some embodiments, the donor DNA encodes a recombinant antigen receptor, a portion thereof, or a component thereof. Recombinant antigen receptors, portions, and components thereof include those described in U.S. Patent Appl. Publ. Nos. 2003/0215427; 2004/0043401; 2007/0166327; 2012/0148552; 2014/0242701; 2014/0274909; 20140314795; 2015/0031624; and International Appl. Publ. Nos.: WO/2000/023573; and WO/2014/134165, the contents of each of which are hereby incorporated by reference in their entireties. Such recombinant antigen receptors can be used for immunotherapy targeting a specific tumor associated or infectious disease associated antigen. In some cases, the methods described herein can be used to knockout an endogenous antigen receptor, such as a T cell receptor, B cell receptor, or a portion, or component thereof. The methods described herein can also be used to knock-in a recombinant antigen receptor, a portion thereof, or a component thereof. In some embodiments, the endogenous receptor is knocked out and replaced with the recombinant receptor (e.g., a recombinant T cell Receptor or a recombinant chimeric antigen receptor). In some cases, the recombinant receptor is inserted into the genomic location of the endogenous receptor. In some cases, the recombinant receptor is inserted into a different genomic location as compared to the endogenous receptor.

As another example, the donor DNA can encode a suicide gene, a reporter gene, or a rheostat gene, or a portion thereof. A suicide gene can be used to remove antigen specific immunotherapy cells from a host after successful treatment. A rheostat gene can be used to modulate the activity of an immune response during immunotherapy. A reporter gene can be used to monitor the number, location, and activity of cells in vitro or in vivo after introduction into a host. In preferred embodiments, the donor DNA contains an attD site capable of site-specifically integrating the donor DNA into cellular DNA.

Exemplary rheostat genes are immune checkpoint genes. An increase or decrease in expression or activity of one or more immune checkpoint genes can be used to modulate the activity of an immune response during immunotherapy. For example, an immune checkpoint gene can be increased in expression resulting in a decreased immune response. Alternatively, the immune checkpoint gene can be inactivated, resulting in an increased immune response. Exemplary immune checkpoint genes include, but are not limited to, CTLA-4, and PD-1. Additional rheostat genes can include any gene that modulates proliferation or effector function of the target cell. Such rheostat genes include transcription factors, chemokine receptors, cytokine receptors, or genes involved in co-inhibitory pathways such as TIGIT or TIMs. In some cases the rheostat gene is a synthetic or recombinant rheostat gene that interacts with the cell signaling machinery. For example, the synthetic rheostat gene can be a drug-dependent or light-dependent molecule that inhibits or activates cell signaling. Such synthetic genes are described in, e.g., Cell 155(6):1422-34 (2013); and Proc Natl Acad Sci USA. 2014 Apr. 22; 111(16):5896-901, the contents of each of which are hereby incorporated by reference in their entireties.

Exemplary suicide genes include, but are not limited to, thymidine kinase, herpes simplex virus type 1 thymidine kinase (HSV-tk), cytochrome P450 isoenzyme 4B1 (cyp4B1), cytosine deaminase, human folylpolyglutamate synthase (fpgs), or inducible casp9. In some embodiments, the suicide gene is chosen from the group consisting of the gene encoding the HSV-1 thymidine kinase (abbreviated to HSV-tk), the splice-corrected HSV-tk (abbreviated to cHSV-tk, see Fehse B et al., Gene Ther (2002) 9(23): 1633-1638), the genes coding for the highly Gancyclovir-sensitive HSV-tk mutants (mutants wherein the residue at position 75 and/or the residue at position 39 are mutated (see Black Me. Et al. Cancer Res (2001) 61(7):3022-3026; and Qasim W et al., Gene Ther (2002) 9(12):824-827), the contents of each of which are hereby incorporated by reference in their entireties. Suicide genes other than thymidine kinase based gene can be used instead. For instance, genes coding for human CD20 (the target of clinical-grade monoclonal antibodies such as Rituximab®; see Serafini M et al., Hum Gene Ther. 2004; 15:63-76), inducible caspases (as an example: modified human caspase 9 fused to a human FK506 binding protein (FKBP) to allow conditional dimerization using a small molecule pharmaceutical; see Di Stasi A et al., N Engl J Med. 2011 Nov. 3; 365(18): 1673-83; Tey S K et al., Biol Blood Marrow Transplant. 2007 August) '3(8):9) '3-24. Epub 2007 May 29) and FCU1 (that transforms a non-toxic prodrug 5-fluorocytosine or 5-FC to its highly cytotoxic derivatives 5-fluorouracil or 5-FU and 5′-fluorouridine-5′monophosphate or 5′-FUMP; Breton E et al., C R Biol. 2010 March; 333 (3): 220-5. Epub 2010 Jan. 25) can be used as suicide gene, the contents of each of which are hereby incorporated by reference in their entireties.

### SEQUENCES

FIG. 33 discloses the amino acid (SEQ ID NOs: 1-5) and corresponding nucleotide sequences (SEQ ID NOs: 6-10) for exemplary LSRs (Dn29, Pf80, Cp36, Nm60, Si74) for use in the LSR-DBD fusions described herein.

Additional LSRs for use in the LSR-DBD fusions include the list of experimentally characterized large serine recombinases as described in Supplemental Table 2 of Durrant, M. G., Fanton, A., Tycko, J. et al. Systematic discovery of recombinases for efficient integration of large DNA sequences into the human genome, Nat Biotechnol 41, 488-499 (2023), the content of which is hereby incorporated by reference in its entirety. Any of these LSRs (Bc30, Bm99, Bs46, Bt24, Bu30, Bxb1, Cb16, Cc91, Cd04, Cd15, Cd16, Cd31, Cp36, Cs56, Ct03, Dn29, Ec03, Ec04, Ec05, Ec06, Ec07, Ef01, Ef02, Efs2, Em12, Enc3, Enc9, Fm04, Fp10, Kp01, Kp03, Kp04, Kp05, Ma05, Ma37, Me99, Nm60, No67, Pa01, Pa03, Pc01, Pc64, Pf13, Pf15, Pf48, Pf80, Ph43, PhiC31, Pp20, Ps40, Ps45, Rb27, Rh64, R109, Sa01, Sa02, Sa10, Sa34, Sa51, Se37, Sh25, Si74, Sm18, Sp56, Td01, Td08, uCb4, Vh19, Vh73, Vp82) can be used in the LSR-DBD fusions described herein. The amino acid sequences of these LSRs are provided as SEQ ID NOs: 432-501, respectively, of the sequence listing accompanying this application. The cognate attP attachment site for these LSRs are provided as SEQ ID NOs: 292-361, respectively, of the sequence listing accompanying this application. The cognate attB attachment site for these LSRs are provided as SEQ ID NOs: 362-431, respectively, of the sequence listing accompanying this application.

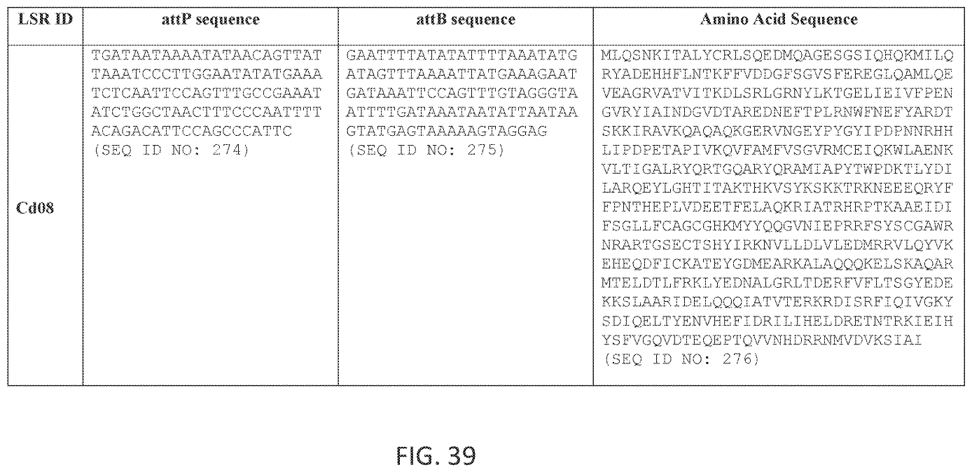

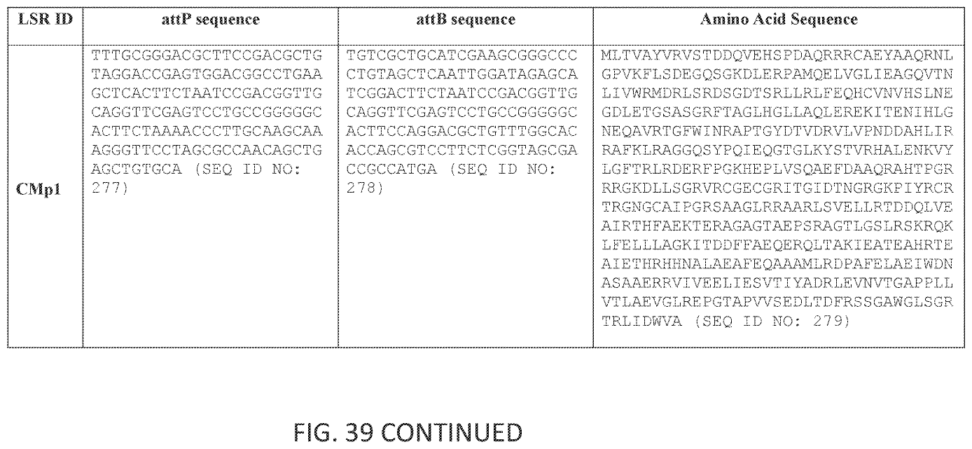

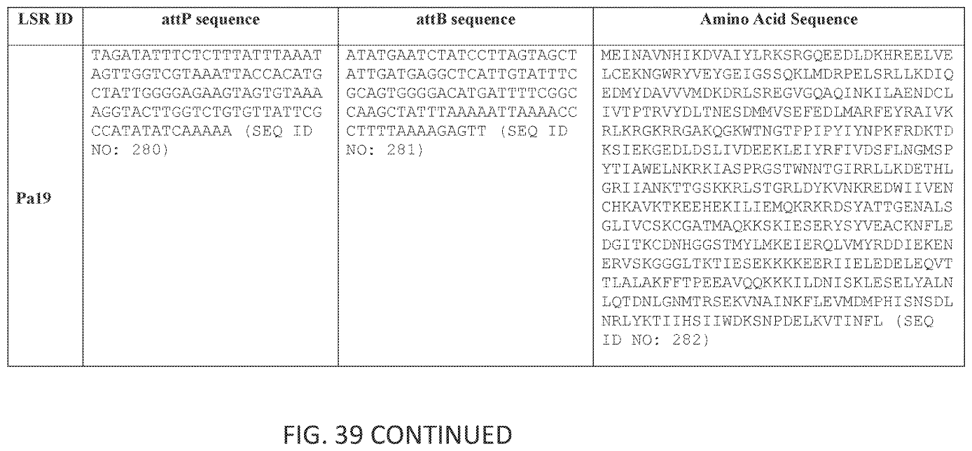

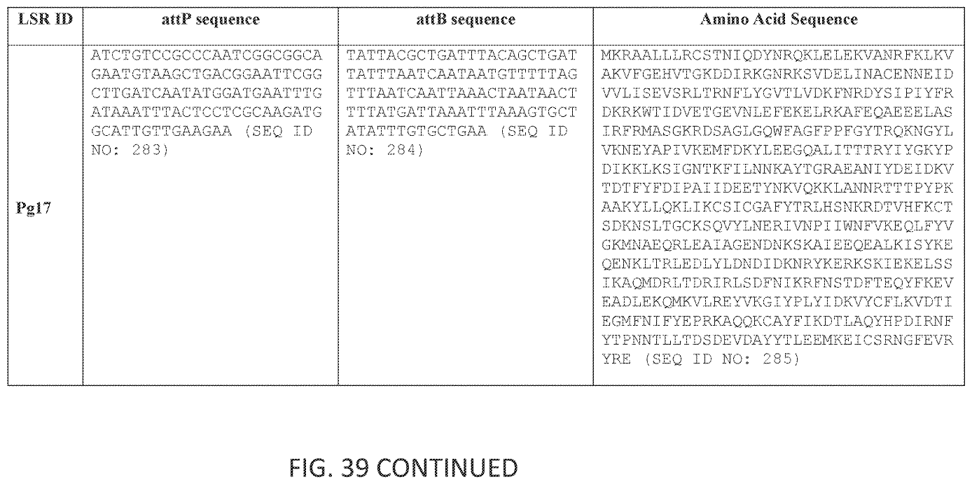

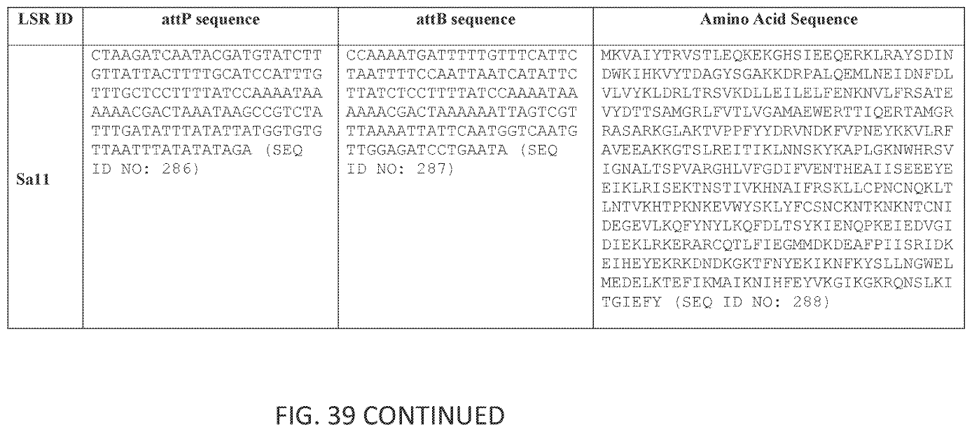

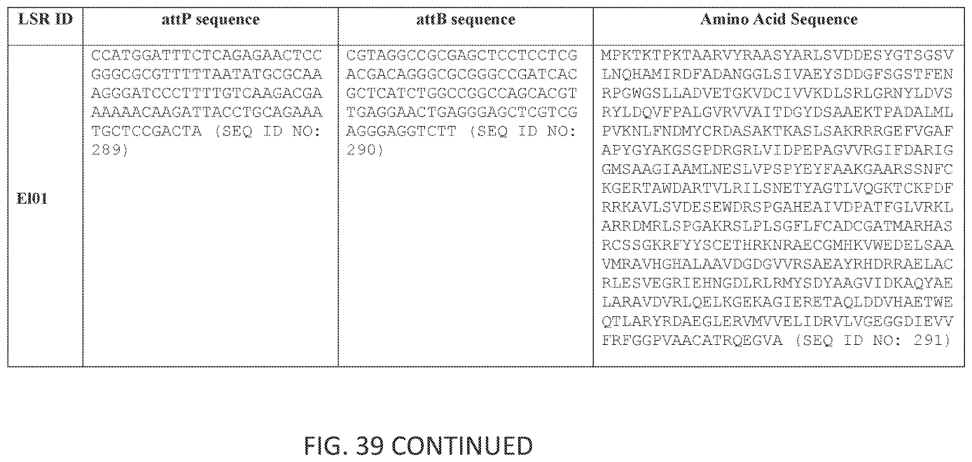

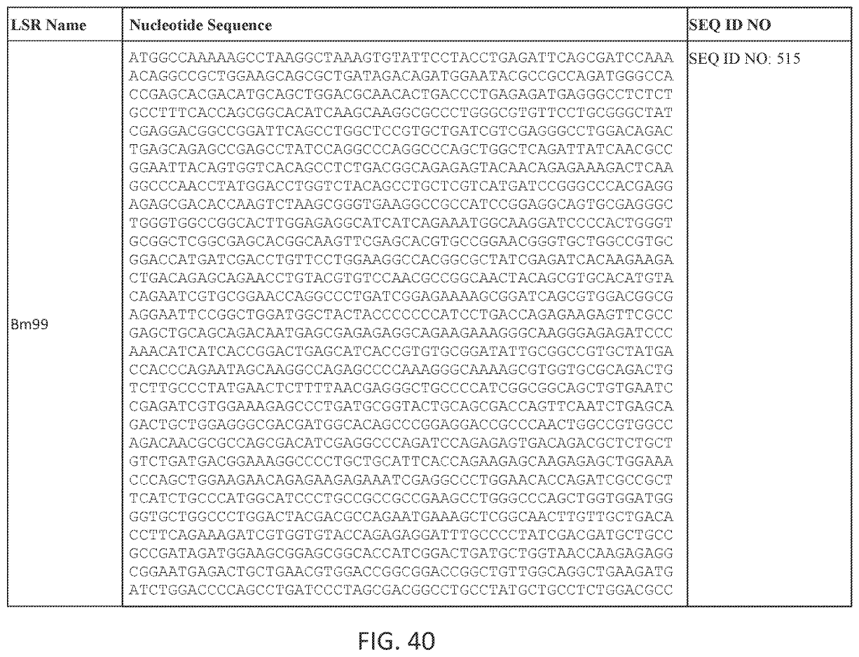

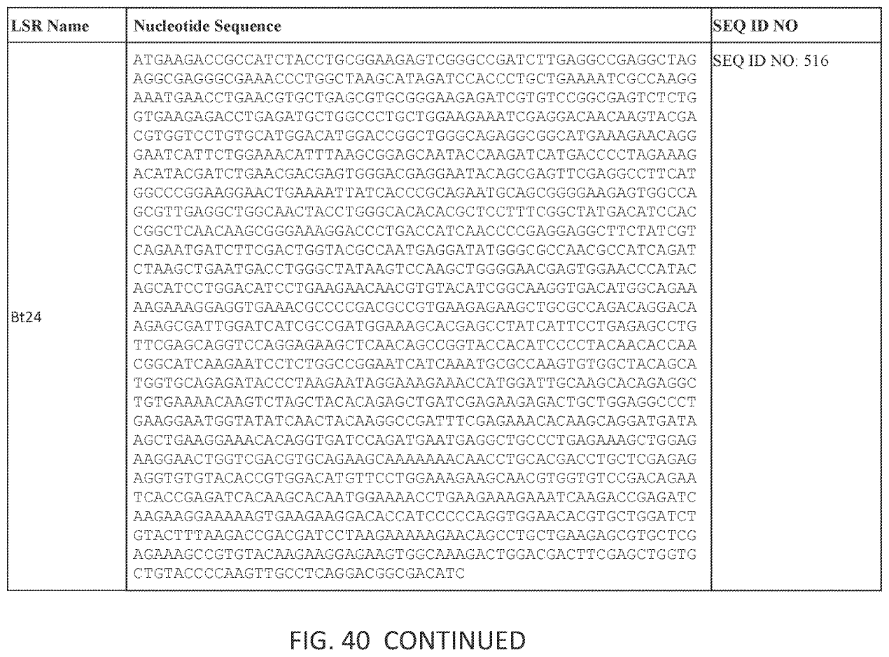

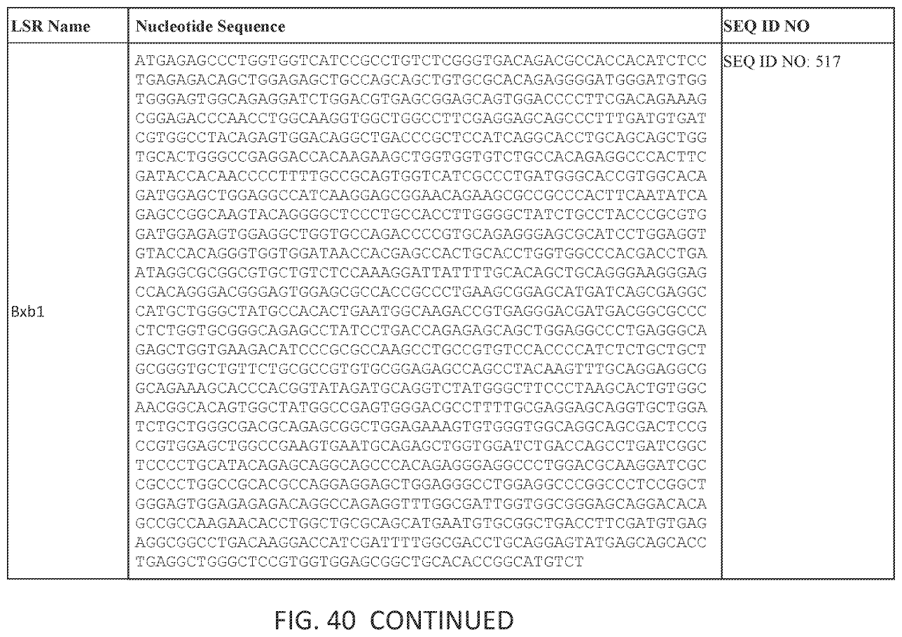

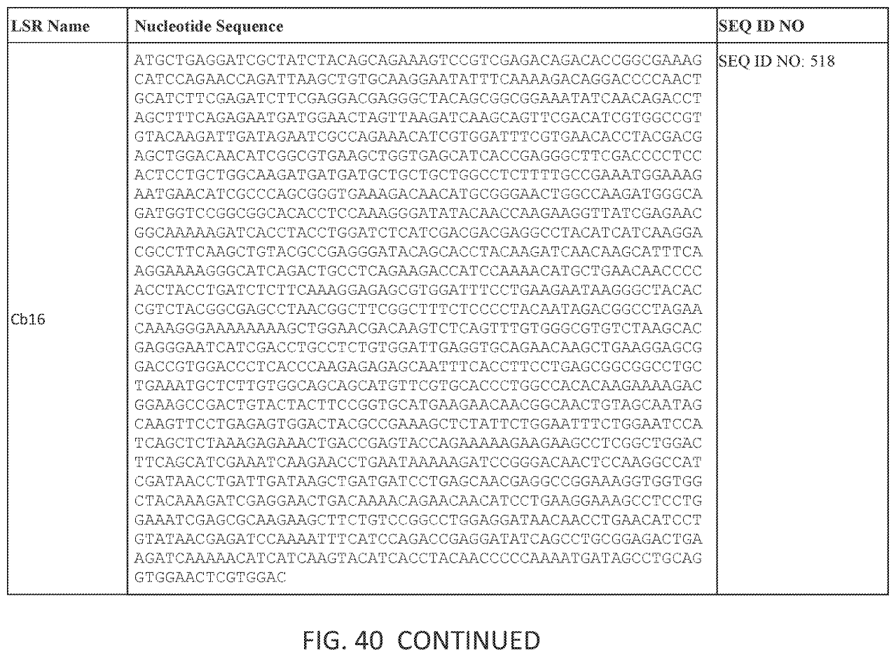

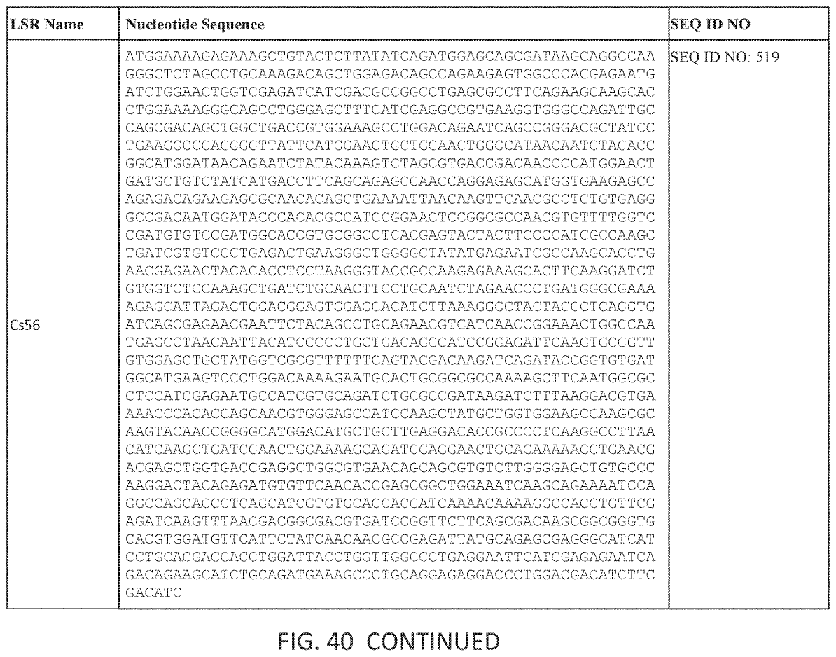

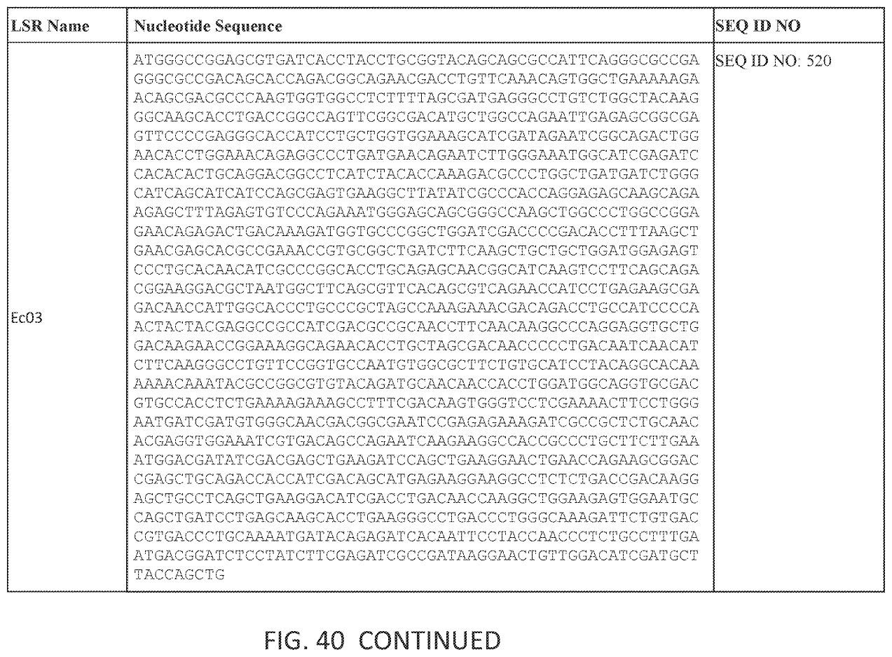

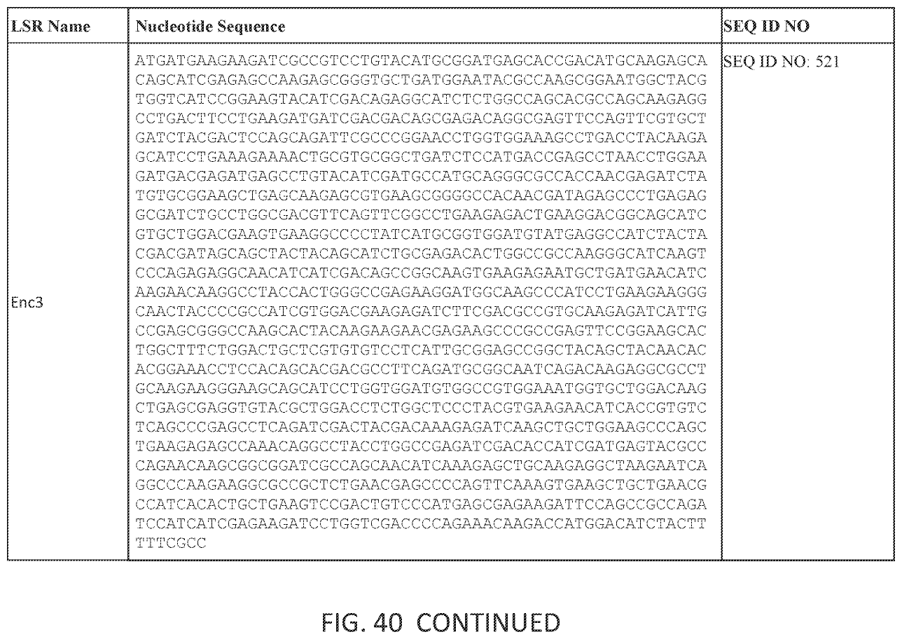

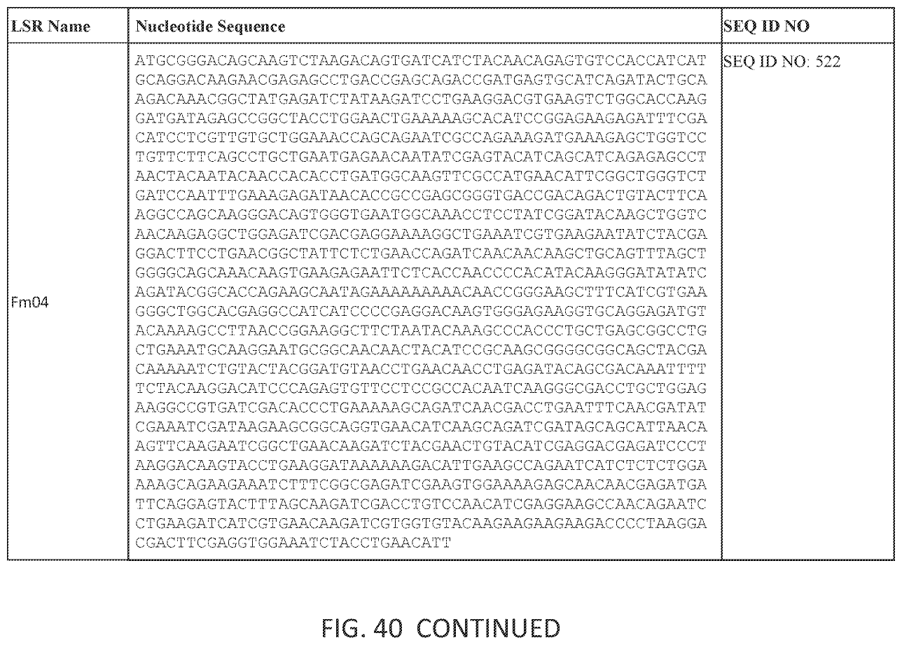

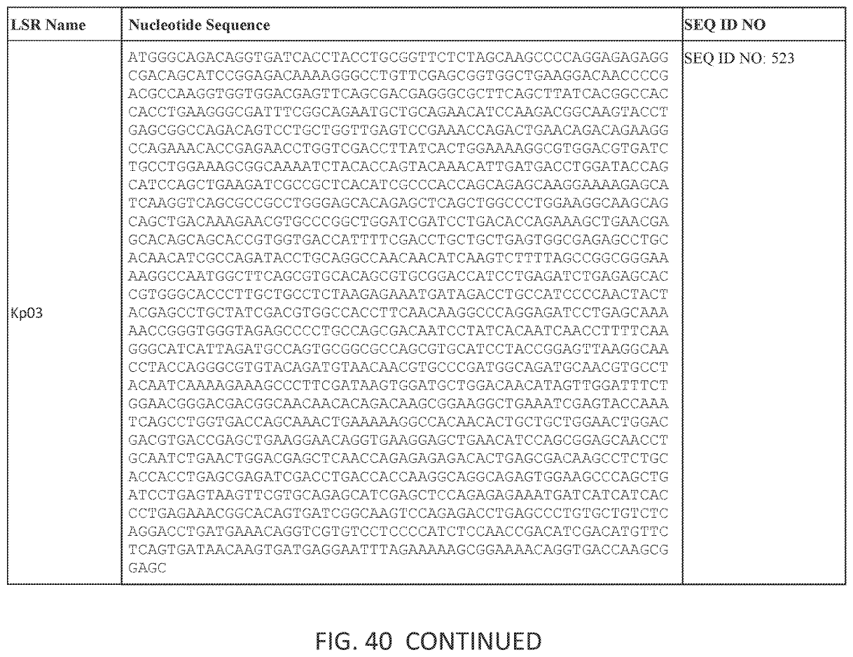

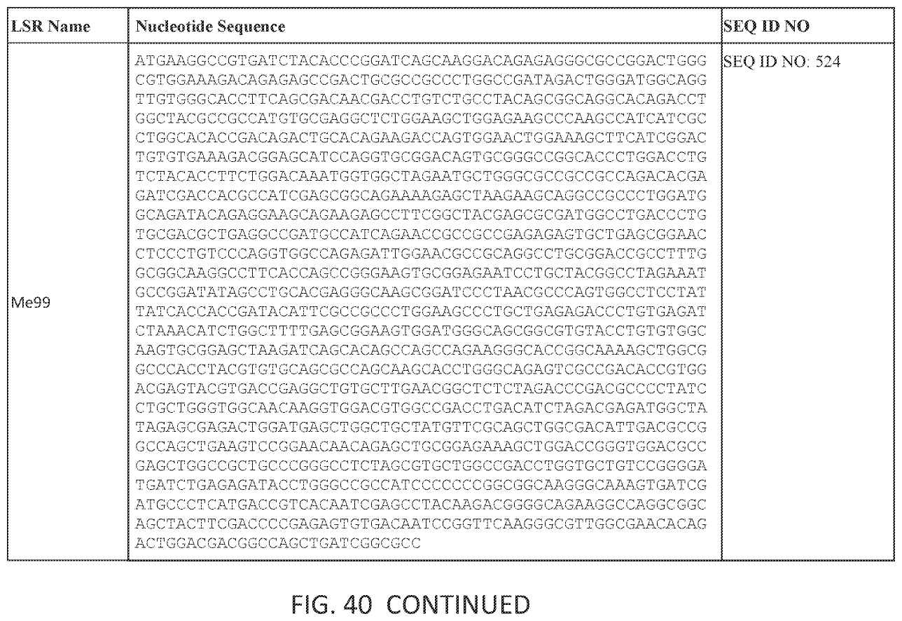

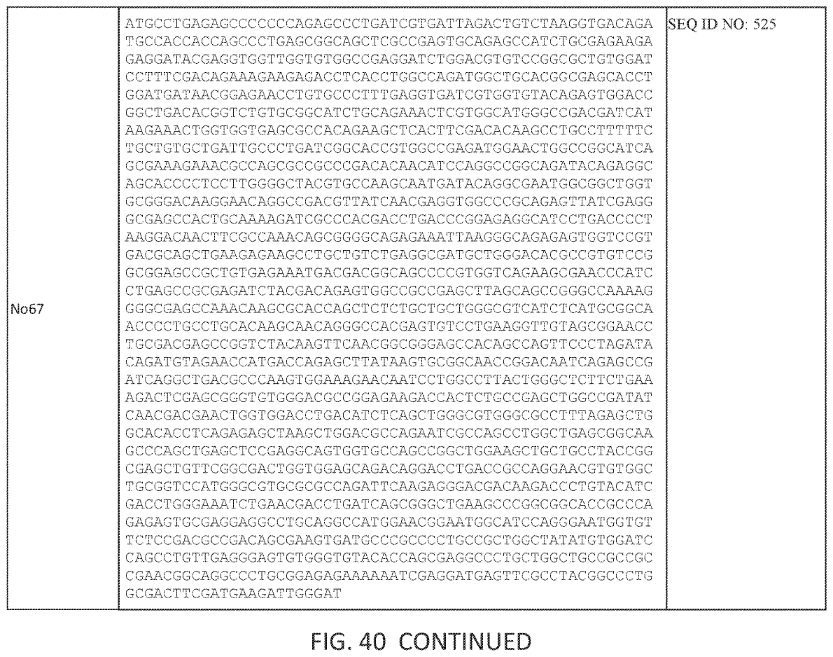

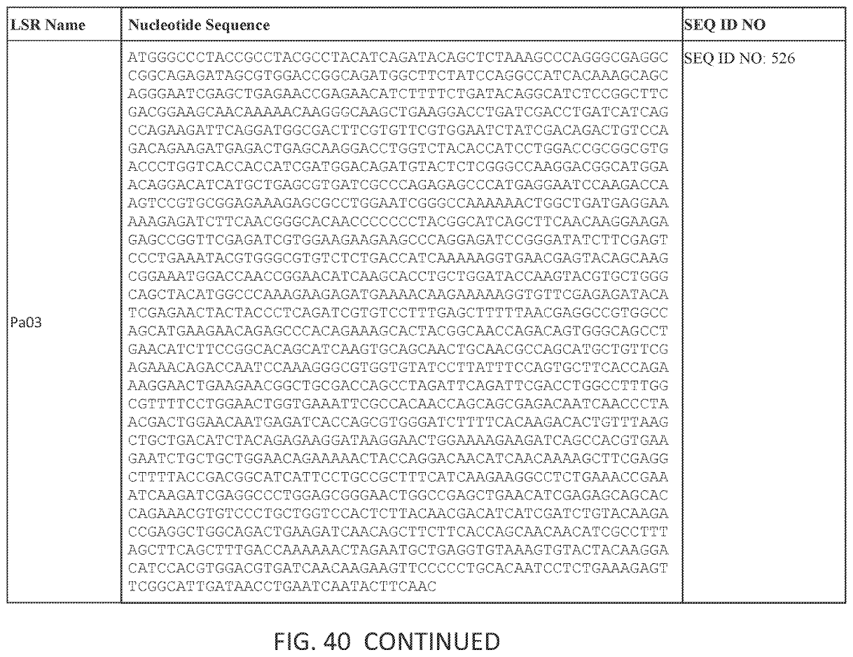

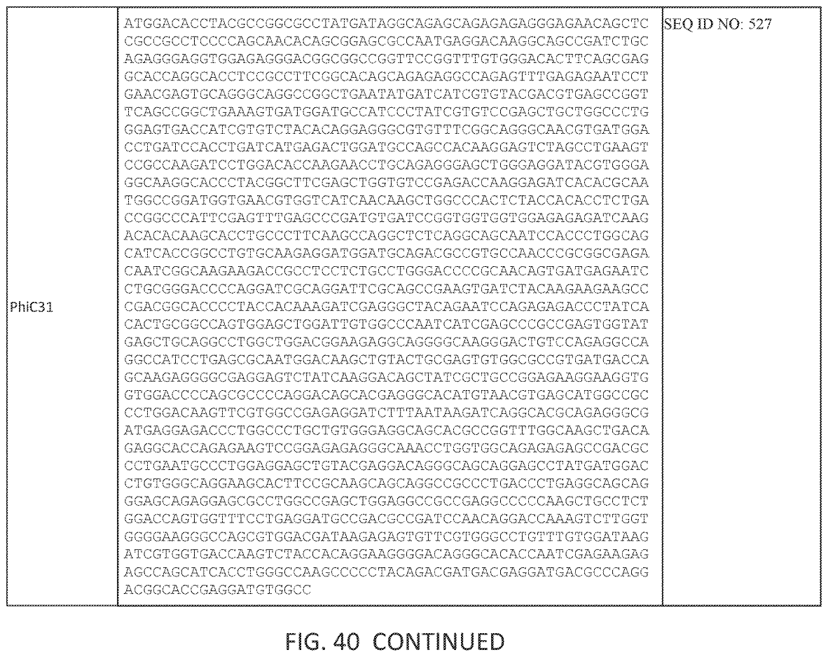

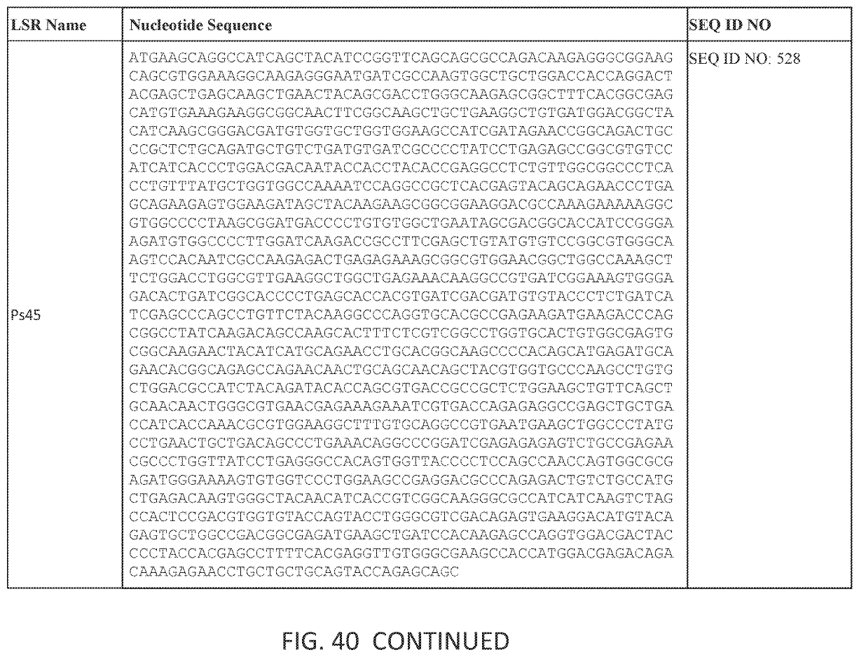

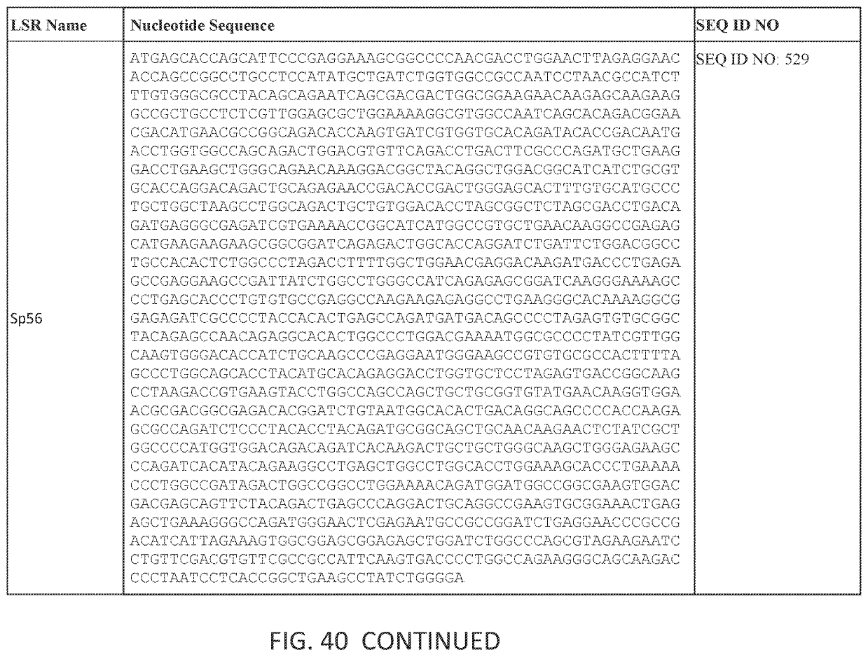

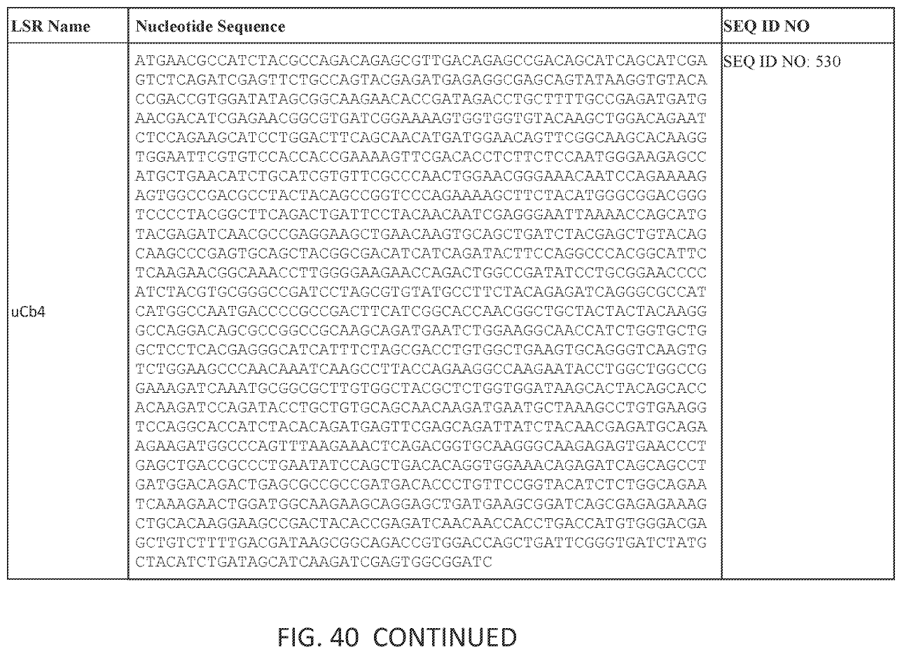

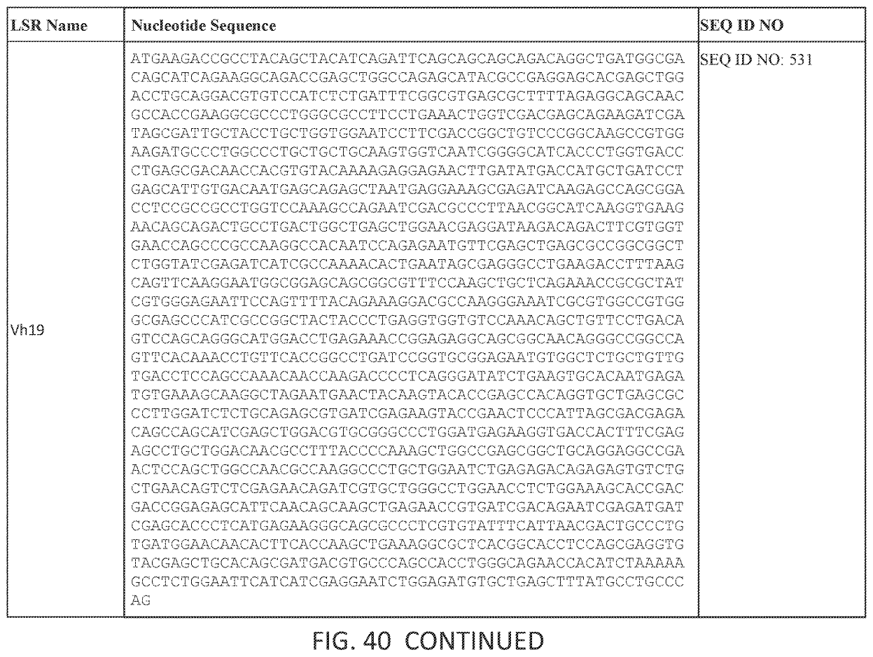

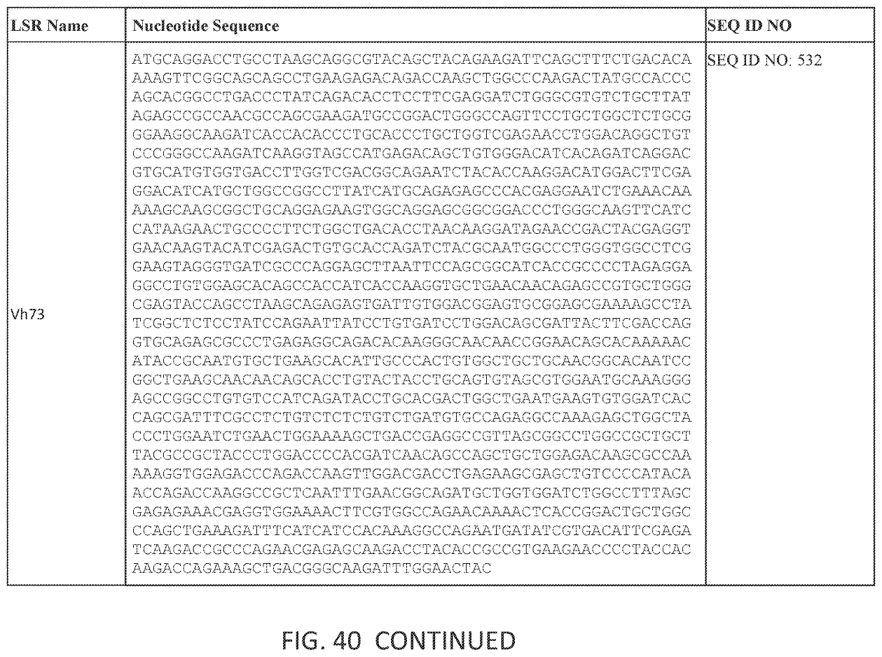

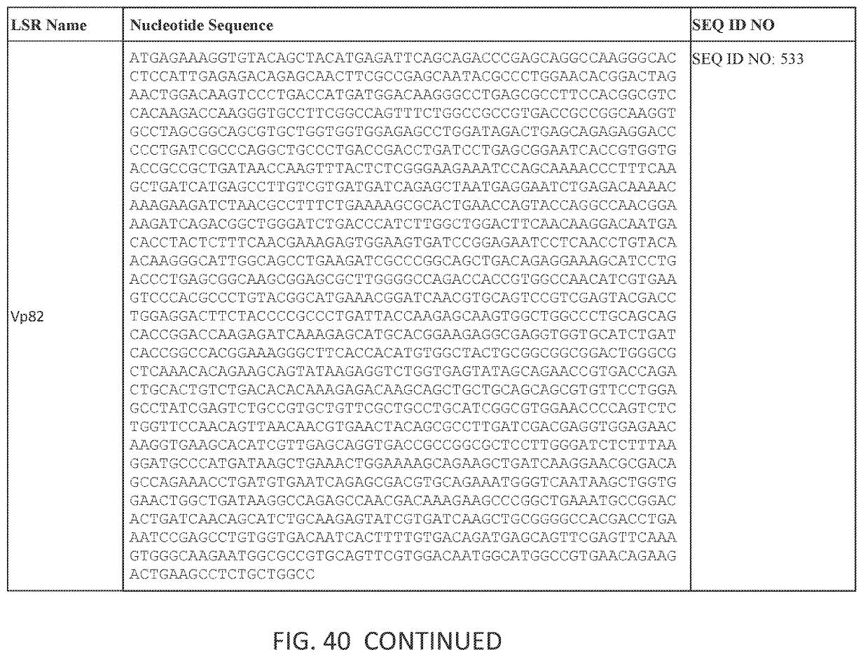

FIG. 39 discloses the amino acid (SEQ ID NOs: 276, 279, 282, 285, 288, and 291) and cognate attP attachment site (SEQ ID NOs: 274, 277, 280, 283, 286, and 289) cognate attB attachment site (SEQ ID NOs: 275, 278, 281, 284, 287, 290) for exemplary LSRs (Cd08, CMp1, E101, Pa19, Pg17, Sal1), respectively, for use in the LSR-DBD fusions described herein. FIG. 40 discloses the nucleic acid sequences (SEQ ID NOs: 515-533) for exemplary LSRs (Bm99, Bt24, Bxb1, Cb16, Cs56, Ec03, Enc3, Fm04, Kp03, Me99, No67, Pa03, PhiC31, Ps45, Sp56, uCb4, Vh19, Vh73, or Vp82) for use in the LSR-DBD fusions described herein.

FIG. 34 discloses amino acid (SEQ ID NOs: 11-19) and corresponding nucleotide sequences (SEQ ID NOs: 20-28) for exemplary linkers for use in the LSR-DBD fusions described herein.

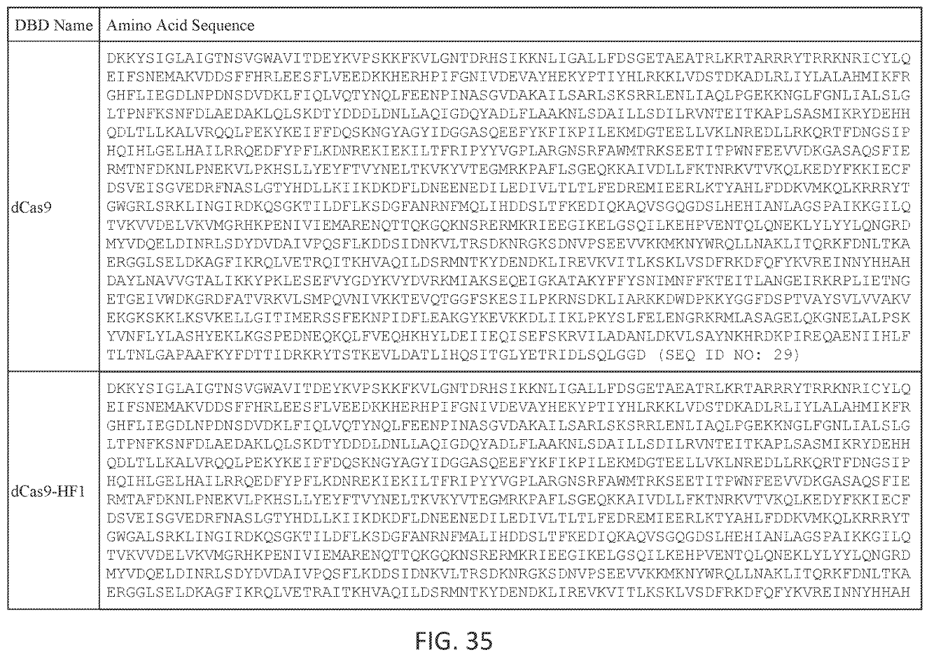

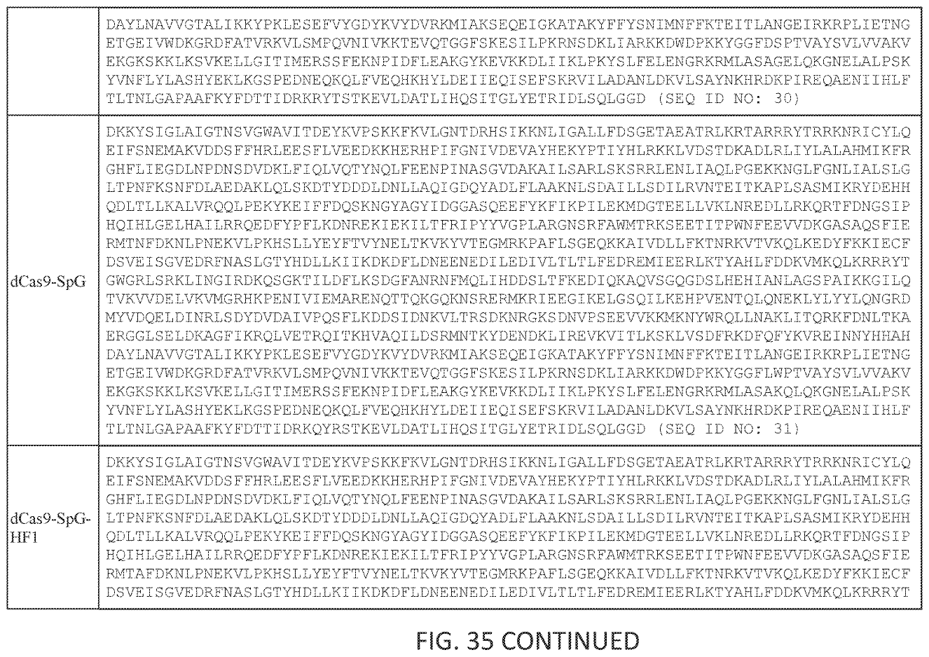

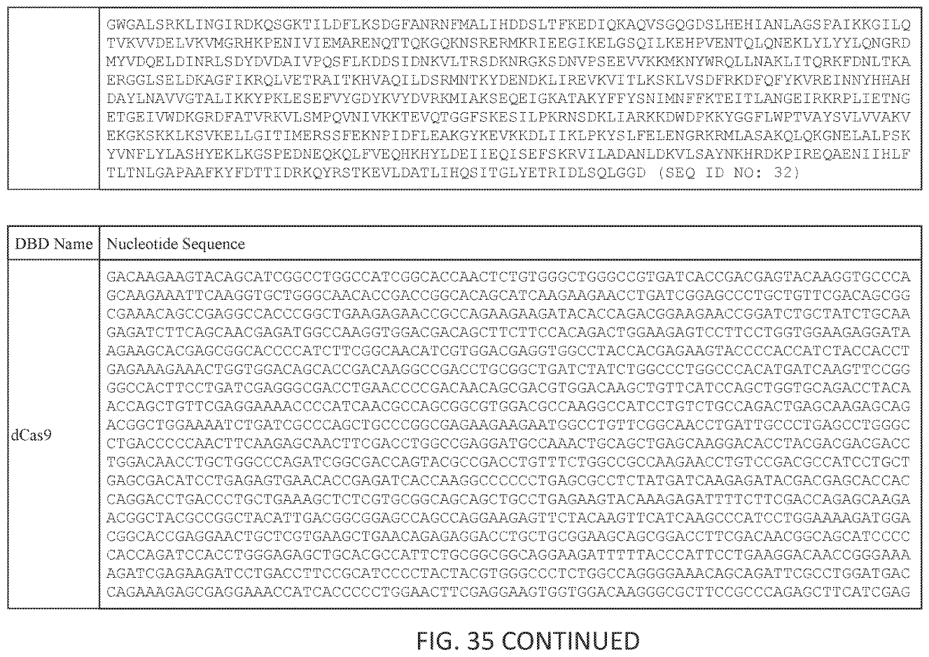

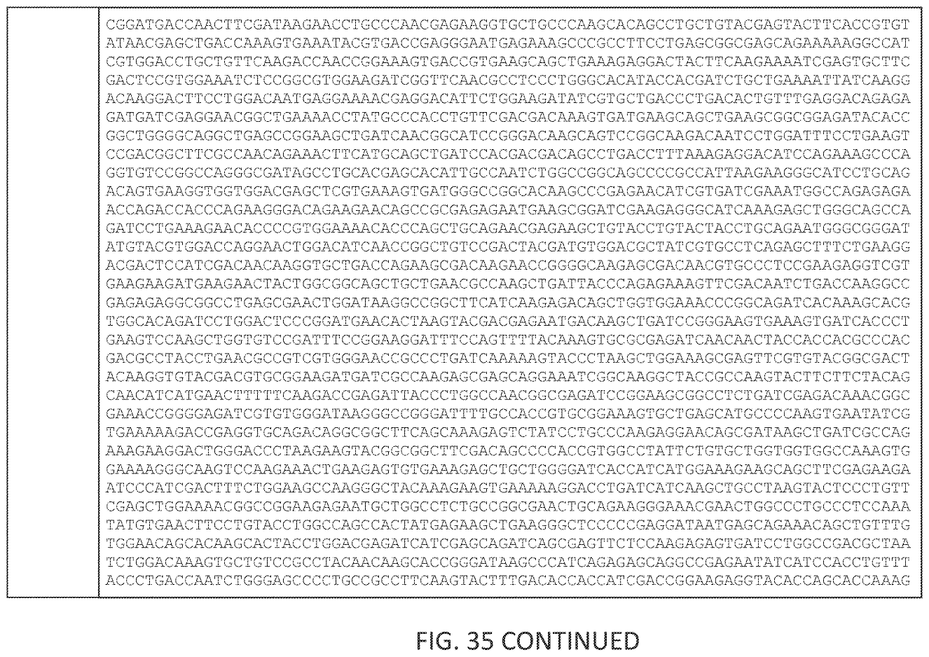

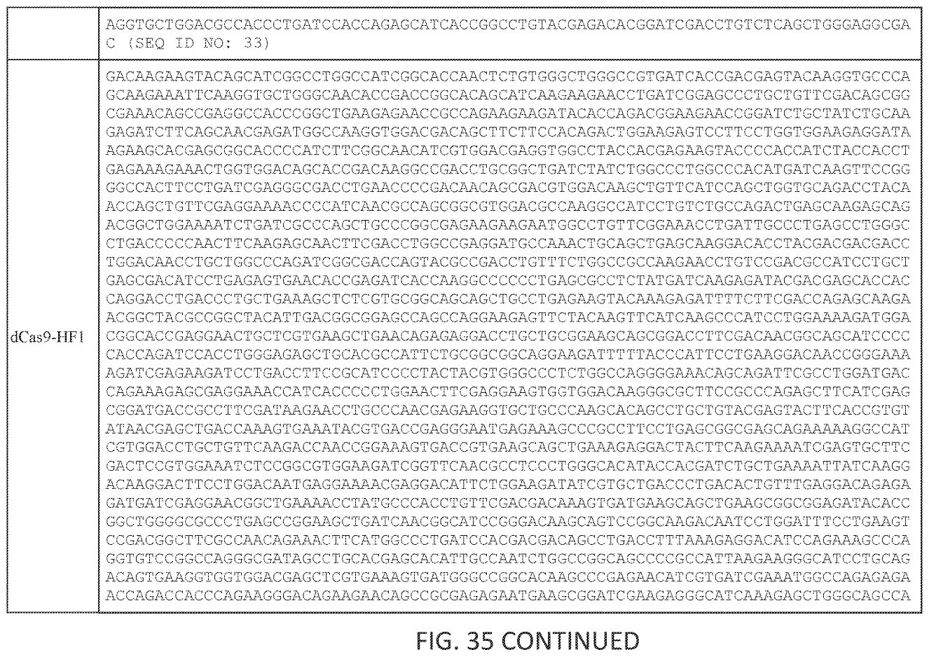

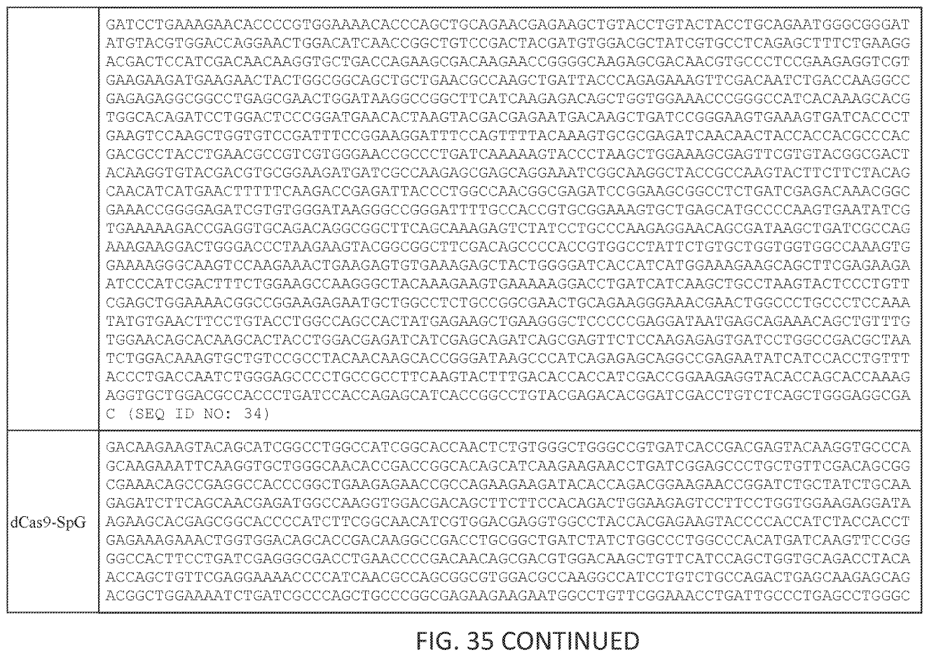

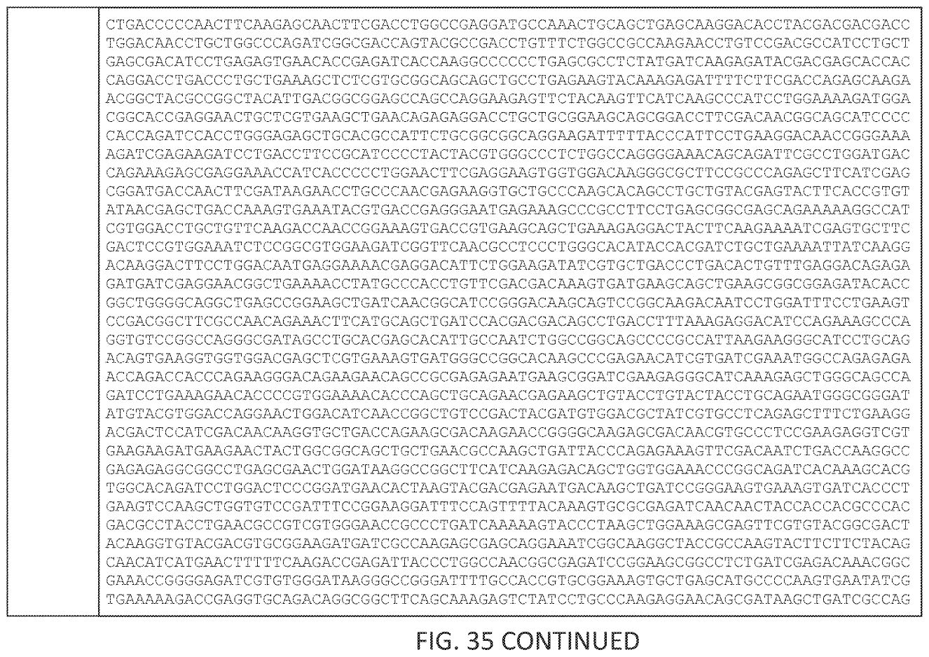

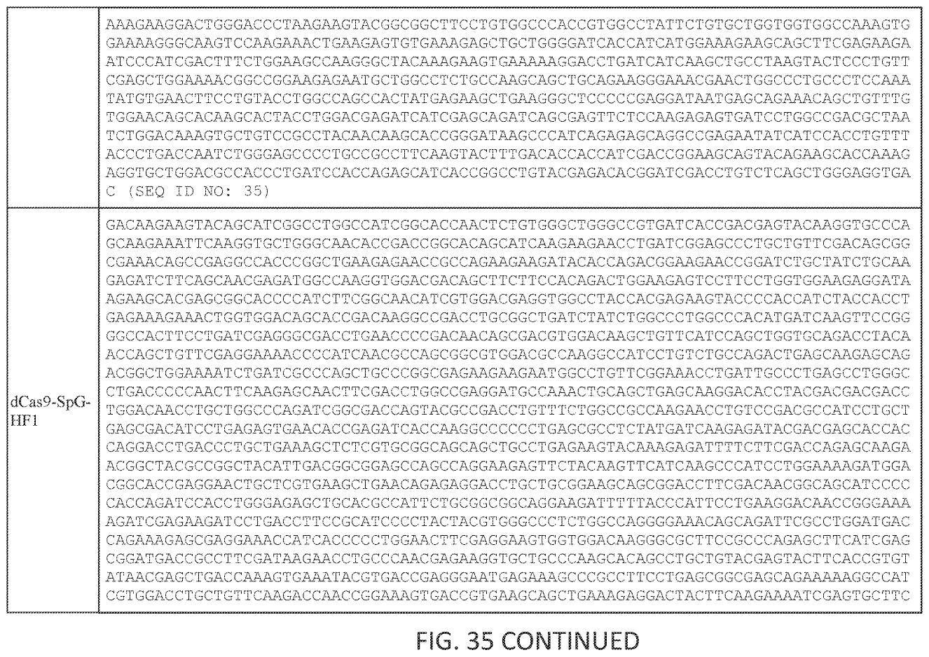

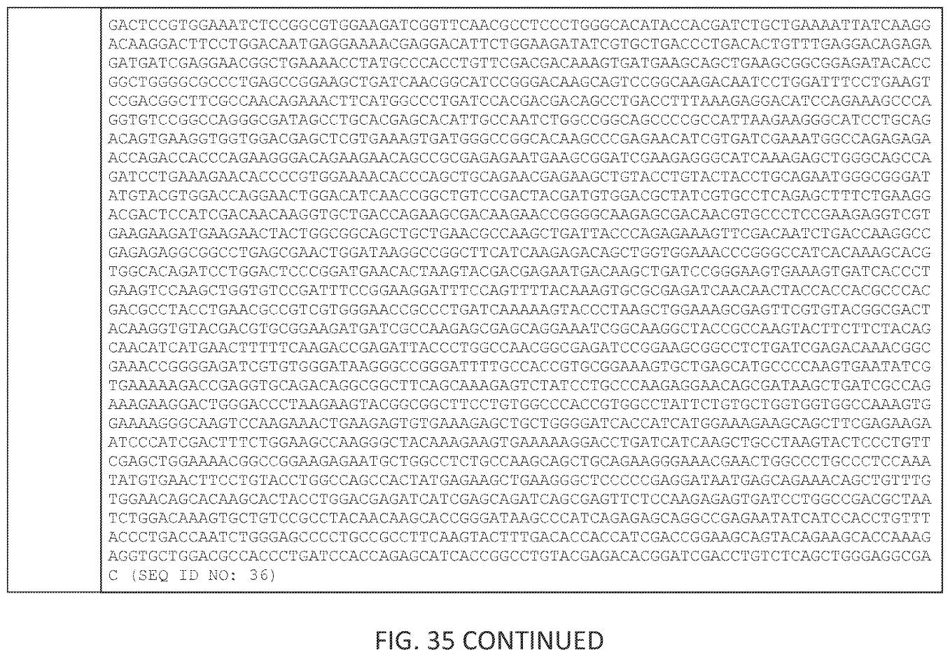

FIG. 35 discloses amino acid (SEQ ID NOs: 29-32) and corresponding nucleotide sequences (SEQ ID NOs: 33-36) for exemplary DBDs (dCas9, dCas9-HF1, dCas9-SpG, dCas9-Spg-HF1) for use in the LSR-DBD fusions described herein.

FIG. 36 discloses amino acid sequences (SEQ ID NOs: 37-42) of exemplary LSR-DBD fusions described herein.

FIG. 37 discloses exemplary gRNA sequences with target site (provided as chromosomal locus according to human genome assembly GRCh38, available at www.ncbi.nlm.nih.gov/genome/guide/human/) that the gRNA spacer is proximal to, overlapping with, or within, the target DNA sequence (SEQ ID NOs: 43-97, 540-550), the corresponding gRNA spacer (SEQ ID NOs: 98-152, 551-561, and an exemplary gRNA scaffold (SEQ ID NO: 153) for use with the LSR-DBD fusions described herein.

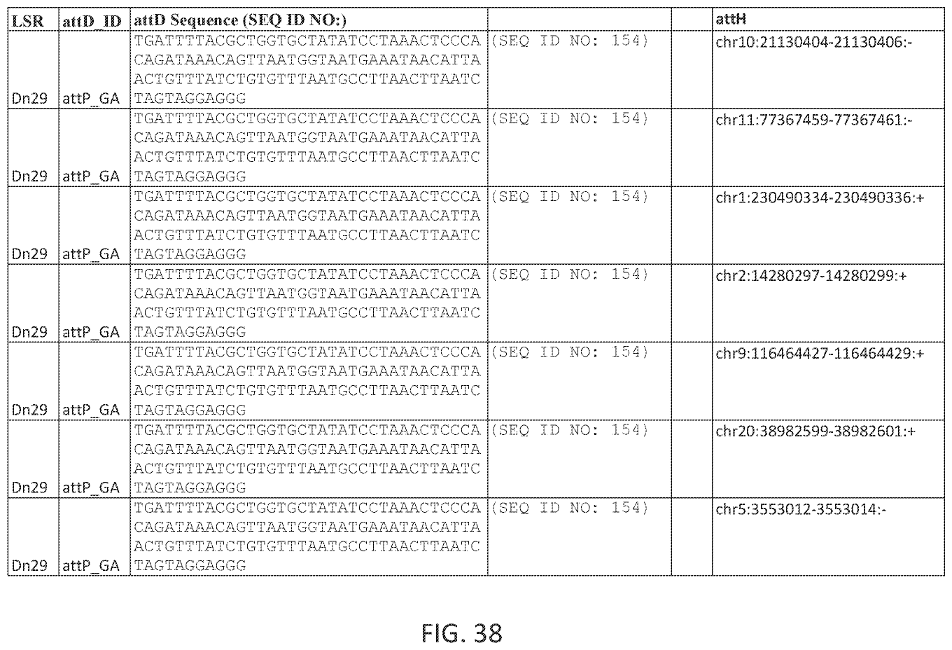

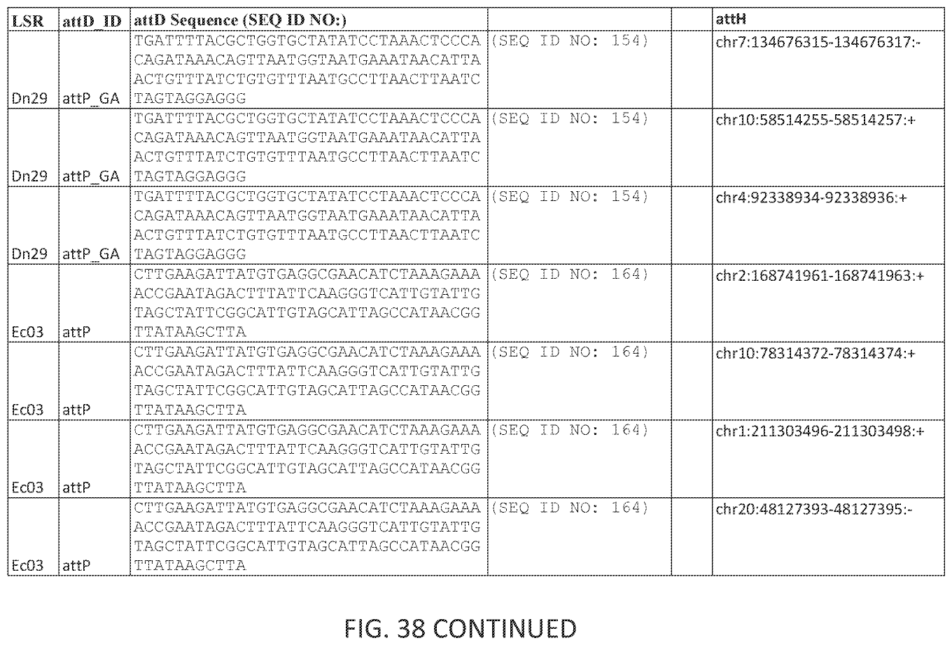

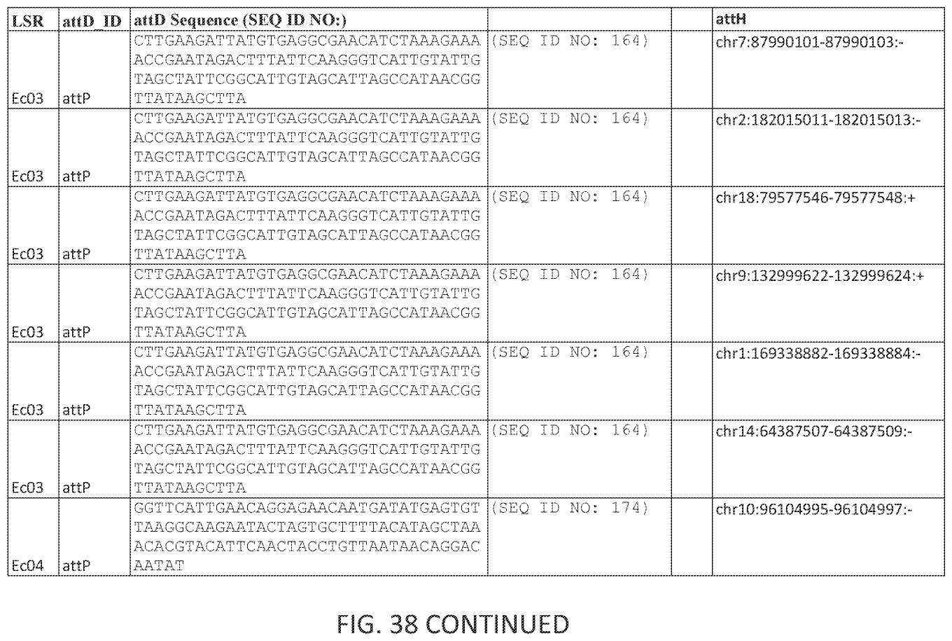

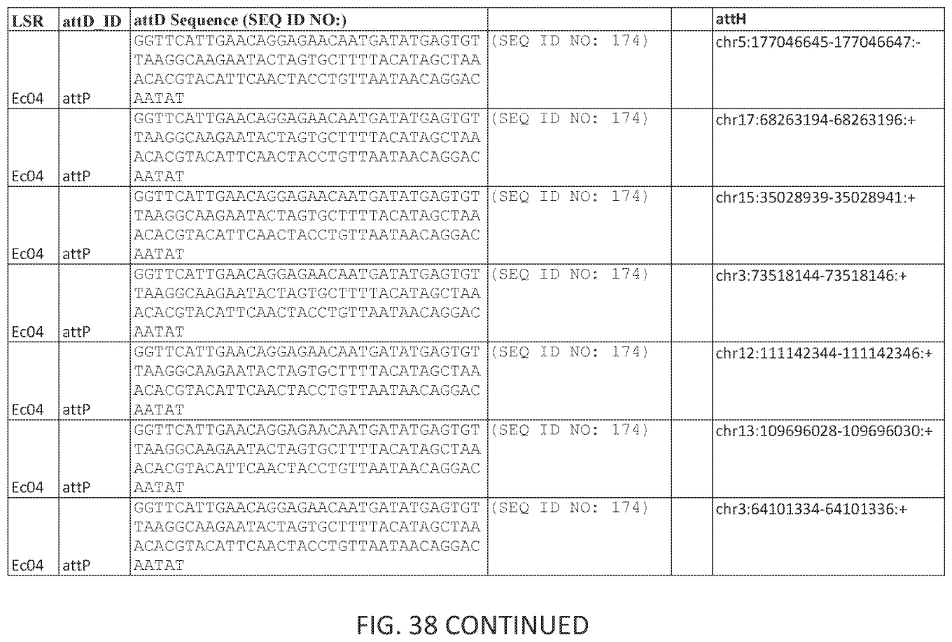

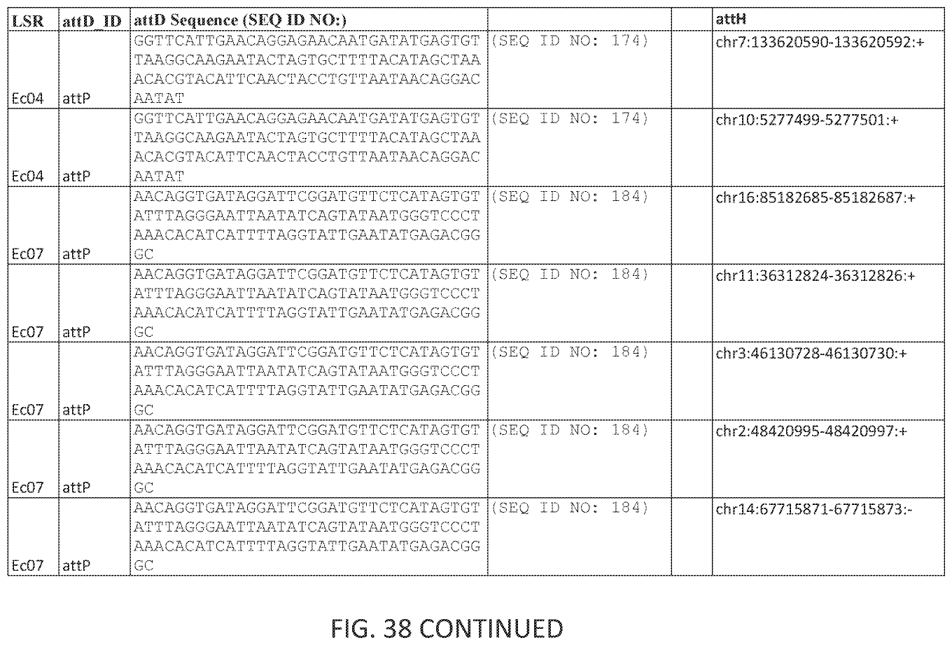

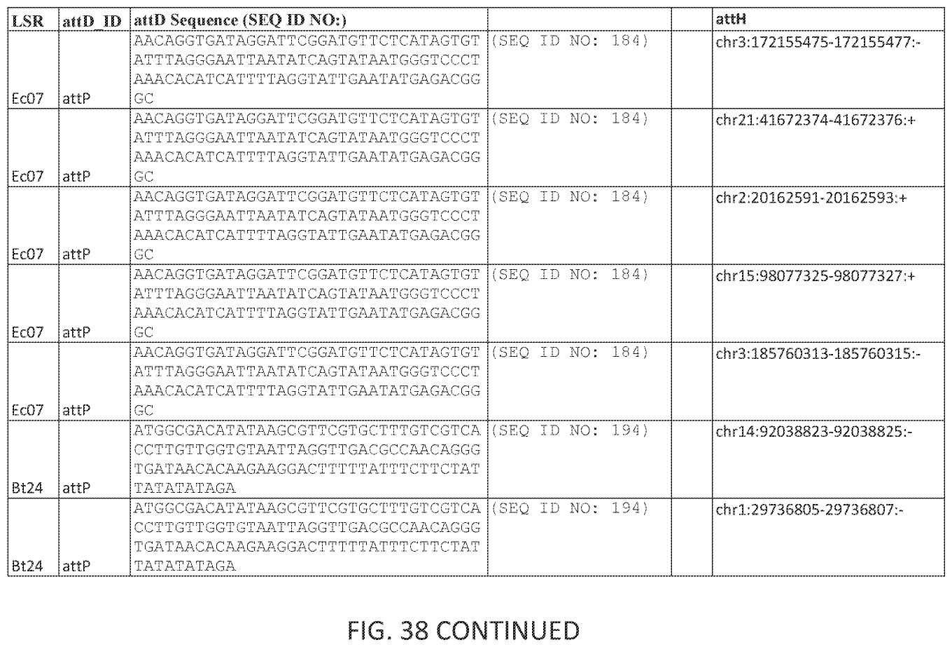

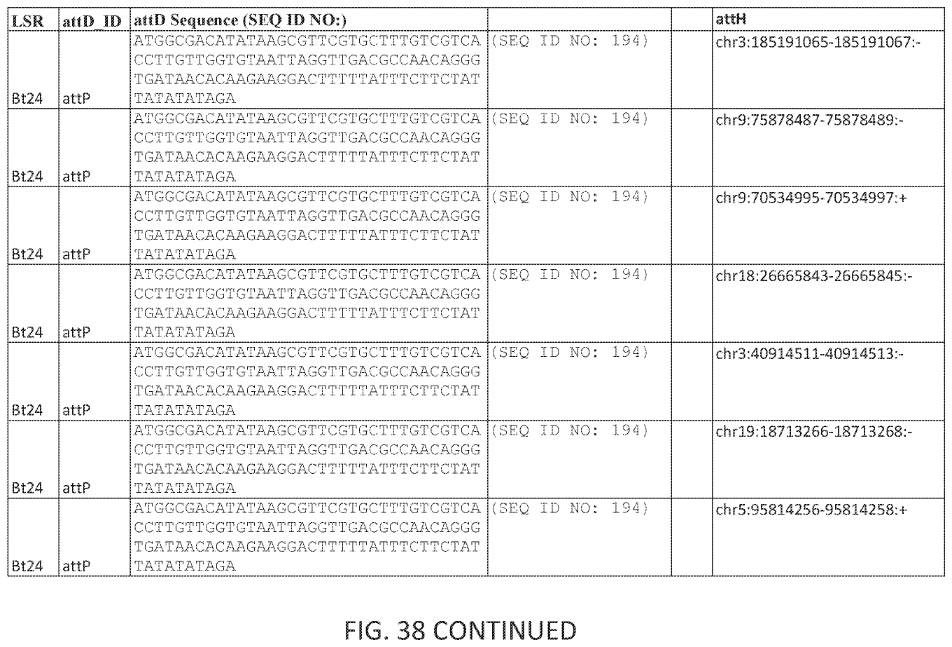

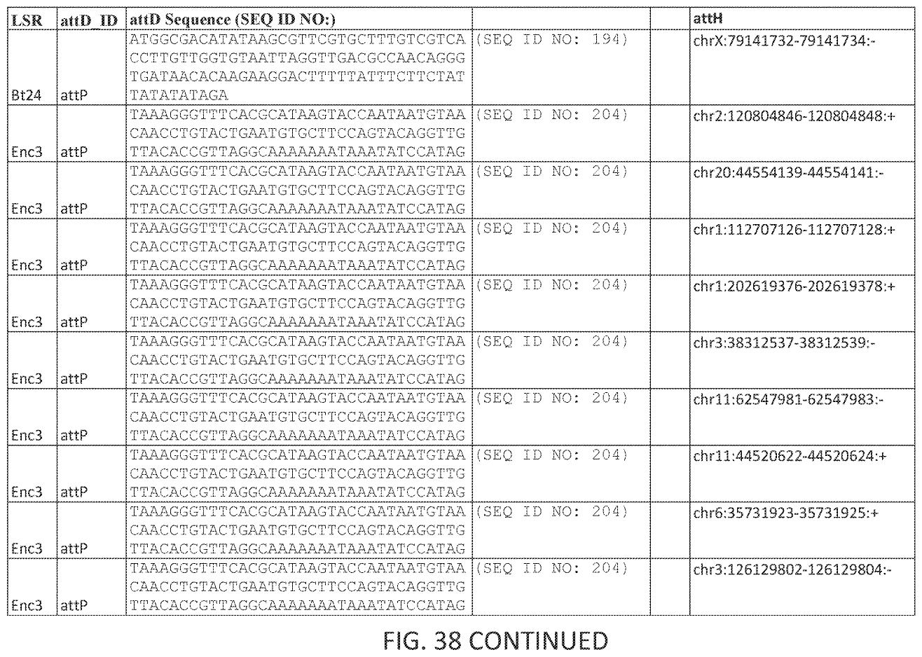

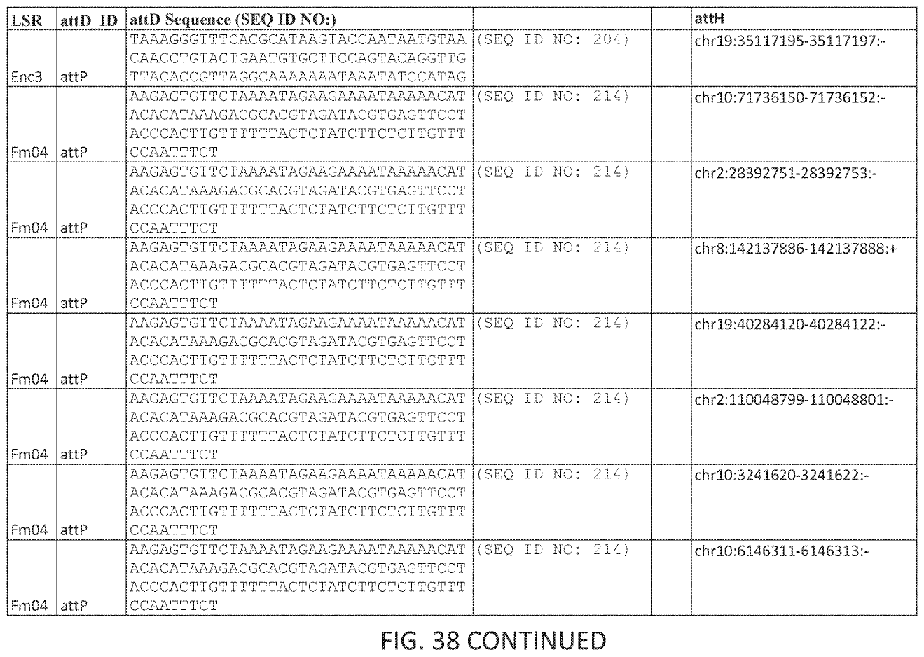

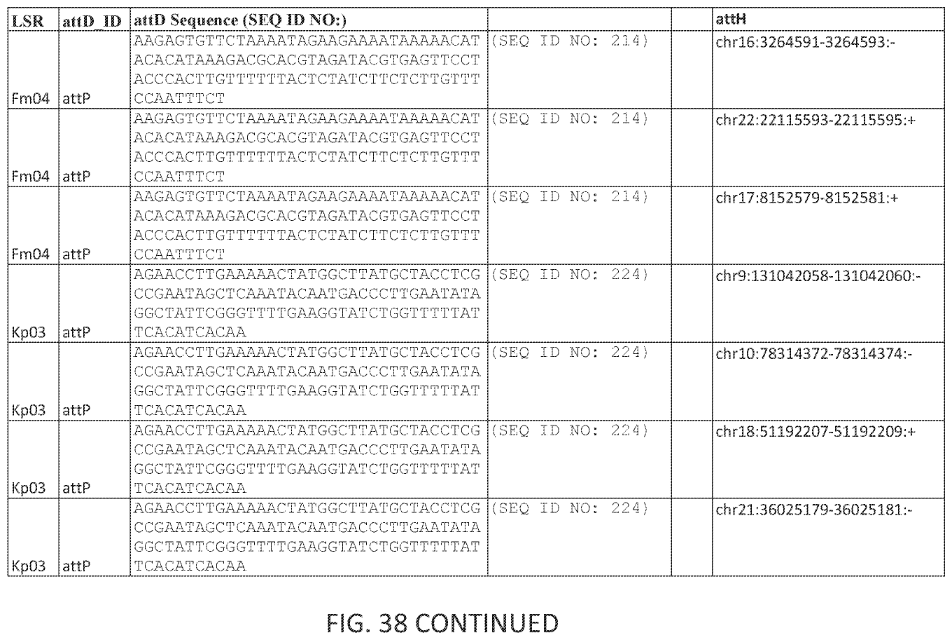

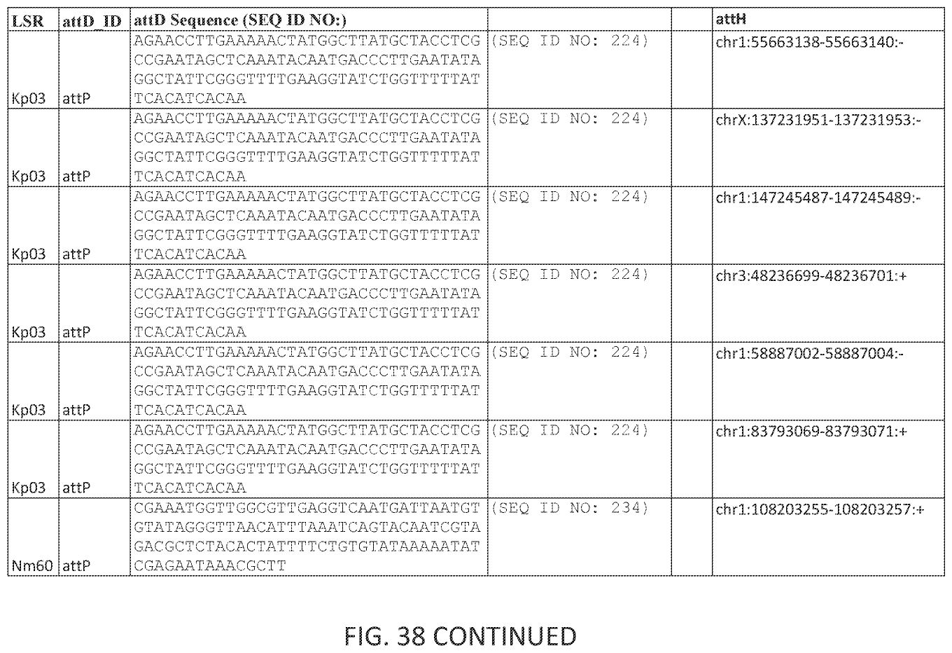

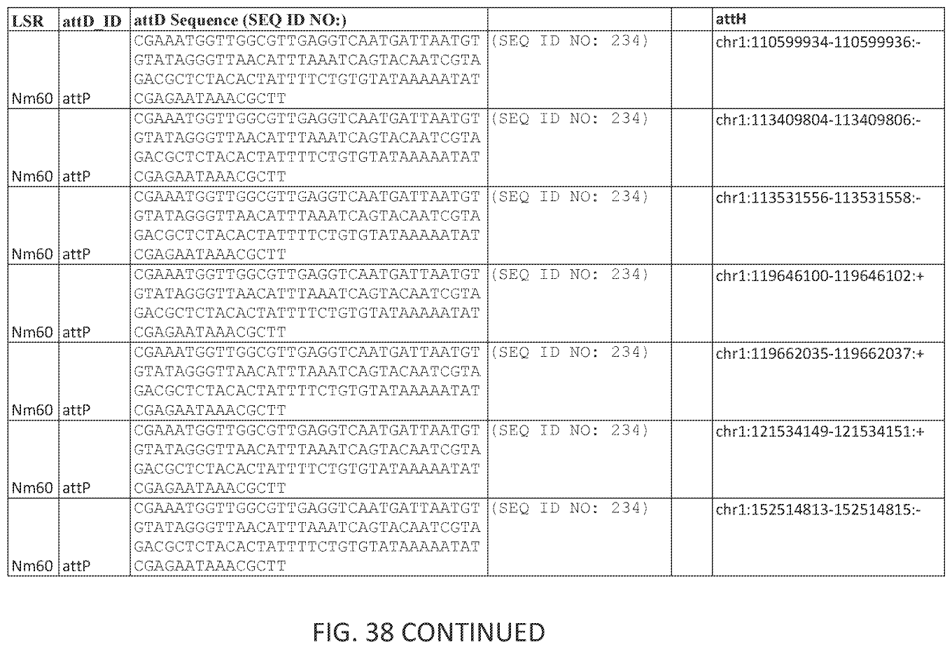

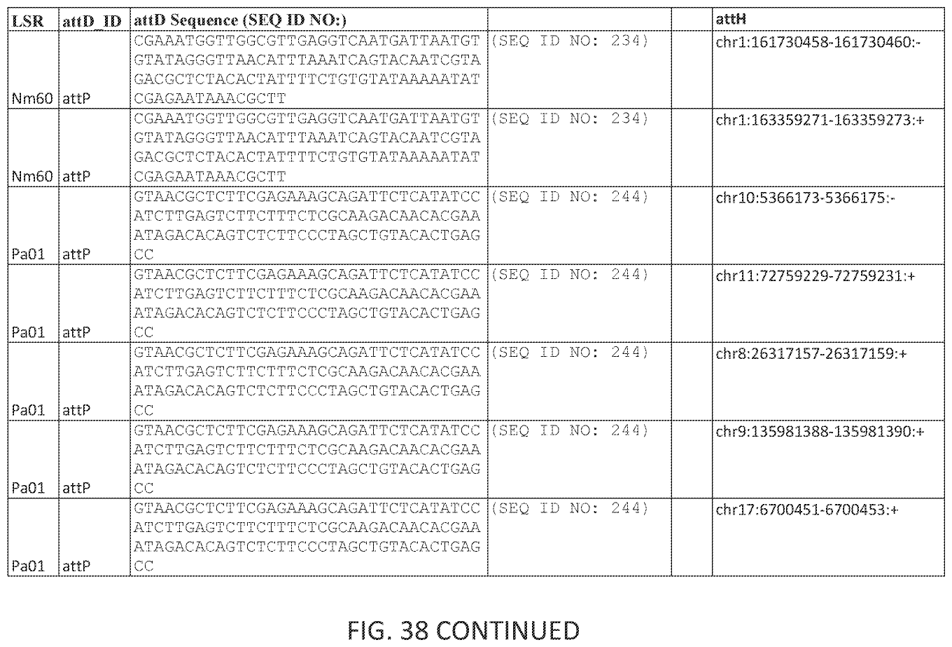

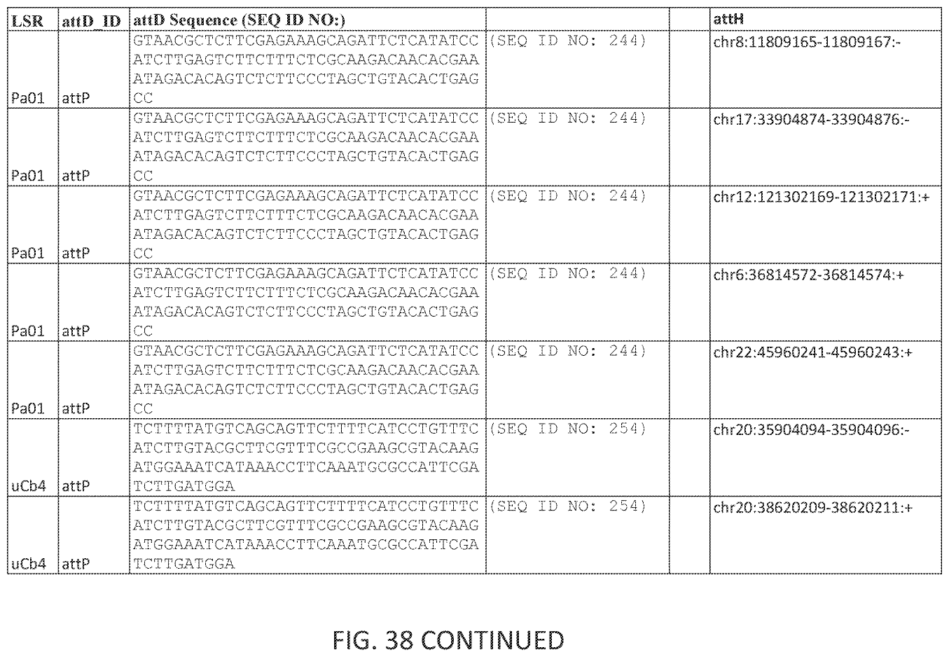

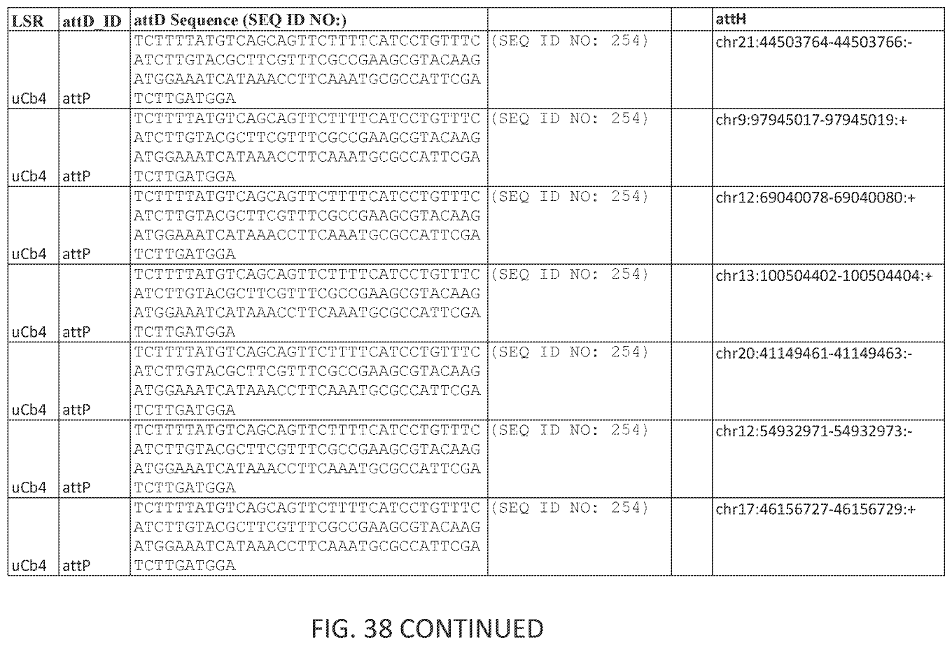

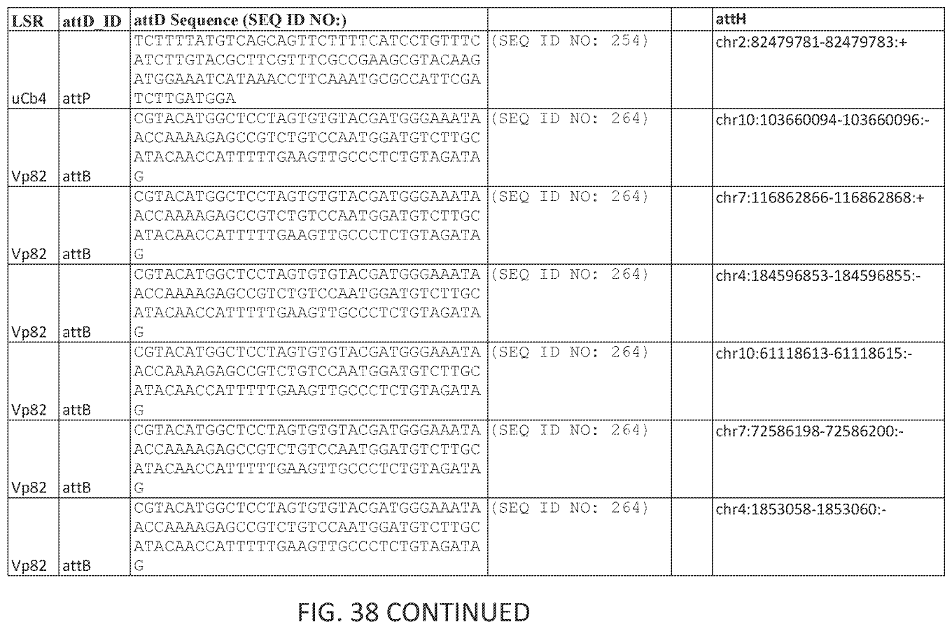

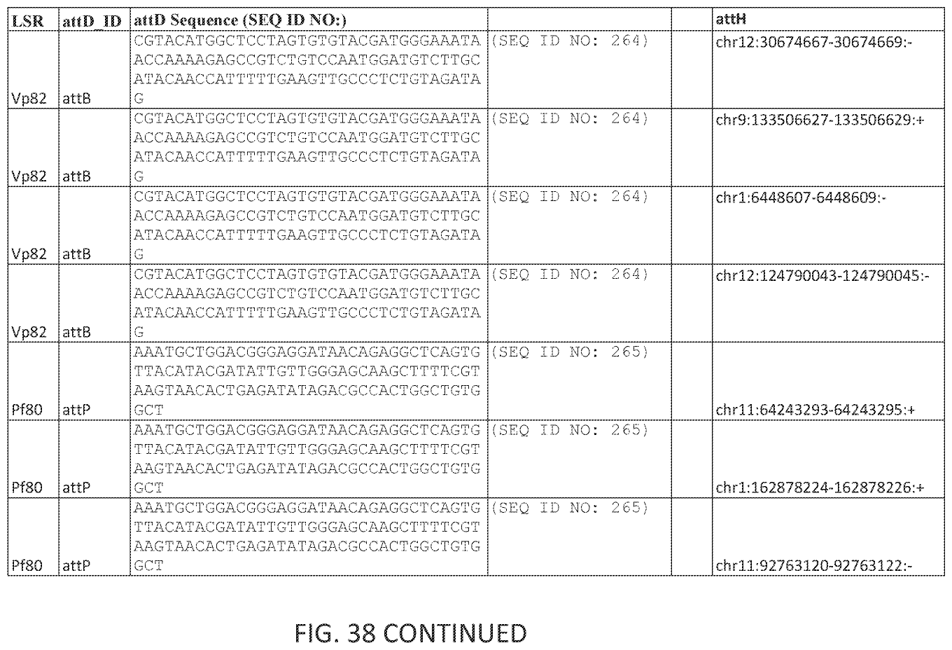

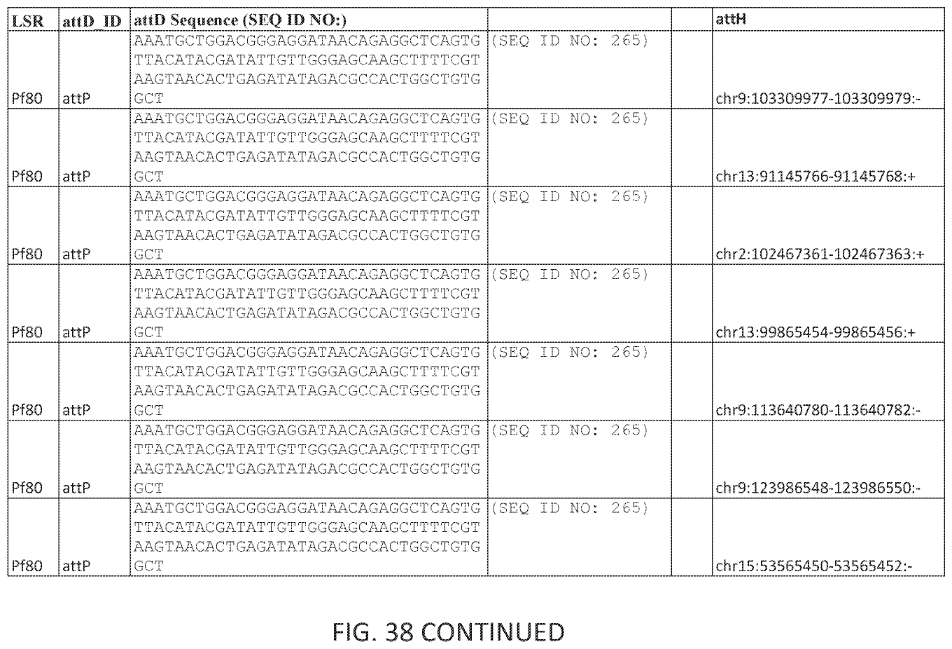

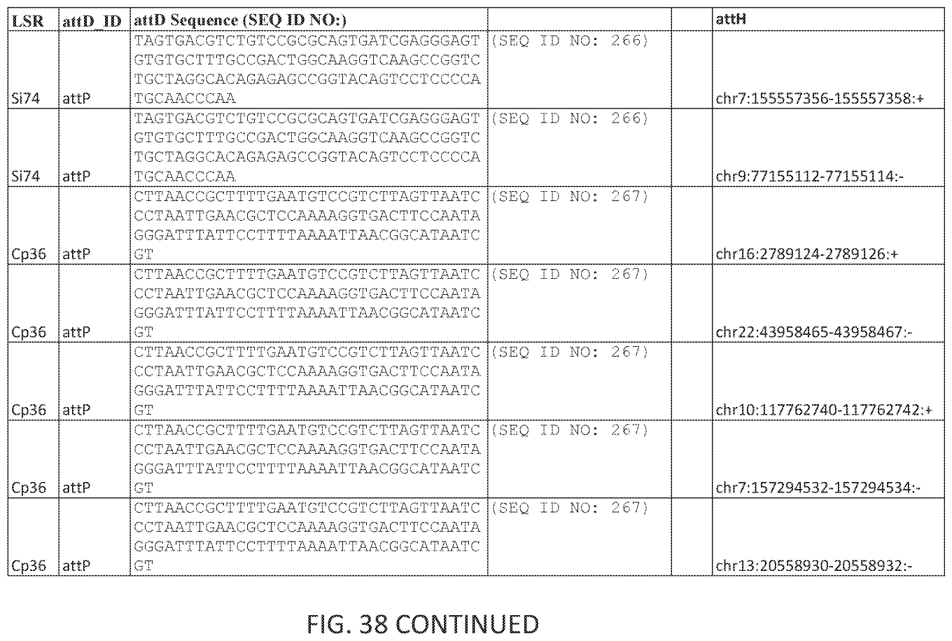

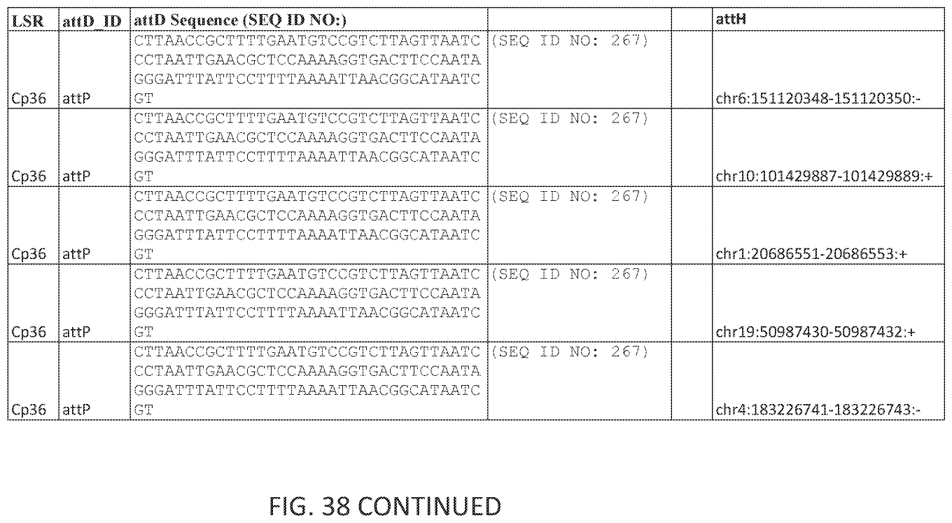

FIG. 38 discloses exemplary attD sequences (SEQ ID NOs: 154, 164, 174, 184, 194, 204, 214, 224, 234, 244, 254, 264-267) and corresponding attH pseudosites (provided as chromosomal locus according to human genome assembly GRCh38, available at www.ncbi.nlm.nih.gov/genome/guide/human/) for various LSRs as indicated.

The invention is further described by the following non-limiting Examples.

### EXAMPLES

Examples are provided below to facilitate a more complete understanding of the invention. The following examples serve to illustrate the exemplary modes of making and practicing the invention. However, the scope of the invention is not to be construed as limited to specific embodiments disclosed in these Examples, which are illustrative only.

### Example 1: Methods

### LSR Integration Site Mapping:

To determine the location and efficiency of integration into pseudosites in the human genome, various LSR orthologs and donor plasmid containing their cognate attD were transfected into cells and their integration sites were mapped with next generation sequencing. In K562 cells, 1.0×10⁶ cells were electroporated in 100 μL Amaxa solution (Lonza Nucleofector SF, program FF-120), with 3000 ng LSR plasmid and 2000 ng pseudosite attD plasmid. As a non-matching LSR control, 3000 ng of Bxb1 was substituted for the correct LSR plasmid. The cells were cultured between 2×10⁵ cells/mL and 1×10⁶ cells/mL for 2-3 weeks. Genomic DNA was extracted using the Quick-DNA Miniprep Kit (Zymo) and quantified by Qubit HS dsDNA Assay (Thermo). Tn5 tagmentation, nested PCR enrichment of the integration site, NGS sequencing, and computational analysis of integration sites was performed as described in Durrant et al., NBT 2022.

### Construct Design and Cloning:

Fusion proteins consisting of a catalytically dead Cas9 fused to an LSR-P2A-GFP were constructed by Gibson cloning individual parts into a pUC19-derived plasmid containing the Ef1a promoter and a SV40 poly-A tail. Variable linkers, including a (GGS)₈ (SEQ ID NO: 11), (GGGGS)₆ (SEQ ID NO: 598), XTEN16, XTEN32-(GGSS)₂ (SEQ ID NO: 14), and XTEN48-(GGSS)₂ (SEQ ID NO: 15), were tested to link the dCas9 to the LSR, in both N and C terminus fusions. Spacers targeting loci proximal to the experimentally determined LSR integration site with NGG PAMs and non-targeting controls were cloned into an sgRNA expressing plasmid under a U6 promoter. Donor plasmids contained the LSR's cognate attD sequence, Ef1a promoter, mCherry, and puromycin resistance gene.

### Transfection and Genomic DNA Extraction:

20,000 HEK293FT cells were plated into each well of a 96 well plate. One day later, 375 ng of effector plasmid, 100 ng sgRNA plasmid, and 250 ng donor plasmid were transfected per well using Lipofectamine 2000. In some transfections, a 5:1:1 ratio of donor:effector:guide plasmid was used, resulting in delivery of 389 ng donor plasmid, 259 ng effector plasmid, and 76 ng sgRNA plasmid. 3 days post-transfection, the genomic DNA was harvested. From each well the media was aspirated, 50 μL of QuickExtract DNA I was added, cells were mixed via pipetting, transferred to a qPCR plate, vortexed, and thermocycled with the following protocol: 65 C for 15 min, 68 C for 15 min, 98 C for 10 min. Genomic DNA was purified using AmpureXP following manufacturer's protocol and 0.9× bead volume.

On-Target Integration Quantification Via ddPCR:

PCR primers and FAM-BHQ1 taqman probes were designed to span the donor-genome junction at attH1. For each target site, a reference set of primers and HEX-BHQ1 probes were designed to target proximally on the same chromosome. ddPCR droplets were generated, amplified, and measured on the QX200 AutoDG Droplet Digital PCR System (Biorad). Integration efficiency was calculated by taking the ratio of the number of FAM positive droplets over HEX positive droplets.

On-Target Integration Quantification Via qPCR:

PCR primers and FAM-BHQ1 taqman probes were designed to span the donor-genome junction at attH1. For each target site, a reference set of primers and HEX-BHQ1 probes were designed to target proximally on the same chromosome. Multiplexed qPCR was conducted using Taqman Fast Advanced MasterMix (Thermo) to quantify integration efficiency. Delta Ct was calculated in comparison to the reference primer/probe set.

### Total Integration Quantification Via Flow Cytometry:

20,000 HEK293FT cells were plated into each well of a 96 well plate. One day later, 375 ng of effector plasmid (LSR or dCas9-LSR fusions), 100 ng sgRNA plasmid, and 250 ng donor plasmid were transfected using Lipofectamine 2000. A non-matching LSR (Bxb1) and an empty sgRNA plasmid were transfected with the donor plasmid as a Donor Only control. Cells were cultured for 17 days to allow un-integrated donor plasmid to dilute out. Flow cytometry measurements were taken at various timepoints on the Attune NXT Flow Cytometer (Thermo).

### Example 2: Designing and Optimizing a Dn29-dCas9 Fusion Construct

LSRs bind attP and attB in a tetrameric complex. FIG. 4.

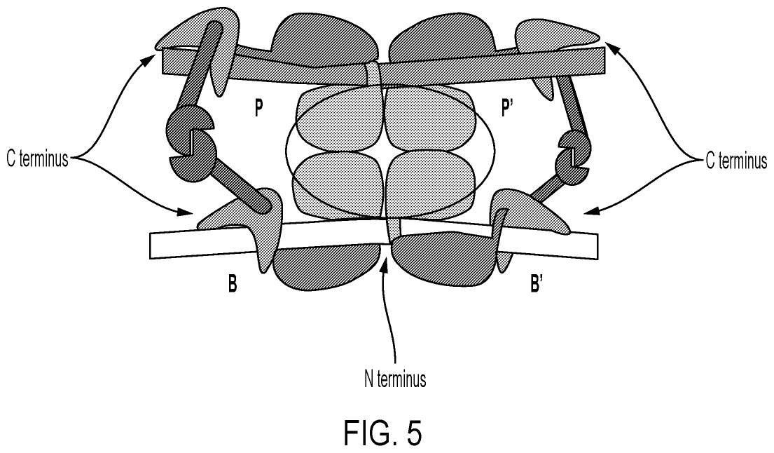

The LSR N terminus is critical for tetrameric complex formation, subunit rotation, cleavage, and ligation. FIG. 5

Designs of exemplary Dn29-dCas9 fusion constructs are shown in FIG. 6. N vs C terminal fusions were tested and long, flexible linkers prioritized. Examples of linkers tested are: (GGS)₈ (SEQ ID NO: 11), (GGGGS)₆ (SEQ ID NO: 598), XTEN16, XTEN32-(GGSS)₂ (“(GGSS)₂” disclosed as SEQ ID NO: 585), and XTEN48-(GGSS)₂ (“(GGSS)₂” disclosed as SEQ ID NO: 585). To check whether the resulting fusion products are competent for recombination, a plasmid expressing each fusion construct was co-transfected into HEK293FT cells with a donor plasmid containing an attD and a non-targeting guide RNA expressing plasmid. After 3 days, the integration efficiency at attH1 was determined via qPCR. The results show that Dn29-linker-dCas9 fusions are active for recombination at levels similar to or higher than the wildtype Dn29, and the dCas9-linker-Dn29 fusion constructs have reduced recombination capabilities.

In each condition, 725 ng of DNA is transfected. Because the fusion effector plasmid is 10 kb, vs the wildtype Dn29 effector plasmid size of 6 kb, and the same mass of effector plasmid is used across the two conditions, cells transfected with the fusion effector receive a lower molar concentration of effector plasmid and a higher molar ratio of donor plasmid to effector plasmid. This factor may explain why the fusion constructs have a higher integration efficiency than the wildtype construct, even when transfected with a non-targeting gRNA. The dCas9-linker-Dn29 fusions may have reduced recombination because of steric hindrances caused by the bulky dCas9 domain interfering with tetrameric complex formation or subunit rotation.

Although all of the Dn29-linker-dCas9 conditions resulted in recombination capable proteins, longer, more flexible linkers such as the XTEN32
-(GGSS)₂ linker (“(GGSS)₂” disclosed as SEQ ID NO: 585) (FIG. 6) result in higher integration rates.

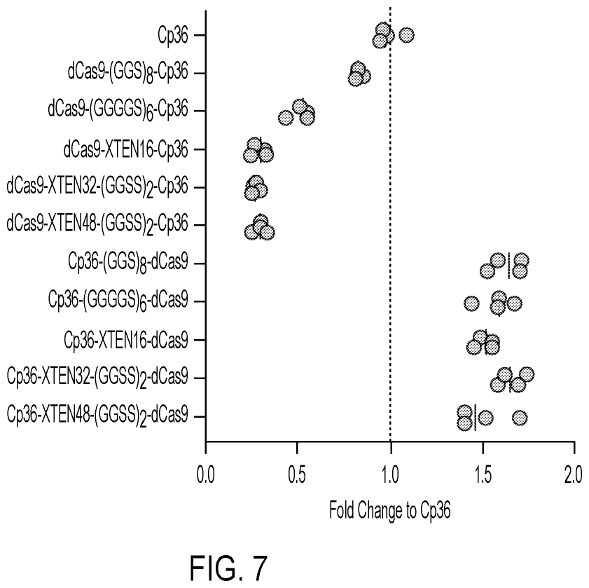

Construct architecture is generalizable to another LSR (Cp36) (FIG. 7).

### Example 3: Proof-of-Concept Pseudosite Targeting with a Single Guide RNA

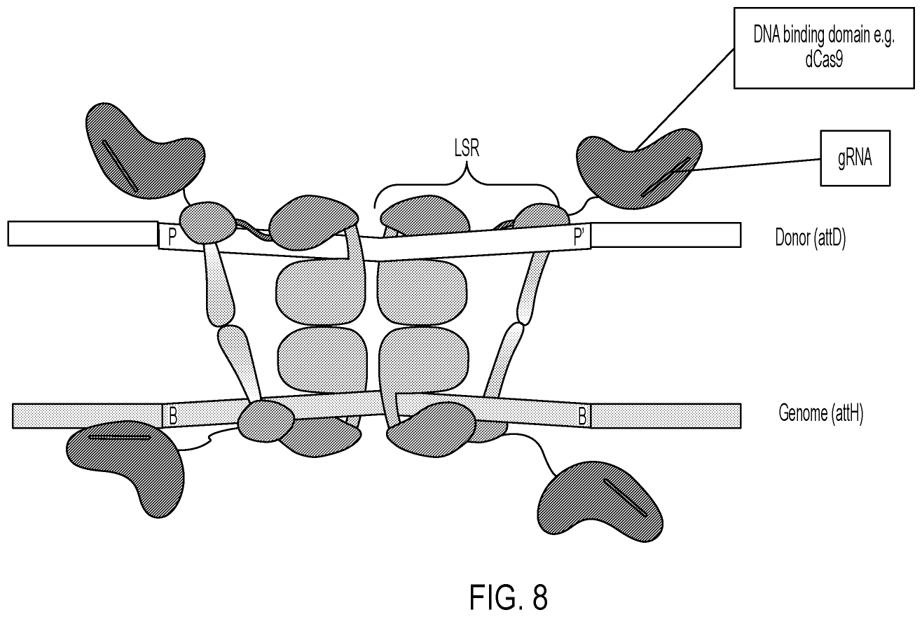

A single guide RNA complementary to DNA proximal to a pseudosite can direct an LSR-dCas9 monomer to the pseudosite, increasing integration efficiency at this site (FIG. 8). A proof of concept of this system is exemplified using a fusion of Dn29 and dCas9 and various guide RNAs targeting attH1 and attH3. AttH1 is Dn29's most efficient pseudosite, located at chromosome 10:21,130,404 within the intron of NEBL (cardiac nebulette). attH3 is the 3rd top pseudosite. It is intergenic, on chromosome 1. The nearest genes are: LOC105373164 (non-coding RNA) and PGDB5 (piggyBac transposable element derived 5).

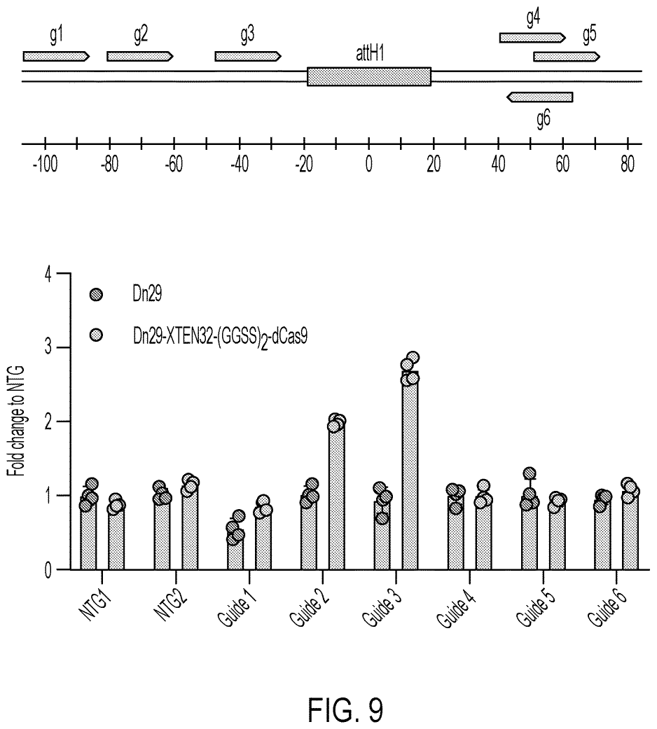

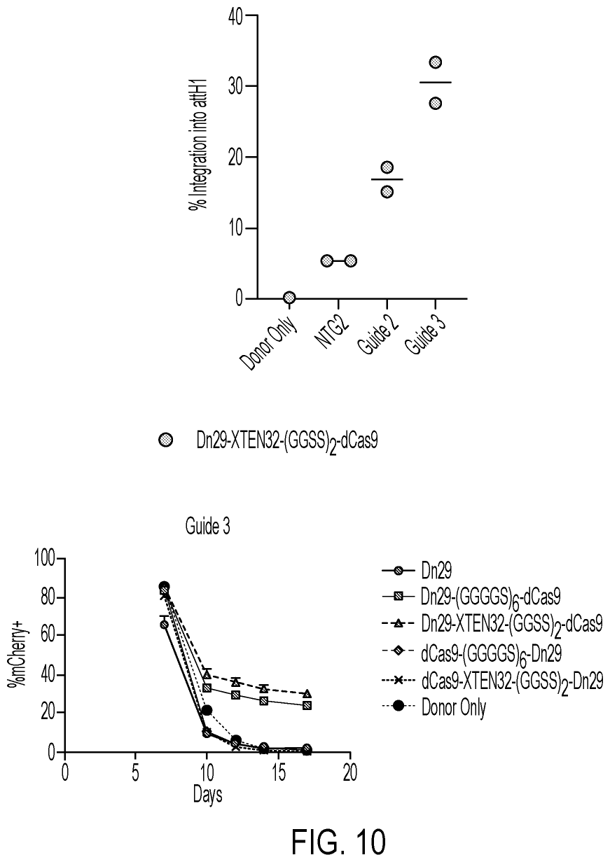

FIG. 9 shows Dn29-dCas9 targeting to attH1. Six gRNAs were designed to target proximally to attH1, as shown in the top schematic. HEK293FT cells were transfected with the Dn29-dCas9 fusion effector plasmid, an attD containing donor plasmid, and a gRNA plasmid. After 3 days, integration efficiency is read out by qPCR. Two gRNAs (2 and 3) were identified to increase integration efficiency significantly over a non-targeting guide. This integration efficiency was validated with orthogonal readouts methods, including ddPCR (FIG. 10, top) and flow cytometry of stably integrated mCherry expression (FIG. 10, bottom).

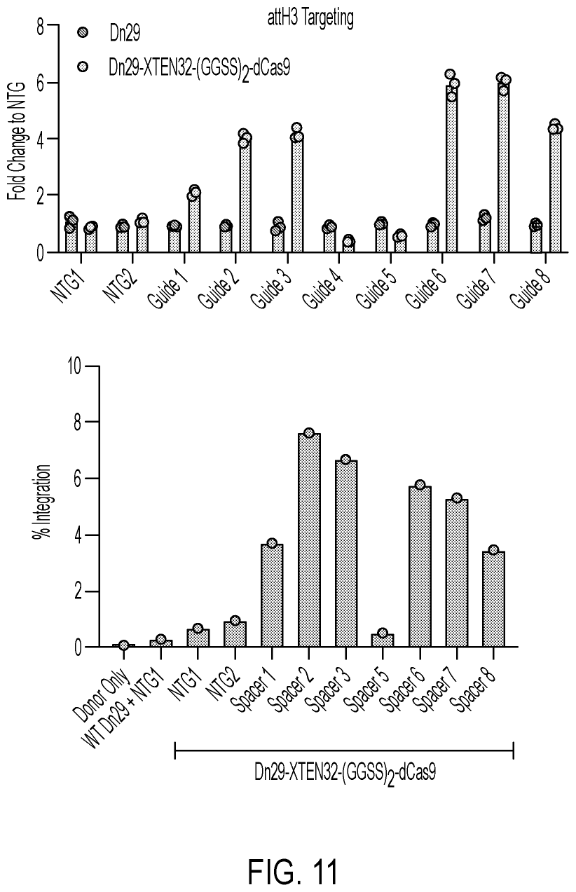

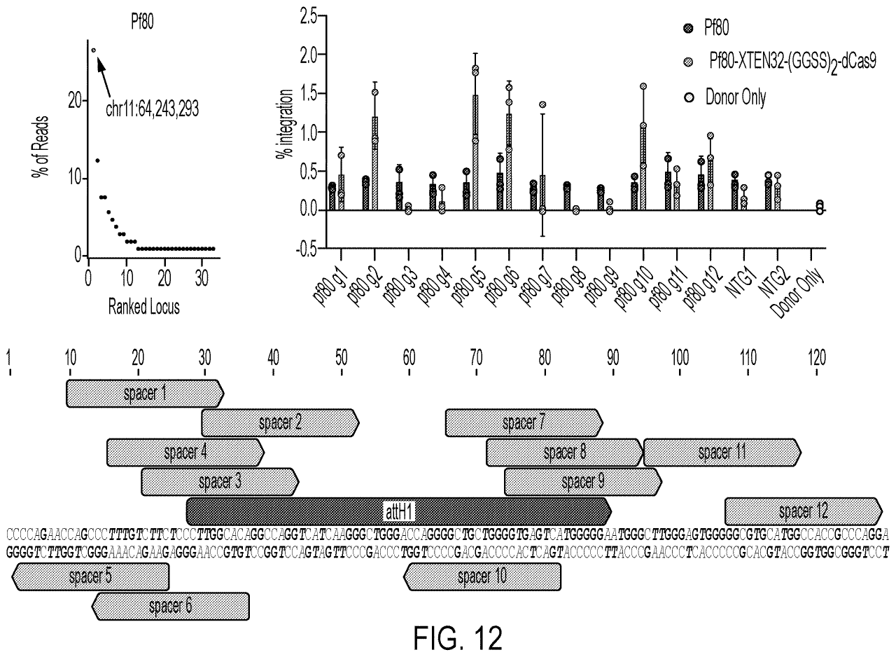

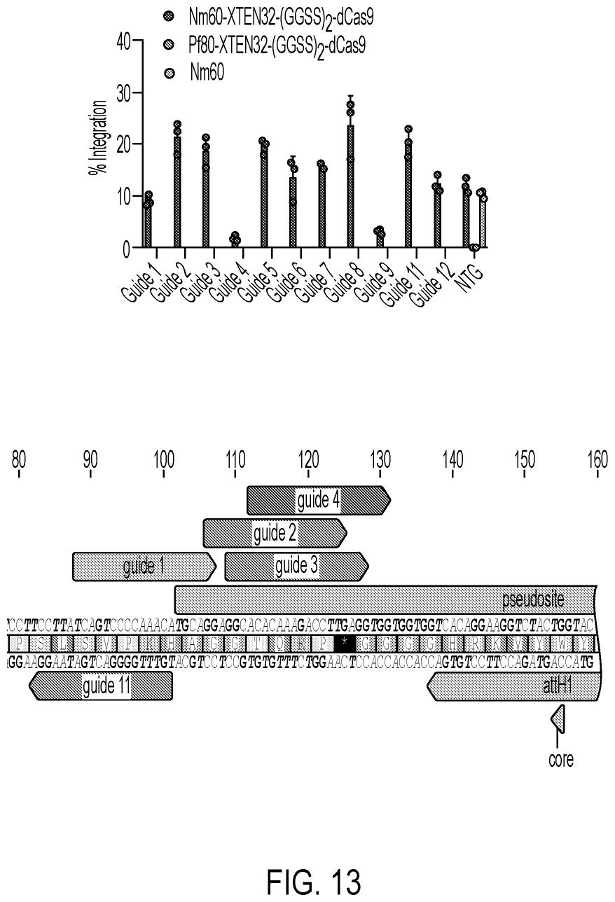

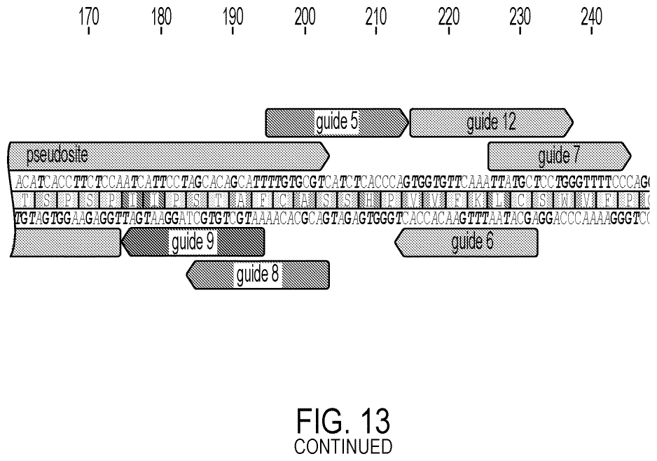

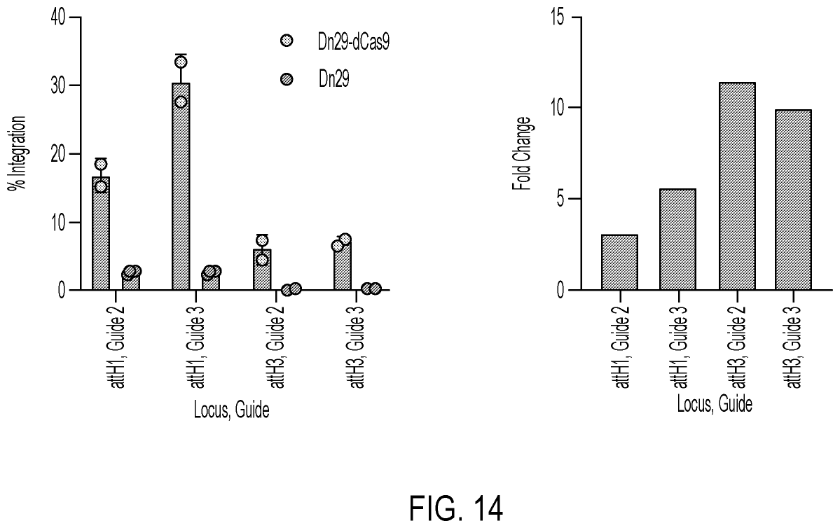

This method of targeting Dn29-dCas9 to a pseudosite was further validated with another pseudosite, attH3. Eight gRNAs were designed to target attH3, and six of these gRNAs resulted in up to six-fold increased integration efficiency compared to a non-targeting gRNA (FIG. 11), as shown by qPCR (top) and ddPCR (bottom). Finally, we showed that this method of LSR-dCas9 pseudosite targeting can be generalized to other LSR orthologs, Pf80 and Nm60. Pf80, another human genome targeting LSR, was fused to dCas9 and delivered into HEK293FT cells with an attD donor plasmid and various attH1 targeting gRNAs, whose spacer locations are illustrated in the bottom schematic of FIG. 12. attH1 was determined by the integration site mapping assay, (FIG. 12, left), and is located at chromosome 11, locus 64,243,293. qPCR results (right) show that various gRNAs can increase Pf80 integration efficiency at attH1. Similarly, Nm60-dCas9 fusions, shown in FIG. 13, increase integration efficiency up to 25% at attH1 when using various gRNAs whose spacer locations are illustrated on the bottom schematic of FIG. 13. dCas9 fusions increase integration efficiency up to 30% at attH1 and 8% at attH3, with fold change of successful guides over a non-targeting guide ranging from 3-11 (FIG. 14). The difference between the absolute integration efficiency of attH1 and attH3 illustrate that the maximum integration efficiency may be limited by the starting insertion efficiency.

### Example 4: Mechanism of Action

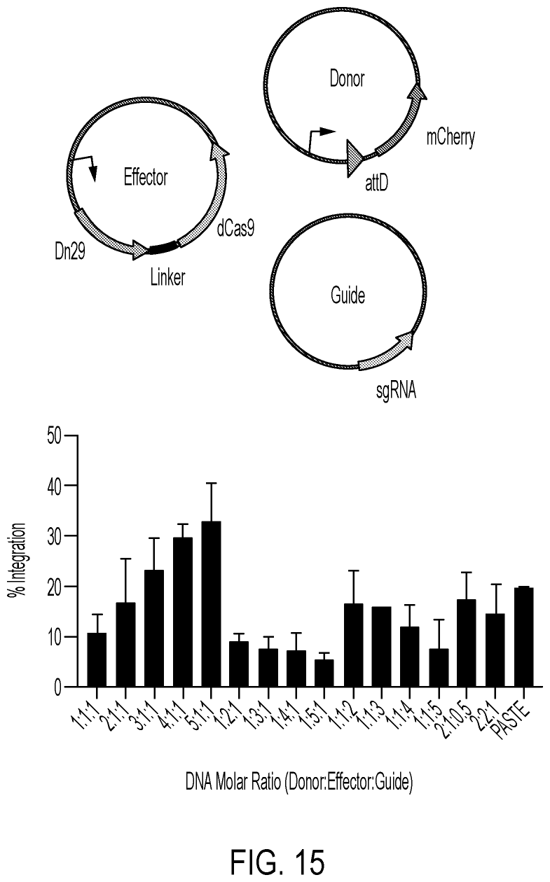

We sought to determine the limiting reagent for the reaction. FIG. 15 shows a schematic of a non-limiting embodiment of the plasmids that can be used to effectuate DNA insertion (top). The bottom panel shows the percentage integration upon transfection of different molar ratios of the three plasmids.

Donor plasmid is a limiting reagent. Strategies to increase the molarity of donor plasmid in the nucleus, including using minicircles, bDNA nuclear import signals, and donor gRNA targeting, can be used to improve efficiency.

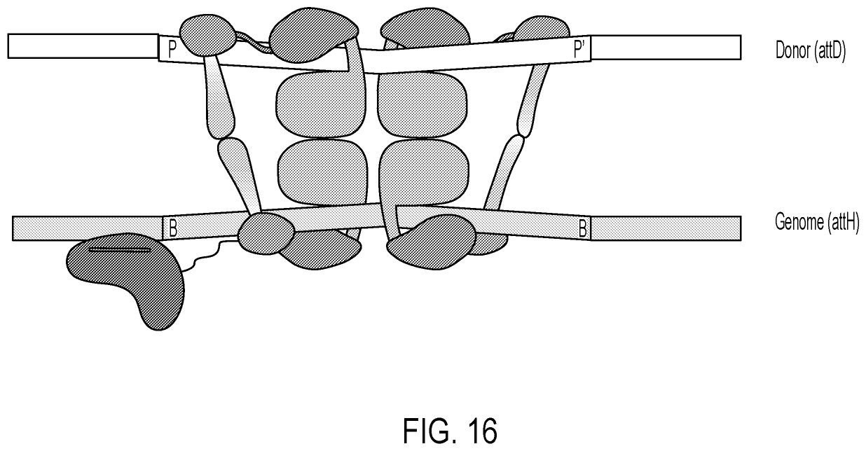

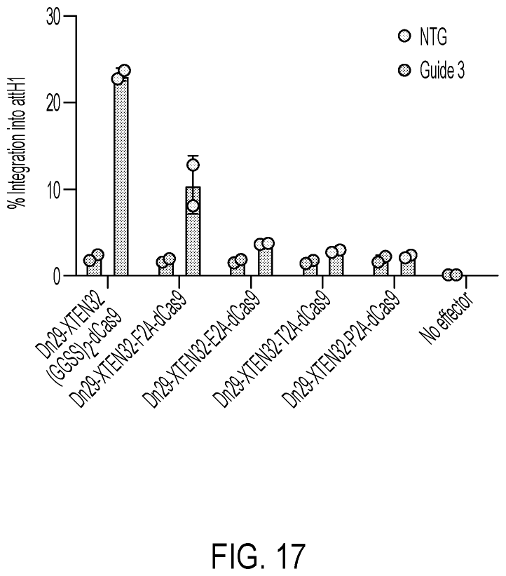

It was investigated whether the untargeted dCas9 monomers sterically hinder recombination. We hypothesized that the three un-targeted dCas9 monomers may be sterically hindering the rotation/recombination mechanism. FIG. 16 shows a schematic of this idea, where the three un-targeted LSR proteins in the tetrameric complex are not fused with dCas9. We tested this hypothesis by delivering LSR-dCas9 fusion proteins linked with various self cleaving 2a peptides. These constructs should result in a mixed population of LSR monomers and LSR-dCas9 fusion proteins in a range of around 50% cleavage (F2a) to nearly complete cleavage (P2a). FIG. 17 shows that delivering mixed populations of LSR and LSR-dCas9 at the ratios provided do not improve recombination efficiency, and in fact partial cleavage of the fusion construct reduces recombination efficiency, indicating that these extra dCas9 domains are not sterically hindering recombination. This however does not rule out the possibility that untested ratios of LSR-dCas9 fusion mixed with unfused LSR may still increase efficiency. This data also indicates that direct fusion between LSR and dCas9 is required to see the effects, as complete cleavage (P2a) is not significant compared to a non-targeting guide. This provides evidence for the hypothesis that direct tethering of the LSR proximally to a pseudosite drives the increase in efficiency rather than other effects caused by dCas9/DNA binding, such as unwinding of the locus or effects on chromatin such as local nucleosome breathing.

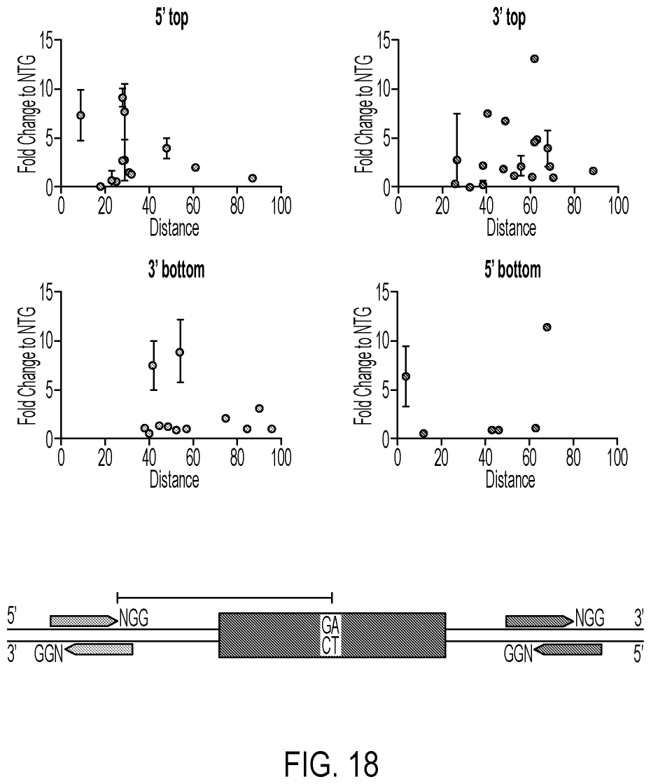

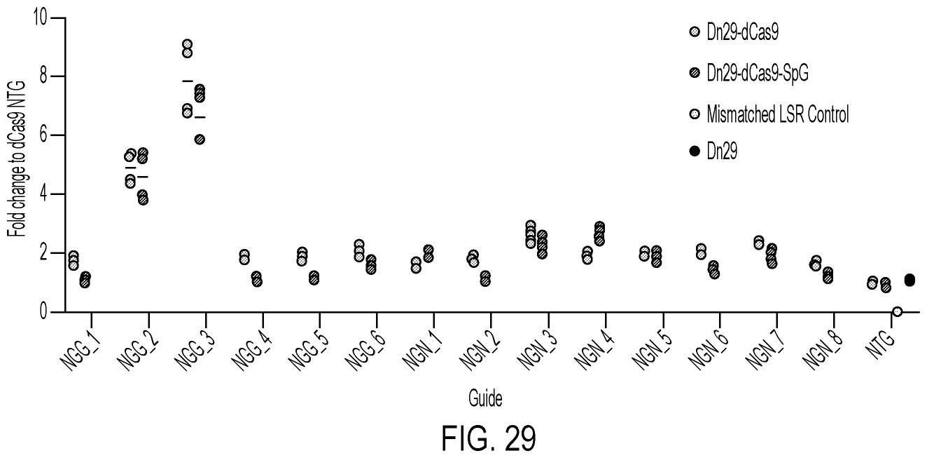

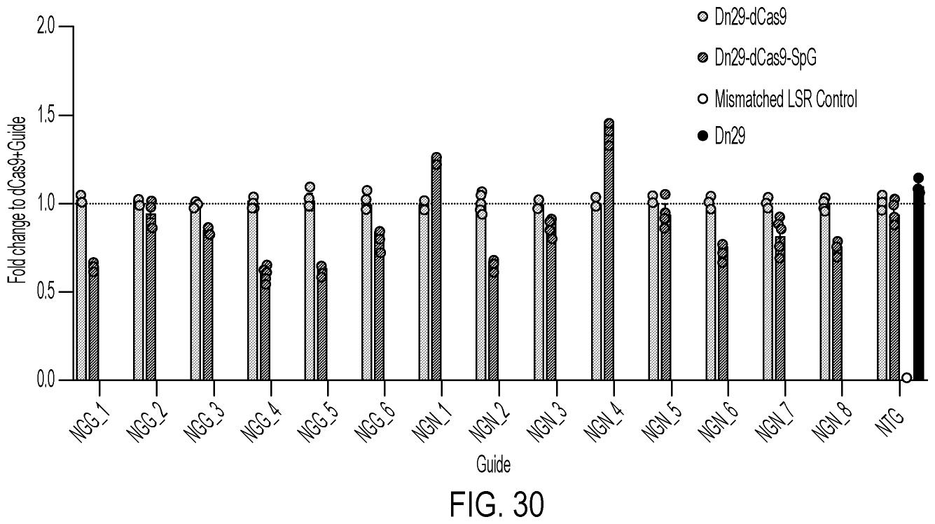

It was investigated how the gRNA distance to a pseudosite affects integration. FIG. 18 shows integration efficiency as a factor of distance from the core, with the distance being measured between the center of the dinucleotide core and the location between the protospacer and the PAM. In some embodiments, the distance from the core is <80 bp, including embodiments with functional guides proximal or directly outside the pseudosite sequence. This data indicates that the spacing between the PAM and the pseudosite will affect the ability to find functional guides to target new pseudosites. Given this insight, we next tested LSR fusions with a PAM flexible dCas9 variant called SpG, which has a NGN PAM specificity (FIG. 29).

In summary, we found that donor plasmid is the limiting reagent in these transfections, direct tethering between LSR and dCas9 is required, there does not appear to be steric hindrance caused by the non targeted dCas9s in the tetrameric complex, and a preferred gRNA position is directly proximal to the pseudosite.

In some embodiments, PAM-flexible Cas variants can be used to expanded guide RNA target choice.

### Example 5: Design Modifications to Optimize Integration Efficiency

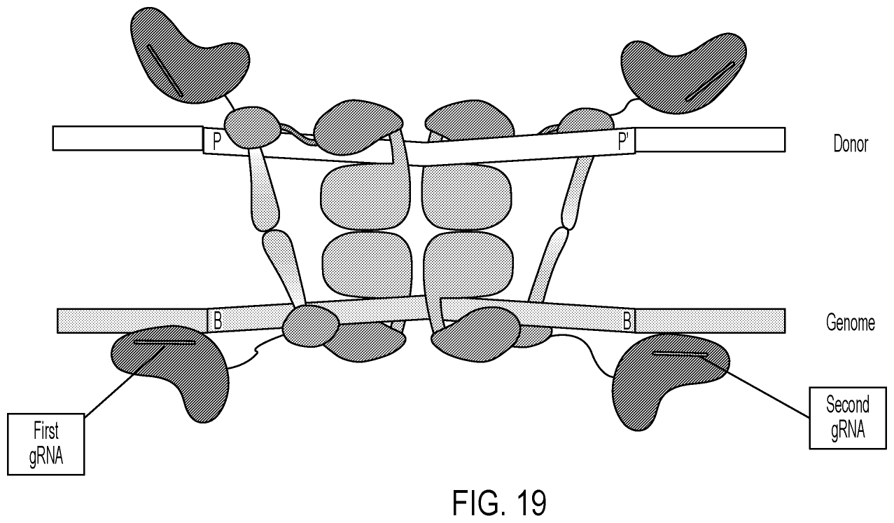

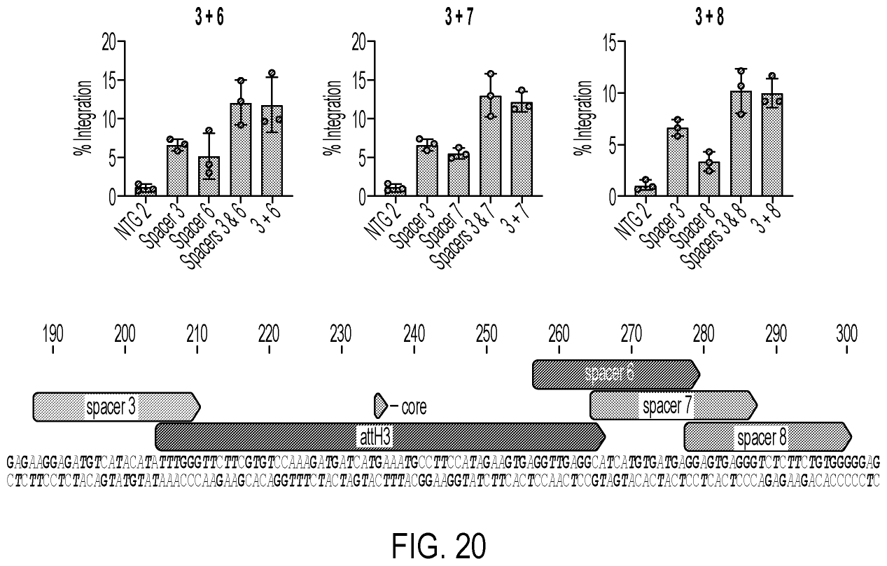

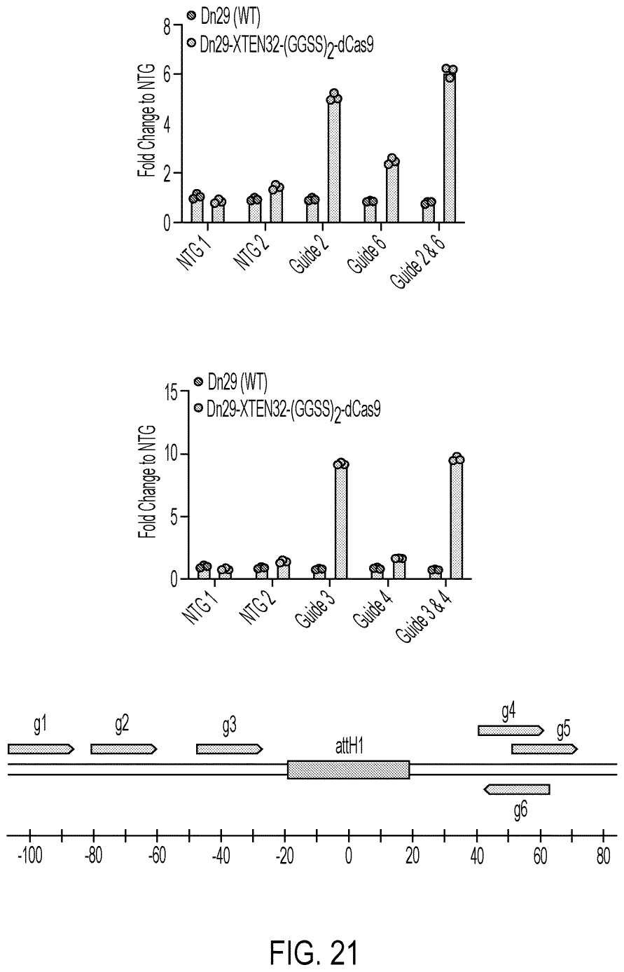

Various design modifications were tested to optimize the integration efficiency. In one embodiment, two guide RNAs which target upstream and downstream of the pseudosite are delivered, with the goal of increasing dimer formation on the genomic attachment site. A model of the tetrameric complex is shown in FIG. 19, in which two dCas9s are bound proximally to a pseudosite and two dCas9 monomers are unbound. Using this design, we show that delivering two target binding gRNAs has what appears to be an additive effect on integration, increasing integration at attH3 from ˜5-8% with a single guide to ˜10-13% with two guide RNAs (FIG. 20). Similarly for attH1, we show that multiplexing guides increases integration efficiency (FIG. 21).

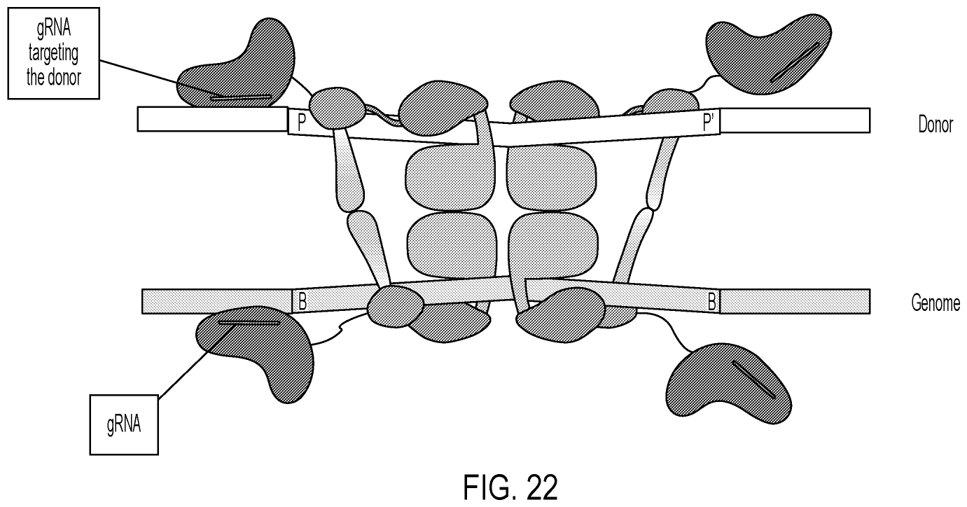

Another design modification for increased efficiency is the inclusion of a second gRNA that targets the donor plasmid. This guide may assist in recruitment of donor plasmid into the nucleus and/or facilitate dimer formation on the donor plasmid. A model of this tetrameric complex is shown in FIG. 22. Full length (20 bp) and truncated (16 bp) spacers were designed to target upstream and downstream of the attD on the donor plasmid. Truncated spacers will have reduced binding affinity, to potentially reduce the phenomenon of donor plasmid acting as a protein “sink” as [donor target]>>[genome target].

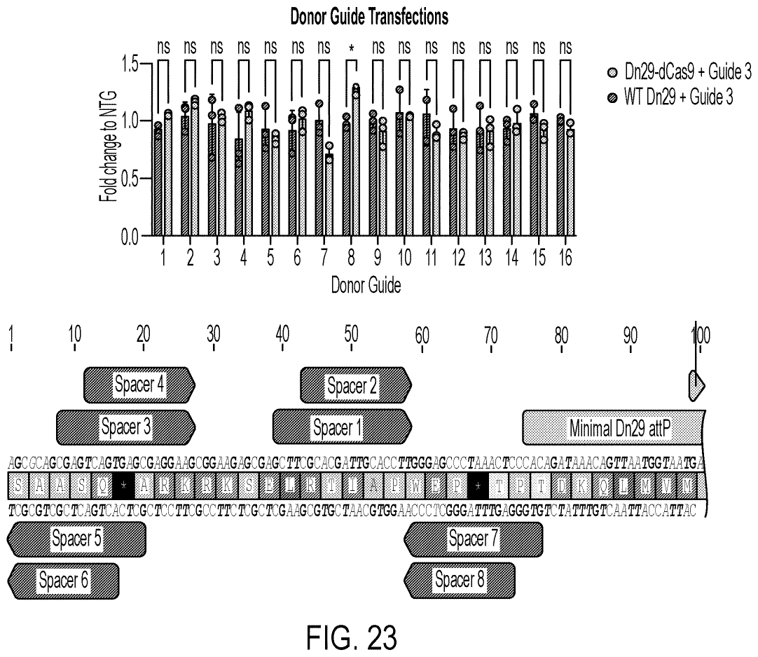

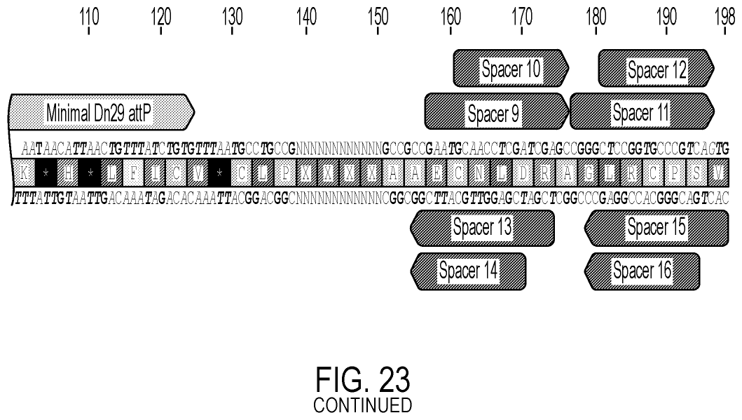

FIG. 23 shows guides targeting the donor slightly increase integration efficiency.

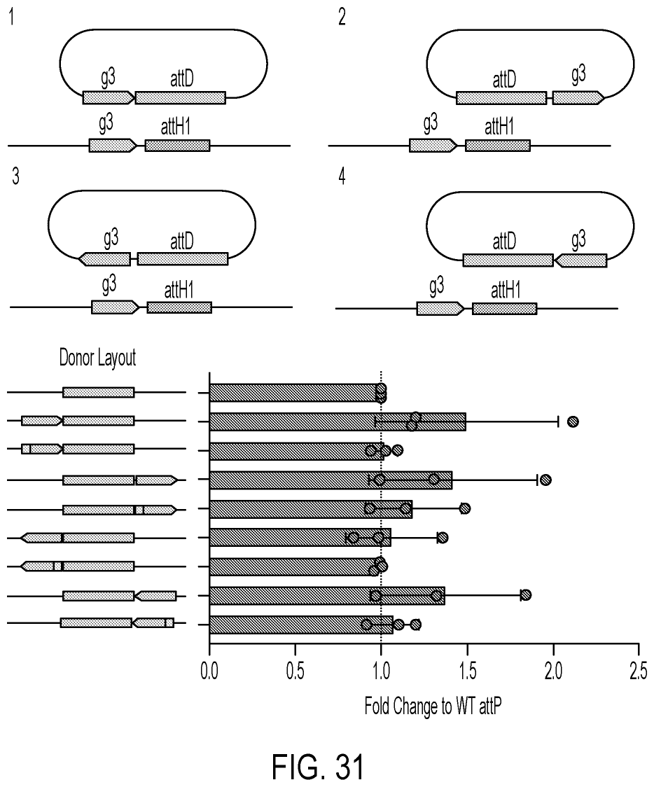

A modification of this approach is shown in FIG. 31, where the donor plasmid and gRNA is designed such that the target sequence of the gRNA spacer is found proximal to the attH1 and the attD, so a single guide will target the LSR-dCas9 to bind to both the target and the genome. A schematic of the possible orientations of this strategy is shown on top. The target sequence on the donor plasmid can either be a full length (20 bp) or truncated (16 bp). The bottom panel shows the increased efficiency resulting from this single guide dual targeting approach. With this design, a full length target sequence located proximally to the attD on the donor plasmid results in an up to 1.5 fold increase in efficiency over the standard donor without the target sequence.

In summary, multiplexed guides targeting the genomic pseudosite or the donor significantly increase integration efficiency. Pseudosites that are best candidates for guide multiplexing have functional guides both upstream and downstream. Guides targeting the donor plasmid have a modest positive effect on integration, with the preferable design being inclusion of a genomic target sequence for the gRNA on the donor such that a single gRNA will have dual targeting of the genome and the donor. Targeting the donor with a full length gRNA is preferable to a truncated guide where the last four bases are mismatches.

### Example 6: Measuring Effects of dCas9 Fusions on Specificity

Since targeting a Dn29 monomer to a single pseudosite increases efficiency, it will also increase specificity (on-target/off-target). To measure integration specificity, we developed an integration site mapping assay. HEK293FT cells are transfected with an effector (LSR or LSR-dCas9 fusion) plasmid, a UMI containing donor plasmid, and, in cases with a dCas9 fusion, a guide RNA plasmid. Genomic integration events are mapped with NGS as described in the methods, such that each unique integration event is counted with the UMI. The percent of UMIs at each locus measures the relative preference for integration at that site vs all other sites, illustrating the specificity profile for that effector.

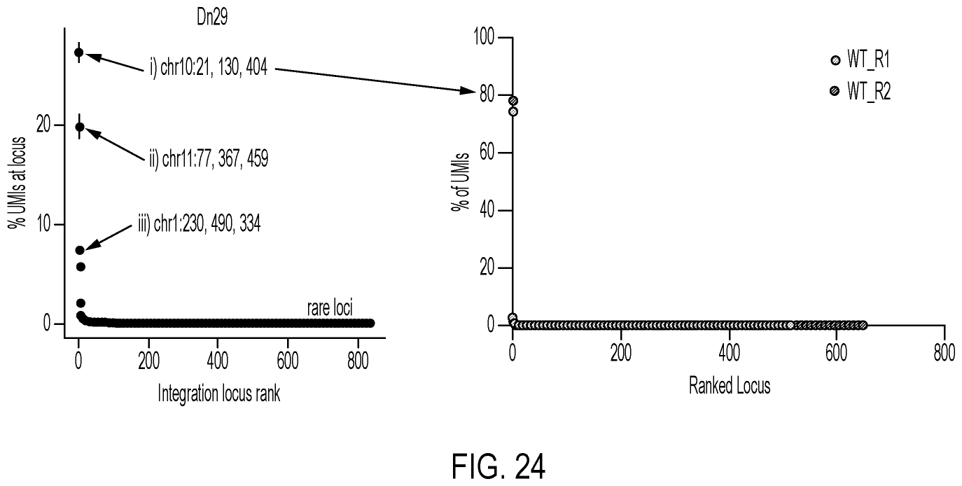

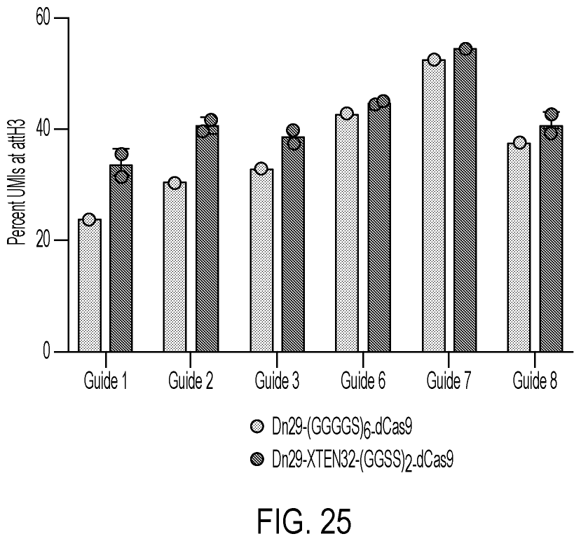

In FIG. 24, the specificity profile for Dn29 (left) vs Dn29-dCas9 (right) is shown. With the fusion system, specificity at attH1 increases from 30% to nearly 80%. For attH3, specificity increases from <10% to over 50% (FIG. 25). To look at the relationship between efficiency and specificity, we plotted the ddPCR result for each guide at attH1 and attH3 versus the specificity to the on-target site. The results indicate that guides with higher integration efficiency have fewer off-target integrations (FIG. 26), indicating that increasing efficiency to a single pseudosite is important for increased specificity.

### Example 7: Dn29-dCas9 Mediated Integration of a Plasmid Donor at attH1 In H1 Human Embryonic Stem Cells and in HepG2 Hepatocellular Carcinoma Cell Line

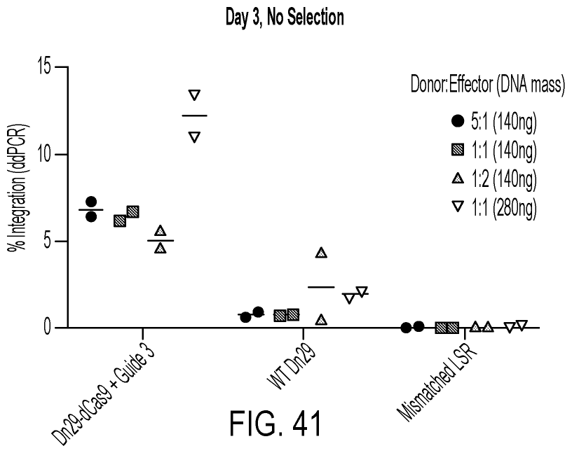

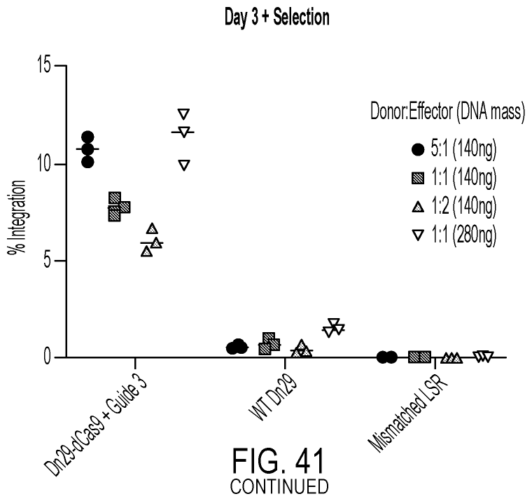

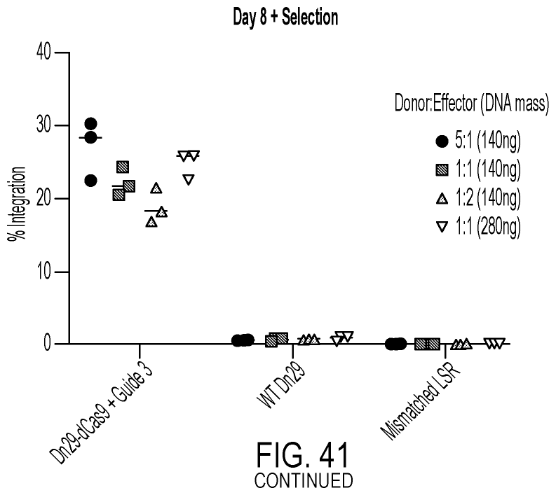

FIG. 41 shows Dn29-dCas9 mediated integration of a plasmid donor at attH1 in H1 human embryonic stem cells. Cells were transfected with a puromycin-expressing donor plasmid and an effector plasmid expressing both the Dn29-dCas9 effector and Guide 3 using the FuGENE Transfection reagent at the indicated Donor: Effector molar ratio with a total mass of 140 or 280 ng/well. As controls, WT Dn29 and a mismatched LSR were transfected as the effector with the same Dn29 donor plasmid. On day 1, the cells were split, and half were put on puromycin selection. Three days after transfection, the attH1 integration was measured by ddPCR from the no selection plate. Three and eight days after transfection, the attH1 integration percentage of the selected plate was measured by ddPCR. The results show that using selection can enrich for integrations. In this example, the LSR-DBD fusion (Dn29-dCas9) and the guide RNA were expressed from the same plasmid, with effector expression driven by Ef-1a and guide expression driven by U6.

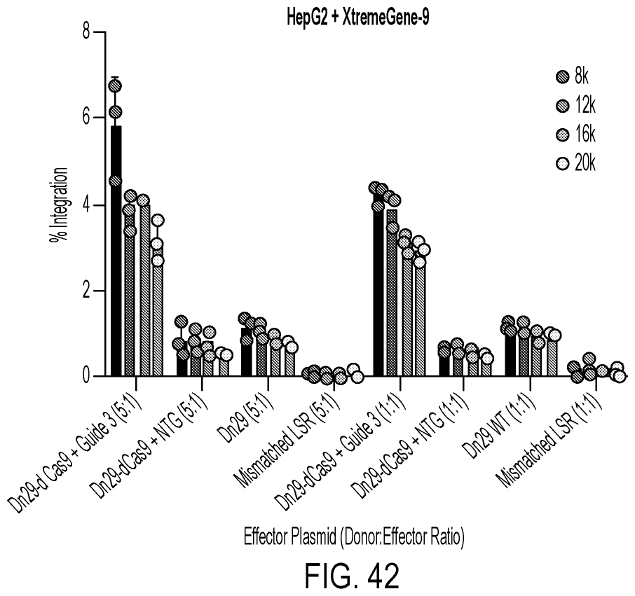

FIG. 42 shows Dn29-dCas9 mediated integration of a plasmid donor at attH1 in HepG2 hepatocellular carcinoma cell line. Cells were transfected with a puromycin-expressing donor plasmid and an effector plasmid expressing both the Dn29-dCas9 effector and Guide 3 using the XtremeGene-9 Transfection reagent at the specified molar ratio into cells seeded between 8-20 k cells/well as indicated in the figure legend. After 3 days, integration at attH1 was measured by ddPCR. In this example, the LSR-DBD fusion (Dn29-dCas9) and the guide RNA were expressed from the same plasmid, with effector expression driven by Ef-1a and guide expression driven by U6.

1. A nucleic acid comprising a sequence encoding a fusion polypeptide, wherein the fusion polypeptide comprises a large serine recombinase (LSR) portion and a DNA binding domain (DBD) portion.

2. (canceled)

3. (canceled)

4. The nucleic acid of claim 1, wherein the fusion polypeptide encoded by the nucleic acid further comprises a peptide linker positioned between the LSR portion and the DBD portion, wherein the LSR portion is fused N-terminal to the DBD portion by the peptide linker.

5. (canceled)

6. The nucleic acid of claim 4, wherein the peptide linker encoded by the nucleic acid comprises 2 to 100 amino acids and comprises glycine and serine residues, one or more XTEN16 repeats, or a combination thereof.

7. (canceled)

8. (canceled)

9. The nucleic acid of claim 6, wherein the peptide linker encoded by the nucleic acid comprises GGS, GGSS (SEQ ID NO: 584), GGGS (SEQ ID NO: 572), or GGGGS (SEQ ID NO: 596) repeats, one or more XTEN16 repeats, or a combination thereof.

10. (canceled)

11. The nucleic acid of claim 4, wherein the peptide linker encoded by the nucleic acid comprises one XTEN16 repeat, two XTEN16 repeats, or three XTEN16 repeats or comprises the amino acid sequence of SEQ ID NOs:11-15.

12. (canceled)

13. The nucleic acid of claim 4, wherein the nucleic acid sequence encoding the peptide linker comprises SEQ ID NOs:20-24.

14. The nucleic acid of claim 1, wherein the LSR portion encoded by the nucleic acid comprises an amino acid sequence at least 90% identical to SEQ ID NOs: 1-5, 432-443, 445-446, 448-467, 469-476, 478-492, 494-501, 276, 279, 282, 285, 288, or 291.

15. The nucleic acid of claim 14, wherein the LSR portion encoded by the nucleic acid comprises an amino acid sequence of SEQ ID NOs: 1-5, 432-443, 445-446, 448-467, 469-476, 478-492, 494-501, 276, 279, 282, 285, 288, or 291.

16. The nucleic acid of claim 15, wherein the LSR portion encoded by the nucleic acid comprises Dn29 (SEQ ID NO:1), Pf80 (SEQ ID NO:2), Cp36 (SEQ ID NO:3), Nm60 (SEQ ID NO:4), or Si74 (SEQ ID NO:5).

17. The nucleic acid of claim 16, wherein the nucleic acid sequence encoding the LSR portion comprises a nucleic acid sequence at least 90% identical to SEQ ID NOs:6-10.

18. The nucleic acid of claim 16, wherein the nucleic acid sequence encoding the LSR portion comprises a nucleic acid sequence of SEQ ID NOs:6-10.

19. The nucleic acid of claim 1, wherein the fusion polypeptide encoded by the nucleic acid further comprises one or more nuclear localization signals (NLSs).

20. The nucleic acid of claim 1, wherein the DBD portion encoded by the nucleic acid comprises Cas9, Cpf1, Cas12b, Cas12c, Cas12d, Cas12e, Cas12f, Cas12h, Cas12i, or Cas12g.

21. The nucleic acid of claim 20, wherein the Cas9, Cpf1, Cas12b, Cas12c, Cas12d, Cas12e, Cas12f, Cas12h, Cas12i, or Cas12g lack nuclease activity, nickase activity, or both nuclease and nickase activity.

22. The nucleic acid of claim 21, wherein the DBD portion encoded by the nucleic acid comprises dCas9.

23. The nucleic acid of claim 1, wherein the DBD portion encoded by the nucleic acid comprises an amino acid sequence at least 90% identical to dCas9 (SEQ ID NO:29), dCas9-HF1 (SEQ ID NO:30), dCas9-SpG (SEQ ID NO:31), or dCas9-SpG-HF1 (SEQ ID NO:32).

24. The nucleic acid of claim 23, wherein the DBD portion encoded by the nucleic acid comprises an amino acid sequence of dCas9 (SEQ ID NO:29), dCas9-HF1 (SEQ ID NO:30), dCas9-SpG (SEQ ID NO:31), or dCas9-SpG-HF1 (SEQ ID NO:32).

25. The nucleic acid of claim 24, wherein the nucleic acid sequence encoding the DBD portion comprises a nucleic acid sequence at least 90% identical SEQ ID NOs:33-36.

26. The nucleic acid of claim 24, wherein the nucleic acid sequence encoding the DBD portion comprises a nucleic acid sequence of SEQ ID NOs:33-36.

27. The nucleic acid of claim 1, wherein the fusion polypeptide encoded by the nucleic acid comprises Dn29 (SEQ ID NO: 1) and dCas9 (SEQ ID NO: 29), Pf80 (SEQ ID NO:2) and dCas9 (SEQ ID NO: 29), Cp36 (SEQ ID NO:3) and dCas9 (SEQ ID NO: 29), Nm60 (SEQ ID NO:4) and dCas9 (SEQ ID NO: 29), or Si74 (SEQ ID NO:5) and dCas9 (SEQ ID NO: 29).

28. The nucleic acid of claim 27, wherein the fusion polypeptide encoded by the nucleic acid further comprises a peptide linker positioned between the LSR portion and the DBD portion wherein the LSR portion is fused N-terminal to the DBD portion by the peptide linker and the peptide linker encoded by the nucleic acid comprises (GGS)₈ (SEQ ID NO: 11), (GGGGS)₆ (SEQ ID NO: 598), S(GGGGS)₆S (SEQ ID NO: 12), XTEN16 (SEQ ID NO: 13), XTEN32-(GGSS)₂ (SEQ ID NO: 14), or XTEN48-(GGSS)₂ (SEQ ID NO: 15).

29. The nucleic acid of claim 1, wherein the fusion polypeptide encoded by the nucleic acid comprises an amino acid sequence at least 90% identical to SEQ ID NOs: 37-42.

30. The nucleic acid of claim 1, wherein the fusion polypeptide encoded by the nucleic acid comprises an amino acid sequence of SEQ ID NOs: 37-42.

31. The nucleic acid of claim 1, wherein the DBD portion of the fusion polypeptide encoded by the nucleic acid binds to a guide RNA (gRNA).

32. A vector comprising the nucleic acid of claim 1.

33. A host cell comprising the vector of claim 32.

34. A nucleic acid editing system comprising a first nucleic acid according to claim 1 and a second nucleic acid encoding a gRNA.

35. The nucleic acid editing system of claim 34, wherein the gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides upstream or downstream of a dinucleotide core of an attachment site of the LSR portion of the fusion polypeptide on a DNA of interest.

36. The nucleic acid editing system of claim 35, wherein the gRNA encoded by the nucleic acid is an sgRNA comprising a spacer sequence portion 16 to 20 nucleotides long and a tracr RNA portion comprising SEQ ID NO: 153 and wherein immediately 3′ to the target nucleic acid sequence on the DNA of interest is a PAM sequence.

37.-47. (canceled)

48. The nucleic acid editing system of claim 35, wherein the target nucleic acid sequence is within 80 nucleotides upstream of a dinucleotide core of an attachment site of the LSR portion of the fusion polypeptide on a DNA of interest and the nucleic acid editing system further comprises a third nucleic acid encoding a second gRNA, wherein the second gRNA encoded by the nucleic acid comprises a spacer sequence portion and a tracr RNA portion, wherein the nucleic acid sequence of the spacer sequence portion is the same as a target nucleic acid sequence, except that T in the target nucleic acid sequence is U in the spacer sequence portion, and wherein the target nucleic acid sequence is within 80 nucleotides downstream of a dinucleotide core of an attachment site of the LSR portion of the fusion polypeptide on a DNA of interest.

49. The nucleic acid editing system of claim 48, wherein the second gRNA encoded by the nucleic acid is an sgRNA comprising a spacer sequence portion 16 to 20 nucleotides long and a tracr RNA portion comprising SEQ ID NO: 153 and wherein immediately 3′ to the target nucleic acid sequence on the DNA of interest is a PAM sequence.

50. (canceled)

51. (canceled)

52. The nucleic acid editing system of claim 35, further comprising a third nucleic acid comprising a donor DNA sequence which comprises an attD attachment site of the LSR portion of the fusion polypeptide, a nucleic acid sequence for insertion into the target DNA of interest, and a portion that has the same target nucleic acid sequence for the gRNA as the target DNA of interest.

53. (canceled)

54. (canceled)

55. (canceled)

56. (canceled)

57. A vector comprising any of the nucleic acids of the nucleic acid editing system of claim 34.

58. A host cell comprising any of the vector(s) of claim 57.

59. (canceled)

60. A method of integrating a donor DNA sequence into a target DNA of interest of a cell, the method comprising introducing into the cell: a nucleic acid editing system according to claim 34.

61.-68. (canceled)

69. A method of inverting a DNA sequence of a target DNA of interest, the method comprising introducing into a cell: a nucleic acid editing system according to claim 34, wherein attD and attA attachment sites of the LSR portion of the fusion polypeptide are present on the same DNA target molecule of interest in reverse orientation.

70. (canceled)

71. (canceled)

72. (canceled)

73. A method of excising a DNA sequence of a target DNA of interest, the method comprising introducing into a cell: a nucleic acid editing system according to claim 34, wherein attD and attA attachment sites of the LSR portion of the fusion polypeptide are present on the same DNA target molecule of interest in the same orientation.

74. (canceled)

75. (canceled)

76. (canceled)

77. A method of integrating DNA sequences between two target DNA molecules of interest, the method comprising introducing into a cell: a nucleic acid editing system according to claim 34, wherein an attD attachment site of the LSR portion of the fusion polypeptide is present on a first target DNA molecule and an attA attachment site of the LSR portion of the fusion polypeptide is present on a second target DNA molecule.

78. (canceled)

79. (canceled)

80. (canceled)

81. A fusion polypeptide encoded by the nucleic acid of claim 1.

82. A ribonucleoprotein complex comprising the fusion polypeptide of claim 81 and a gRNA.


SEQUENCE LISTING (631 sequences)

SEQ ID NO: 1 - protein, 519 aa, Desulfotomaculum nigrificans
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINS

SEQ ID NO: 2 - protein, 532 aa, Pseudomonas fluorescens
MKQAISYVRFSSDRQRHGSSVERQEGMIADWLKRHPDYEMSDLKFSDLGKSGYHGEHIKEAGGFGKLLKALEDGFIRAGDVVLVEAIDRTGRLEPMDMLTLVINPILKAGVSIITLDDNTTYSKESVNTAQIFLLVAKIQAAHGYSAALSTRVADSYKKRRKDAAKYGIVPRRITPVWLNPDGTVREDVAPWIKTAFELYVSGVGKSTIAKRMRESGVERLAKASGPGVEGWLRNKAVIGKWETLEGTPDHQIIDDIYPAIIEPSLFYKAQVHAEKMKTQRPIKTASHFLVGLVRCGECGKNYIVQNLHGKPHSMRCRTRQSQNECTNSHCVPKPVLDAIYRYTSVTAAIEAVQQQQMGVNDKEIVTREAELLAITKRVDGLVQALTQTGPIPEVIEALKQSRIEREAAESALVILRSTVVPSAGTHWQEMGKVWKLEAADAQRLAAMLKLVDYHITVAPTGEITASHSEVLYRYIGVDRALDKYKLLANGKLMLIPKGYVDDFPYHEPFQEMQSENTWDEADYDNLRQQHQ

SEQ ID NO: 3 - protein, 544 aa, Clostridium perfringens
MKQLNIQSSSKITALYCRLSRDDELQGTSNSILNQKMMLEKYARDNNFTNLEFFIDDGYSGTNFNRPDWSRLQSLIDEGKIGCIIVKDMSRLGRDYLRVGYYTDIVFPEADIRFIAINNGIDSNESTENDLTPFINIINEFYAKDTSKKIRAVFKAKGESGKPLATIPPYGYLKDKEDKYKWVIDEEASKVVKKIFQLCVQGYGPSQIASELIKEGIPTPTEHFDKLGINVSSPLSEIKGNWQPKTISLILEKMEYLGHTVNFKTYKKSYKSKKKLENPKEKWQIFENTHEAIIDQETFDIVQRIRQGRRVRNNLGEMPALSGMLYCADCGAKLYQVRGKGWEHEKEYFVCASYRKHKGLCTSHQIKNVQVEELLLHELKKITEYARQYEDDFVKLVQSKTQNELNKSLKESKKDLVHVKERINKLDTIIQRLYEDMVEGKLSEDRFQKLSSNYETEQCELEKKATMLEKIIHDTEQTTLNTTAFLKQVREHTTINKLTPEIIRMFVDKIIVEKPEKIEGTRTKKQTIWIYWNYIGILDIEKTA

SEQ ID NO: 4 - protein, 515 aa, unidentified
MSRPTGLTIDIYLRKSRKDLEEEKKASESGETYDTLERHRRTLFAVAKKERHNIANIYEEVVSGESVSERPQIQAMLRNLETSHIEGVLVMDLDRLGRGDMLDQGLLDRAFRYSGAKILTPTEVYDPESETWELVFGIKSLVAREELKAITRRMQRGRVASAGEGKSISKVPPYGYLRDENLRLYPDPETAWVVKKMFEMMRDGHGRIAVAQELDKLGIKPPNDKRRSWAPSSITAIIKNEVYLGTIIWGQVKYSKRNGKYKKTKLPRSKWTIKENAHEPIVSRELFEAANRAHTGRWRPSTNATKTLSNPLAGVLKCDVCGFTMLYQPRPNRPNDFIRCTQPTCQAVQKGATLALVEQHILDGLKQFAQELELQTEVPELDNDKDIAVKKSLVGNKQEEIAQLETQKSKLHDLLERGIYDVDTFLERQQNLNNRINGLQDDIRNIESEIKKEEVRNSSVLNLLPQLQTVISEYENADTESKNRLLKSVLEKATYLRKKEWTKRDQFIIQLYPKI

SEQ ID NO: 5 - protein, 555 aa, Streptomyces ipomoeae
MQPNLRYLACLRLSADSDGSTSIEWQRGVIRHHVSSPHLSGVLVGEAEDTDVSGSLSPFKRPKLGKWLTAKADEFDVIIAAKMDRLTRRSMHFNELLEWAQQNGKFIVCVEEGFDLSTPQGKMMARMTAVFAEAEWDTIQARILNGVQTRLENRSWLVGAPPTGYRIKTVEGGKRKILEIDQDFYPYVEEIFRRIREGQSTHRIARDFNGRSVLTWGDHLRKLKGEEPKGTQWQATIINKFIRSSWVPGLYTYKGEAVLDDQGDPVILPETPLATMDEWTDLVDRIKPAPKPEGATGGSRNSAKSLLSGVAHCGECGAPFTSLMDSGYKRKDGTKVPGHRRYRCSNKFKGGDCKNGSYVRADVLDSWVDQAIRDSIGQEDMYERAGKGPSQARELQETKARLAKLEADYESGKYDGEGQDESYWRMNKNLSAKVAHLAKQEAERANPTFKATGKKYGEVWEAKDQEDRRDFLRTYGVKVFVWGEGADKKDRGYAMNLGDIKTMAEELFPNRDRARFKLVHTHNAPEGYLSKIGIAVGLLKYGHPLEVKLRSPENS

SEQ ID NO: 6 - DNA, 1557 bp, Desulfotomaculum nigrificans
  source 1..1557: mol_type = genomic DNA
atggaacgggtgctcatgcaccttagaaagtccagagccgatctggaagctgaggccaggggcgagggcgaaaccctggctaaacatagaaacatcctgctgaagctggccaaagaaagaaatctgaacatcgtgaagatcagagaggaagtgggcatcagcggcgagtctctgatccacagacctgagatgctggagacactgaaggaggtggaagagggactgtacgacgctgtgctgtgcatggacatcgacagactgggcagaggcaataccaaggagcagggcattatcctggaggccttcaagaatagcaacaccaagatcatcacccctctgaaaacatacgacctgaacaacgagttcgacgaggaatacgccgagtttggcgccttcatggccaggcgggaattcaagtttatcaccagaagactgcagcggggcagagtggccacagttgaggagggaaattacatcgctccaagaccaccttacggctacatcatcgagaaaaacaacaaggagcggtacctgattcctcaccctgagcaggcccctatcgtgaagatgatctttgactggtacacccacgacgatcctaacgtgcggatgggcgccagcaagatcgccaacgagctgaacaagttctgcaagagccctacaggcatcgcctggaagggctccaccgtgttgtctatcctgaagaatgccgtgtacgccggcagaatccagtggcggaagaaggaggaaaaaaaatctatcacaccaggcaaaaaaaaggatgtccggatgcggcccaaggaacaatggatcgacgtggagggcaagcacgaacctctggtgtccatggaaacctacctgaaggcccaagagatcctggagcggaagtaccacgtgccctatcagatccagaacggcatcactaaccccctggccggactggtcaagtgcggaatttgtggtagcagcatggtgtatagaccttacacccaccagcgccacccccacctgatctgctacaacagatactgtaccaacaagtctagcagattcgagtacgtggaaagcaaaattatccaaggcctgcaccagctgctggcacagtacaaggccgattggttcaaacatcagagacccaaggtgaacgacgacagcgtggacctgagacagaaagccctccaccggctggagaaggaactgaatgacctgtacaagcagaaggacaacctgcacaatctgctggagaagggcgtgtacagcatcgatacattcctggaaagaagcaacatcctggctgaacgcatcgatgataccaagaaggccatccacgacgccgagaaggctctggccgaggaggtgcagcggaacaaagtgaagaaggatatcatccccacagtcgagaacgtgcttgaactgtactacaagacccaggaccctgccaagaagaacaacctgctgaagagcgtgctggactacgctgtttacagaaaagaaaagcaccagagagacgatgacttcaccttggtgctttatcctaagctgcctcagatcaacagc

SEQ ID NO: 7 - DNA, 1596 bp, Pseudomonas fluorescens
  source 1..1596: mol_type = genomic DNA
atgaagcaggccatcagctacgtgcggttcagcagcgacagacagagacacggcagcagcgtggaaagacaagagggcatgatcgccgactggctgaagagacaccccgactacgagatgagcgacctgaagttcagcgacctgggcaagtctggctatcacggcgagcacatcaaagaggctggcggctttggcaagctgctgaaggctctggaagatggcttcatcagagccggcgacgtggtgctggtggaagccatcgatagaaccggcagactggaacccatggacatgctgaccctggtcatcaaccccatcctgaaagccggcgtgtccatcatcaccctggacgacaataccacctacagcaaagaatccgtgaatacggcccagatcttcctgctggtggccaagattcaggccgctcacggatatagcgccgctctgtctacaagagtggccgacagctacaagaagcggagaaaggacgccgccaaatacggcatcgtgcccagacggattacccctgtgtggctgaatcctgacggcaccgtcagagaagatgtggccccttggatcaagaccgccttcgagctgtatgtgtccggcgtgggcaagagcacaatcgccaagcggatgagagaaagcggcgtcgagagactggccaaggcttctggacctggcgttgaaggctggctgagaaacaaggccgtgatcggcaagtgggagacactggaaggcacccctgaccaccagatcatcgacgacatctaccccgccatcatcgagcccagcctgttctataaggcccaggtgcacgccgagaagatgaagacccagcggcctatcaagacagccagccactttctcgtgggcctcgtcagatgtggcgagtgcggcaagaactacatcgtgcagaacctgcacggcaagccccacagcatgagatgcagaaccagacagagccagaacgagtgcaccaacagccactgtgtgcccaagcctgtgctggacgccatctacagatacacctctgtgaccgccgccatcgaagctgtgcagcagcagcaaatgggcgtgaacgacaaagaaatcgtgaccagagaggccgagctgctggccatcaccaagcgagttgatggactggtgcaggccctgacacagacaggccctattccagaagtgatcgaggccctgaagcagagccggattgaaagagaagccgccgagagcgccctcgtgatcctgagatctacagtggtgcctagcgccggcacacactggcaagagatgggcaaagtgtggaagctggaagccgctgacgcccaaagactggccgctatgctgaagctggtggactaccacatcacagtggcccctaccggcgagatcacagcctctcatagcgaggtgctgtaccgctacatcggcgttgacagagccctggacaagtacaagctgctcgccaacggcaagctgatgctgatccctaagggctacgtggacgacttcccctaccacgagcctttccaagagatgcagtccgagaacacctgggacgaagccgattacgacaacctgagacagcagcaccag

SEQ ID NO: 8 - DNA, 1632 bp, Clostridium perfringens
  source 1..1632: mol_type = genomic DNA
atgaagcagctgaacatccagagcagcagcaagatcacagccctgtactgcagactgagccgggacgatgaactgcagggcaccagcaacagcatcctgaaccagaaaatgatgctcgagaagtacgcccgggacaacaacttcaccaacctggaattcttcatcgacgacggctacagcggcaccaacttcaacagacccgactggtccagactgcagagcctgatcgatgagggcaagatcggctgcatcatcgtgaaggacatgagccggctgggcagagactatctgagagtgggctactacaccgacatcgtgttccccgaggccgacatccggtttatcgccatcaacaacggcatcgacagcaacgagagcaccgagaacgatctgacccctttcatcaacatcatcaacgagttctacgccaaggacaccagcaagaaaatccgggccgtgttcaaggccaagggcgagtctggaaagcccctggccacaattcctccatacggctacctgaaggacaaagaggacaagtacaaatgggttatcgacgaagaggccagcaaggtcgtgaagaaaatcttccagctgtgcgtgcaaggctacggcccttctcagattgccagcgagctgatcaaagagggcatccccacacctaccgagcacttcgataagctgggcatcaacgtgtcctctccactgagcgagatcaaaggcaactggcagcccaagaccatcagcctgatcctggaaaagatggaatacctgggccacaccgtgaacttcaagacctacaagaagtcctacaagagcaagaagaagctcgagaaccccaaagagaagtggcagatcttcgagaacacccacgaggccatcatcgaccaagagacattcgatatcgtgcagcggatccggcagggcagaagagtgcgaaacaatctgggcgagatgcccgctctgagcggcatgctgtattgtgccgattgtggcgccaagctgtaccaagtgcgcggcaaaggatgggagcacgagaaagaatacttcgtgtgcgccagctaccggaagcacaagggcctgtgtacaagccaccagatcaagaacgtgcaggtcgaggaactgctgctgcacgagctgaagaagatcaccgagtacgccagacagtacgaggacgacttcgtgaagctggtgcagagcaagacccagaatgagctgaacaagagcctgaaagagtccaagaaagacctggtgcacgtgaaagagcggatcaacaagctggacaccatcatccagcggctgtacgaggatatggtggaaggcaagctgagcgaggaccggttccagaagctgagcagcaactacgagacagagcagtgcgagctggaaaaaaaggccaccatgctcgaaaagatcatccacgacaccgagcagacaaccctgaacaccaccgcctttctgaagcaagtgcgggaacacaccaccattaacaagctgacccctgagatcatccgcatgttcgtggacaagatcatcgtggaaaagcccgagaagatcgaaggcacccggaccaagaagcagaccatctggatctactggaactacatcggcatcctggacatcgagaaaaccgcc

SEQ ID NO: 9 - DNA, 1545 bp, unidentified
  source 1..1545: mol_type = unassigned DNA
atgagcagacctacaggcctgaccattgacatctacctgcggaaatctagaaaggatctggaagaagagaagaaagccagcgagagcggcgaaacctacgatacactggaaagacacagacggaccctcttcgccgtggctaagaaagagcggcacaacatcgctaacatctatgaggaagtcgtgtccggcgagtccgtgtctgagcgaccacagatccaagctatgctgagaaacctggaaaccagccacatcgagggcgtgctggttatggacctggatagactgggcagaggcgacatgctggaccagggactgctggacagagccttcagatacagcggagccaagatcctgacccctaccgaggtgtacgaccccgagagcgagacatgggagctggtgttcggcatcaagtctctggtggccagagaggaactgaaagccatcacaagaaggatgcagagaggaagagtggcctctgccggcgagggaaagtccatcagcaaggtgccaccttacggctatctgcgcgatgagaacctgagactgtaccctgatcctgaaaccgcctgggtggtgaagaaaatgttcgagatgatgagagatggccacggcaggatcgccgtggcccaggagctggacaagctgggaatcaaacctcctaatgataagcggcggagctgggcccctagcagcatcaccgccatcatcaagaacgaggtctacctgggcaccatcatttggggccaggtgaagtacagcaagcggaacggcaagtacaagaagaccaagctgcctagatctaagtggaccatcaaggaaaacgctcatgagcctatcgtgtccagagaactcttcgaggccgctaatcgggcccacacaggcagatggcggccttcaacaaacgccacaaagaccctgagcaaccccctggctggcgttctgaagtgcgacgtgtgcggcttcaccatgctgtaccagcccagacctaaccggcccaatgacttcatccggtgcacccagcctacctgtcaggccgtgcagaagggcgccaccctggccctggtggaacagcacatcctggacggcctgaagcaatttgcccaggagctggaactgcaaacagaagtgcccgagctggacaacgacaaggacatcgcagtgaaaaagagcctggtggggaacaagcaggaggaaatcgcccagctggaaacacagaaaagcaagctgcacgacctgcttgaacgcggcatctacgacgtggacaccttcctggaaagacagcagaacctgaataaccggatcaacggcctgcaggacgacatcagaaacatcgagtctgagatcaagaaagaagaggtgcggaacagcagcgtgctgaatctgctgccgcaacttcagaccgtgatcagcgagtacgagaacgccgataccgaaagcaagaacagactgctgaagagcgtgctggaaaaggccacatacctgagaaagaaagagtggacaaagagagatcagtttatcattcagctgtaccccaagatc

SEQ ID NO: 10 - DNA, 1665 bp, Streptomyces ipomoeae
  source 1..1665: mol_type = genomic DNA
atgcaacctaatctgcggtacctggcatgcctgagactcagcgccgatagcgacggcagcacatctatcgagtggcaaagaggcgtgattagacaccacgtgtccagcccccacctgagcggagttctggtgggcgaagccgaggacaccgatgtgtccggctctctgtccccatttaagcggcccaagctgggcaagtggctgacggccaaggctgacgagttcgacgtgatcatcgccgccaagatggacagactgaccagaagaagcatgcacttcaatgagctgctggaatgggcccagcagaacggcaaattcatcgtgtgcgtcgaagagggcttcgatctgtccacacctcagggaaagatgatggccagaatgaccgctgtgtttgccgaggccgagtgggacacaatccaggccagaatcctgaacggcgtccagaccagactcgaaaaccggagctggctggtgggagccccacctacaggctacagaatcaagaccgtggaaggcggcaagagaaagatcctggagatcgaccaggacttctacccatacgtggaagaaatcttccggcggatcagagagggccagagcacccacagaatcgccagagattttaacggaagaagcgtgctgacctggggcgaccatctgagaaagctgaaaggagaagaacctaagggcacacagtggcaggctaccatcatcaacaagtttattcggagctcttgggtgcctggactgtacacctacaagggcgaggccgtgctggacgaccagggcgatcctgtgatcctgcctgaaacccctctggctaccatggacgagtggaccgacctggtggaccgcatcaagcctgctcctaagcccgagggcgccaccggcggttctagaaactctgctaaatccctgctgagcggcgtcgcccactgtggcgagtgcggcgcccctttcacctctctgatggattctggctacaagaggaaggatggcaccaaggtgcccggccaccggcgatatcggtgcagcaacaaattcaagggcggagattgtaaaaacggcagctacgtgcgggccgatgtgcttgatagctgggtcgaccaagccatccgggacagcatcggccaggaggatatgtacgagagagccggcaagggccctagccaggccagggagctgcaggagacaaaggcccggctggccaaactggaagccgactacgagagcgggaagtacgacggcgagggacaggatgaaagctactggcggatgaacaagaacctcagcgccaaggtggcccatttggccaaacaggaggcagaaagagccaaccccacattcaaagccaccggcaagaagtatggcgaggtgtgggaggctaaggaccaggaagacagaagagacttcctgagaacatacggcgttaaggtgttcgtgtggggcgaaggcgctgacaagaaagaccggggatacgccatgaatctgggtgatatcaagacaatggctgaagagctgttccccaatagagacagagccagattcaagctggtgcacacccacaacgcccctgagggctatctgagcaagatcggaatcgccgtgggcctgctgaagtacggccaccccctggaggtgaagctgagaagccctgagaacagc

SEQ ID NO: 11 - protein, 24 aa, synthetic construct
GGSGGSGGSGGSGGSGGSGGSGGS

SEQ ID NO: 12 - protein, 32 aa, synthetic construct
SGGGGSGGGGSGGGGSGGGGSGGGGSGGGGSS

SEQ ID NO: 13 - protein, 17 aa, synthetic construct
SGSETPGTSESATPESS

SEQ ID NO: 14 - protein, 42 aa, synthetic construct
SGSETPGTSESATPESSGSETPGTSESATPESSGGSSGGSSS

SEQ ID NO: 15 - protein, 66 aa, synthetic construct
SGGSSGGSSGSETPGTSESATPESSGSETPGTSESATPESSGSETPGTSESATPESSGGSSGGSSS

SEQ ID NO: 16 - protein, 58 aa, synthetic construct
SGSETPGTSESATPESSGSETPGTSESATPESGSGVKQTLNFDLLKLAGDVESNPGPS

SEQ ID NO: 17 - protein, 56 aa, synthetic construct
SGSETPGTSESATPESSGSETPGTSESATPESGSGQCTNYALLKLAGDVESNPGPS

SEQ ID NO: 18 - protein, 55 aa, synthetic construct
SGSETPGTSESATPESSGSETPGTSESATPESGSGATNFSLLKQAGDVEENPGPS

SEQ ID NO: 19 - protein, 54 aa, synthetic construct
SGSETPGTSESATPESSGSETPGTSESATPESGSGEGRGSLLTCGDVEENPGPS

SEQ ID NO: 20 - DNA, 75 bp, synthetic construct
  source 1..75: mol_type = other DNA
ggggggtcaggtggatccggcggaagtggcggatccggtggatctggcggcagtggcgggagtggaggttctagc

SEQ ID NO: 21 - DNA, 96 bp, synthetic construct
  source 1..96: mol_type = other DNA
tcaggtggtggcggttcaggcggaggtggctctggcggtggcggatcgggtgggggcggatccggtgggggaggctctggcggcggcgggagtagc

SEQ ID NO: 22 - DNA, 51 bp, synthetic construct
  source 1..51: mol_type = other DNA
tctggtagcgagacacccggtaccagtgaaagcgccacgccagaaagcagc

SEQ ID NO: 23 - DNA, 126 bp, synthetic construct
  source 1..126: mol_type = other DNA
tctggtagcgagacacccggtaccagtgaaagcgccacgccagaaagcagtgggagtgagactccgggtacatctgaatcagcgacaccggaatcaagtggcggcagcagcggcggcagcagcagc

SEQ ID NO: 24 - DNA, 198 bp, synthetic construct
  source 1..198: mol_type = other DNA
tctggaggatctagcggaggatcctctggcagcgagacaccaggaacaagcgagtcagcaacaccagagagctctggtagcgagacacccggtaccagtgaaagcgccacgccagaaagcagtgggagtgagactccgggtacatctgaatcagcgacaccggaatcaagtggcggcagcagcggcggcagcagcagc

SEQ ID NO: 25 - DNA, 174 bp, synthetic construct
  source 1..174: mol_type = other DNA
tctggtagcgagacacccggtaccagtgaaagcgccacgccagaaagcagtgggagtgagactccgggtacatctgaatcagcgacaccggaatcaggctcgggcgtgaagcagaccctgaacttcgacctgctgaagctggccggagatgtggagagcaaccccggccctagc

SEQ ID NO: 26 - DNA, 168 bp, synthetic construct
  source 1..168: mol_type = other DNA
tctggtagcgagacacccggtaccagtgaaagcgccacgccagaaagcagtgggagtgagactccgggtacatctgaatcagcgacaccggaatcaggctcgggccagtgtactaattatgctctcttgaaattggctggagatgttgagagcaacccaggtcccagc

SEQ ID NO: 27 - DNA, 165 bp, synthetic construct
  source 1..165: mol_type = other DNA
tctggtagcgagacacccggtaccagtgaaagcgccacgccagaaagcagtgggagtgagactccgggtacatctgaatcagcgacaccggaatcaggcagcggcgccacaaacttctctctgctaaagcaagcaggtgatgttgaagaaaaccccgggcctagc

SEQ ID NO: 28 - DNA, 162 bp, synthetic construct
  source 1..162: mol_type = other DNA
tctggtagcgagacacccggtaccagtgaaagcgccacgccagaaagcagtgggagtgagactccgggtacatctgaatcagcgacaccggaatcaggctccggcgagggcaggggaagtcttctaacatgcggggacgtggaggaaaatcccggcccaagc

SEQ ID NO: 29 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 30 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 31 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 32 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 33 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 34 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccgccttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcgccctgagccggaagctgatcaacggcatccgggacaagcagtccggcaagacaatcctggatttcctgaagtccgacggcttcgccaacagaaacttcatggccctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccgggccatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctactggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 35 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggtgac

SEQ ID NO: 36 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccgccttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcgccctgagccggaagctgatcaacggcatccgggacaagcagtccggcaagacaatcctggatttcctgaagtccgacggcttcgccaacagaaacttcatggccctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccgggccatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcctgtggcccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccaagcagctgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagcagtacagaagcaccaaagaggtgctggacgccaccctgatccaccagagcatcaccggcctgtacgagacacggatcgacctgtctcagctgggaggcgac

SEQ ID NO: 37 - protein, 1944 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINSGPKKKRKVSGGGGSGGGGSGGGGSGGGGSGGGGSGGGGSSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 38 - protein, 1937 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINSGPKKKRKVGGSGGSGGSGGSGGSGGSGGSGGSSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 39 - protein, 1954 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINSGPKKKRKVSGSETPGTSESATPESSGSETPGTSESATPESSGGSSGGSSSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 40 - protein, 1978 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINSGPKKKRKVSGGSSGGSSGSETPGTSESATPESSGSETPGTSESATPESSGSETPGTSESATPESSGGSSGGSSSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 41 - protein, 1929 aa, synthetic construct
MERVLMHLRKSRADLEAEARGEGETLAKHRNILLKLAKERNLNIVKIREEVGISGESLIHRPEMLETLKEVEEGLYDAVLCMDIDRLGRGNTKEQGIILEAFKNSNTKIITPLKTYDLNNEFDEEYAEFGAFMARREFKFITRRLQRGRVATVEEGNYIAPRPPYGYIIEKNNKERYLIPHPEQAPIVKMIFDWYTHDDPNVRMGASKIANELNKFCKSPTGIAWKGSTVLSILKNAVYAGRIQWRKKEEKKSITPGKKKDVRMRPKEQWIDVEGKHEPLVSMETYLKAQEILERKYHVPYQIQNGITNPLAGLVKCGICGSSMVYRPYTHQRHPHLICYNRYCTNKSSRFEYVESKIIQGLHQLLAQYKADWFKHQRPKVNDDSVDLRQKALHRLEKELNDLYKQKDNLHNLLEKGVYSIDTFLERSNILAERIDDTKKAIHDAEKALAEEVQRNKVKKDIIPTVENVLELYYKTQDPAKKNNLLKSVLDYAVYRKEKHQRDDDFTLVLYPKLPQINSGPKKKRKVSGSETPGTSESATPESSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 42 - protein, 1967 aa, synthetic construct
MKQAISYVRFSSDRQRHGSSVERQEGMIADWLKRHPDYEMSDLKFSDLGKSGYHGEHIKEAGGFGKLLKALEDGFIRAGDVVLVEAIDRTGRLEPMDMLTLVINPILKAGVSIITLDDNTTYSKESVNTAQIFLLVAKIQAAHGYSAALSTRVADSYKKRRKDAAKYGIVPRRITPVWLNPDGTVREDVAPWIKTAFELYVSGVGKSTIAKRMRESGVERLAKASGPGVEGWLRNKAVIGKWETLEGTPDHQIIDDIYPAIIEPSLFYKAQVHAEKMKTQRPIKTASHFLVGLVRCGECGKNYIVQNLHGKPHSMRCRTRQSQNECTNSHCVPKPVLDAIYRYTSVTAAIEAVQQQQMGVNDKEIVTREAELLAITKRVDGLVQALTQTGPIPEVIEALKQSRIEREAAESALVILRSTVVPSAGTHWQEMGKVWKLEAADAQRLAAMLKLVDYHITVAPTGEITASHSEVLYRYIGVDRALDKYKLLANGKLMLIPKGYVDDFPYHEPFQEMQSENTWDEADYDNLRQQHQGPKKKRKVSGSETPGTSESATPESSGSETPGTSESATPESSGGSSGGSSSPKKKRKVEASDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGPKKKRKV

SEQ ID NO: 43 - DNA, 20 bp, synthetic construct
  source 1..20: mol_type = other DNA
ttgctaaggtgtatttaatg

SEQ ID NO: 44 - DNA, 20 bp, synthetic construct
  source 1..20: mol_type = other DNA
gtcttggtgaatgtctcatg

SEQ ID NO: 45 - DNA, 20 bp, synthetic construct
  source 1..20: mol_type = other DNA
agtgtgtattctgctgttgt

SEQ ID NO: 46 - DNA, 20 bp, synthetic construct
  source 1..20: mol_type = other DNA
ttctgcctgctggatctgtc

SEQ ID NO: 47 - DNA, 20 bp, synthetic construct
  source 1..20: mol_type = other DNA
gatctgtctggtactgatag

SEQ ID NO: 48 - DNA, 20 bp, synthetic construct
  source 1..20: mol_type = other DNA
cagcaggcagaaaatcagca

SEQ ID NO: 49 - DNA, 20 bp, synthetic construct
  source 1..20: mol_type = other DNA
attgcaattgacttctgaga

SEQ ID NO: 50 - DNA, 20 bp, synthetic construct
  source 1..20: mol_type = other DNA
aaggagatgtcatacatatt

SEQ ID NO: 51 - DNA, 20 bp, synthetic construct
  source 1..20: mol_type = other DNA
aggagatgtcatacatattt

SEQ ID NO: 52 - DNA, 20 bp, synthetic construct
  source 1..20: mol_type = other DNA
cttccatagaagtgaggttg

SEQ ID NO: 53 - DNA, 20 bp, synthetic construct
  source 1..20: mol_type = other DNA
atgcctcaacctcacttcta

SEQ ID NO: 54 - DNA, 20 bp, synthetic construct
  source 1..20: mol_type = other DNA
ggttgaggcatcatgtgatg

SEQ ID NO: 55 - DNA, 20 bp, synthetic construct
  source 1..20: mol_type = other DNA
catcatgtgatgaggagtga

SEQ ID NO: 56 - DNA, 20 bp, synthetic construct
  source 1..20: mol_type = other DNA
ggagtgagggtctcttctgt

SEQ ID NO: 57 - DNA, 20 bp, synthetic construct
  source 1..20: mol_type = other DNA
cagccctttgtcttctccct

SEQ ID NO: 58 - DNA, 20 bp, synthetic construct
  source 1..20: mol_type = other DNA
tggcacaggccaggtcatca

SEQ ID NO: 59 - DNA, 20 bp, synthetic construct
  source 1..20: mol_type = other DNA
cttctcccttggcacaggcc

SEQ ID NO: 60 - DNA, 20 bp, synthetic construct
  source 1..20: mol_type = other DNA
tttgtcttctcccttggcac

SEQ ID NO: 61 - DNA, 20 bp, synthetic construct
  source 1..20: mol_type = other DNA
gaagacaaagggctggttct

SEQ ID NO: 62 - DNA, 20 bp, synthetic construct
  source 1..20: mol_type = other DNA
tgtgccaagggagaagacaa

SEQ ID NO: 63 - DNA, 20 bp, synthetic construct
  source 1..20: mol_type = other DNA
tgtgccaagggagaagacaa

SEQ ID NO: 64 - DNA, 20 bp, synthetic construct
  source 1..20: mol_type = other DNA
ggggtgagtcatgggggaat

SEQ ID NO: 65 - DNA, 20 bp, synthetic construct
  source 1..20: mol_type = other DNA
agtcatgggggaatgggctt

SEQ ID NO: 66 - DNA, 20 bp, synthetic construct
  source 1..20: mol_type = other DNA
tgactcaccccagcagcccc

SEQ ID NO: 67 - DNA, 20 bp, synthetic construct
  source 1..20: mol_type = other DNA
cttgggagtgggggcgtgca

SEQ ID NO: 68 - DNA, 20 bp, synthetic construct
  source 1..20: mol_type = other DNA
ggcgtgcatggccaccgccc

SEQ ID NO: 69 - DNA, 20 bp, synthetic construct
  source 1..20: mol_type = other DNA
gcttcgcacgattgcacctt

SEQ ID NO: 70 - DNA, 20 bp, synthetic construct
  source 1..20: mol_type = other DNA
cgaccgcacgattgcacctt

SEQ ID NO: 71 - DNA, 20 bp, synthetic construct
  source 1..20: mol_type = other DNA
cgagtcagtgagcgaggaag

SEQ ID NO: 72 - DNA, 20 bp, synthetic construct
  source 1..20: mol_type = other DNA
gttatcagtgagcgaggaag

SEQ ID NO: 73 - DNA, 20 bp, synthetic construct
  source 1..20: mol_type = other DNA
gctcactgactcgctgcgct

SEQ ID NO: 74 - DNA, 20 bp, synthetic construct
  source 1..20: mol_type = other DNA
agcgactgactcgctgcgct

SEQ ID NO: 75 - DNA, 20 bp, synthetic construct
  source 1..20: mol_type = other DNA
gtgggagtttagggctccca

SEQ ID NO: 76 - DNA, 20 bp, synthetic construct
  source 1..20: mol_type = other DNA
aactgagtttagggctccca

SEQ ID NO: 77 - DNA, 20 bp, synthetic construct
  source 1..20: mol_type = other DNA
gaatgcaacctcgatcgagc

SEQ ID NO: 78 - DNA, 20 bp, synthetic construct
  source 1..20: mol_type = other DNA
cttcgcaacctcgatcgagc

SEQ ID NO: 79 - DNA, 20 bp, synthetic construct
  source 1..20: mol_type = other DNA
cgggctccggtgcccgtcag

SEQ ID NO: 80 - DNA, 20 bp, synthetic construct
  source 1..20: mol_type = other DNA
gtatctccggtgcccgtcag

SEQ ID NO: 81 - DNA, 20 bp, synthetic construct
  source 1..20: mol_type = other DNA
tcgatcgaggttgcattcgg

SEQ ID NO: 82 - DNA, 20 bp, synthetic construct
  source 1..20: mol_type = other DNA
ggcttcgaggttgcattcgg

SEQ ID NO: 83 - DNA, 20 bp, synthetic construct
  source 1..20: mol_type = other DNA
cactgacgggcaccggagcc

SEQ ID NO: 84 - DNA, 20 bp, synthetic construct
  source 1..20: mol_type = other DNA
ggtagacgggcaccggagcc

SEQ ID NO: 85 - DNA, 20 bp, synthetic construct
  source 1..20: mol_type = other DNA
tctcatgtgggtttgaaaag

SEQ ID NO: 86 - DNA, 20 bp, synthetic construct
  source 1..20: mol_type = other DNA
tgtgtattctgctgttgttg

SEQ ID NO: 87 - DNA, 20 bp, synthetic construct
  source 1..20: mol_type = other DNA
cccacatgagacattcacca

SEQ ID NO: 88 - DNA, 20 bp, synthetic construct
  source 1..20: mol_type = other DNA
acactcttttcaaacccaca

SEQ ID NO: 89 - DNA, 20 bp, synthetic construct
  source 1..20: mol_type = other DNA
gttgaattcaactatgtcct

SEQ ID NO: 90 - DNA, 20 bp, synthetic construct
  source 1..20: mol_type = other DNA
ctatgtccttgctgattttc

SEQ ID NO: 91 - DNA, 20 bp, synthetic construct
  source 1..20: mol_type = other DNA
aaaatcagcaaggacatagt

SEQ ID NO: 92 - DNA, 20 bp, synthetic construct
  source 1..20: mol_type = other DNA
accagacagatccagcaggc

SEQ ID NO: 93 - DNA, 20 bp, synthetic construct
  source 1..20: mol_type = other DNA
tctccttctcagaagtcaat

SEQ ID NO: 94 - DNA, 20 bp, synthetic construct
  source 1..20: mol_type = other DNA
atgtatgacatctccttctc

SEQ ID NO: 95 - DNA, 20 bp, synthetic construct
  source 1..20: mol_type = other DNA
gaggttgaggcatcatgtga

SEQ ID NO: 96 - DNA, 20 bp, synthetic construct
  source 1..20: mol_type = other DNA
gcatcatgtgatgaggagtg

SEQ ID NO: 97 - DNA, 20 bp, synthetic construct
  source 1..20: mol_type = other DNA
cctcactcctcatcacatga

SEQ ID NO: 98 - RNA, 20 nt, synthetic construct
  source 1..20: mol_type = other RNA
ttgctaaggtgtatttaatg

SEQ ID NO: 99 - RNA, 20 nt, synthetic construct
  source 1..20: mol_type = other RNA
gtcttggtgaatgtctcatg

SEQ ID NO: 100 - RNA, 20 nt, synthetic construct
  source 1..20: mol_type = other RNA
agtgtgtattctgctgttgt

SEQ ID NO: 101 - RNA, 20 nt, synthetic construct
  source 1..20: mol_type = other RNA
ttctgcctgctggatctgtc

SEQ ID NO: 102 - RNA, 20 nt, synthetic construct
  source 1..20: mol_type = other RNA
gatctgtctggtactgatag

SEQ ID NO: 103 - RNA, 20 nt, synthetic construct
  source 1..20: mol_type = other RNA
cagcaggcagaaaatcagca

SEQ ID NO: 104 - RNA, 20 nt, synthetic construct
  source 1..20: mol_type = other RNA
attgcaattgacttctgaga

SEQ ID NO: 105 - RNA, 20 nt, synthetic construct
  source 1..20: mol_type = other RNA
aaggagatgtcatacatatt

SEQ ID NO: 106 - RNA, 20 nt, synthetic construct
  source 1..20: mol_type = other RNA
aggagatgtcatacatattt

SEQ ID NO: 107 - RNA, 20 nt, synthetic construct
  source 1..20: mol_type = other RNA
cttccatagaagtgaggttg

SEQ ID NO: 108 - RNA, 20 nt, synthetic construct
  source 1..20: mol_type = other RNA
atgcctcaacctcacttcta

SEQ ID NO: 109 - RNA, 20 nt, synthetic construct
  source 1..20: mol_type = other RNA
ggttgaggcatcatgtgatg

SEQ ID NO: 110 - RNA, 20 nt, synthetic construct
  source 1..20: mol_type = other RNA
catcatgtgatgaggagtga

SEQ ID NO: 111 - RNA, 20 nt, synthetic construct
  source 1..20: mol_type = other RNA
ggagtgagggtctcttctgt

SEQ ID NO: 112 - RNA, 20 nt, synthetic construct
  source 1..20: mol_type = other RNA
cagccctttgtcttctccct

SEQ ID NO: 113 - RNA, 20 nt, synthetic construct
  source 1..20: mol_type = other RNA
tggcacaggccaggtcatca

SEQ ID NO: 114 - RNA, 20 nt, synthetic construct
  source 1..20: mol_type = other RNA
cttctcccttggcacaggcc

SEQ ID NO: 115 - RNA, 20 nt, synthetic construct
  source 1..20: mol_type = other RNA
tttgtcttctcccttggcac

SEQ ID NO: 116 - RNA, 20 nt, synthetic construct
  source 1..20: mol_type = other RNA
gaagacaaagggctggttct

SEQ ID NO: 117 - RNA, 20 nt, synthetic construct
  source 1..20: mol_type = other RNA
tgtgccaagggagaagacaa

SEQ ID NO: 118 - RNA, 20 nt, synthetic construct
  source 1..20: mol_type = other RNA
tgtgccaagggagaagacaa

SEQ ID NO: 119 - RNA, 20 nt, synthetic construct
  source 1..20: mol_type = other RNA
ggggtgagtcatgggggaat

SEQ ID NO: 120 - RNA, 20 nt, synthetic construct
  source 1..20: mol_type = other RNA
agtcatgggggaatgggctt

SEQ ID NO: 121 - RNA, 20 nt, synthetic construct
  source 1..20: mol_type = other RNA
tgactcaccccagcagcccc

SEQ ID NO: 122 - RNA, 20 nt, synthetic construct
  source 1..20: mol_type = other RNA
cttgggagtgggggcgtgca

SEQ ID NO: 123 - RNA, 20 nt, synthetic construct
  source 1..20: mol_type = other RNA
ggcgtgcatggccaccgccc

SEQ ID NO: 124 - RNA, 20 nt, synthetic construct
  source 1..20: mol_type = other RNA
gcttcgcacgattgcacctt

SEQ ID NO: 125 - RNA, 20 nt, synthetic construct
  source 1..20: mol_type = other RNA
cgaccgcacgattgcacctt

SEQ ID NO: 126 - RNA, 20 nt, synthetic construct
  source 1..20: mol_type = other RNA
cgagtcagtgagcgaggaag

SEQ ID NO: 127 - RNA, 20 nt, synthetic construct
  source 1..20: mol_type = other RNA
gttatcagtgagcgaggaag

SEQ ID NO: 128 - RNA, 20 nt, synthetic construct
  source 1..20: mol_type = other RNA
gctcactgactcgctgcgct

SEQ ID NO: 129 - RNA, 20 nt, synthetic construct
  source 1..20: mol_type = other RNA
agcgactgactcgctgcgct

SEQ ID NO: 130 - RNA, 20 nt, synthetic construct
  source 1..20: mol_type = other RNA
gtgggagtttagggctccca

SEQ ID NO: 131 - RNA, 20 nt, synthetic construct
  source 1..20: mol_type = other RNA
aactgagtttagggctccca

SEQ ID NO: 132 - RNA, 20 nt, synthetic construct
  source 1..20: mol_type = other RNA
gaatgcaacctcgatcgagc

SEQ ID NO: 133 - RNA, 20 nt, synthetic construct
  source 1..20: mol_type = other RNA
cttcgcaacctcgatcgagc

SEQ ID NO: 134 - RNA, 20 nt, synthetic construct
  source 1..20: mol_type = other RNA
cgggctccggtgcccgtcag

SEQ ID NO: 135 - RNA, 20 nt, synthetic construct
  source 1..20: mol_type = other RNA
gtatctccggtgcccgtcag

SEQ ID NO: 136 - RNA, 20 nt, synthetic construct
  source 1..20: mol_type = other RNA
tcgatcgaggttgcattcgg

SEQ ID NO: 137 - RNA, 20 nt, synthetic construct
  source 1..20: mol_type = other RNA
ggcttcgaggttgcattcgg

SEQ ID NO: 138 - RNA, 20 nt, synthetic construct
  source 1..20: mol_type = other RNA
cactgacgggcaccggagcc

SEQ ID NO: 139 - RNA, 20 nt, synthetic construct
  source 1..20: mol_type = other RNA
ggtagacgggcaccggagcc

SEQ ID NO: 140 - RNA, 20 nt, synthetic construct
  source 1..20: mol_type = other RNA
tctcatgtgggtttgaaaag

SEQ ID NO: 141 - RNA, 20 nt, synthetic construct
  source 1..20: mol_type = other RNA
tgtgtattctgctgttgttg

SEQ ID NO: 142 - RNA, 20 nt, synthetic construct
  source 1..20: mol_type = other RNA
cccacatgagacattcacca

SEQ ID NO: 143 - RNA, 20 nt, synthetic construct
  source 1..20: mol_type = other RNA
acactcttttcaaacccaca

SEQ ID NO: 144 - RNA, 20 nt, synthetic construct
  source 1..20: mol_type = other RNA
gttgaattcaactatgtcct

SEQ ID NO: 145 - RNA, 20 nt, synthetic construct
  source 1..20: mol_type = other RNA
ctatgtccttgctgattttc

SEQ ID NO: 146 - RNA, 20 nt, synthetic construct
  source 1..20: mol_type = other RNA
aaaatcagcaaggacatagt

SEQ ID NO: 147 - RNA, 20 nt, synthetic construct
  source 1..20: mol_type = other RNA
accagacagatccagcaggc

SEQ ID NO: 148 - RNA, 20 nt, synthetic construct
  source 1..20: mol_type = other RNA
tctccttctcagaagtcaat

SEQ ID NO: 149 - RNA, 20 nt, synthetic construct
  source 1..20: mol_type = other RNA
atgtatgacatctccttctc

SEQ ID NO: 150 - RNA, 20 nt, synthetic construct
  source 1..20: mol_type = other RNA
gaggttgaggcatcatgtga

SEQ ID NO: 151 - RNA, 20 nt, synthetic construct
  source 1..20: mol_type = other RNA
gcatcatgtgatgaggagtg

SEQ ID NO: 152 - RNA, 20 nt, synthetic construct
  source 1..20: mol_type = other RNA
cctcactcctcatcacatga

SEQ ID NO: 153 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 154 - DNA, 113 bp, Desulfotomaculum nigrificans
  source 1..113: mol_type = genomic DNA
tgattttacgctggtgctatatcctaaactcccacagataaacagttaatggtaatgaaataacattaactgtttatctgtgtttaatgccttaacttaatctagtaggaggg

SEQ ID NO: 155-163 - skipped (9 entries)

SEQ ID NO: 164 - DNA, 113 bp, Escherichia coli
  source 1..113: mol_type = genomic DNA
cttgaagattatgtgaggcgaacatctaaagaaaaccgaatagactttattcaagggtcattgtattgtagctattcggcattgtagcattagccataacggttataagctta

SEQ ID NO: 165-173 - skipped (9 entries)

SEQ ID NO: 174 - DNA, 107 bp, Escherichia coli
  source 1..107: mol_type = genomic DNA
ggttcattgaacaggagaacaatgatatgagtgttaaggcaagaatactagtgcttttacatagctaaacacgtacattcaactacctgttaataacaggacaatat

SEQ ID NO: 175-183 - skipped (9 entries)

SEQ ID NO: 184 - DNA, 104 bp, Escherichia coli
  source 1..104: mol_type = genomic DNA
aacaggtgataggattcggatgttctcatagtgtatttagggaattaatatcagtataatgggtccctaaacacatcattttaggtattgaatatgagacgggc

SEQ ID NO: 185-193 - skipped (9 entries)

SEQ ID NO: 194 - DNA, 112 bp, Bacillus sp.
  source 1..112: mol_type = genomic DNA
atggcgacatataagcgttcgtgctttgtcgtcaccttgttggtgtaattaggttgacgccaacagggtgataacacaagaaggactttttatttcttctattatatataga

SEQ ID NO: 195-203 - skipped (9 entries)

SEQ ID NO: 204 - DNA, 102 bp, Enterocloster clostridioformis
  source 1..102: mol_type = genomic DNA
taaagggtttcacgcataagtaccaataatgtaacaacctgtactgaatgtgcttccagtacaggttgttacaccgttaggcaaaaaaataaatatccatag

SEQ ID NO: 205-213 - skipped (9 entries)

SEQ ID NO: 214 - DNA, 111 bp, Fusobacterium mortiferum
  source 1..111: mol_type = genomic DNA
aagagtgttctaaaatagaagaaaataaaaacatacacataaagacgcacgtagatacgtgagttcctacccacttgttttttactctatcttctcttgtttccaatttct

SEQ ID NO: 215-223 - skipped (9 entries)

SEQ ID NO: 224 - DNA, 113 bp, Klebsiella pneumoniae
  source 1..113: mol_type = genomic DNA
agaaccttgaaaaactatggcttatgctacctcgccgaatagctcaaatacaatgacccttgaatataggctattcgggttttgaaggtatctggtttttattcacatcacaa

SEQ ID NO: 225-233 - skipped (9 entries)

SEQ ID NO: 234 - DNA, 117 bp, unidentified
  source 1..117: mol_type = unassigned DNA
cgaaatggttggcgttgaggtcaatgattaatgtgtatagggttaacatttaaatcagtacaatcgtagacgctctacactattttctgtgtataaaaatatcgagaataaacgctt

SEQ ID NO: 235-243 - skipped (9 entries)

SEQ ID NO: 244 - DNA, 104 bp, Pseudomonas aeruginosa
  source 1..104: mol_type = genomic DNA
gtaacgctcttcgagaaagcagattctcatatccatcttgagtcttctttctcgcaagacaacacgaaatagacacagtctcttccctagctgtacactgagcc

SEQ ID NO: 245-253 - skipped (9 entries)

SEQ ID NO: 254 - DNA, 112 bp, unidentified
  source 1..112: mol_type = unassigned DNA
tcttttatgtcagcagttcttttcatcctgtttcatcttgtacgcttcgtttcgccgaagcgtacaagatggaaatcataaaccttcaaatgcgccattcgatcttgatgga

SEQ ID NO: 255-263 - skipped (9 entries)

SEQ ID NO: 264 - DNA, 103 bp, Vibrio parahaemolyticus
  source 1..103: mol_type = genomic DNA
cgtacatggctcctagtgtgtacgatgggaaataaccaaaagagccgtctgtccaatggatgtcttgcatacaaccatttttgaagttgccctctgtagatag

SEQ ID NO: 265 - DNA, 105 bp, Pseudomonas fluorescens
  source 1..105: mol_type = genomic DNA
aaatgctggacgggaggataacagaggctcagtgttacatacgatattgttgggagcaagcttttcgtaagtaacactgagatatagacgccactggctgtggct

SEQ ID NO: 266 - DNA, 112 bp, Streptomyces ipomoeae
  source 1..112: mol_type = genomic DNA
tagtgacgtctgtccgcgcagtgatcgagggagtgtgtgctttgccgactggcaaggtcaagccggtctgctaggcacagagagccggtacagtcctccccatgcaacccaa

SEQ ID NO: 267 - DNA, 104 bp, Clostridium perfringens
  source 1..104: mol_type = genomic DNA
cttaaccgcttttgaatgtccgtcttagttaatccctaattgaacgctccaaaaggtgacttccaatagggatttattccttttaaaattaacggcataatcgt

SEQ ID NO: 268-273 - skipped (6 entries)

SEQ ID NO: 274 - DNA, 112 bp, Clostridium difficile
  source 1..112: mol_type = genomic DNA
tgataataaaatataacagttattaaatcccttggaatatatgaaatctcaattccagtttgccgaaatatctggctaactttcccaattttacagacattccagcccattc

SEQ ID NO: 275 - DNA, 112 bp, Clostridium difficile
  source 1..112: mol_type = genomic DNA
gaattttatatattttaaatatgatagtttaaaattatgaaagaatgataaattccagtttgtagggtaattttgataaataatattaataagtatgagtaaaaagtaggag

SEQ ID NO: 276 - protein, 557 aa, Clostridium difficile
MLQSNKITALYCRLSQEDMQAGESGSIQHQKMILQRYADEHHFLNTKFFVDDGFSGVSFEREGLQAMLQEVEAGRVATVITKDLSRLGRNYLKTGELIEIVFPENGVRYIAINDGVDTAREDNEFTPLRNWFNEFYARDTSKKIRAVKQAQAQKGERVNGEYPYGYIPDPNNRHHLIPDPETAPIVKQVFAMFVSGVRMCEIQKWLAENKVLTIGALRYQRTGQARYQRAMIAPYTWPDKTLYDILARQEYLGHTITAKTHKVSYKSKKTRKNEEEQRYFFPNTHEPLVDEETFELAQKRIATRHRPTKAAEIDIFSGLLFCAGCGHKMYYQQGVNIEPRRFSYSCGAWRNRARTGSECTSHYIRKNVLLDLVLEDMRRVLQYVKEHEQDFICKATEYGDMEARKALAQQQKELSKAQARMTELDTLFRKLYEDNALGRLTDERFVFLTSGYEDEKKSLAARIDELQQQIATVTERKRDISRFIQIVGKYSDIQELTYENVHEFIDRILIHELDRETNTRKIEIHYSFVGQVDTEQEPTQVVNHDRRNMVDVKSIAI

SEQ ID NO: 277 - DNA, 147 bp, unidentified
  source 1..147: mol_type = unassigned DNA
tttgcgggacgcttccgacgctgtaggaccgagtggacggcctgaagctcacttctaatccgacggttgcaggttcgagtcctgccgggggcacttctaaaacccttgcaagcaaagggttcctagcgccaacagctgagctgtgca

SEQ ID NO: 278 - DNA, 147 bp, unidentified
  source 1..147: mol_type = unassigned DNA
tgtcgctgcatcgaagcgggcccctgtagctcaattggatagagcatcggacttctaatccgacggttgcaggttcgagtcctgccgggggcacttccaggacgctgtttggcacaccagcgtccttctcggtagcgaccgccatga

SEQ ID NO: 279 - protein, 498 aa, unidentified
MLTVAYVRVSTDDQVEHSPDAQRRRCAEYAAQRNLGPVKFLSDEGQSGKDLERPAMQELVGLIEAGQVTNLIVWRMDRLSRDSGDTSRLLRLFEQHCVNVHSLNEGDLETGSASGRFTAGLHGLLAQLEREKITENIHLGNEQAVRTGFWINRAPTGYDTVDRVLVPNDDAHLIRRAFKLRAGGQSYPQIEQGTGLKYSTVRHALENKVYLGFTRLRDERFPGKHEPLVSQAEFDAAQRAHTPGRRRGKDLLSGRVRCGECGRITGIDTNGRGKPIYRCRTRGNGCAIPGRSAAGLRRAARLSVELLRTDDQLVEAIRTHFAEKTERAGAGTAEPSRAGTLGSLRSKRQKLFELLLAGKITDDFFAEQERQLTAKIEATEAHRTEAIETHRHHNALAEAFEQAAAMLRDPAFELAEIWDNASAAERRVIVEELIESVTIYADRLEVNVTGAPPLLVTLAEVGLREPGTAPVVSEDLTDFRSSGAWGLSGRTRLIDWVA

SEQ ID NO: 280 - DNA, 106 bp, Bacillus asahii
  source 1..106: mol_type = genomic DNA
tagatatttctctttatttaaatagttggtcgtaaattaccacatgctattggggagaagtagtgtaaaaggtacttggtctgtgttattcgccatatatcaaaaa

SEQ ID NO: 281 - DNA, 106 bp, Bacillus asahii
  source 1..106: mol_type = genomic DNA
atatgaatctatccttagtagctattgatgaggctcattgtatttcgcagtggggacatgattttcggccaagctatttaaaaattaaaacccttttaaaagagtt

SEQ ID NO: 282 - protein, 518 aa, Bacillus asahii
MEINAVNHIKDVAIYLRKSRGQEEDLDKHREELVELCEKNGWRYVEYGEIGSSQKLMDRPELSRLLKDIQEDMYDAVVVMDKDRLSREGVGQAQINKILAENDCLIVTPTRVYDLTNESDMMVSEFEDLMARFEYRAIVKRLKRGKRRGAKQGKWTNGTPPIPYIYNPKFRDKTDKSIEKGEDLDSLIVDEEKLEIYRFIVDSFLNGMSPYTIAWELNKRKIASPRGSTWNNTGIRRLLKDETHLGRIIANKTTGSKKRLSTGRLDYKVNKREDWIIVENCHKAVKTKEEHEKILIEMQKRKRDSYATTGENALSGLIVCSKCGATMAQKKSKIESERYSYVEACKNFLEDGITKCDNHGGSTMYLMKEIERQLVMYRDDIEKENERVSKGGGLTKTIESEKKKKEERIIELEDELEQVTTLALAKFFTPEEAVQQKKKILDNISKLESELYALNLQTDNLGNMTRSEKVNAINKFLEVMDMPHISNSDLNRLYKTIIHSIIWDKSNPDELKVTINFL

SEQ ID NO: 283 - DNA, 106 bp, Parabacteroides goldsteinii
  source 1..106: mol_type = genomic DNA
atctgtccgcccaatcggcggcagaatgtaagctgacggaattcggcttgatcaatatggatgaatttgataaatttactcctcgcaagatggcattgttgaagaa

SEQ ID NO: 284 - DNA, 106 bp, Parabacteroides goldsteinii
  source 1..106: mol_type = genomic DNA
tattacgctgatttacagctgattatttaatcaataatgtttttagtttaatcaattaaactaataacttttatgattaaatttaaagtgctatatttgtgctgaa

SEQ ID NO: 285 - protein, 563 aa, Parabacteroides goldsteinii
MKRAALLLRCSTNIQDYNRQKLELEKVANRFKLKVAKVFGEHVTGKDDIRKGNRKSVDELINACENNEIDVVLISEVSRLTRNFLYGVTLVDKFNRDYSIPIYFRDKRKWTIDVETGEVNLEFEKELRKAFEQAEEELASIRFRMASGKRDSAGLGQWFAGFPPFGYTRQKNGYLVKNEYAPIVKEMFDKYLEEGQALITTTRYIYGKYPDIKKLKSIGNTKFILNNKAYTGRAEANIYDEIDKVTDTFYFDIPAIIDEETYNKVQKKLANNRTTTPYPKAAKYLLQKLIKCSICGAFYTRLHSNKRDTVHFKCTSDKNSLTGCKSQVYLNERIVNPIIWNFVKEQLFYVGKMNAEQRLEAIAGENDNKSKAIEEQEALKISYKEQENKLTRLEDLYLDNDIDKNRYKERKSKIEKELSSIKAQMDRLTDRIRLSDFNIKRFNSTDFTEQYFKEVEADLEKQMKVLREYVKGIYPLYIDKVYCFLKVDTIEGMFNIFYEPRKAQQKCAYFIKDTLAQYHPDIRNFYTPNNTLLTDSDEVDAYYTLEEMKEICSRNGFEVRYRE

SEQ ID NO: 286 - DNA, 131 bp, Staphylococcus aureus
  source 1..131: mol_type = genomic DNA
ctaagatcaatacgatgtatcttgttattacttttgcatccatttgtttgctccttttatccaaaataaaaaacgactaaataagccgtctatttgatatttatattatggtgtgttaatttatatataga

SEQ ID NO: 287 - DNA, 131 bp, Staphylococcus aureus
  source 1..131: mol_type = genomic DNA
ccaaaatgatttttgtttcattctaattttccaattaatcatattcttatctccttttatccaaaataaaaaacgactaaaaaattagtcgtttaaaattattcaatggtcaatgttggagatcctgaata

SEQ ID NO: 288 - protein, 461 aa, Staphylococcus aureus
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNTCNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 289 - DNA, 103 bp, Eggerthella lenta
  source 1..103: mol_type = genomic DNA
ccatggatttctcagagaactccgggcgcgtttttaatatgcgcaaagggatcccttttgtcaagacgaaaaaacaagattacctgcagaaatgctccgacta

SEQ ID NO: 290 - DNA, 103 bp, Eggerthella lenta
  source 1..103: mol_type = genomic DNA
cgtaggccgcgagctcctcctcgacgacagggcgcgggccgatcacgctcatctggccggccagcacgttgaggaactgagggagctcgtcgagggaggtctt

SEQ ID NO: 291 - protein, 543 aa, Eggerthella lenta
MPKTKTPKTAARVYRAASYARLSVDDESYGTSGSVLNQHAMIRDFADANGGLSIVAEYSDDGFSGSTFENRPGWGSLLADVETGKVDCIVVKDLSRLGRNYLDVSRYLDQVFPALGVRVVAITDGYDSAAEKTPADALMLPVKNLFNDMYCRDASAKTKASLSAKRRRGEFVGAFAPYGYAKGSGPDRGRLVIDPEPAGVVRGIFDARIGGMSAAGIAAMLNESLVPSPYEYFAAKGAARSSNFCKGERTAWDARTVLRILSNETYAGTLVQGKTCKPDFRRKAVLSVDESEWDRSPGAHEAIVDPATFGLVRKLARRDMRLSPGAKRSLPLSGFLFCADCGATMARHASRCSSGKRFYYSCETHRKNRAECGMHKVWEDELSAAVMRAVHGHALAAVDGDGVVRSAEAYRHDRRAELACRLESVEGRIEHNGDLRLRMYSDYAAGVIDKAQYAELARAVDVRLQELKGEKAGIERETAQLDDVHAETWEQTLARYRDAEGLERVMVVELIDRVLVGEGGDIEVVFRFGGPVAACATRQEGVA

SEQ ID NO: 292 - DNA, 103 bp, Bacillus cereus
  source 1..103: mol_type = genomic DNA
cgaaatacatgatggaagaaatgcgttttttacaacaagagcattttgtttgtttatatttaaatacaaaaaatcaagttatacataggcaaacgatctttat

SEQ ID NO: 293 - DNA, 118 bp, Burkholderia multivorans
  source 1..118: mol_type = genomic DNA
cgtaaaatcaggcgatgcgccggcacatcgcaaatgtattttgactctcgttcgggttgccgagcgtgtccgaaaatatattatcagacaactcggtcaggggagcgcgtaaacgaaa

SEQ ID NO: 294 - DNA, 102 bp, Bacillus safensis
  source 1..102: mol_type = genomic DNA
caaaatatgtgatggaagatatgaggtttttacaacaggagaatttcgtttgtctttatctcaattcaaaaaatcaagttcttcataaacatactgtgttta

SEQ ID NO: 295 - DNA, 112 bp, Bacillus sp.; identical to SEQ ID NO: 194
  source 1..112: mol_type = genomic DNA

SEQ ID NO: 296 - DNA, 117 bp, Burkholderia ubonensis
  source 1..117: mol_type = genomic DNA
cacattccaagatgtctcaaaatcagtctcacaatccccgtataggtatagtatccctcgggtgcccaaagtatattgaaaatataccaaggttgaatacctcgtcagctaggctag

SEQ ID NO: 297 - DNA, 52 bp, unidentified
  source 1..52: mol_type = unassigned DNA
gtggtttgtctggtcaaccaccgcggtctcagtggtgtacggtacaaaccca

SEQ ID NO: 298 - DNA, 119 bp, Clostridium botulinum
  source 1..119: mol_type = genomic DNA
attcattacaggtagaacttgttgactaataattagtgtagttttacctgtgctgcacatccagaccagttaaaactccattaaaacacgtgattattttcacacaaaaaaaacctcta

SEQ ID NO: 299 - DNA, 105 bp, Cellulosimicrobium cellulans
  source 1..105: mol_type = genomic DNA
acggcgcacgtcgttccggtctgggagcctatgcacaacgagggataacggacgttctcccatgctgcgcatagggcaggcgtgcgtgcggttgcgcgtcgtagg

SEQ ID NO: 300 - DNA, 105 bp, Clostridium difficile
  source 1..105: mol_type = genomic DNA
gtaatcctcccgtcagaccgttaccctgtgtagtcccttgtaaactgtactttaggtcagtttacaaggaactacacgcagaccgtgaaacgggctgctgacaac

SEQ ID NO: 301 - DNA, 122 bp, Clostridium difficile
  source 1..122: mol_type = genomic DNA
tctttgttaaattacgcaaaatttcattcccaacttcatatccaaataaatcatttatatatttaaatttattgacatccaattgtactaagtaaaaattatcttgataataattctttaaa

SEQ ID NO: 302 - DNA, 106 bp, Clostridium difficile
  source 1..106: mol_type = genomic DNA
ttaaaaaagtctgctaataaaggcaataattctatatctgggtaagaatttccactctcccacttagatactgctggtgttgacacacctataaactttgctaatt

SEQ ID NO: 303 - DNA, 102 bp, Clostridium difficile
  source 1..102: mol_type = genomic DNA
aagccgcatggttacggcattttccgtgttgtgcatatattgacggagcagtagagccgtgtatttatgcacaacattagattttcctttgttttgagtagg

SEQ ID NO: 304 - DNA, 104 bp, Clostridium perfringens; identical to SEQ ID NO: 267
  source 1..104: mol_type = genomic DNA

SEQ ID NO: 305 - DNA, 114 bp, Cronobacter sakazakii
  source 1..114: mol_type = genomic DNA
taatgaacatagcacaacaaaaccaaaaaacagattttacaaggttttcccgtttagtgttagcgaaatactaaaacctgataaaaaccctctccagttgttttttcttgcccc

SEQ ID NO: 306 - DNA, 106 bp, Clostridium thermobutyricum
  source 1..106: mol_type = genomic DNA
aaaaaggagaagtttatcttcaagatccaataggggttgataaagaaggaaatgaaatttgtttaatagatgttttaagtagtgaaaaagatttagttttagaaaa

SEQ ID NO: 307 - DNA, 113 bp, Desulfotomaculum nigrificans; identical to SEQ ID NO: 154
  source 1..113: mol_type = genomic DNA

SEQ ID NO: 308 - DNA, 113 bp, Escherichia coli; identical to SEQ ID NO: 164
  source 1..113: mol_type = genomic DNA

SEQ ID NO: 309 - DNA, 107 bp, Escherichia coli; identical to SEQ ID NO: 174
  source 1..107: mol_type = genomic DNA

SEQ ID NO: 310 - DNA, 104 bp, Escherichia coli
  source 1..104: mol_type = genomic DNA
tactatataaactaaataataaatattcttgttcactttgaaactatattgtgatattgttgcaaagaagcaaaattgatactctcttatactttactggggtg

SEQ ID NO: 311 - DNA, 117 bp, Escherichia coli
  source 1..117: mol_type = genomic DNA
atcaattgaagaagatgtaagatgaacatctaaagaaaaccgaatagacttaaatcaagggtcattgtattgtagctattcggcattgtagcattagccataacgtttataagttca

SEQ ID NO: 312 - DNA, 104 bp, Escherichia coli; identical to SEQ ID NO: 184
  source 1..104: mol_type = genomic DNA

SEQ ID NO: 313 - DNA, 105 bp, Enterococcus faecium
  source 1..105: mol_type = genomic DNA
aaaatatacttgcgattagtcgttatttttcatagacaagtaaatcaatcatgcacatggtagcatgagtgttctatgaaaaaagagggtaggagcgatcagcta

SEQ ID NO: 314 - DNA, 107 bp, Enterococcus faecium
  source 1..107: mol_type = genomic DNA
taacagtaaatttccttatataatttatgtgtactaacttatatattccggaaggataatataggttagtacacgtataaaaatatcttttatattgacacaattta

SEQ ID NO: 315 - DNA, 110 bp, Enterococcus faecalis
  source 1..110: mol_type = genomic DNA
agtttaatactaaaagaggtatattaattttatttaaaaattgacattaatgactcttaaccaaatgctctgcttgtgaaaaaagctttataaaactttataaataggtt

SEQ ID NO: 316 - DNA, 103 bp, unidentified
  source 1..103: mol_type = unassigned DNA
gacacactttcgagtgtgtcttttttattacctgaataaaaccaagaactaaagtacctagttttattcaggtaagtgtaaaaaacggctaaatctagccgtt

SEQ ID NO: 317 - DNA, 102 bp, Enterocloster clostridioformis; identical to SEQ ID NO: 204
  source 1..102: mol_type = genomic DNA

SEQ ID NO: 318 - DNA, 104 bp, Enterocloster clostridioformis
  source 1..104: mol_type = genomic DNA
tgtcagcgttaatgataagttggtttctatacttccttatcactgtaaacatcaaggtttgcggtgataaggaagtaatttcagattaggcggtatagccccat

SEQ ID NO: 319 - DNA, 111 bp, Fusobacterium mortiferum; identical to SEQ ID NO: 214
  source 1..111: mol_type = genomic DNA

SEQ ID NO: 320 - DNA, 105 bp, Faecalibacterium prausnitzii
  source 1..105: mol_type = genomic DNA
tgattgtcgcaggactatccgactgggggtgtgttttgataccaaccgggctcccgaacggtatcgaaacacacccccacacgaaagtgcggggacaggcttaat

SEQ ID NO: 321 - DNA, 111 bp, Klebsiella pneumoniae
  source 1..111: mol_type = genomic DNA
ccgttgaagaactacgctaaaagtattaaatcaaaatgttcccatagtatacgaaaggacgcataccataatcaatgggaacatttgaattttcgtaaaaaaaagaggcca

SEQ ID NO: 322 - DNA, 113 bp, Klebsiella pneumoniae; identical to SEQ ID NO: 224
  source 1..113: mol_type = genomic DNA

SEQ ID NO: 323 - DNA, 105 bp, Klebsiella pneumoniae
  source 1..105: mol_type = genomic DNA
taagtgatttcagtctgagagggaaagtgtatcaatagaaaggtctcttcaggattacacctttcttatgatacacttcttcaatccttcataactcatcataaa

SEQ ID NO: 324 - DNA, 108 bp, Klebsiella pneumoniae
  source 1..108: mol_type = genomic DNA
tgaattattgatgtaagaggctttttgttctttagtgttcttaacttccttaatgtctgctattgtagtgctatctacactacggttaagcgacctctctaatgaaaa

SEQ ID NO: 325 - DNA, 110 bp, unidentified
  source 1..110: mol_type = unassigned DNA
cacccgtcctagtactcgcatatggcgagtctacaaccgttcccatacgacaagtcgtagtacaactattgtagatgggtgtttagggtacgaaaaaagcccccaggcca

SEQ ID NO: 326 - DNA, 114 bp, unidentified
  source 1..114: mol_type = unassigned DNA
gcattgcgtggtgggctggccatatcctaattgttgcacggttgcggaatgtggtggaattccgcaccggattaacaactgccaaaaaatagaaacccgcagctcacggcatat

SEQ ID NO: 327 - DNA, 109 bp, unidentified
  source 1..109: mol_type = unassigned DNA
gggtgtagatgactgccttcacctgcatagttaaaacggtagcagtgaagctacgatctgtcatagcgtccgagctcactgttttaggctctaaccggtgcgggacgtg

SEQ ID NO: 328 - DNA, 117 bp, unidentified; identical to SEQ ID NO: 234
  source 1..117: mol_type = unassigned DNA

SEQ ID NO: 329 - DNA, 115 bp, Nocardia otitidiscaviarum
  source 1..115: mol_type = genomic DNA
tgattacgatcagtgccctgggaggcgattccggcatggcttatatccaacaccaccgagagcgctgttgtcgagcgtgtaagccaggacgaggacgagcacgcccacgggcacg

SEQ ID NO: 330 - DNA, 104 bp, Pseudomonas aeruginosa; identical to SEQ ID NO: 244
  source 1..104: mol_type = genomic DNA

SEQ ID NO: 331 - DNA, 102 bp, Pseudomonas aeruginosa
  source 1..102: mol_type = genomic DNA
gttcaaccgtcctagaagaccttgatgtgtgagattcacccctaccattcgagactggcaggtggtattctcacacttcctaagatctcagcaggaagcccg

SEQ ID NO: 332 - DNA, 114 bp, Paenibacillus campinasensis
  source 1..114: mol_type = genomic DNA
cgtttatcgggcggtaatattttaaagtattgcgctacactcggcacccgacacatgtggagtgctgtgtgtcgctcgtatggaagtaatgattaggagccgcatttacctttc

SEQ ID NO: 333 - DNA, 105 bp, Bacillus sp.
  source 1..105: mol_type = genomic DNA
ttggaatgacaatcaacaataagacagaaataaccattaatacgattagcatttcgattaacgtaaaccccttttcattcttcattgtccttccctcctataaat

SEQ ID NO: 334 - DNA, 108 bp, Pseudomonas fluorescens
  source 1..108: mol_type = genomic DNA
tgacagcgccatttgctgggagagagtgatggtgtaacaagcgaattacttgggatcagatcatctgaatgttacactgccaccttttcgacgaaggtgtgtggtttc

SEQ ID NO: 335 - DNA, 102 bp, Pseudomonas fluorescens
  source 1..102: mol_type = genomic DNA
gctgctcaaaagggcagcatacatcacagtgtcacttagcacatagctgttcacaggtattttatatgttacaccaccagcccattctgctggcaatactag

SEQ ID NO: 336 - DNA, 106 bp, Pseudomonas fulva
  source 1..106: mol_type = genomic DNA
tcgatcaggtcgaccagcgtgctggcttcgagtttgtgcatgaggcgtttcttgtaagtgctgaccgtcttgctgcttagaaacatgccctttgcaatctccttgt

SEQ ID NO: 337 - DNA, 105 bp, Pseudomonas fluorescens; identical to SEQ ID NO: 265
  source 1..105: mol_type = genomic DNA

SEQ ID NO: 338 - DNA, 105 bp, Prochlorothrix hollandica
  source 1..105: mol_type = genomic DNA
agttgatccaggtggaaaaggcgatcgaagttcattgaggggttctatttttgaacgagttaactgagtttaacccataccattctcagggattaagaggatatt

SEQ ID NO: 339 - DNA, 34 bp, Streptomyces sp.
  source 1..34: mol_type = genomic DNA
gtgccagggcgtgcccttgggctccccgggcgcg

SEQ ID NO: 340 - DNA, 106 bp, Pseudomonas putida
  source 1..106: mol_type = genomic DNA
atcgcacgctcttcgtggcagggaaagccgcagtgtaacattcagagaaactggtcacaacattttcttttgttacaccacgttgatggctagccacctatgcacc

SEQ ID NO: 341 - DNA, 104 bp, Paeniclostridium sordellii
  source 1..104: mol_type = genomic DNA
taataaagaatgaaaataaatctactattttagttacccatgatatatcagaagctatttctatgtcagataaggtcgcagttttatccaaacgtcctgcatct

SEQ ID NO: 342 - DNA, 105 bp, Pseudomonas syringae
  source 1..105: mol_type = genomic DNA
atttgctggctgagatggtaacaggggatcagtgttacatgtgaaaacgttgggagcaagctctttgtaagtaacactgagaagtacctaacatgagagtaaccc

SEQ ID NO: 343 - DNA, 148 bp, unidentified
  source 1..148: mol_type = unassigned DNA
caatgagatatgcgttagttagaacaataggttgtcgggcgcttctgccaattgaaaatccgcgtgtcggtggttcaaatccgcctccgggcaccattaacttgctgaaaatgctaagtttttggaaagctctttgatcccgcttgtg

SEQ ID NO: 344 - DNA, 116 bp, Rhodococcus equi
  source 1..116: mol_type = genomic DNA
cctggagaagccgattccatgtcatgtacggtagcttgttgtgtacaggttgatgttccacgagccggacagcaacctccacaaaacctgaacagcccccggcggtggtgcgaaca

SEQ ID NO: 345 - DNA, 102 bp, Ruminococcus lactaris
  source 1..102: mol_type = genomic DNA
agaaactttactcaaatgtatttctgttgccagtgcaaatgatgcctgtgttgctatggctgaatatatttcaggaaatgaagaggaatttgtccgtcagat

SEQ ID NO: 346 - DNA, 109 bp, Staphylococcus aureus
  source 1..109: mol_type = genomic DNA
caccacctattaatttaggagtgtggttgtttttgttggaagtgtgtatcaggtaacagcatagttattccgaacttccaattaataaaactctatacccgtaatcttc

SEQ ID NO: 347 - DNA, 106 bp, Staphylococcus aureus
  source 1..106: mol_type = genomic DNA
cctattaatttatgagtgtggttggtttttaatgaatgttttgtaactattgcgttctttctagttacataacactcattaatatttgaaatgtatttcattgatt

SEQ ID NO: 348 - DNA, 129 bp, Staphylococcus aureus
  source 1..129: mol_type = genomic DNA
aatttgagttttaatttatttttttctttcgcaattctaaatttttgtaacatttgtagttcctccttcattcgaaatcatcgatagttaattctgaaactctcttttcatagatatataaataatagt

SEQ ID NO: 349 - DNA, 102 bp, Streptococcus agalactiae
  source 1..102: mol_type = genomic DNA
cagttccaatggttctcagaaaatttcaagttaaagcatttactgttttggagagccttattgtattatcagtagtggcatttatgacgttagtattttcaa

SEQ ID NO: 350 - DNA, 128 bp, Staphylococcus arlettae
  source 1..128: mol_type = genomic DNA
gcgtcatgctcatcgaacaaatctacagagcctttaagatcatgaaaggcgaagcatatcacaaataaaactaaaaaatagattgtgtataatatattttaaatataaaaaggattgattttatgtta

SEQ ID NO: 351 - DNA, 102 bp, Streptococcus equinus
  source 1..102: mol_type = genomic DNA
gtacaaataaaagcatcaagacaccgataattaacaggacaatcaacatctccacaagtgtgaaagcttcaacagattttttacgtaattttttccatagtt

SEQ ID NO: 352 - DNA, 110 bp, Staphylococcus hominis
  source 1..110: mol_type = genomic DNA
acttaatataagggagattacttttaaattttaatagtagtagtgttacagggtacgtagtgcttgtaacactatttttatgtataaaaaaagaccacgctcataagaac

SEQ ID NO: 353 - DNA, 112 bp, Streptomyces ipomoeae; identical to SEQ ID NO: 266
  source 1..112: mol_type = genomic DNA

SEQ ID NO: 354 - DNA, 102 bp, Streptococcus mitis
  source 1..102: mol_type = genomic DNA
gaaaaaaatgatgacattattgaaaaaagcgaaggttaaagctttcacacttattgagatgttggttgtcttgctcattatcagtgtgcttctcttgctctt

SEQ ID NO: 355 - DNA, 110 bp, Salinispora sp.
  source 1..110: mol_type = genomic DNA
tactccctggtgtcctcccagcaagcgcactactgggttaggatgttcactgacaccagtacagtatcagaagcataagtggcaggacgaatacccaactgaaatcagta

SEQ ID NO: 356 - DNA, 133 bp, Treponema denticola
  source 1..133: mol_type = genomic DNA
cggagggtgattttaaagagtttttcataattaatactttgcactctgttattttttttataatttactatttttttcaaataataactataaatatctttgcgtagacaacctttaaatttcgctcagaacc

SEQ ID NO: 357 - DNA, 108 bp, Tenacibaculum dicentrarchi
  source 1..108: mol_type = genomic DNA
aattgccatggaaaaactaaacctgtcggcaagagcctatgatcgaattttaaaagtatcaagaactattgccgatttagcatccgaagaaaatataaaatcggaaca

SEQ ID NO: 358 - DNA, 112 bp, unidentified; identical to SEQ ID NO: 254
  source 1..112: mol_type = unassigned DNA

SEQ ID NO: 359 - DNA, 111 bp, Vibrio sp.
  source 1..111: mol_type = genomic DNA
catatagttaatgtgtggtttgtttttttgtacggaagtgtctaaaaagcgtcgcaatttgcgggggttccgtacataataatagtcattcgggtacatccatttagtgga

SEQ ID NO: 360 - DNA, 102 bp, Vibrio harveyi
  source 1..102: mol_type = genomic DNA
ttaaaaagaaaggctttaagtttgtgggcgagacgatttgctacgccttcatgcaagcagtaggcatggtcgatgaccacattgttggctgtcctaaaaagc

SEQ ID NO: 361 - DNA, 103 bp, Vibrio parahaemolyticus; identical to SEQ ID NO: 264
  source 1..103: mol_type = genomic DNA

SEQ ID NO: 362 - DNA, 103 bp, Bacillus cereus
  source 1..103: mol_type = genomic DNA
acgctgtgctcttttgttttgtaatttttcgtatttacgtgaactttatatgtgtaaatgtaacataaacactaataaaattctatatctaatacttctgtaa

SEQ ID NO: 363 - DNA, 118 bp, Burkholderia multivorans
  source 1..118: mol_type = genomic DNA
gcgtcagatcgacgatcgtcggcagcagcgcgagatagaacagcatgatcttcgggttgccgagcgtgaccagcgtgccggcgacgaacatccgtatcggcgattgcccgcgcggcag

SEQ ID NO: 364 - DNA, 102 bp, Bacillus safensis
  source 1..102: mol_type = genomic DNA
tgcgtctaatctacggttataagattttttgtgtttatgttatgtttacatgcttaaacctgacataaatactaataaaattctatatgagtgattattatt

SEQ ID NO: 365 - DNA, 112 bp, Bacillus sp.
  source 1..112: mol_type = genomic DNA
cttgaccgaatgggacgaagtagatgttttttgtggccattaggcgcatgaggttgacgccattaagccctaaagcatcattcgtcgaaacggccaatacgacaggcttgcc

SEQ ID NO: 366 - DNA, 117 bp, Burkholderia ubonensis
  source 1..117: mol_type = genomic DNA
tgccgccacgccccacattcaagatgtcccaatcccccaaagtagggttcgtatccctcgggtgcccgaaagcgctggtcgactccgaacaaatcatcacccagctgcgcgccgagg

SEQ ID NO: 367 - DNA, 38 bp, unidentified
  source 1..38: mol_type = unassigned DNA
ggcttgtcgacgacggcggtctccgtcgtcaggatcat

SEQ ID NO: 368 - DNA, 119 bp, Clostridium botulinum
  source 1..119: mol_type = genomic DNA
gttttttcatataaagttacatcagcacctgcctttttagctgttatggctgctgcacatccagaccagccacctccaattattataatcttcatattagtcttctcctttcaaaaaca

SEQ ID NO: 369 - DNA, 105 bp, Cellulosimicrobium cellulans
  source 1..105: mol_type = genomic DNA
tcgtcgacgcctcgctgcccggcggcgagcggctccacgtggtgatccccgacgtcacgcgcaccgcgtgggcggtcaacatccgcaagcacgtggtccgtgcgt

SEQ ID NO: 370 - DNA, 105 bp, Clostridium difficile
  source 1..105: mol_type = genomic DNA
aataattttaattttttataaaaaatattcatatattctttatattaaagtttagatatctaaaaatacttttagaatttattatattatgttaatttttttata

SEQ ID NO: 371 - DNA, 122 bp, Clostridium difficile
  source 1..122: mol_type = genomic DNA
cctgtgatatatgaattaatatcttattcccttcagcatatccaaacatctcatttatatatttaaatttatcaatatctatatataataaagcatactttacttgagtatttttacatagt

SEQ ID NO: 372 - DNA, 106 bp, Clostridium difficile
  source 1..106: mol_type = genomic DNA
aagtaggcggtcatgggttgtatttatcaatattttaaaattagtgaaactccactttcccttagttaaagatatgaaccctatttattagtctactttcccaata

SEQ ID NO: 373 - DNA, 102 bp, Clostridium difficile
  source 1..102: mol_type = genomic DNA
ctcctgttgctcctgttgggcctgttactccatctgctcctgttgcccctgttgggcctgttgggcctgttactcctgttgctccatctgctcctgttgctc

SEQ ID NO: 374 - DNA, 104 bp, Clostridium perfringens
  source 1..104: mol_type = genomic DNA
ataatttatatattaataaaattatactcttaaaacaaaaaagaagctctaaaaagagcttctttttataattaaatttatattattttaatctttcttctaac

SEQ ID NO: 375 - DNA, 114 bp, Cronobacter sakazakii
  source 1..114: mol_type = genomic DNA
agaatggcgcccattttctttgaatcacagaatagcaaattatgagcgtacgtttagtgttagccaaagggcgcgagaaatctctcctgcgccgccatccctgggtcttttccg

SEQ ID NO: 376 - DNA, 106 bp, Clostridium thermobutyricum
  source 1..106: mol_type = genomic DNA
taggaaggaatttttttataatacataattacatacaacatatagtatgtaatgaataacactcaatatgatgtatgtaataaatccaaagcttagtaattagaat

SEQ ID NO: 377 - DNA, 113 bp, Desulfotomaculum nigrificans
  source 1..113: mol_type = genomic DNA
aagttaaagcggaggtttctctgtacgaccccattggtgtagacaaggaaggtaatgaaataagtttgatagatattttgggtaccgacccggaagtggtggcggacatggtg

SEQ ID NO: 378 - DNA, 113 bp, Escherichia coli
  source 1..113: mol_type = genomic DNA
catgaatgcagaccgaaagtaacgtcggccaggggaagcggcgaggtaaatcaagggtcattgaagtcataatcatttcagtagaaaaaccaggtctcgattttaaatgcaaa

SEQ ID NO: 379 - DNA, 107 bp, Escherichia coli
  source 1..107: mol_type = genomic DNA
ctacggaatagagataacacgaggagtggttagaaatggctaaagttctggtgctttattattccatgtacggacatattgaaacgatggcacgcgcagtcgctgag

SEQ ID NO: 380 - DNA, 104 bp, Escherichia coli
  source 1..104: mol_type = genomic DNA
caaatttgacggaaatgttttcaaacaacggcttactgccgaactgcatggtgacgttactggaaactaacacaggcgtatcctgaaaagagatatgacaaacc

SEQ ID NO: 381 - DNA, 117 bp, Escherichia coli
  source 1..117: mol_type = genomic DNA
gcaccatgaatgcagaccgaaagtaacgtcggccaggggaagcggcgaggtaaatcaagggtcattgaagtcataatcatttcagtagaaaaaccaggtctcgattttaaatgcaaa

SEQ ID NO: 382 - DNA, 104 bp, Escherichia coli
  source 1..104: mol_type = genomic DNA
gcttggcgttaccggtcactccgccctgaatatgtggtccggtattgtcttcagcattacatttttattttcggccatcgcctcaccgttttggggtggactcg

SEQ ID NO: 383 - DNA, 105 bp, Enterococcus faecium
  source 1..105: mol_type = genomic DNA
cgacaccaactggcttggcttctgcttggattttacgccatccagccaatatgcaagtgatcgccggtacgatgaacgtagggcgaatcaaggaaatcgctcaag

SEQ ID NO: 384 - DNA, 107 bp, Enterococcus faecium
  source 1..107: mol_type = genomic DNA
gtatccttttggtaaaattcatatcctgctgcgatggaataacattaccagaaggatgattatgcgctaaaacgatccttgctgcacaataacgtacagcataatgg

SEQ ID NO: 385 - DNA, 110 bp, Enterococcus faecalis
  source 1..110: mol_type = genomic DNA
tgaaaagctattttatacaacgggggcatagctcagttggttagagcatctgactcttaatcagagggtctagggttcgaatccctatgcccccattgggtgccaaaccc

SEQ ID NO: 386 - DNA, 103 bp, unidentified
  source 1..103: mol_type = unassigned DNA
tggaatcggtgggaatgaaaatcaccgttaattatggagaaaaggatgggaaaattgtcaagggttctaagacctacggcgatgtcaagaaagaagtgacaga

SEQ ID NO: 387 - DNA, 102 bp, Enterocloster clostridioformis
  source 1..102: mol_type = genomic DNA
aaggaagggacgtgctgggaatcatgcccactggggccggaaaatccctttgttatcaaatccctgcccttatgatggatggaatcacgctggtcatttccc

SEQ ID NO: 388 - DNA, 104 bp, Enterocloster clostridioformis
  source 1..104: mol_type = genomic DNA
ataaatttactgaggctttatcggttaaggttccttgaaaaaacaacaaaatcaggttaaattaggttttttaaggcagatgcaatctgtttgtgctgccaata

SEQ ID NO: 389 - DNA, 111 bp, Fusobacterium mortiferum
  source 1..111: mol_type = genomic DNA
tagcacctatcttattggcattgattggtgcgttaaatactccacctgtagtagatacgtgtgtcccaataaatttatttatctttctattttccattaaataattctcct

SEQ ID NO: 390 - DNA, 105 bp, Faecalibacterium prausnitzii
  source 1..105: mol_type = genomic DNA
ggagaatagcgggatcgaaccgctgacctcttgcatgccatgcaagcgctctcccagctgagctaatcccccacatattcggttggtgtcctgcgacgtgagtta

SEQ ID NO: 391 - DNA, 111 bp, Klebsiella pneumoniae
  source 1..111: mol_type = genomic DNA
tccgggggcccccactattcatatgaacggctctcaacctgtgctaaaaaacgaaaggacggcatgccatgaatatattcgatcactatcgccagcgctacgaagctgcca

SEQ ID NO: 392 - DNA, 113 bp, Klebsiella pneumoniae
  source 1..113: mol_type = genomic DNA
tttgcatttaaaatcggagcatcatttttcaacagaaacgactatgagcgcaatgacccttgatttacctcgccgctttccgtggccaacgctgctgtccgtggctatccacg

SEQ ID NO: 393 - DNA, 105 bp, Klebsiella pneumoniae
  source 1..105: mol_type = genomic DNA
cgatcatcgccggactggtggccgcagcgctatggaccgggcggctgtcgaggatcacgcggttgacgttacgctcgtgcagcccgcggttgagctgctgttccg

SEQ ID NO: 394 - DNA, 108 bp, Klebsiella pneumoniae
  source 1..108: mol_type = genomic DNA
cgaggatgagttatgaagctggaagaaatcgtagcccttagtgtaaagcataatgtctctgatctacacctgtgcaattccgccgcaccacgctggcggcggcagggc

SEQ ID NO: 395 - DNA, 110 bp, unidentified
  source 1..110: mol_type = unassigned DNA
ggctggccggtacgtcccggccccctgcggaacatctgcacggcgcacgccaagtcgtaggtgatcacgccgtcgagcgccagcgctgccaccgtcttggtgcccatacc

SEQ ID NO: 396 - DNA, 114 bp, unidentified
  source 1..114: mol_type = unassigned DNA
ccggaaaggtccggatggaaggcaaggaatacgtcatggttgacggtgacgtggtggaattccggtttaacgtctaggcggaagcgctactaatgccgcgccgccgcgaacttc

SEQ ID NO: 397 - DNA, 109 bp, unidentified
  source 1..109: mol_type = unassigned DNA
cggctgacgctggcgctgtcgccggccacgctgccgaagatggggtcggtctacgatctcgcgctggcgctggccgtgctgtcggcggagaagaaaaccgagtggcccc

SEQ ID NO: 398 - DNA, 117 bp, unidentified
  source 1..117: mol_type = unassigned DNA
gctggtcgaggagttaatgaagatctccaacatgttcggctacggtgaggtaaatcagtacaatcgtcactcgaccctaatgtctgcctataaaaacatcaaagatgaacatttccg

SEQ ID NO: 399 - DNA, 115 bp, Nocardia otitidiscaviarum
  source 1..115: mol_type = genomic DNA
gtgacccgttccgccgtcgagaacgccacctccgtggcgcgcatggtgctcaccaccgagagcgccgtggtcgacaagccggccgaggaagagcccgcgaacggccaccacggcc

SEQ ID NO: 400 - DNA, 104 bp, Pseudomonas aeruginosa
  source 1..104: mol_type = genomic DNA
ccggtttcccttcgcacccgcaccgcggcttcgagaccgtgacctacatgctcgaagggcgtatgcgccacgaagaccacctcggcaatcgcggcctgctcaag

SEQ ID NO: 401 - DNA, 102 bp, Pseudomonas aeruginosa
  source 1..102: mol_type = genomic DNA
aactgccggagttcgagcgcaaggtcctggaggtcctgcgcgagccgctggaaagcggcgagatcgtcattgcccgggccaacggccgggtacgtttcccgg

SEQ ID NO: 402 - DNA, 114 bp, Paenibacillus campinasensis
  source 1..114: mol_type = genomic DNA
ttgcccggtatcaggaaactttacttatgatagttgacactgcttattcaacacatgtggagtgtgtggtgttgttggttaggaatgacagataattgaggtatactgacgtgt

SEQ ID NO: 403 - DNA, 105 bp, Bacillus sp.
  source 1..105: mol_type = genomic DNA
cgaaaaaaaggaatacctctataaaataatatgggtattctctaatatttatttctataaatatagagaaatacccatattttcgcatataaaagaataaattat

SEQ ID NO: 404 - DNA, 108 bp, Pseudomonas fluorescens
  source 1..108: mol_type = genomic DNA
gtggcaaaccttttcagtgcgtgattggcaccggccgggtgatcaagggctgggatcaagggctgatggggatgcaagtgggcggcaagcgcaaactgctggtgccgg

SEQ ID NO: 405 - DNA, 102 bp, Pseudomonas fluorescens
  source 1..102: mol_type = genomic DNA
gcacgaacaagcgacgcaccccgcccacctgcatgcccatcaggccctggtcccagcccttgatcacgcgccctgtgccgatcacgcactggaaaggcttgc

SEQ ID NO: 406 - DNA, 106 bp, Pseudomonas fulva
  source 1..106: mol_type = genomic DNA
cgggcagggtctacctaagcctttacatttgtgtacatctgaaattgttgcttgtaggtatctcatatgtttacaatttgcacccaagattctttcagagggcgcc

SEQ ID NO: 407 - DNA, 105 bp, Pseudomonas fluorescens
  source 1..105: mol_type = genomic DNA
gtggcaaacctttccagtgcgtgattggcactggccgcgtgatcaagggctgggatcaggggctgatgggcatgcaggtgggcggtaaacgcaaactgctggtgc

SEQ ID NO: 408 - DNA, 105 bp, Prochlorothrix hollandica
  source 1..105: mol_type = genomic DNA
aatcacgatctattggtctcaattctccattcgtgattgtatggttattgttgaataagttaacaatcgcgaatttatgaaaattgcctatgcccgtgtctcaag

SEQ ID NO: 409 - DNA, 39 bp, Streptomyces sp.
  source 1..39: mol_type = genomic DNA
ccccaactggggtaacctttgagttctctcagttggggg

SEQ ID NO: 410 - DNA, 106 bp, Pseudomonas putida
  source 1..106: mol_type = genomic DNA
gccggcaccagcagcttgcgcttgccgccgacctgcatgcccatcaggccctggtcccagcccttgattacccgaccggtgccgatcacgcactgaaacgccttgc

SEQ ID NO: 411 - DNA, 104 bp, Paeniclostridium sordellii
  source 1..104: mol_type = genomic DNA
ccaagcaatactatagcttcaggtaaataggaactttaatacatttatctgaagatatatgtattaaagtaaaactttttaattatgatgtcaattaattctta

SEQ ID NO: 412 - DNA, 105 bp, Pseudomonas syringae
  source 1..105: mol_type = genomic DNA
gtggcaagccatttcagtgtgtgatcggcaccggtcgcgtcatcaagggctgggaccagggcctgatgggcatgaaagtcggcggcaagcgtcaattgttcgtcc

SEQ ID NO: 413 - DNA, 148 bp, unidentified
  source 1..148: mol_type = unassigned DNA
agcggcacagaaaccaagcgacgaattcatcaagaagataacttgaaagaaatggtgcccggaggcggatttgaaccaccgacacgcggattttcaatccgctgctctaccaactgagctatccgggcacttcaggtccttgaagaac

SEQ ID NO: 414 - DNA, 116 bp, Rhodococcus equi
  source 1..116: mol_type = genomic DNA
gctcggcggacggcgacccgtacctggcaagctgaacgccgtgttccgagtgatgttccacgagccccgcatcccacccaatacgggaaacgcgatccgcatggtcgccgggacgg

SEQ ID NO: 415 - DNA, 102 bp, Ruminococcus lactaris
  source 1..102: mol_type = genomic DNA
catatgagtcaagcaaataaagtgtatctcatttttaaacgaaacatgtattacatttaaaaataggaattttaccccatatgatattatacattaactaag

SEQ ID NO: 416 - DNA, 109 bp, Staphylococcus aureus
  source 1..109: mol_type = genomic DNA
tgattgttttaagtgggactttttatattgcaaaaaataaatggcggacgaggtaacaggatacctcatctgccaattaaaatttgttaatttaataattaaataaaaa

SEQ ID NO: 417 - DNA, 106 bp, Staphylococcus aureus
  source 1..106: mol_type = genomic DNA
aacatgaggttattgttgctaatattaataagttatattggaggaacgtgtgcgttagaagtcgtaccattcatgtccttacgagataaattaactaaacacgtat

SEQ ID NO: 418 - DNA, 129 bp, Staphylococcus aureus
  source 1..129: mol_type = genomic DNA
acgtcaattaagttttcgtgttttttattgaatagccttcttagtagtttcatttgtagttcctccttcattcgaaatcttcaattgacaaggtttcaattcgtttttggtaacgatataaataaaagt

SEQ ID NO: 419 - DNA, 102 bp, Streptococcus agalactiae
  source 1..102: mol_type = genomic DNA
tgtctgaaataacagacactaaatatataagtgttttatgtacatttattgaaataagtgtaagttaaacactctattttttaaataaaatttccatgtcct

SEQ ID NO: 420 - DNA, 128 bp, Staphylococcus arlettae
  source 1..128: mol_type = genomic DNA
ataaatttctgtagttatttttcaaaaaccgcatcattaactgataagcagaagcatatcacaaataaaactaaaaaaacgatgttgaacaataatattcattatgaattttttgagtaaatcttagg

SEQ ID NO: 421 - DNA, 102 bp, Streptococcus equinus
  source 1..102: mol_type = genomic DNA
gacagtatcaaaattttatggaaaatttaacaaatttagtatcattcatttcaatcaaatatattaaatttgtttattaaataggaagaaaaagacggtcat

SEQ ID NO: 422 - DNA, 110 bp, Staphylococcus hominis
  source 1..110: mol_type = genomic DNA
gtggtgcttgttacataactcttatgtatattttatatgggggttatagggggtacgtagcaccggtaccccccatagttgcaacgaacatttgtgtaccaggtgtaata

SEQ ID NO: 423 - DNA, 112 bp, Streptomyces ipomoeae
  source 1..112: mol_type = genomic DNA
ttccgacgcagtttccgacgagtacgaggacgaggacagacgtgcctaccggcaaggtcaagtggttcaacagcgagaagggcttcggctttctctcccgcgacgacggcgg

SEQ ID NO: 424 - DNA, 102 bp, Streptococcus mitis
  source 1..102: mol_type = genomic DNA
cattcattgcagatgtatgagatggaaaaaagaaataattttactatcctttgtggaaatgtaggttactaaaattacttatattttccacttgatgacaat

SEQ ID NO: 425 - DNA, 110 bp, Salinispora sp.
  source 1..110: mol_type = genomic DNA
tcagcagcgcggccacctgctcgtcggcgaaggactcgtgcgccatgacgtgacaccagtggcaggcggcgtaaccgaccgaaatcatgatcgggacgtctcggcggcgg

SEQ ID NO: 426 - DNA, 133 bp, Treponema denticola
  source 1..133: mol_type = genomic DNA
taatttaacaaggcagataatttaaccgcaggggacgcaaaggacgctaaattttttttataatttactatttttttcaaataattcttataaatataatggggatgggaaaatattaaaaaataataggaga

SEQ ID NO: 427 - DNA, 108 bp, Tenacibaculum dicentrarchi
  source 1..108: mol_type = genomic DNA
ataatgtactggttaaaagtaatttatgagcaatatataaaaaataatactaaaagtaattaatttttatatattgctcatatttaaaaaaaaatataaatataagct

SEQ ID NO: 428 - DNA, 112 bp, unidentified
  source 1..112: mol_type = unassigned DNA
acgtagtagacatttttctcgtccaggcggtccttggcgaggcgcaggccttcgccgaagcgctggaagtgtacgatctcgcgcgcacgcaggaacttgatgacgtcgctca

SEQ ID NO: 429 - DNA, 111 bp, Vibrio sp.
  source 1..111: mol_type = genomic DNA
tcaaacgctaggtcttctgcttctagccattttttggcttttttaattgtgtcgcaatttgggatgccgaacatggtaatcgtcatattatttccttttctttttattttt

SEQ ID NO: 430 - DNA, 102 bp, Vibrio harveyi
  source 1..102: mol_type = genomic DNA
actccaagcaggtaggccgtttttctaaactatgctaagcaatttctttaattaaagtttttgctttgtatggtttaggtgataagctctgcacccctttaa

SEQ ID NO: 431 - DNA, 103 bp, Vibrio parahaemolyticus
  source 1..103: mol_type = genomic DNA
tgtcttgtaagcacccatccctgtagttatgcactgtacatttgctttcagtctaatgtatagtgataacttacattttatggtatggtgtaacaatgagaaa

SEQ ID NO: 432 - protein, 465 aa, Bacillus cereus
MTVGIYIRVSTEEQARDGFSISAQREKLKAYCVAQDWDNFKFYVDEGVSAKDTNRPQLSILLNHIQQGLITTVLVYRLDRLTRSVMDLYKLLDTFDKYNCAFKSATEVYDTSTAMGRMFITIVAALAQWERENLGERVRMGQLEKARQGEYSAKAPFGFDKNKHNKLVINEIESKVVLDMVRKIEEGYSIRQLAIHLDSYIKPIRGYKWHIRTILDILSNNAMYGAIKWSNEIIEGAHEGILTKERFIQLQKILSSRQNIKKRQTHSIFIYQMKLICPNCGNRLSSERSRYYRKKDEQHVECNQYRCQSCALNKHTTKPFATSERKVESALMNYISNLQFEQVPKINNENNELEILKKQVKKVEKQREKYQKAWSNDLMTDDEFTERMNETKILLNSAKKKLQTLEVNNHQEIDVDVIKEKVNNIKKNWFHLSPDEKKQFMSMFIENIKIDKKDGVTEVLDIEFY

SEQ ID NO: 433 - protein, 560 aa, Burkholderia multivorans
MAKKPKAKVYSYLRFSDPKQAAGSSADRQMEYAARWATEHDMQLDATLTLRDEGLSAFHQRHIKQGALGVFLRAIEDGRIQPGSVLIVEGLDRLSRAEPIQAQAQLAQIINAGITVVTASDGREYNRERLKAQPMDLVYSLLVMIRAHEESDTKSKRVKAAIRRQCEGWVAGTWRGIIRNGKDPHWVRLGEHGKFEHVPERVLAVRTMIDLFLEGHGAIEITRRLTEQNLYVSNAGNYSVHMYRIVRNQALIGEKRISVDGEEFRLDGYYPPILTREEFAELQQTMSERGRRKGKGEIPNIITGLSITVCGYCGRAMTTQNSKARAPKGKSVVRRLSCPMNSFNEGCPIGGSCESEIVERALMRYCSDQFNLSRLLEGDDGTARRTAQLAVARQRASDIEAQIQRVTDALLSDDGKAPAAFTRRARELETQLEEQRREIEALEHQIAASSAHGIPAAAEAWAQLVDGVLALDYDARMKARQLVADTFRKIVVYQRGFAPIDDAAADRWKRSGTIGLMLVTKRGGMRLLNVDRRTGCWQAEDDLDPSLIPSDGLPMLPLDA

SEQ ID NO: 434 - protein, 472 aa, Bacillus safensis
MEDSSNKSVGIYVRVSTDEQAKEGFSISAQKEKLKAYCVSQGWANFKFYVDEGKSAKDTHRPSLELLLRHIEQGIIDTVLVYRLDRLTRSVRDLYTLLDYFDKYNAVFRSATEVYDTGSATGRLFITLVAAMAQWERENLGERVKMGQNEKARQGQFSAPAPFGFIKEGKSLVKNHEQGEILLEIIDKVKKGYSTRQIANYLDDSGLLPIRGYRWHPGTILTLLKNPILYGSFRWGDEIIEDTHEGYISKDEFDRIQEILKERSIVKKRDSYSVFIFQSKIVCAGCGNRLASERSKYFRKKDKQYVETNNYRCQTCAQNRKPSIMGSEKKFQKALVKYMQNVTPKLEPKIPEEKKHDYEKVHQKILNLEKQRKKYQKAWSLDLMTDEEFEQLMYETKEALKSAQNELAAAHSSDSQNSQIDIERAKEIVKMFNENWSVLTNEEKRSIVQELIKHINFTKEDGEIIITHIEFY

SEQ ID NO: 435 - protein, 515 aa, Bacillus sp.
MKTAIYLRKSRADLEAEARGEGETLAKHRSTLLKIAKEMNLNVLSVREEIVSGESLVKRPEMLALLEEIEDNKYDVVLCMDMDRLGRGGMKEQGIILETFKRSNTKIMTPRKTYDLNDEWDEEYSEFEAFMARKELKIITRRMQRGRVASVEAGNYLGTHAPFGYDIHRLNKRERTLTINPEEASIVRMIFDWYANEDMGANAIRSKLNDLGYKSKLGNEWNPYSILDILKNNVYIGKVTWQKRKEVKRPDAVKRSCARQDKSDWIIADGKHEPIIPESLFEQVQEKLNSRYHIPYNTNGIKNPLAGIIKCAKCGYSMVQRYPKNRKETMDCKHRGCENKSSYTELIEKRLLEALKEWYINYKADFEKHKQDDKLKETQVIQMNEAALRKLEKELVDVQKQKNNLHDLLERGVYTVDMFLERSNVVSDRITEITSTMENLKKEIKTEIKKEKVKKDTIPQVEHVLDLYFKTDDPKKKNSLLKSVLEKAVYKKEKWQRLDDFELVLYPKLPQDGDI

SEQ ID NO: 436 - protein, 550 aa, Burkholderia ubonensis
MAAKSRVYSYLRFSDPKQAAGGSIDRQLEYAARWAADREMELDASLSLRDEGLSAYHQRHVRQGALGVFLRAIEDGQVPAGSVLVVEGLDRLSRAEPLQAQAQLAQIVNAGITVVTASDGREYNRERLKAQPMDLVYSLLVMIRAHEESDTKSKRVKAAIRRQCEGWIAGTWRAPIRVGKDPHWVRETEGGGFELAADRASAVRLVIEMFRQGHGAVRIVRELAERGLQISETGRTHSSNIYKILANRMLIGEKSVEVDGETYQLDNYYPALLTPAEFADLRYLAEQRGRRKGKGEIPGVVTGLGITYCGYCSAAIVAQNLMGRKRLPDGRPYPGHRRLHCVTYSQSVGCKISGSCSVVPVERALMLYCSDQMNLTRLLEGDAGTASISAQLASARQRVAELEAQIRRVTDALLFDDGDAPAAVLRRVRELESQLASERRDVESFEHHLAASASNVAPAAADAWRELVQGVEQLDYDARLMARQLVADTFSRIVIYQSGFQPETDHGTIGVVLVGKRGNTRMLNVDRRTGEWRSAEDIELSGLATIPLPA

SEQ ID NO: 437 - protein, 500 aa, unidentified [sequence omitted]

SEQ ID NO: 438 - protein, 509 aa, Clostridium botulinum
MLRIAIYSRKSVETDTGESIQNQIKLCKEYFKRQDPNCIFEIFEDEGYSGGNINRPSFQRMMELVKIKQFDIVAVYKIDRIARNIVDFVNTYDELDNIGVKLVSITEGFDPSTPAGKMMMLLLASFAEMERMNIAQRVKDNMRELAKMGRWSGGTPPKGYTTKKVIENGKKITYLDLIDDEAYIIKDAFKLYAEGYSTYKINKHFKEKGIRLPQKTIQNMLNNPTYLISSKESVDFLKNKGYTVYGEPNGFGFLPYNRRPRTKGKKSWNDKSQFVGVSKHEGIIDLPLWIEVQNKLKERTVDPHPRESNFTFLSGGLLKCSCGSSMFVHPGHTRKDGSRLYYFRCMKNNGNCSNSKFLRVDYAESSILEFLESISSKEKLTEYQKKKKPRLDFSIEIKNLNKKIRDNSKAIDNLIDKLMILSNEAGKVVATKIEELTKQNNILKESLLEIERKKLLSGLEDNNLNILYNEIQNFIQTEDISLRRLKIKNIIKYITYNPQNDSLQVELVD

SEQ ID NO: 439 - protein, 486 aa, Cellulosimicrobium cellulans
MSRRRALAPVPDTPPRAVAYLRQSTYREESISLELQETACRDYAARHGYQIVAVEKDPGISGRTWNRPAVQRVMEMIESRDADVIVLWRWSRLSRNRKDWAIAADRVDVAGGRIESATEPNDTATAAGRFARGVMTELAAFESERISEQWKEVHTSRVARGLPPGGKLPWGWRWVDGAVRPDPETAPYIVEAYRRYLAGAGNRDLADWFNGSGVRPMHAKEWYFSTITQCLDSPIHAGLIAYHGQTLPGAHDGIIDVATWEAYRRERERRAGERQVKRRYLLSGIAHCPCGEPMLGFTQDKEGRPRTGRTRSPWSCYRCSSLGKPEGHGPWNISLRFVEPVVMDWLHQVAADVENKVPRAAARRDDAHRESQRIAREITALDAQLTALTGHLASGLVPEAAYVTTRDEILARRAELERGLAEAERLVLHVPDDPSAIAREALADWDTLPIETKRATLRQLIRTVLVDYEHRTAHVVPVWEPMHNEG

SEQ ID NO: 440 - protein, 503 aa, Clostridium difficile
MNNRIDAIYARQSVDKKDSISIESQIEFCKYELKGGNCKEYTDKGYSGKNTDRPKFQELVRDIKRGLIAKVIVYKLDRISRSILDFANMMELFQQYDVEFVSSTEKFDTSTPMGRAMLNICIVFAQLERETIQKRVTDAYYSRSQRGFKMGGKAPYGFHTEPIKMDGINTKKLVVNPDEAANIRLIFEMYAQPTTSYGDITRYFAEQGILFYGKELIRPTLAQMLRNPVYVQADLDVYEFFKSQGTVIVNDAADFTGTNGCYLYQGRDVKPSKKNDLKDQMLVLAPHEGIVPADIWLACRKKLMNNMKIQSARKATHTWLAGKIKCGNCGYALMSIFNPSGRQYLRCTKRLDNKSCPGCGKIITSELEAVVYQQMVKKLEKHKTLTGRKKAAKANPKIAALQVELLHVDSEIEKLVDSLTGANNVLLSYVNVKIAELDGRKQELVKQIAELTVETISPGQVNQISGYLDTWDDVSFDDKRRVVDLMITTVAATSDSLNITWKI

SEQ ID NO: 441 - protein, 459 aa, Clostridium difficile
VVTKDLSRLGRNYIMTGQYTELYFPSHNVRYIAIDDGVDSEKGESEIAPFKNIINEWVARDTSRKVKSAFKTKFAEGAYYGAYAPLGYKKHPDIKGKLLVDEETKWIVEKIFSLAYQGYGSAKITKILREEKVPTASWLNFTRYGTFAHIFEGKPESKRYEWTIAHVKAILKSEVYIGNSVHNRQSTVSFKSKKKVRKPESEWFRVENTHEPIIDKEVFYRVQEQIKSRRRQTKEKATPIFAGLVKCADCGWSMRFATNKANKTPYSYYSCSFYGQFGKGYCSMHYIRYDVLYQAVLERLQYWAKAVQQDEEKVLNKIQKVGNAERIREKKKKASALKKAENRQNEIDRLFAKMYEDRACEKITERNFIMLSGKYQKEQIELEQQITNLREELSKMEQDMIGAEKWIELIKEYSVPKELTAPLLNAMIEKILIHEATTNEENERIQEIEIYYRFIGKVD

SEQ ID NO: 442 - protein, 537 aa, Clostridium difficile
MKQQIYNTALYLRLSRDDELQGESSSITTQKSMLRLYAKEHHLNVIDEYIDDGWSGTNFERPSFQRMIEDIEAGKINCVVTKDLSRFGRNYIMTGQYTELYFPSHNVRYIAIDDGVDSEKGESEIAPFKNIINEWVARDTSRKVKSAFRTKFAEGAHYGAYAPLGYKKHPDIKGKLLIDDETKWIVEKIFSLAYQGYGSAKITKILREEKVPTASWLNFTRYGTFAHIFEGKPESKRYEWTIAHVKAILKSEVYIGNSVHNMQSTVSFKSKKKVRKPESEWFRVENTHEPIIDKEVFYRVQEQIKSRRRQTKEKATPIFAGLVKCADCGWSMRFGTNTANKTPYSYYACSFYGQFGKGYCSMHYIRYDVLYQAILERLQYWAKAVQQDEEKVLHKIQKAGNAERIREKKKKASALKKAENRQNEIDRLFAKMYEDRACEKITERNFVMLSGKYQKEQIELEQQITSLREELSKMEQDMIGAEKWVELIKEYSVPKELTAPLLNAMIEKILIHEATTNEDNERIQEIEIYYRFIGKVD

SEQ ID NO: 443 - protein, 560 aa, Clostridium difficile
MPRIRKDKMARSYAEPFWKIGLYIRLSREDDNEDESESVINQEKILRDFVDSYFEPGTYVIVDVFADDGLTGTDTARPNFKRLEGCIVRKEVNCMIIKSLARGFRNLADQQKFLEEFIPINGARFICTGTPFIDTYANPHSASGLEVPIRGMFNEQFAATTSEEIRKTFKMKRERGEFIGAFAPYGYKKDPNDKNSLIVDEEAAEVVKSIYHWFVNEGYSKMGIAKRLNQMGEPNPEAYKKKKGFKYNNPNSDKNDGLWSASTIARILQNEVYTGVMVQGRNRVISYKVHKQINVPEEEWFVVPNTHEAIIDRETFEKAQALHKRDTRTAPGKQEVYLMSGFVRCADCKKAMRRKTARDIAYYSCRSYTDKKICSKHSIRQDKLENAVLAALQMQIALVDRLAEEIERINNAPVINRESKRLSYSLKQAEKQLKQYNDASDSLYLDWKSGEITKEEYRRLKGKIAEQIQQLEANISYLKEEMQVMADGIGTDDPYLTAFLKHKNIQSLNRGIMVELVKAVWVHENGEITVDFNFADEYQRIIDYIENNHNVIQVIENKAI

SEQ ID NO: 444 - protein, 544 aa, Clostridium perfringens; identical to SEQ ID NO: 3

SEQ ID NO: 445 - protein, 562 aa, Cronobacter sakazakii
MEKRKLYSYIRWSSDKQAKGSSLQRQLETARRVAHENDLELVEIIDAGLSAFRSKHLEKGSLGAFIEAVKVGQIASDSWLTVESLDRISRDAILKAQGLFMELLELGITIYTGMDNRIYTKSSVTDNPMELMLSIMTFSRANQESMVKSQRQKSATQLKINKFNASVRADNGYPHAIRNSGANVFWSDVSDGTVRPHEYYFPIAKLIVSLRLKGWGYMRIAKHLNENYTPPKGTAKRKHFKDLWSPKLICNFLQSRTLMGEKSIRVDGVEHILKGYYPQVISENEFYSLQNVINRKLANEPNNYIPLLTGIRRFKCGCGAAMVAFFQYDKIRYRCDGMKSLDKRMHCGAKSFNGASIENAIVQICADKIFKDVKTHTSNVGAIQAMLVEAKRKYNRGMDMLLEDTAPQGLNIKLIELEKQIEELQKKLNDELVTEAGVNSSVSWGAVPKDYRDVFNTERLEIKQKIQASTLSIVCTTIKTKGHLFEIKFNDGDVIRFFSDKRRVHVDVHSINNAEIMQSEGIILHDHLDYLVGPEEFIERIRQKHLQMKALQERTLDDIFDI

SEQ ID NO: 446 - protein, 504 aa, Clostridium thermobutyricum
MENVCIYLRKSRADEEMEKTLGHGETLSKHRKALLKIAKEKNLHIVEIKEEIVSGESLFFRPKMLELLKEVEDKKYNGVLVMDMQRLGRGDMKEQGIILETFKNAKTKIITPNKIYDLNNDFDEEYSEFEAFMSRKELKMITRRMQGGRVRSVEDGNYIASAPPYGYDIDYILKSRTLKINEHEAEGVKIIFDSYINGNGASAISEKLNNLGFKTKLGNNFSPSSVLTIIKNPVYIGKVTWKKKEIKKSKTLGKVKDTRTRDKSEWIIANGKHKPIISEEVWNNTQEVLKNKYHIPYQLTNAPINPLAGLLVCGVCGKAMVMRPSRGILRVMCVHKCGNKSVRFDYVEKAIIDSLEQYYSNKKLEVKKQKTIQNTSNEEKELILLENELSTLNKQRLSLFDFLERGIYTEDVFLERSKNIDSRINLVESEMKKISEKIKFKKTKKDTKALLQTLNNAIENYKSSSDVITKNSYLKSILNDITYIKTPEQKRNSFSITLNPKLRF

SEQ ID NO: 447 - protein, 519 aa, Desulfotomaculum nigrificans; identical to SEQ ID NO: 1

SEQ ID NO: 448 - protein, 506 aa, Escherichia coli
MRSVITYLRYSSAIQGAEGADSTRRQNDLFKQWLKKNSDAQVVASFSDEGLSGYKGKHLTGQFGDMLARIESGEFPEGTILLVESIDRIGRLEHLETEALMNRILGNGIEIHTLQDGLIYTKDALADDLGISIIQRVKAYIAHQESKQKSFRVSQKWEQRAKLALAGEQRLTKMVPGWIDPDTFKLNEHAETVRLIFKLLLDGESLHNIARHLQSNGIKSFSRRKDANGFSVHSVRTILRSETTIGTLPASQRNDRPAIPNYYEAAIDAATFNKAQEVLDKNRKGRTPASDNPLTINIFKGLFRCQCGASVHPTGTKNKYAGVYRCNNHLDGRCDVPPLKRKPFDKWVLENFLGMIDVGNDGESERKIAALQHEVEIVTARIKKATALLLEMDDIDELKIQLKELNQKRTELQTTIDSMRRKASLTDKELPQLKDIDLTTKAGRVECQLILSKHLKGLTLGKDSVTVTLQNDTEITIPTNPLPLNDGSPIFEIADKELLDIDAYQL

SEQ ID NO: 449 - protein, 547 aa, Escherichia coli
MKLLVTYIRWSTKEQDSGDSLRRQTNLIDAFYSKHKNDYYLLPAHRYVDKGKSGFHQQHKAQGSDFRRMFENVMSGAIPEGSLIVVENFDRFSRADIDTAIDDVRQILRKGVSILTLGDGELYDKSALTDPVKLIKHIIIAERAHQESLVKQKRIAQVWNHKTQLARELKKPMGKQAPGWLELSEDGSHYIVDEDKASLVNIIYDKRLSGMSMFAICKWLNEQGYPTINQRKVRISKTKKPDGNWSALSVKHILTSRSVLGYLPAKISTEDRKTVLREEIEGFYPQIVTDSKFYAVQQLLEETGKGKTSSGEHWLYVNILKGLIRCKCGLVMTPTGIRKPVYQGTYRCNGNKESRCSYGTVSRKLLDTQLCSRLFSKLSQLHDEATDTAKLDELQRRLNTVDSELEKLTETLIQLPNITQIQEALRVKQEEKDELIVQLSREKARVKSVSSLDLSGLDMESVEGRTEAQIIIKRLVKEIVVSGNEKLVDIYLHNGNMIRGFPLDGKDDHTLTLEEATDEMQSLDDMLIFGEPVTRIYPAGDMEEVDA

SEQ ID NO: 450 - protein, 499 aa, Escherichia coli
MKQNGKAYSYIRFSSKKQEQGDSVRRQIALAEHYAHANNLILSDKNFQDLGISAFKEGNRPSLGDMLEAIEQGQIEQGSTIIIESLDRLSRRGIDVTQQIIKSILQKNVFIASLVDGLLLNRDSVNDLVSVIRIALAADLAYKESEKKSKRLRETKGQQRQRALKGEVINKILPFWLERKQNKYIFSNRLATVKRIIELRQKGLGTNKIAKILNEEGHKPLRSKGWNHTTIGKTINSVALYGAYQTSETTQDRKVILLDIVENYYPAVISKEEFMLLQSDHKQNKPGYKSEHNAFAGLLKHECGGALVRKFHVASGKTYQYHVCANARDGKCNVTKNFKNIEVALYQIMKELKLEKKTSFDKTLLEERNSVKTKIQELNNMLLELPKVPLSVLQTINNLEEKLQELEEKIKHQDNIIASEKTFNINTLRETKDPQQLNMMLKRVIENIIVFNIEKRWRIKILYRNKHSQSFIWDGSNITFVSDTKKLLELVKHTPEESK

SEQ ID NO: 451 - protein, 506 aa, Escherichia coli
MRGVITYLRYSSAIQGAEGADSTRRQNDLFKQWLKKNSDAQVVASFNDEGLSGYKGKHLSGQFGDMLSRIESGEFPEGTILLVEAIDRIGRLEHLETEALMNRIIAHGIEIHTLQDGLIYTRDALSDDLGISIIQRVKSYIAHQESKQKSFRVSQKWEQRAKLALAGEQRLTRNVPGWIDPETFKLNEHAETVRLIFKLLLDGESLHNIARHLQANNISSFSRRKDANGFSVHSVRTILRSETTIGTLPASQRNDRPAILNYYEAAIDASTFNKAQEILSKNRKGRTPASDNPLTINIFKGLFRCQCGASVHPTGTKNKYAGVYRCNNHLDGRCDVPPLKRKPFDKWMIDNFLGMIDVVSDGESERKIAALQHEVETVTARIKKATALLLEMDDITELKAQVKELNQKRTELQTTIDTLKRKTSLTDKELPQLKDIDLTTKVGRVECQLILSKHLKGLTLGKDSVTVTLQNDTEIIIPTDPLPLNDGTSILEIAEKELLGIDVYQL

SEQ ID NO: 452 - protein, 509 aa, Escherichia coli
MRRAISYIRFSSERQLKGDSVRRQSKLVTDWLDKNPEFYLDSSLSFKDLGKSAFSGKHLKGGLGDFLTAIEKGLVKAGDTLLIESLDRLSRQDIDIASELLRRILRAGVDIVTLSDGEHYTRESLKDPLSLIKSILIMQRAHEESLRKSERVQAAWNRKKELISEGIKVSRRCPAWLRLNDDRRTFTIIPDKVEVVKRAFDLRLQGLSFWAITRTLNDEGHLSLNQYTPKQKGWSDTAVKKLLRNRAVIGCFTPAGREEVQGYYPAIISESLFYRVQQLNTGQYGRASVSSNPLSVNLFRGIIKCSECGATIVLGGYALKRFGMYRCPNRSANRCSAKAISRKQTDTTILYMLALCDRFETENTDTIDSLKLQREDLQRKISKMAELAIELDDMTIITEKLRDMKNALSKLNHDIEEETKRIKAITTGSLKDIDRTTKEGMIETQLLIKGVLKEIVIDAAKRRCKVTFHNGKVIDLSITENPSEDVTEAIQSLSEVTERGLIDVDEVII

SEQ ID NO: 453 - protein, 448 aa, Enterococcus faecium
MRTGLYVRVSTAEQEKHGYSIKVQLEKLRAFASAKDYTVVKEYIDAAQSGAKLERPGLKQLIEDVENNALDCVLVYRLDRLSRSQKDTMYLIEDVFLKNSVAFVSLQESFDTTSSFGRAMIGMLSVFAQLERDNITERLFSGRAHRAKRGFHHGGGIIPFGYRYDVETGELKRFENESNEVKAMFEMIANGKSVSSVAKEFNTYDTTIRRRIANSVYIGKIQFDGETFDGQHEPIISKELFDKANVRMNARASNLPFKRTYLLSGLIYCGKCGERCSAYESRSKHNDKEYRRAYYRCNARTWKYKQKHGRTCEQPHIRVDELEQAVMEQVKRLPLKHKVKKRAFDFKPVENKIATIDKQKERLLDLYLNEHLDNEMFNKKSKELDKSRDKLAKQLERMRMQAADSVESYQWLDGIDWDALDKDTLREVLERIIERIVIRDKDVEIYFK

SEQ ID NO: 454 - protein, 450 aa, Enterococcus faecium
MKRVALYMRVSTEQQAKHGDSLREQKETLYEYIEQHKDLKVVNEYVDGGISGQKINRDEFQKLLQDVKENKIDLILFTKLDRWFRNLRHYLNTQEILEKHNVSWNAVSQQYYDTTTAYGRTFIAQVMSFAELEAQIDSERIKAVMANKIAQGEVVSGKTPLGYSIENKKLVINDDAPIVIDIFNYFLSSGSLRKTVYYLGSQYGIVRDYQSVKNMLTNKKYIGELRNNKNYCPPIIDKKLFYAVQKALPKNLKTNAKRDYIFKGLLKCSDCQGSVAGQTIKARYKKKDGTESIYERTCYRCVKRRNNKLRCTNKRAFYEKNLERYIFEATKQKFEQIQINYSKKQPKIIKKKNSKKNIENKLDRLKKAYLNEVIDLEEYKKDREALMKELNEIEVEPAKIDIKNVEFILSKEFDEIYKESSEEEKNALWRSIIDNIIVFPDGNITVNFLI

SEQ ID NO: 455 - protein, 387 aa, Enterococcus faecalis
MSKRTRRTFSQEFKQQIVNLYLDGKPRVEIIREYELTASAFDKWVKQSKTSGSFKEKDNLTPEQKELLELRKRNQQLEMENDILKQAALIFGPKRQVIDANKHLYPISAMCRILGLSRQSYYYQSKPKKDESELEEVVAEEFIRSRKAYGSRKIKKALSKRGIKISRRKISRIMKNRGLKSSYTVAYFKIHHSTCNEAKTTNVLNRKFLRDNPLEAIVTDLTYVRVGKKWNYVCFNLDLFNREILGYSCGEHKDAVLVKKAFSRIKQPLTEVEIFHTDRGKEFDNQTIDELLTTFDINRSLSHKGCPFDNAVAESTYKSLKVEFVYQYTFETLQQLDLELFDYVNWWNHLRLHGTLGYETPVGYRNQRLAQRILDNELGCANASEAV

SEQ ID NO: 456 - protein, 464 aa, unidentified
MKKRAGLYIRVSTEEQVDNYSIPEQKRRLEAYCQSHDWAIAEEYIDGGFSGAKLDRPAMQKMIADSKAGNLDVVVSIKLDRLSRSQKDTLHLIEDIFLPYHVDYVSVNESFDTGTSFGRAMVGILSVFAQLEREQILERMHSGMEARAKTGLYHGSKPPYGYALEKGVLKINPTEALAVRKVFDLWLKGYSYNKISEIMEETYHGEKAWMHPSAINQLLTNPAYTGKIRFAGEVIEGQHEPIIDEITFKKANLRLETRAANRGRQTTYLLTGVIWCGNCGSRFGINMSTCNGVRYTYYTCSPNRRKKGNEGIRCCGNKPIPTKKLDPLIIEKIKRLAISKDFFEEIQKPDASPSDAIASLESAAAEIDKRIGKLMELYSMDGIPTDTLSQQINTLYAQKKDLESQISEKQSGFKQTYEDYKEVFDKVDYAFKSGTIEEQKSIVRSLIKRIDILNQEITITWNFQ

SEQ ID NO: 457 - protein, 469 aa, Enterocloster clostridioformis
MMKKIAVLYMRMSTDMQEHSIESQERVLMEYAKRNGYVVIRKYIDRGISGQHASKRPDFLKMIDDSETGEFQFVLIYDSSRFARNLVESLTYKSILKENCVRLISMTEPNLEDDEMSLYIDAMQGATNEIYVRKLSKSVKRGHNDRALRGDLPGDVQFGLKRLKDGSIVLDEVKAPIMRWMYEAIYYDDSSYYSICETLAAKGIKSQRGNIIDSRQVKRMLMNIKNKAYHWAEKDGKPILKKGNYPAIVDEEIFDAVQEIIAERAKHYKKNEKPAEFRKHWLSGLLVCPHCGAGYSYNTRKPPQHDAFRCGNQTRGACKKGSSILVDVAVEMVLDKLSEVYAGPLAPYVKNITVSQPEPQIDYDKEIKLLEAQLKRAKQAYLAEIDTIDEYAQNKRRIASNIKELQEAKNQAQEGAALNEPQFKVKLLNAITLLKSDCPMSEKIPAARSIIEKILVDPRNKTMDIYFFA

SEQ ID NO: 458 - protein, 533 aa, Enterocloster clostridioformis
MTALYARLSQEDTLEGDSNSIVNQKAVLSKYAADNGFSNPVFFIDDGVSGVTFDRPNFNRMIAEIEAGNVATVIVKDMSRLGRDYLKVGYYTEIFFVERDVRYIAINDGVDSAKGDNDFTPFCNLFNDFYAKDTSKKVRAIKRAQGQAGEHLTKPPYGYMVSPTDKKQWIVDEEAAAVVKQIFDLCIGGKGPMQIAKILKEDKVLTVKAHYAEKKGKTFPDNPYNWNENSIVAILERMDYCGHTVNFKSYSKSHKLKKRIPTTKEQQAIFFNTHEAIVEDAVFERVQELRANKRRPTKADRQGMFSGLVYCADCGSKLHFATCKSFNGSQDHYRCSNYKSNTGSCTAHFIREEVLKQIIWSRIFDVTALFFDDIIAFKEMMYQQRSTETEKEMKRRKREVMQAQKRIVELDRIFKRIYEDDISGAISHDRFLKLSAEYEAEQRELEEKVKSEQQEVDTYEQNMSDFDSFSAIIRKYVGIKELTPAIVNEFIKKIIVHAPEKADGKRVQKVDIVFNFVGEINFLSATQPKRQGA

SEQ ID NO: 459 - protein, 477 aa, Fusobacterium mortiferum
MRDSKSKTVIIYNRVSTIMQDKNESLTEQTDECIRYCKTNGYEIYKILKDVKSGTKDDRAGYLELKKHIRRRDFDILVVLETSRIARKMKELVLFFSLLNENNIEYISIREPNYNTTTPDGKFAMNIRLGLIQFERDNTAERVTDRLYFKASKGQWVNGKPPIGYKLVNKRLEIDEEKAEIVKNIYEDFLNGYSLNQINNKLQFSWGSKQVKRILTNPTYKGYIRYGTRSNRKKNNREAFIVKGWHEAIIPEDKWEKVQEMYKSLNRKASNTKPTLLSGLLKCKECGNNYIRKRGGSYDKNLYYGCNLNNLRYSDKFFYKDIPECSSATIKGDLLEKAVIDTLKKQINDLNFNDIEIDKKRQVNIKQIDSSINKFKNRLNKIYELYIEDEIPKDKYLKDKKDIEARIISLEKQKKSFGEIEVEKSNNEMIQEYFSKIDLSNIEEANRILKIIVNKIVVYKKKKTPKDDFEVEIYLNI

SEQ ID NO: 460 - protein, 532 aa, Faecalibacterium prausnitzii
MTENNNRVCCLYRVSTDKQVDFNSNHEADLPMQRKACHKFAESKGWVIVHEEQEEGVSGHKVRAAARDKLQIIKDYARRGKFDILLVFMFDRIGRIADETPFVVEWFVRNGIRVWSTQEGEQRFDNHTDKLLNYIRFWQADGESEKTSVRTRTSLRQLVEEGHFKGGNAPYGYDLVRSGRINKRKHELYELHINEQEAAVVRIVFDKYVYEGYGPQHIATYLNNSGYRARSGKCWHPSSIRGMVQNLTYTGVLRCGDARSELMPDLQIVPQEQFENAQRIRNERSVRSTAEAENRLPLNIHGKSLLAGNAYCGHCGAKLELTSSRKWRKMADGSLDDTLRIRYTCYGKLRKQTNCTGQTGYTVHILDEIIDKAVRQIFSKMRGIPKEQIVTKRYEKENTERKNHLQDLQTQRNKAEKDLLALKTEVLACIKGESVLPRGTLAEMITEQEEKLAELENLCESATEELEKTAELMDKVSRLYDELISYADLYDSANFEAKKMIVNQLIRRVDVYRGYQINISFNFDLTPYIEGE

SEQ ID NO: 461 - protein, 644 aa, Klebsiella pneumoniae
MLRPICYERVSSIQQIEGGGGLDDQRSALEGYLDRNSDKFSNDRIFIQDRGVSAFKNSNISSESQLGIFLQDVQNRKYGEGDALIVMSLDRISRRSSWAEDTIRFIVNSGIEVHDISASTVLRKDDPHSKLIMELIQMRSHNESLMKSVRAKAAWDRKIIEAVQNGTVISNKMPMWLKNVDNRYQVIQEKADLIIRCFEWYRDGFSTGEIVKRIADPKWQMVTVSRLVRDRRLLGEHKRYNDEIIHNVYPKVIDDDLFLTANRMMDRVMLEKNKPAEDLLLESDVVQEIFQLYESGLGSGAIVKRLPKGWSTVNVLRVLRDKNVVTQKIIDNLTFERVNQKLSMNGVANRIRKDITIAQDDYITNLFPKILKCGCCGGNIAIHYNHVRTKYVICRNREERKICDAKSIQYIRIEKNILKCVKNVDFQKLMIESTGSETSVLDGLREELSSLRREESSYNDKINERKLAGKRVGIHLNDGLTEVQDRIEEIEKEIISAQTVREIPKFDFDMDEVLDPMNIELRAKVRKQLRLVLKAVKYWMFDKRIFIQLEYFNDVLSHMLVIDNKRGGGEVMYEMSIEEKKGERIYTVHENGYAVFIASVTIGTELWSLALSRTRTIDSVGNYLSLLAREGFEIFVNEDQIDWF

SEQ ID NO: 462 - protein, 523 aa, Klebsiella pneumoniae
MRQVITYLRFSSKPQERGDSIRRQKGLFERWLKDNPDAKVVDEFSDEGASAYHGHHLKGDFGRMLQNIQDGKYLSGQTVLLVESETRLNRQKARNTENLVDLITGKGVDVICLESGKIYTSTNIDDLDTSIQLKIAAHIAHQQSKEKSIKVSAAWEHRAQLALEGKQQLTKNVPGWIDPDTRKLNEHSSTVVTIFDLLLSGESLHNIARYLQANNIKSFSRREKANGFSVHSVRTILRSESTVGTLAASKRNDRPAIPNYYEPAIDVATFNKAQEILSKNRVGRAPASDNPITINLFKGIIRCQCGASVHPTGVKATYQGVYRCNNVPDGRCNVPTIKRKPFDKWMLDNIVGFLERDDGNNTDKRKAEIEYQISLVTSKLKKATTLLLELDDVTELKEQVKELNIQRSNLQSELDELNQRETLSDKPLHHLSEIDLTTKAGRVEAQLILSKFVQSIELQREMIIITLRNGTVIGKSRDLSPVLSQDLMKQVVSSPSPTDIDMFSVITSDEEFRKSGKQVTKRS

SEQ ID NO: 463 - protein, 539 aa, Klebsiella pneumoniae
MPKAISYIRFSTKIQSVGDSTKRQSKYINDWLKRNPDYYLDESLRFQDLGISGFSGANAKSGAFGEFLAAVESGYIEAGSVLLVESLDRVSRQDIDTAGEQLRKILRSGVEVVTLVDNEWYTKESLKDSLSMIKAMLVMERAHEESAMKSTRLRSVWAAKRERAAKGEIMSKRCAAWLKVSEDRSRFEFIPENVKAVQRVFQLRLEGLSHVKIAKQMNDEGFSTLNQFKSVTGGWSQSSVTELLSNRSVIGFKVPSKSMAVKGVSEIPNYYPSIITDEQFYSVQQLKQGSGRKPSSDLPLLTNLFKGVLRCSECGFIVVVAGVSAKRSGIYKCSMKSEGRCNSVGFSRLQTDRALVQGLLYNTNRLSLNRDNGSAIGTLQSELEQLQKQRERLIKLAMLADDTESMAKDLKALNSQIKDAEKAVSEVHQREQSSQLETISHLDLTVKKDRIESQIIIKRIVKEIRLNTAGKKCDVFLHNGLKLYNFPLDRVVDGAQWLEILPLIDGDEFDFEGFTTKPRHIALEEAPEWVKEMEEQPKQ

SEQ ID NO: 464 - protein, 599 aa, Klebsiella pneumoniae
MKKIIPYGYLRVSSLEQVRSGGGLEAQDEEVRRYITQKSDVFDIDKMVMMSDEGLSAYSGRNIKEGELGRFLADVDAGLIPAGSALVCYSVDRLSRQNPWVGTQLISTLIGAGIEIHSVAENQILRSDDPVGAIMSTIYLMRANNESVIKSERAKHGYTKRLNESIANNKVLTRQMPRWLYDNDGKYAIDPNMQKVIDFTFDSYIAGQSTGYIAKKLNDMGLKYGDTSWRGSYVAKLIRDERLIGKHIRYSKQIKGVKREIIETIPDFYPVTIDTDKFHIANNMLTSVAKNIRGRTRMTYGDISILRNLFNGVIKCGVCSGETSVVQNTRRKITNGVVTYVPYKTFLRCRNRYELKTCTQGDIRYEVIERAILNHLMGLDITTLLAAPVDNKIERYRTELELCKAEEEELQAIIKERENEGKRPRPQTLKSYEDVADRIDELTQLIESHVEDNFIPHFNVDLDSITDVSNVSERSLIKKGIATIADSICYKRISDFILVEIKYRNLNDKHVLVIDNKNSEMVVNFSIEYNEENKVYICNSFVMEYDNLSCEFTVSKTTMEDYAHMMNFVDYVSDDESYNAKEFLVKNFTHIKFIDKSNE

SEQ ID NO: 465 - protein, 472 aa, unidentified
MRVLGRVRLSRLTEESTSVERQRELIKKWSEMNDHTIVGWAEDVDVSGSIDPFEAPELGPWFQEDKRGDWDILVAWKLDRIGRRAIPLNKVFGWMLEHEKTLVCVSDNIDLSTWVGRLVANVIAGVAEGELEAIRERTKASRKKLLESGRWTGGPVPYWLIPEKLPEGGWVLSLNTETAPILRRAIDEVLDGTAVHTVAERLNDQGVPSPGGKKWTSQTLWRILQHKYLKGHSTDRGKTVRDSSGVPISNCEALLTPSEWDRLQAVLTQWKLPETSNRVKNTSPLLGVVVCYICDKPLYYRSYTRNYGKGLYRSYYCRNHRTPGIKADMLDEYLEENLMREVGDKNVLERYFVPAENHQIELDEAIRATEELTALLGTMTSATMRSSLTAQLAALDSRIASLEKLPTSESRWEYRELPRTYREMWESDDDPQFRRELLLKSGITLAATMTGGQKLHLHIPDDILERMALKGE

SEQ ID NO: 466 - protein, 455 aa, unidentified
MASPPRNAALYLRISLDQTGEGLAIDRQREECERIAAQRGWTVVGVYEDRSISATQANKKRPGYEQLVSAYQAGQFDAMVCYDLDRLTRQPRQLEDWIEAAEGRGLALVTANGEADLTTDGGRMFARVKLAVARSEVERKSARQRTAAHQRASLGRPPLGTRLTGYTPKGETIPAEAEVIRRIFKLFQAGETLRSITRMLTEEGVTTRRGNAWNPSTIYGTLTNPRYAGRAVYQGKPTGQLGNWEPLVSPEVFDLIQARLADPRRKTNRVGTDRKHLGSGLYVCAVCEQPTTSWSQGRYRCKDSHVNRAQSQVDSYVLDTIAARLRRGDIATLLAPAKADLAPLLDDIERLTARQATIDADYDAGLIDGTRHAAATATVRAELIAVQQQMAAADKGSALGELLTSPDPAQAFLDAGLMTQRSAIDALAVVRLHRGHRYSRTFDPETVEVDWRRPR

SEQ ID NO: 467 - protein, 476 aa, unidentified
MKAVIYTRISKDREGAGLGVERQRADCAALADRLGWQVVGTFSDNDLSAYSGRHRPGYAAMCEALEAGEAQAIIAWHTDRLHRRPVELESFIGLCERRSIQVRTVRAGTLDLSTPSGQMVARMLGAAARHEIDHAIERQKRAKKQAALDGRYRGSRRAFGYERDGLTLCDAEADAIRTAAERVLSGTSLSQVARDWNAAGLRTAFGGKAFTSREVRRILLRPRNAGYSLHEGKRIPNAQWPPIITTDTFAALEALLRDPVRSKHLAFERKWMGSGVYLCGKCGAKISTASQKGTGKSWRPTYVCSASKHLGRVADTVDEYVTEAVLERLSRPDAPILLGGNKVDVADLTSRRDGYRARLDELAAMFAAGDIDAGQLKSGTTELRRKLDRVDAELAAARASSVLADLVLSGDDLRDTWAAIPPGGKGKVIDALMTVTIEPTRRGRRPGGSYFDPESVTIRFKGVGEHRLDDGQLIGA

SEQ ID NO: 468 - protein, 515 aa, unidentified; identical to SEQ ID NO: 4

SEQ ID NO: 469 - protein, 605 aa, Nocardia otitidiscaviarum
MPESPPRALIVIRLSKVTDATTSPERQLAECRAICEKRGYEVVGVAEDLDVSGAVDPFDRKKRPHLARWLHGEHLDDNGEPVPFEVIVVYRVDRLTRSVRHLQKLVAWADDHKKLVVSATEAHFDTSLPFSAVLIALIGTVAEMELAGISERNASAARHNIQAGRYRGSTPPWGYVPSNDTGEWRLVRDKEQADVINEVARRVIEGEPLQKIAHDLTRRGILTPKDNFAKQRGREIKGREWSVTQLKRSLLSEAMLGHAVSGGAAVRNDDGSPVVRSEPILSREIYDRVAAELSSRAKRGEPNKRTSSLLLGVISCGNPCLHKQQGHECPEGCSGTCDEPVYKFNGGSHSQFPRYRCRTMTRAYKCGNRTIRADQADAQVERTILALLGSSERLERVWDAGEDHSAELADINDELVDLTSQLGVGAFRAGTPQRAKLDARIASLAERQAQLSSEAVVPAGWKLLPTGELFGDWWSRQDLTARNVWLRSMGVRARFKRDDKTLYIDLGNLNDLISGLKPGGTAQRVRGGLQAMERNGIQGMVFSDADSEVMPAPAAGYMWIQPVEGVWVYTSEALLAAAAERQALRREKIEDEFAYGPGDFDEDWD

SEQ ID NO: 470 - protein, 525 aa, Pseudomonas aeruginosa
MPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 471 - protein, 551 aa, Pseudomonas aeruginosa
MPTAYAYIRYSSKAQGEAGRDSVDRQMASIQAITKQQGIELRTENIFSDTGISGFDGSNKNKGKLKDLIDLIISQKIQDGDFVFVESIDRLSRQKMRLSKDLVYTILDRGVTLVTTIDGQMYSRAKDGMEQDIMLSVIAQRAHEESKTKSVRRKSAWNRAKKLADEEKEIFNGHNPPYGISFNKEESRFEIVEEEAQEIRDIFESLKYVGVSLTIKKVNEYSKRKWTNRNIKHLLDTKYVLGSYMAQRRDENKKKVFERYIENYYPQIVSFELFNEAVASMKNRAHRKHYGNQTVGSLNIFRHSIKCSNCNASMLFEKQTNPKGVVYPYFQCFTRKELKNGCDQPRFRFDLAFGVFLELVKFATTSSETINPNDWNNEITSVGSFHKTLFKLLTSTEKDKELEKKISHVKNLLLEQKNYQDNINKSFEAFTDGIIPAAFIKKASETEIKIEALERELAELNIESSTRNVSLLVHSYNDIIDLYKTEAGRLKINSFFTSNNIAFSFSFDQKTRMLRCKVYYKDIHVDVINKKFPLHNPLKEFGIDNLNQYFN

SEQ ID NO: 472 - protein, 510 aa, Paenibacillus campinasensis
MRAAIVRRVSTLKEVQENSLQNQKDFFEDYVQSKGWDIAEIYTETETGTKFSRKEMNRLIADAKAKKFDIILAKELSRLARNQRLALEIKEVIEKHGIHLVTLDGAIDTTTGNTHMFGLFAWIYEQEAQRTSERIKMALETKAKKGLYKGSNPPYGYVVREGRLYVSDDGSSEIVERIFQSYLGGKGFDAIARELFEEGVPTPSMIAGKKNQTIYWHGSSVRKILENPHYTGDMVQGRQSTISVTNKSRKEKPKTEFIVVKNTHESIISGEVFESVQQLIAFNRKKSIDNPDVCSRPHQNVHLFTGVIFCPDCGSGFHYKRNGACYICGRSDKLGDKACSKHRIREDALVAIIRWDLQRLSKLLDDQSFYNTVKDKFVKAKSKLEKELKACAGKIENIKNLKSKALSKYLEESITKSDYDDYIAAQDAEIQKLLHNKEKLDSAISASVDVDVLGKIKGIVASSLEFQEINREVINRFIEKIEVTADGNVKLYYRFAGTSKILNELMTDVN

SEQ ID NO: 473 - protein, 509 aa, Bacillus sp.
MRVALYYRVSTKLQEDKYSLAAQKEELTKYAKSQGWNIVGEFKDVESGGKLEKKGLTALLDLVDEGGVDVVLVVDQDRLSRLDTVAWEYLKSVLRDNNVKIAEPGNIIDLNNEDDEFISDIKNLIAKREKRAIVRRMMRGKRQRMREGKGWGLPPFEYQYDRQTGKYKAKPEWKWVIPFIDNLYLNEQLGMKAISDRLNEISKTPTGKSWNEHLVHTRLVSKAFHGVMEKTFANGETISIPGIYEPLRTEETYRKIQEEREKRGEQFSVSGRKGSEKHILKRTKITCGECGRKIQIATHGTKEKPIYYAKHGRKERVDGSVCDININTVRFDKNIMTALKEILSSEEQAKKYINLDIDQNELNALKKNIKTLNKRISKLQESLDRLLDLYLAGGLKKEKYLEKQKQLESQIEIYKKELDQNELKLKTVESNMWNYEYLYEYLEVIADLEKELTPLERAQLVGKIFPTATLYREKLILTADVKGIPVDIEIPIDPDPYPWHPKKRNTSIK

SEQ ID NO: 474 - protein, 562 aa, Pseudomonas fluorescens
MPKAISYIRFSTGRQSLGSSHERQRQAVTRWLEKHPDYTLYDKPYDDLGRSGYSGDHVDNAFGHLLAAIEDGTIPKGSTILIEQIDRVGRMEPFEMFPLLSRIVNAGVDLVTLDDGITYNRQSVNNNHLFLLVAKVQAAWGYSKTLSERTKASYAIRREKAKNGEPIKRFAVAWLTTDGKLKSHLVPYVKQVFDLYISGVGKNTIANRLRASGMPELASISSPTIDAWLQNKTAIGFWNDIPGVYKPVVTPEVFMQAQKRRQEVKTQSRSRTSKNFLVGMVKCGVCNANYIIHNKEGKPNNMRCLTHHRLKDAGCTNSETIPYQVVHFIYLSSAPSWIDKAMKVIQLTDNEKRKLYLVTEREELTASILRMAKLLARTDSPELESEFDLANERRASIDIELSVLDRKADNGVESKSTSIFVGYEATLEHDRLAFHDPIQLSALLKQAGYSITVQPGRKLYVAESNVPWVYTGVARKGNTALGYRIQDGEMEYTISNVIPEAVDVQAYKNNPDGEMQHVADRSYKHVKSPTLLNPTGLRNTNVMTIEKFESANAAMQRLTSGV

SEQ ID NO: 475 - protein, 581 aa, Pseudomonas fluorescens
MRSAIPYIRFSSARQTTGSSAERQQQMVTQWLTEHPEYTLSDLTYKDFGKSGYHGEHVKDGGGFAKLLAAVEAGDIKAGDVVLVEAIDRTGRLHPLDMLNKVITPILAAGVSIITLDDKVTYTHESAASGHLFLLVAKIQAAYGYSKQLSERTKASYAIRLEQAKEGNKVKRNTPVWLHSDGRLNDDVAPYIKQAFELYVSGVGKTAIANRLRASGVPELVKCSGPTVEAWLRNQAAIGNWEYGKDDTDKPSQIILGVYPPVISNELFLQAQHRKSAVATKPRERTSKNFLVGIVKCGVCGANLIIHNKDGKPNNMRCLTHHRLKDAGCTNKETIPYQVVHFVYLQTAPAWIDKAMKVIQLTDNEKRKLTLTTERNEVTASIQRLAKKIAKVDSVELEAEFDLVNERRAAIDIELNILGRTDDDGAESKSKSNYVGYESNLEHDRLAFRDPIQLSALLKQAGYSIVVQPGRKLYLPNDNHPWVYAGVVRKGNMTLGYRIRNSEEEFTISQAIPEVPDVRLYGNIPNGDLVHVAERSYKYAKPPTLLNSSDKHSRKGVFVLRFESADIAMEYMKSGIETDSK

SEQ ID NO: 476 - protein, 524 aa, Pseudomonas fulva
MPTAFSYARFSSATQKKGSSLERQRAMVARWLVAHPDYSLSDQTFQDLGKSGWKGEHIKEGGGFAELLVAVQAGLIQKGDAVLVEAIDRTGRLPVLDMLSIIVSPILRAGVSIVTLEDNLVFTEASLNEGGHIYILVGKIQAARQYSDNLSRRLTASYDSRRRLAKEGKAPKRNTPVWLTSNGDVIGEVAEQIRLAFELYTSGLGKAVIAKRMRESGVPALAKTSGPGVEGWLRNEAAIGRWNGSEVYEPIVDLSRYQLAQIEGERRKTTPTAKTATHFYVGLVKCGSCGGNFIMRTIKGVQVSMRCRKRQELKGCDNKKVIPKVVIDAVYRHTSTPAARKLVAHERRSVNEKAIIAAEAKVLELAKEIEAMVLAFSGAMAIPEVVGRIQALHAEREAAERELALLKVTVERPPTDWRVQGRVDDLGRTDPQRLAAMLRSVGYTITIDSDGRLCTSESKTVYRYAGVNRREDMYRLAVAGGKELLISKIPVEVFDEWWEAEDGDEVVTSEWDADNPDAMRSRHG

SEQ ID NO: 477 - protein, 532 aa, Pseudomonas fluorescens; identical to SEQ ID NO: 2

SEQ ID NO: 478 - protein, 486 aa, Prochlorothrix hollandica
MKIAYARVSSREQSENSHALEQQIARLESSQVDRVIQDVESGSKNSKSPGFRELMDLVKEGKVAEIVVTRLDRLTRSLSSLQKTMEILKAHGVALVSLDDSIDTSTAAGVFHLNMLGALAEMEVGRLSERVRHGWSHLRDRRVAMNPPFGYRKENDQHVLDTTPFLCLISDRSEWSRAKISRYYIDTFLQERSIRLTLRVVYPHFGIQVYRSRRRGPHATRLIRFSPSAFNEWLTNPVLQGHLSYRRNASGNRKREDPKTWQIIPNTHEPLITAEEAAMIKQILSRNRQAKGFGSPKRRHSVSGLVFCGECRSACYHQSGCQNYARSKRLGIPQIIRRYYQCKNWRSRACPQKAMVSLDIIEEAVIAALISRAEDLAKMADTPAPTPEPLALRELRSQLADLNRMAYNPAIENAKAQIKNQIAGMELDLTHTTQESSRLGQLISALADPDFWKEGLEPNEKSQLFQDLVSQVIIKDGAVLEVKLKV

SEQ ID NO: 479 - protein, 604 aa, Streptomyces sp.
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVA

SEQ ID NO: 480 - protein, 562 aa, Pseudomonas putida
MLHCGFPCHEERAMPSAVPYIRFSSARQTTGSSAERQRQMVTQWLTQNPDYILSELTYEDLGRSGYHGEHLNDDNGFAKLLQAVEAGSIKAGDVVLVEAIDRAGRLSPMQMLKRVIIPIIEAGVSIITLDDNVTYDESSVEGGHLFLLVAKIQAAHNYSKQLSDRTKASYAIRREQAKATGKVKRHTPIWLTSEGEVIEHVAVHVRQAFELYVSGVGKTTIANRLRASGVPELATCSGPTVEAWLRNQAAIGNWEYGKDDPDKPSEIIRGVYPAVVSDELFLQAQLRKKAAATKPRERTSKHFLVGLVKCGVCGSNYIIHNKGGKPNNMRCGTYHRLKKAGCTNDETIPYQVAVYIYSETATHWVDKALQQVQLTVNDKRKLVLTTERDALTTSITNLTEKAAALNIPELWKKLEEESNRRKVVEDELAVLERTPDAGGESGFSAALSQDQMMIHDPIQLSALLKQVEYSIVVYPNKLFTVSGEVYPWLYLGPKRKPKSNVTLGYRMLYLGDEIIISPDVPVTLDWGAPTDNPVEQMRYMLRRAYKMVSAPKPYEYNDDVAE

SEQ ID NO: 481 - protein, 530 aa, Paeniclostridium sordellii
MIAAIYSRKSKFTGKGESIENQIQLCMDYAKNLGINEFLVYEDEGFSGKSMDRPKFKEMLKDAKDKKFDYLICYRLDRISRNISDFSTLIEDLNKLNISFVSIKEQFDTSTPMGRAMMYISSVFAQLERETIAERVRDNMYELARTGRWLGGMPPYGFISTQINYYDENMNQRKMYKLKVDEDTIEIVKLIFDKYLELRSLSKLYKYMYENGIKGTRGGNLDPSALSLILKNPAYVKADKSVVDYLRKSNIDVMGDIDNIHGILTYAKNTDSPIAAVAKHKGVIDSDKWIEAQRLLNANKAKAPRAGTGSKALLSGLLKCSKCGSNMRITYKNSKSGTIYYYICGTKKSLGVSACDCRNIRSDKAESKVIDELKNKSIKSIMSSYKDSKLENSKNIKNIKTEINSINSQIKEKETYIDNLVMQLAKVTESSASTFIINKLESLNNDLSNLKSQLESLNTISMENKQVDININMLIDNLNKFNKEIDNSDINKKRLLLSTVVDYMTWDSDTDTIKVNLIGINPSNTIASGK

SEQ ID NO: 482 - protein, 534 aa, Pseudomonas syringae
MKQAISYIRFSSARQEGGSSVERQEGMIAKWLLDHQDYELSKLNYSDLGKSGFHGEHVKEGGNFGKLLKAVMDGYIKRDDVVLVEAIDRTGRLPALQMLSDVIAPILRAGVSIITLDDNTTYTEASVGGPHLFMLVAKIQAAHEYSRTLSRRVEDSYKKRRKDAKEKGVAPKRMTPVWLNSDGTIREDVAPWIKTAFELYVSGVGKSTIAKRLRESGVERLAKASGPGVEGWLRNKAVIGKWETLIGTPEHHVIDDVYPLIIEPSLFYKAQVHAEKMKTQRPIKTAKHFLVGLVHCGECGKNYIMQNLHGKPHSMRCRTRQSQNNCSNSYVVPKPVLDAIYRYTSVTAALEAVQLQQLGVNEKEIVTREAELLTITKRVEGFVQAVNEAGPMPELLTALKQARIERESAENALVILRATVVTPPANQWREMGKVWSLEAEDAQRLSAMLRQVGYNITVGKGAIIKSSHSDVVYQYLGVDRVKDMYRVLADGEMKLIHKSQVDDYPYHEPFHEVVGEATMDETDKENLLLQYQSS

SEQ ID NO: 483 - protein, 544 aa, unidentified
MQEHSPSNHSSGPVRAYSYVRMSTHKQLRGDSLRRQLERSKAFADEHSLLLDDSLRDIGISAWKGRNFKTGALGRFLSMVESGEIPKGSYLLIESLDRLSREAVPDALTLFMAIINAGITIATLGDDRQIYSRDILNGDWTKLIIGLAVMSRGHEESQTKSERVRAAIHRKRENAREGKGQITGLTPAWIDAERIGANRYTFTLNHHTETVRAIYEMAARGLGATVIARKLNAEGVPAFKSKDGWYQSIIKALLSRHDVIGTFQPHRVQDGKRVPDGDPIESYFPAAIDKDLFLRVQSMRSNPGRPGRKGDMFTNLFTGLCHCSHCGGPMTMKLSRVKGNENGRYLVCSNYVRGHRCTEGNRHFRYEPFETAILDHVRELNLAEAIATTMTNEAITGINETIAALTLQLDELRRKEQRLAMALEDDNQPIDSIIDLLRQRQQERHAIEAGLQYHQQERHRLTVRHNDPAQTCDRIGQLRTAWEQADEATRYGLRSEAHAAIRELITEISFDSGSHSAIVIVANGISAYRLQDGLINGRFHAFAA

SEQ ID NO: 484 - protein, 612 aa, Rhodococcus equi
MESASPGLRVLGRLRLSRLTDESTSIARQRETIQRWAEIQGHTVIGWAEDADVSGAVDPFDTPQLGQWLNHRVEDYDVIAAWKLDRLGRNAIQLNKLFGWCIDHNKTLVSCSESIDLGSWSGRMLAGVIAGLAEGELEAIRERARSSRVKLREAARFAGGKPPFGYRKVRRDGGGWMLEIDEPAAELVGKIVADVLDGKPVSRVVMALNEGGYRTPRDYYETVKAGKPALKLAAGERRNSEWRSTALRNLLTNPALRGYVHHKGQIARGDDGMPLQLAEEPLVDADEWEGLQAIFNGHRERRSHYRRPDASPLVGLVYCYWCHSPLWHNRNVSRGHEYFYYRCPNIQKHERASMIPADQAEKAVADSFLDQAGDLPVMERVWVPGDSTERELRDAVAALDELTEVLGTLGSATARQRITRQITALDERIRDLEAQPVREARWEYKQLGGTYREAWESLGEAERWQLIMKSGMTFAFGLTDRGRGPNSVWVSSVYTPEPLKQTLVSGVTQRRTLADADPHRDVNSADHTKSLPEHWATMRKHGVEGIEVHEVKSVFVSKPGERVEIPHWLHEVGISEITNDDDVRIIWTQDGRGWYQHSDGEWIEVPLGELEE

SEQ ID NO: 485 - protein, 527 aa, Ruminococcus lactaris
MEKAVLYLRLSKEDIDKISEGDDSASIKNQRLLLTDYALKHDYQIVDVYSDDNESGLYDDRPDFERMIQDAKLGKFSIIIAKTQARFSRNMEHIERYLHHDFPILGIRFIGVTDGVDTADSSNKKARQINGLVNEWYCEDLSKNIRSAFKAKMKDGQYLGSSCPYGYIKDPTNHNHLIVDDYAADIVREIYKLYLQGIGKGRIGRILSDRGVLIPSLYKRNVQGINYHNANAKAETHLWSYQTIHQILNNQMYLGNMVQYRTTTLSYKDKTKKLRLPSEWIIVEGTHEPIISYAIFQRVQELQKIRTKEVNTEQKYTNIFSGLLYCADCNHTMNRNYTRKGVFCGFICSTYKRHGNKAGCRSHRVDYDALCDAVLESIKLEARKILSDKDVDELKKSRLISSREEKIENEIRILENECEKLKQYKQKTYEAYLDDLITMNEYKVYIKKYDTELSDTCKKIDKLYAEKQVTDSLDQKYKEWVNMFSDYINITELTRTVVIELIKRIDVYEDGNIKIHYRFKNPYESSK

SEQ ID NO: 486 - protein, 461 aa, Staphylococcus aureus
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKQFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 487 - protein, 463 aa, Staphylococcus aureus
MKVAIYTRVSSAEQANEGYSIHEQKRKLISFCEVNDWDRYEVFSDPGVSGGSMKRPSLQKLFDRLEEFDLVLVYKLDRLTRNVRDLLEMLEVFEKNNIAFKSATELFDTTSAIGKLFITMVGAMAEWERETIRERSLIGARAAVRSGKYIKVQPFCYDLVDQKLKPNQYAEYIRFIVDKLLSGKSANEVVRLLESKKKPPGITKWNRKTVLGWMRNPILRGHTKHGDLLIKNTHEPIISEDEHSKMLDIIDKRTHKSKTKHNSIFRGVIECPQCQNKLYLVSSIQKRANGGSYEVRRYTCATCHKNKEVKDVSFNESEIEREFINTLLKKGTDNFMVNIPKPKDYDIENNKEKILEQRANYTRAWSLGYIKDEEYFVLMDETDKLLKDIEEKESPRINIELNEQQIRSVKNLLIKGFKMATAENKEELITSTVDLIKIDFIPRRLNKEGNINTVKINEIHFKY

SEQ ID NO: 488 - protein, 461 aa, Staphylococcus aureus
MKVAIYTRVSTLEQKEKGHSIEEQERKLRAYSDINDWKIHKVYTDAGYSGAKKDRPALQEMLNEIDNFDLVLVYKLDRLTRSVKDLLEILELFENKNVLFRSATEVYDTTSAMGRLFVTLVGAMAEWERTTIQERTAMGRRASARKGLAKTVPPFYYDRVNDKFVPNEYKKVLRFAVEEAKKGTSLREITIKLNNSKYKAPLGKNWHRSVIGNALTSPVARGHLVFGDIFVENTHEAIISEEEYEEIKLRISEKTNSTIVKHNAIFRSKLLCPNCNQKLTLNTVKHTPKNKEVWYSKLYFCSNCKNTKNKNACNIDEGEVLKHFYNYLKQFDLTSYKIENQPKEIEDVGIDIEKLRKERARCQTLFIEGMMDKDEAFPIISRIDKEIHEYEKRKDNDKGKTFNYEKIKNFKYSLLNGWELMEDELKTEFIKMAIKNIHFEYVKGIKGKRQNSLKITGIEFY

SEQ ID NO: 489 - protein, 478 aa, Streptococcus agalactiae
MNKVAIYVRVSTTNQADEGYSIDEQIDKLKAYCEIKDWVVYKVFTDAGFTGSNIDRPAMTNLISAAKKRQFDTILVYKLDRLSRSQKDTLYLIEEIFIKNGIDFLSLSENFDTSSAFGKAMIGILSVFAQLEREQIKERMMLGKVGRAKSGKTMMWAHPAYGYTYNKETSSLDIVPAEAALIKKIYELYIKGKSISKLRDYLNDNKIFVNKSVPWSHRTISYALTNPVYCGMIRYEGKLYDGLHEPIITKELFNKTQEVLAERRMEASKKNPRPFQSKYMLSGIIRCGCCNAPMKSLLGMPRKDGTRTRRYQCINRFPRKTKPVTVYNDNKKCDSGYYYMEDVEHAVLHRISTLYSDEIEASEFFEDEITFDIQKVKDEITKIESKINKLNDLYINDFISLDSLKKQSANLINEKKIIENEIEKENSKQVNNLKEDALKILATNNIHDLDYEMQSYVVKSLIDKVFVTKEDMEILFKK

SEQ ID NO: 490 - protein, 558 aa, Staphylococcus arlettae
MKGKIALYSRVSTSEQSEHGYSIHEQEQVLIKEVVNNYPGYDYETYIDSGISGKNIEGRPAMKRLLQDVKDNKIEMVLSWKLNRISRSMRDVFNIIHEFKEHGVGYKSISENIDTSNASGEVLVTMFGLIGSIERSTLVSNVKMSMNAKARSGEAITGRVLGYKLSLNPLTQKNDLVIDENEANIVREIFGLYLNHNKGLKAITTILNQKGYRTINQKPFSVFGVKYILNNPVYKGFVRFNNHQNWAVQRRSGKSDENDVILVKGKHEAIISEDVFDQVHEKLASKSFKPGRPIGGDFYLRSLIKCPECGNNMVCRRTYYKTKKSKERTIKRYYICSLFNRSGSSACHSNAINAEVVERVINVHLNRILSQPDVIKQIASNVIEELKQKHSNQTEIKYDIDSLEKQKAKLKTQQERLLELFLDDQMDSEMLKAKQSQMNEQLEMLDKQIKETQQARESQAEVPDFDKLKSRLTMMISRFSIYLRKATPEAKNQLMKMLIDSIEITTDKQVKLVRYKIDESLIPQSLKKDWGSFFMPKFQFEIDGRKNYFIDQITTFTT

SEQ ID NO: 491 - protein, 478 aa, Streptococcus equinus
MNKVAIYVRVSTTNQAEEGYSIEEQIDKLKAYCMIKDWSVYDIYVDAGFSGSNIKRPAIQKLIKDTKRKVFDTVLVYKLDRLSRSQKDTLYLIEDVFLENKIDFVSLLENFDTSTAFGKAMVGILSVFAQLDREQIKERMQLGKLGRAKSGKPMMWAKVAYGYTYHIGTGKMTVNQSEAIIVKEVFSSYLNGRSITKLRDDLNEKYPKTPAWSYRTIRQMLDNPVYCGYNKYKGQVYPGNHAPIISKEIYNQVQDELKIRQQKAYEHNNNYRPFQSKYMLSGIAQCGYCKAPLKITLGTIRKDGTRFKRYQCVQRTPRKTKGATVYNNNEKCNSGFYEKDDIEAYVLESISKLQTDSNCIDELFNDEPEKLDRDALNKEIETLSNKISRLNDLYINNLITLDDLKTKTDTLQSKIDILKEKLEKDPALERQKNKQKMLKKLDTKDIFKMDYEEQKMLVRALINKVQVTADSIKILWKI

SEQ ID NO: 492 - protein, 465 aa, Staphylococcus hominis
MKVAIYTRVSSYEQATEGYSIHEQERKLKAFCEVQNWNEFKVFTDAGVSGGSMNRPALKRIMDNLEYYDLVLVYKLDRLTRNVKDLLEMLETFEKYNVAFKSATEVFDTTTAIGKLFITMVGAMAEWERATIRERALFGSRAAVREGNYIREAPFCYDNVDGKLVPNKYKWIIDYLVEQFKHGVSGNEIARQMNVKKVNVPKVKKWNRTSIIRLMKNPVLRGHTKYGDMYIENTHEPVLSESDYKRIIDVIENKTHRSKVKHHAIFRGVLTCPQCHNKLHLYAGKITDKKGYSYEVRRYKCDTCSKDKNVQTISFNESEVEDKFIELLKTYDMNKFKVDIVEESTPKLDYDIDKIMKQREKLTRSWSLGYIEDDEYFSLMDETKEILDEVERAGTEVESTQTVTNEQLNMIDNILIKGWSKLNVEQKEELILSTVKEIVFDFVPRKYNENGKVNTLNIREITFKF

SEQ ID NO: 493 - protein, 555 aa, Streptomyces ipomoeae; identical to SEQ ID NO: 5

SEQ ID NO: 494 - protein, 481 aa, Streptococcus mitis
MITTNKVAIYVRVSTTSQAEEGYSIEEQRDKLEAYCKIKDWSVYDVYTDGGFSGSNTNRPAIERLIKDAKNKKFDTILVYKLDRLSRSQKDTLYLIEDIFIKNNIAFLSLQENFDTSTPFGKAMIGLLSVFAQLEREQIKERMQLGKLGRAKSGKSMMWAKTSYGYNYHKETGTVTINPAQALAVKFIFKSYLAGRSITKLRDDLNEKFPKEIAWNYRAVRNILDNPVYCGYNQYLGEIYKGNHESIISKEDYDKTQNELKIRQRTAAENVNPRPFQAKYILSGIGQCGYCGAPLKIMLGVKRKDGSRLKKYQCHQRHPRTLRGITTYNDNKKCDSGFYYKDDLETYVLTEISKLQNDTNYLEQIFSEDNTETIDRDSYQKQIDELSKKLGRLNDLYIDDRITLEELQTKSAEFTSMRSSLETKLGNDPALKQKDRKKGMINILNQRDILTMNYEEQKVVVRSLIDKVQVTAEDIVIKWKI

SEQ ID NO: 495 - protein, 534 aa, Salinispora sp.
MSTSIPEESGPNDLELRGTPAGLPPYADLVAANPNAIFVGAYSRISDDWRKNKSKKAAASRWSAGKGVANQHRRNDMNAGRHQVIVVHRYTDNDLVASRLDVFRPDFAQMLKDLKLGRTKDGYRLDGIICVHQDRLQRTDTDWEHFVHALLAKPGRLLWTPSGSSDLTDEGEIVKTGIMAVLNKAESMKKKRRIRDWHQDLILDGLPHSGPRPFGWNEDKMTLRAEEADYLAWAIRERIKGKALSTLCAEAKKRGLKGTKGGEIAPTTLSQMMTAPRVCGYRANRGTLALDENGAPIVGKWDTICKPEEWEAVCATFSPGSTYMHRGPGAPRVTGKPKTVKYLASQLLRCMNKVERDGETRICNGTLTGSPTKSARSPYTYRCGSCNKNSIAGPMVDRQITRLLLGKLGEAQITYRRPELAWHLESTLKTLADRLAGLENRWMAGEVDDEQFYRLSPGLQAEVRKLRAERARWELENAAGSEEPADIIRKWRSGELDLAQRRRILFDVFAAIQVTPGQKGSKTPNPHRLKPIWG

SEQ ID NO: 496 - protein, 593 aa, Treponema denticola
MLAIYARTSTDKAENSTIEQQVKAGIEFASKNNMNPKVFQDKGVSGYKIEEDENKNPFENRPAFTQMIEDIKKGTIDAVWVWEHSRISRNQYASAYIFNIFSKYKIRLYEKDKEYDLNDPNTQLLRTMLDAVAQYERQLIVKRTTRGLHNAIDNGKRSYPSLLGYRKTIKNSKGNYIWEPVESELLQVKNWFTRYKNGESLKNIVFSQNSNENKASHILKRTTHLSRTLQHYVYTGYSLNTKGLDYLKKFDNFEIDNLQMLHNPDYWVKSIPYSLEIINREDWIEVKERLRIYKEKHKKNTNRRAEKSIGTGLITCGYCGAKFFYQVQAHKRKKGLVLYPYYFHMSCLDRTCLQSPKSVSQDKIDTIFKIFVLYSTITSDSKSKFLKERLFQEDIEVKAIKEKVKILKRDHQKTETQISKFKTALETTEDVGAITVLAKQIDNTETTLTEIKNSIISGEAELQERQEAMNKTRSQLMHYSICDLLTQFFEKWNIEEQRNHLLKIVDNAVITGTTLNIKSGEYTYIFDTNKKYEFPTVVYNEMLKEAKEDIDYSSFFRNKPDDHFERRMWSILVMSESVWHICEWRDKEKQLIF

SEQ ID NO: 497 - protein, 497 aa, Tenacibaculum dicentrarchi
MKAVAYIRISSSEQEYKRQHEELSELARFKNLNLVKVFADVVSGSKTKAKERASFEIMDKYLLDNSDVKNLLILETSRLGRKKLDVLNTIEDFFLRGVNIHIKDLNLCTMENGKRSITTDILVSLLSIMADEETRLLSERIKSGKMSKAKENKAFGAKVIGYKKGKDGTPIIDEKEAPIIKRIFELASQGLGMRKISSIIESEFNREFAIGTLSSIIKNSFHKGKRKYKDLILDVPPIVSKGLWQKANDSINSRSKFGSRKYVNTNVVQGKIKCGVCNSVMYQKVIPKGRINSFVCKDTKCKNSINRPWLFRMIRLIVDKHALKNKDEKVREKIKLQITSHKAELQINNKLLAKLKRRSEKIRILWLDDEITDARYKSDISNVNKEIKLCNTKSKEIEKAIVIAEKSLKNDIEHYSKELSVFKTEIQDVLSHVIIDKERVLINIFGWREYDLSKPNSIKLGWEARKPISERYKNEKLPLRKPISDEDLNLMIDNYTL

SEQ ID NO: 498 - protein, 497 aa, unidentified
MNAIYARQSVDRADSISIESQIEFCQYEMRGEQYKVYTDRGYSGKNTDRPAFAEMMNDIENGVIGKVVVYKLDRISRSILDFSNMMEQFGKHKVEFVSTTEKFDTSSPMGRAMLNICIVFAQLERETIQKRVADAYYSRSQKSFYMGGRVPYGFRLIPTTIEGIKTSMYEINAEEAEQVQLIYELYSKPECSYGDIIRYFQAHGILKNGKPWGRTRLADILRNPIYVRADPSVYAFYRDQGAIMANDPADFIGTNGCYYYKGQDSAGRKQMNLEGNHLVLAPHEGIISSDLWLKCRVKCLEAQQIKPYQKAKNTWLAGKIKCGACGYALVDKHYSTTRSRYLLCSNKMNAKACEGPGTIYTDEFEQIIYNEMQKKMAQFKKLRRCKGKRVNPELTALNIQLTQVETEISSLMDRLSAADDTLFRYISGRIKELDGKKQELMKRISERKLHKEADYTEINNHLTMWDELSFDDKRQTVDQLIRVIYATSDSIKIEWRI

SEQ ID NO: 499 - protein, 542 aa, Vibrio sp.
MKTAYSYIRFSSSRQADGDSIRRQTELARAYAEEHELDLQDVSISDFGVSAFRGSNATEGALGAFLKLVDEQKIDSDCYLLVESFDRLSRQAVEDALALLLQVVNRGITLVTLSDNHVYKRGELDMTMLILSIVTMSRANEESEIKSQRTSAAWSKARIDALNGIKVKNSRLPDWLSWNEDKTDFVVNQPAKATIQRMFELSAGGSGIEIIAKTLNSEGLKTFKQFKEWRSSGVSKLLRNRAIVGEFQFYRKDAKGNRVAVGEPIAGYYPEVVSKQLFLTVQQGMDLRNRRGSGNRAGQFTNLFTGLIRCGECGSAVVTSSQTTKTPQGYLKCTMRCESKARMNYKYTEPQVLSALGSLQSVIEKYRTPISDETASIELDVRALDEKVTTFESLLDNAFTPKLAERLQEAELQLANAKALLESERQRVSAEQSREQIVLGLEPLESTDDRRAFNSKLRTVIDRIEMIEHPHEKGSALVYFINDCPVMEQHFTKLKGAHGTSSEVYELHSDDVPSHLGRTTSKKPLEFIIEESGDVLSFMPAQ

SEQ ID NO: 500 - protein, 516 aa, Vibrio harveyi
MQDLPKQAYSYRRFSFLTQKFGSSLKRQTKLAQDYATQHGLTLSDTSFEDLGVSAYRAANASEDAGLGQFLLALREGKITTPCTLLVENLDRLSRAKIKVAMRQLWDITDQDVHVVTLVDGRIYTKDMDFEDIMLAGLIMQRAHEESETKSKRLQEKWQERRTLGKFIHKNCPFWLTPNKDRTDYEVNKYIETVHQIYAMALGGLGSRVIAQELNSSGITAPRGGLWSTATITKVLNNRAVLGEYQPKQRVIVDGVRSEKPIGSPIQNYPVILDSDYFDQVQSALRGRHKGNNRNSTKTYRNVLKHIAHCGCCNGTIRLKQQQHLYYLQCSVECKGSRPVSIRYLHDWLNEVWITSDFASVSLSDVPEAKELATLESELEKLTEAVSGLAAAYAATLDPTINSQLLETSAKKVETQTKLDDLRSELSPYNQTKAAQFERQMLVDLAFSERNEVENFVARTKLTGLLAQLKDFIIHKGQNDIVTFEIKTAQNESKTYTAVKNPYHKTRKLTGKIWNY

SEQ ID NO: 501 - protein, 529 aa, Vibrio parahaemolyticus
MRKVYSYMRFSRPEQAKGTSIERQSNFAEQYALEHGLELDKSLTMMDKGLSAFHGVHKTKGAFGQFLAAVTAGKVPSGSVLVVESLDRLSREDPLIAQAALTDLILSGITVVTAADNQVYSREEIQQNPFKLIMSLVVMIRANEESETKQRRSNAFLKSALNQYQANGKIRRLGSDPSWLDFNKDNDTYSFNERVEVIRRILNLYNKGIGSLKIARQLTEESILTLSGKRSAWGQTTVANIVKSHALYGMKRINVQSVEYDLEDFYPALITKSKWLALQQHRTKRSKSMHGRGEVVHLITGHGKGFTTCGYCGGGLGAQTQKQYKRSGEYSRTVTRLHCLTHKETSSCCSSVFLEPIESAVLFAACIGVEPQSLVPTVNNVNYSALIDEVENKVKHIVEQVTAGAPWDLFKDAHDKLKLEKQKLIKERDSQKPDVNQSDVQKWVNKLVELADKARANDKEARLKCRTLINSICKSIVIKLRGHDLKSEPVVTITFVTDEQFEFKVGKNGAVQFVDNGMAVNRRLKPLLA

SEQ ID NO: 502-514 - skipped (13 entries)

SEQ ID NO: 515 - DNA, 1680 bp, Burkholderia multivorans
  source 1..1680: mol_type = genomic DNA
atggccaaaaagcctaaggctaaagtgtattcctacctgagattcagcgatccaaaacaggccgctggaagcagcgctgatagacagatggaatacgccgccagatgggccaccgagcacgacatgcagctggacgcaacactgaccctgagagatgagggcctctctgcctttcaccagcggcacatcaagcaaggcgccctgggcgtgttcctgcgggctatcgaggacggccggattcagcctggctccgtgctgatcgtcgagggcctggacagactgagcagagccgagcctatccaggcccaggcccagctggctcagattatcaacgccggaattacagtggtcacagcctctgacggcagagagtacaacagagaaagactcaaggcccaacctatggacctggtctacagcctgctcgtcatgatccgggcccacgaggagagcgacaccaagtctaagcgggtgaaggccgccatccggaggcagtgcgagggctgggtggccggcacttggagaggcatcatcagaaatggcaaggatccccactgggtgcggctcggcgagcacggcaagttcgagcacgtgccggaacgggtgctggccgtgcggaccatgatcgacctgttcctggaaggccacggcgctatcgagatcacaagaagactgacagagcagaacctgtacgtgtccaacgccggcaactacagcgtgcacatgtacagaatcgtgcggaaccaggccctgatcggagaaaagcggatcagcgtggacggcgaggaattccggctggatggctactacccccccatcctgaccagagaagagttcgccgagctgcagcagacaatgagcgagagaggcagaagaaagggcaagggagagatcccaaacatcatcaccggactgagcatcaccgtgtgcggatattgcggccgtgctatgaccacccagaatagcaaggccagagccccaaagggcaaaagcgtggtgcgcagactgtcttgccctatgaactcttttaacgagggctgccccatcggcggcagctgtgaatccgagatcgtggaaagagccctgatgcggtactgcagcgaccagttcaatctgagcagactgctggagggcgacgatggcacagcccggaggaccgcccaactggccgtggccagacaacgcgccagcgacatcgaggcccagatccagagagtgacagacgctctgctgtctgatgacggaaaggcccctgctgcattcaccagaagagcaagagagctggaaacccagctggaagaacagagaagagaaatcgaggccctggaacaccagatcgccgcttcatctgcccatggcatccctgccgccgccgaagcctgggcccagctggtggatggggtgctggccctggactacgacgccagaatgaaagctcggcaacttgttgctgacaccttcagaaagatcgtggtgtaccagagaggatttgcccctatcgacgatgctgccgccgatagatggaagcggagcggcaccatcggactgatgctggtaaccaagagaggcggaatgagactgctgaacgtggaccggcggaccggctgttggcaggctgaagatgatctggaccccagcctgatccctagcgacggcctgcctatgctgcctctggacgcc

SEQ ID NO: 516 - DNA, 1545 bp, Bacillus sp.
  source 1..1545: mol_type = genomic DNA
atgaagaccgccatctacctgcggaagagtcgggccgatcttgaggccgaggctagaggcgagggcgaaaccctggctaagcatagatccaccctgctgaaaatcgccaaggaaatgaacctgaacgtgctgagcgtgcgggaagagatcgtgtccggcgagtctctggtgaagagacctgagatgctggccctgctggaagaaatcgaggacaacaagtacgacgtggtcctgtgcatggacatggaccggctgggcagaggcggcatgaaagaacagggaatcattctggaaacatttaagcggagcaataccaagatcatgacccctagaaagacatacgatctgaacgacgagtgggacgaggaatacagcgagttcgaggccttcatggcccggaaggaactgaaaattatcacccgcagaatgcagcggggaagagtggccagcgttgaggctggcaactacctgggcacacacgctcctttcggctatgacatccaccggctcaacaagcgggaaaggaccctgaccatcaaccccgaggaggcttctatcgtcagaatgatcttcgactggtacgccaatgaggatatgggcgccaacgccatcagatctaagctgaatgacctgggctataagtccaagctggggaacgagtggaacccatacagcatcctggacatcctgaagaacaacgtgtacatcggcaaggtgacatggcagaaaagaaaggaggtgaaacgccccgacgccgtgaagagaagctgcgccagacaggacaagagcgattggatcatcgccgatggaaagcacgagcctatcattcctgagagcctgttcgagcaggtccaggagaagctcaacagccggtaccacatcccctacaacaccaacggcatcaagaatcctctggccggaatcatcaaatgcgccaagtgtggctacagcatggtgcagagataccctaagaataggaaagaaaccatggattgcaagcacagaggctgtgaaaacaagtctagctacacagagctgatcgagaagagactgctggaggccctgaaggaatggtatatcaactacaaggccgatttcgagaaacacaagcaggatgataagctgaaggaaacacaggtgatccagatgaatgaggctgccctgagaaagctggagaaggaactggtcgacgtgcagaagcaaaaaaacaacctgcacgacctgctcgagagaggtgtgtacaccgtggacatgttcctggaaagaagcaacgtggtgtccgacagaatcaccgagatcacaagcacaatggaaaacctgaagaaagaaatcaagaccgagatcaagaaggaaaaagtgaagaaggacaccatcccccaggtggaacacgtgctggatctgtactttaagaccgacgatcctaagaaaaagaacagcctgctgaagagcgtgctcgagaaagccgtgtacaagaaggagaagtggcaaagactggacgacttcgagctggtgctgtaccccaagttgcctcaggacggcgacatc

SEQ ID NO: 517 - DNA, 1500 bp, unidentified
  source 1..1500: mol_type = unassigned DNA
atgagagccctggtggtcatccgcctgtctcgggtgacagacgccaccacatctcctgagagacagctggagagctgccagcagctgtgcgcacagaggggatgggatgtggtgggagtggcagaggatctggacgtgagcggagcagtggaccccttcgacagaaagcggagacccaacctggcaaggtggctggccttcgaggagcagccctttgatgtgatcgtggcctacagagtggacaggctgacccgctccatcaggcacctgcagcagctggtgcactgggccgaggaccacaagaagctggtggtgtctgccacagaggcccacttcgataccacaaccccttttgccgcagtggtcatcgccctgatgggcaccgtggcacagatggagctggaggccatcaaggagcggaacagaagcgccgcccacttcaatatcagagccggcaagtacaggggctccctgccaccttggggctatctgcctacccgcgtggatggagagtggaggctggtgccagaccccgtgcagagggagcgcatcctggaggtgtaccacagggtggtggataaccacgagccactgcacctggtggcccacgacctgaataggcgcggcgtgctgtctccaaaggattattttgcacagctgcagggaagggagccacagggacgggagtggagcgccaccgccctgaagcggagcatgatcagcgaggccatgctgggctatgccacactgaatggcaagaccgtgagggacgatgacggcgcccctctggtgcgggcagagcctatcctgaccagagagcagctggaggccctgagggcagagctggtgaagacatcccgcgccaagcctgccgtgtccaccccatctctgctgctgcgggtgctgttctgcgccgtgtgcggagagccagcctacaagtttgcaggaggcggcagaaagcacccacggtatagatgcaggtctatgggcttccctaagcactgtggcaacggcacagtggctatggccgagtgggacgccttttgcgaggagcaggtgctggatctgctgggcgacgcagagcggctggagaaagtgtgggtggcaggcagcgactccgccgtggagctggccgaagtgaatgcagagctggtggatctgaccagcctgatcggctcccctgcatacagagcaggcagcccacagagggaggccctggacgcaaggatcgccgccctggccgcacgccaggaggagctggagggcctggaggcccggccctccggctgggagtggagagagacaggccagaggtttggcgattggtggcgggagcaggacacagccgccaagaacacctggctgcgcagcatgaatgtgcggctgaccttcgatgtgagaggcggcctgacaaggaccatcgattttggcgacctgcaggagtatgagcagcacctgaggctgggctccgtggtggagcggctgcacaccggcatgtct

SEQ ID NO: 518 - DNA, 1527 bp, Clostridium botulinum
  source 1..1527: mol_type = genomic DNA
atgctgaggatcgctatctacagcagaaagtccgtcgagacagacaccggcgaaagcatccagaaccagattaagctgtgcaaggaatatttcaaaagacaggaccccaactgcatcttcgagatcttcgaggacgagggctacagcggcggaaatatcaacagacctagctttcagagaatgatggaactagttaagatcaagcagttcgacatcgtggccgtgtacaagattgatagaatcgccagaaacatcgtggatttcgtgaacacctacgacgagctggacaacatcggcgtgaagctggtgagcatcaccgagggcttcgacccctccactcctgctggcaagatgatgatgctgctgctggcctcttttgccgaaatggaaagaatgaacatcgcccagcgggtgaaagacaacatgcgggaactggccaagatgggcagatggtccggcggcacacctccaaagggatatacaaccaagaaggttatcgagaacggcaaaaagatcacctacctggatctcatcgacgacgaggcctacatcatcaaggacgccttcaagctgtacgccgagggatacagcacctacaagatcaacaagcatttcaaggaaaagggcatcagactgcctcagaagaccatccaaaacatgctgaacaaccccacctacctgatctcttcaaaggagagcgtggatttcctgaagaataagggctacaccgtctacggcgagcctaacggcttcggctttctcccctacaatagacggcctagaacaaagggaaaaaaaagctggaacgacaagtctcagtttgtgggcgtgtctaagcacgagggaatcatcgacctgcctctgtggattgaggtgcagaacaagctgaaggagcggaccgtggaccctcacccaagagagagcaatttcaccttcctgagcggcggcctgctgaaatgctcttgtggcagcagcatgttcgtgcaccctggccacacaagaaaagacggaagccgactgtactacttccggtgcatgaagaacaacggcaactgtagcaatagcaagttcctgagagtggactacgccgaaagctctattctggaatttctggaatccatcagctctaaagagaaactgaccgagtaccagaaaaagaagaagcctcggctggacttcagcatcgaaatcaagaacctgaataaaaagatccgggacaactccaaggccatcgataacctgattgataagctgatgatcctgagcaacgaggccggaaaggtggtggctacaaagatcgaggaactgacaaaacagaacaacatcctgaaggaaagcctcctggaaatcgagcgcaagaagcttctgtccggcctggaggataacaacctgaacatcctgtataacgagatccaaaatttcatccagaccgaggatatcagcctgcggagactgaagatcaaaaacatcatcaagtacatcacctacaacccccaaaatgatagcctgcaggtggaactcgtggac

SEQ ID NO: 519 - DNA, 1686 bp, Cronobacter sakazakii
  source 1..1686: mol_type = genomic DNA
atggaaaagagaaagctgtactcttatatcagatggagcagcgataagcaggccaagggctctagcctgcaaagacagctggagacagccagaagagtggcccacgagaatgatctggaactggtcgagatcatcgacgccggcctgagcgccttcagaagcaagcacctggaaaagggcagcctgggagctttcatcgaggccgtgaaggtgggccagattgccagcgacagctggctgaccgtggaaagcctggacagaatcagccgggacgctatcctgaaggcccaggggttattcatggaactgctggaactgggcataacaatctacaccggcatggataacagaatctatacaaagtctagcgtgaccgacaaccccatggaactgatgctgtctatcatgaccttcagcagagccaaccaggagagcatggtgaagagccagagacagaagagcgcaacacagctgaaaattaacaagttcaacgcctctgtgagggccgacaatggatacccacacgccatccggaactccggcgccaacgtgttttggtccgatgtgtccgatggcaccgtgcggcctcacgagtactacttccccatcgccaagctgatcgtgtccctgagactgaagggctggggctatatgagaatcgccaagcacctgaacgagaactacacacctcctaagggtaccgccaagagaaagcacttcaaggatctgtggtctccaaagctgatctgcaacttcctgcaatctagaaccctgatgggcgaaaagagcattagagtggacggagtggagcacatcttaaagggctactaccctcaggtgatcagcgagaacgaattctacagcctgcagaacgtcatcaaccggaaactggccaatgagcctaacaattacatccccctgctgacaggcatccggagattcaagtgcggttgtggagctgctatggtcgcgttttttcagtacgacaagatcagataccggtgtgatggcatgaagtccctggacaaaagaatgcactgcggcgccaaaagcttcaatggcgcctccatcgagaatgccatcgtgcagatctgcgccgataagatctttaaggacgtgaaaacccacaccagcaacgtgggagccatccaagctatgctggtggaagccaagcgcaagtacaaccggggcatggacatgctgcttgaggacaccgcccctcaaggccttaacatcaagctgatcgaactggaaaagcagatcgaggaactgcagaaaaagctgaacgacgagctggtgaccgaggctggcgtgaacagcagcgtgtcttggggagctgtgcccaaggactacagagatgtgttcaacaccgagcggctggaaatcaagcagaaaatccaggccagcaccctcagcatcgtgtgcaccacgatcaaaacaaaaggccacctgttcgagatcaagtttaacgacggcgacgtgatccggttcttcagcgacaagcggcgggtgcacgtggatgttcattctatcaacaacgccgagattatgcagagcgagggcatcatcctgcacgaccacctggattacctggttggccctgaggaattcatcgagagaatcagacagaagcatctgcagatgaaagccctgcaggagaggaccctggacgacatcttcgacatc

SEQ ID NO: 520 - DNA, 1521 bp, Escherichia coli
  source 1..1521: mol_type = genomic DNA
atgggccggagcgtgatcacctacctgcggtacagcagcgccattcagggcgccgagggcgccgacagcaccagacggcagaacgacctgttcaaacagtggctgaaaaagaacagcgacgcccaagtggtggcctcttttagcgatgagggcctgtctggctacaagggcaagcacctgaccggccagttcggcgacatgctggccagaattgagagcggcgagttccccgagggcaccatcctgctggtggaaagcatcgatagaatcggcagactggaacacctggaaacagaggccctgatgaacagaatcttgggaaatggcatcgagatccacacactgcaggacggcctcatctacaccaaagacgccctggctgatgatctgggcatcagcatcatccagcgagtgaaggcttatatcgcccaccaggagagcaagcagaagagctttagagtgtcccagaaatgggagcagcgggccaagctggccctggccggagaacagagactgacaaagatggtgcccggctggatcgaccccgacacctttaagctgaacgagcacgccgaaaccgtgcggctgatcttcaagctgctgctggatggagagtccctgcacaacatcgcccggcacctgcagagcaacggcatcaagtccttcagcagacggaaggacgctaatggcttcagcgttcacagcgtcagaaccatcctgagaagcgagacaaccattggcaccctgcccgctagccaaagaaacgacagacctgccatccccaactactacgaggccgccatcgacgccgcaaccttcaacaaggcccaggaggtgctggacaagaaccggaaaggcagaacacctgctagcgacaaccccctgacaatcaacatcttcaagggcctgttccggtgccaatgtggcgcttctgtgcatcctacaggcacaaaaaacaaatacgccggcgtgtacagatgcaacaaccacctggatggcaggtgcgacgtgccacctctgaaaagaaagcctttcgacaagtgggtcctcgaaaacttcctgggaatgatcgatgtgggcaacgacggcgaatccgagagaaagatcgccgctctgcaacacgaggtggaaatcgtgacagccagaatcaagaaggccaccgccctgcttcttgaaatggacgatatcgacgagctgaagatccagctgaaggaactgaaccagaagcggaccgagctgcagaccaccatcgacagcatgagaaggaaggcctctctgaccgacaaggagctgcctcagctgaaggacatcgacctgacaaccaaggctggaagagtggaatgccagctgatcctgagcaagcacctgaagggcctgaccctgggcaaagattctgtgaccgtgaccctgcaaaatgatacagagatcacaattcctaccaaccctctgcctttgaatgacggatctcctatcttcgagatcgccgataaggaactgttggacatcgatgcttaccagctg

SEQ ID NO: 521 - DNA, 1407 bp, Enterocloster clostridioformis
  source 1..1407: mol_type = genomic DNA
atgatgaagaagatcgccgtcctgtacatgcggatgagcaccgacatgcaagagcacagcatcgagagccaagagcgggtgctgatggaatacgccaagcggaatggctacgtggtcatccggaagtacatcgacagaggcatctctggccagcacgccagcaagaggcctgacttcctgaagatgatcgacgacagcgagacaggcgagttccagttcgtgctgatctacgactccagcagattcgcccggaacctggtggaaagcctgacctacaagagcatcctgaaagaaaactgcgtgcggctgatctccatgaccgagcctaacctggaagatgacgagatgagcctgtacatcgatgccatgcagggcgccaccaacgagatctatgtgcggaagctgagcaagagcgtgaagcggggccacaacgatagagccctgagaggcgatctgcctggcgacgttcagttcggcctgaagagactgaaggacggcagcatcgtgctggacgaagtgaaggcccctatcatgcggtggatgtatgaggccatctactacgacgatagcagctactacagcatctgcgagacactggccgccaagggcatcaagtcccagagaggcaacatcatcgacagccggcaagtgaagagaatgctgatgaacatcaagaacaaggcctaccactgggccgagaaggatggcaagcccatcctgaagaagggcaactaccccgccatcgtggacgaagagatcttcgacgccgtgcaagagatcattgccgagcgggccaagcactacaagaagaacgagaagcccgccgagttccggaagcactggctttctggactgctcgtgtgtcctcattgcggagccggctacagctacaacacacggaaacctccacagcacgacgccttcagatgcggcaatcagacaagaggcgcctgcaagaagggaagcagcatcctggtggatgtggccgtggaaatggtgctggacaagctgagcgaggtgtacgctggacctctggctccctacgtgaagaacatcaccgtgtctcagcccgagcctcagatcgactacgacaaagagatcaagctgctggaagcccagctgaagagagccaaacaggcctacctggccgagatcgacaccatcgatgagtacgcccagaacaagcggcggatcgccagcaacatcaaagagctgcaagaggctaagaatcaggcccaagaaggcgccgctctgaacgagccccagttcaaagtgaagctgctgaacgccatcacactgctgaagtccgactgtcccatgagcgagaagattccagccgccagatccatcatcgagaagatcctggtcgaccccagaaacaagaccatggacatctactttttcgcc

SEQ ID NO: 522 - DNA, 1431 bp, Fusobacterium mortiferum
  source 1..1431: mol_type = genomic DNA
atgcgggacagcaagtctaagacagtgatcatctacaacagagtgtccaccatcatgcaggacaagaacgagagcctgaccgagcagaccgatgagtgcatcagatactgcaagacaaacggctatgagatctataagatcctgaaggacgtgaagtctggcaccaaggatgatagagccggctacctggaactgaaaaagcacatccggagaagagatttcgacatcctcgttgtgctggaaaccagcagaatcgccagaaagatgaaagagctggtcctgttcttcagcctgctgaatgagaacaatatcgagtacatcagcatcagagagcctaactacaatacaaccacacctgatggcaagttcgccatgaacattcggctgggtctgatccaatttgaaagagataacaccgccgagcgggtgaccgacagactgtacttcaaggccagcaagggacagtgggtgaatggcaaacctcctatcggatacaagctggtcaacaagaggctggagatcgacgaggaaaaggctgaaatcgtgaagaatatctacgaggacttcctgaacggctattctctgaaccagatcaacaacaagctgcagtttagctggggcagcaaacaagtgaagagaattctcaccaaccccacatacaagggatatatcagatacggcaccagaagcaatagaaaaaaaaacaaccgggaagctttcatcgtgaagggctggcacgaggccatcatccccgaggacaagtgggagaaggtgcaggagatgtacaaaagccttaaccggaaggcttctaatacaaagcccaccctgctgagcggcctgctgaaatgcaaggaatgcggcaacaactacatccgcaagcggggcggcagctacgacaaaaatctgtactacggatgtaacctgaacaacctgagatacagcgacaaatttttctacaaggacatcccagagtgttcctccgccacaatcaagggcgacctgctggagaaggccgtgatcgacaccctgaaaaagcagatcaacgacctgaatttcaacgatatcgaaatcgataagaagcggcaggtgaacatcaagcagatcgatagcagcattaacaagttcaagaatcggctgaacaagatctacgaactgtacatcgaggacgagatccctaaggacaagtacctgaaggataaaaaagacattgaagccagaatcatctctctggaaaagcagaagaaatctttcggcgagatcgaagtggaaaagagcaacaacgagatgattcaggagtactttagcaagatcgacctgtccaacatcgaggaagccaacagaatcctgaagatcatcgtgaacaagatcgtggtgtacaagaagaagaagacccctaaggacgacttcgaggtggaaatctacctgaacatt

SEQ ID NO: 523 - DNA, 1572 bp, Klebsiella pneumoniae
  source 1..1572: mol_type = genomic DNA
atgggcagacaggtgatcacctacctgcggttctctagcaagccccaggagagaggcgacagcatccggagacaaaagggcctgttcgagcggtggctgaaggacaaccccgacgccaaggtggtggacgagttcagcgacgagggcgcttcagcttatcacggccaccacctgaagggcgatttcggcagaatgctgcagaacatccaagacggcaagtacctgagcggccagacagtcctgctggttgagtccgaaaccagactgaacagacagaaggccagaaacaccgagaacctggtcgaccttatcactggaaaaggcgtggacgtgatctgcctggaaagcggcaaaatctacaccagtacaaacattgatgacctggataccagcatccagctgaagatcgccgctcacatcgcccaccagcagagcaaggaaaagagcatcaaggtcagcgccgcctgggagcacagagctcagctggccctggaaggcaagcagcagctgacaaagaacgtgcccggctggatcgatcctgacaccagaaagctgaacgagcacagcagcaccgtggtgaccattttcgacctgctgctgagtggcgagagcctgcacaacatcgccagatacctgcaggccaacaacatcaagtcttttagccggcgggaaaaggccaatggcttcagcgtgcacagcgtgcggaccatcctgagatctgagagcaccgtgggcacccttgctgcctctaagagaaatgatagacctgccatccccaactactacgagcctgctatcgacgtggccaccttcaacaaggcccaggagatcctgagcaaaaaccgggtgggtagagcccctgccagcgacaatcctatcacaatcaaccttttcaagggcatcattagatgccagtgcggcgccagcgtgcatcctaccggagttaaggcaacctaccagggcgtgtacagatgtaacaacgtgcccgatggcagatgcaacgtgcctacaatcaaaagaaagcccttcgataagtggatgctggacaacatagttggatttctggaacgggacgacggcaacaacacagacaagcggaaggctgaaatcgagtaccaaatcagcctggtgaccagcaaactgaaaaaggccacaacactgctgctggaactggacgacgtgaccgagctgaaggaacaggtgaaggagctgaacatccagcggagcaacctgcaatctgaactggacgagctcaaccagagagagacactgagcgacaagcctctgcaccacctgagcgagatcgacctgaccaccaaggcaggcagagtggaagcccagctgatcctgagtaagttcgtgcagagcatcgagctccagagagaaatgatcatcatcaccctgagaaacggcacagtgatcggcaagtccagagacctgagccctgtgctgtctcaggacctgatgaaacaggtcgtgtcctccccatctccaaccgacatcgacatgttctcagtgataacaagtgatgaggaatttagaaaaagcggaaaacaggtgaccaagcggagc

SEQ ID NO: 524 - DNA, 1428 bp, unidentified
  source 1..1428: mol_type = unassigned DNA
atgaaggccgtgatctacacccggatcagcaaggacagagagggcgccggactgggcgtggaaagacagagagccgactgcgccgccctggccgatagactgggatggcaggttgtgggcaccttcagcgacaacgacctgtctgcctacagcggcaggcacagacctggctacgccgccatgtgcgaggctctggaagctggagaagcccaagccatcatcgcctggcacaccgacagactgcacagaagaccagtggaactggaaagcttcatcggactgtgtgaaagacggagcatccaggtgcggacagtgcgggccggcaccctggacctgtctacaccttctggacaaatggtggctagaatgctgggcgccgccgccagacacgagatcgaccacgccatcgagcggcagaaaagagctaagaagcaggccgccctggatggcagatacagaggaagcagaagagccttcggctacgagcgcgatggcctgaccctgtgcgacgctgaggccgatgccatcagaaccgccgccgagagagtgctgagcggaacctccctgtcccaggtggccagagattggaacgccgcaggcctgcggaccgcctttggcggcaaggccttcaccagccgggaagtgcggagaatcctgctacggcctagaaatgccggatatagcctgcacgagggcaagcggatccctaacgcccagtggcctcctattatcaccaccgatacattcgccgccctggaagccctgctgagagaccctgtgagatctaaacatctggcttttgagcggaagtggatgggcagcggcgtgtacctgtgtggcaagtgcggagctaagatcagcacagccagccagaagggcaccggcaaaagctggcggcccacctacgtgtgcagcgccagcaagcacctgggcagagtcgccgacaccgtggacgagtacgtgaccgaggctgtgcttgaacggctctctagacccgacgcccctatcctgctgggtggcaacaaggtggacgtggccgacctgacatctagacgagatggctatagagcgagactggatgagctggctgctatgttcgcagctggcgacattgacgccggccagctgaagtccggaacaacagagctgcggagaaagctggaccgggtggacgccgagctggccgctgcccgggcctctagcgtgctggccgacctggtgctgtccggggatgatctgagagatacctgggccgccatcccccccggcggcaagggcaaagtgatcgatgccctcatgaccgtcacaatcgagcctacaagacggggcagaaggccaggcggcagctacttcgaccccgagagtgtgacaatccggttcaagggcgttggcgaacacagactggacgacggccagctgatcggcgcc

SEQ ID NO: 525 - DNA, 1815 bp, Nocardia otitidiscaviarum
  source 1..1815: mol_type = genomic DNA
atgcctgagagcccccccagagccctgatcgtgattagactgtctaaggtgacagatgccaccaccagccctgagcggcagctcgccgagtgcagagccatctgcgagaagagaggatacgaggtggttggtgtggccgaggatctggacgtgtccggcgctgtggatcctttcgacagaaagaagagacctcacctggccagatggctgcacggcgagcacctggatgataacggagaacctgtgccctttgaggtgatcgtggtgtacagagtggaccggctgacacggtctgtgcggcatctgcagaaactcgtggcatgggccgacgatcataagaaactggtggtgagcgccacagaagctcacttcgacacaagcctgcctttttctgctgtgctgattgccctgatcggcaccgtggccgagatggaactggccggcatcagcgaaagaaacgccagcgccgcccgacacaacatccaggccggcagatacagaggcagcacccctccttggggctacgtgccaagcaatgatacaggcgaatggcggctggtgcgggacaaggaacaggccgacgttatcaacgaggtggcccgcagagttatcgagggcgagccactgcaaaagatcgcccacgacctgacccggagaggcatcctgacccctaaggacaacttcgccaaacagcggggcagagaaattaagggcagagagtggtccgtgacgcagctgaagagaagcctgctgtctgaggcgatgctgggacacgccgtgtccggcggagccgctgtgagaaatgacgacggcagccccgtggtcagaagcgaacccatcctgagccgcgagatctacgacagagtggccgccgagcttagcagccgggccaaaaggggcgagccaaacaagcgcaccagctctctgctgctgggcgtcatctcatgcggcaacccctgcctgcacaagcaacagggccacgagtgtcctgaaggttgtagcggaacctgcgacgagccggtctacaagttcaacggcgggagccacagccagttccctagatacagatgtagaaccatgaccagagcttataagtgcggcaaccggacaatcagagccgatcaggctgacgcccaagtggaaagaacaatcctggccttactgggctcttctgaaagactcgagcgggtgtgggacgccggagaagaccactctgccgagctggccgatatcaacgacgaactggtggacctgacatctcagctgggcgtgggcgcctttagagctggcacacctcagagagctaagctggacgccagaatcgccagcctggctgagcggcaagcccagctgagctccgaggcagtggtgccagccggctggaagctgctgcctaccggcgagctgttcggcgactggtggagcagacaggacctgaccgccaggaacgtgtggctgcggtccatgggcgtgcgcgccagattcaagagggacgacaagaccctgtacatcgacctgggaaatctgaacgacctgatcagcgggctgaagcccggcggcaccgcccagagagtgcgaggaggcctgcaggccatggaacggaatggcatccagggaatggtgttctccgacgccgacagcgaagtgatgcccgcccctgccgctggctatatgtggatccagcctgttgagggagtgtgggtgtacaccagcgaggccctgctggctgccgccgccgaacggcaggccctgcggagagaaaaaatcgaggatgagttcgcctacggccctggcgacttcgatgaagattgggat

SEQ ID NO: 526 - DNA, 1656 bp, Pseudomonas aeruginosa
  source 1..1656: mol_type = genomic DNA
atgggccctaccgcctacgcctacatcagatacagctctaaagcccagggcgaggccggcagagatagcgtggaccggcagatggcttctatccaggccatcacaaagcagcagggaatcgagctgagaaccgagaacatcttttctgatacaggcatctccggcttcgacggaagcaacaaaaacaagggcaagctgaaggacctgatcgacctgatcatcagccagaagattcaggatggcgacttcgtgttcgtggaatctatcgacagactgtccagacagaagatgagactgagcaaggacctggtctacaccatcctggaccgcggcgtgaccctggtcaccaccatcgatggacagatgtactctcgggccaaggacggcatggaacaggacatcatgctgagcgtgatcgcccagagagcccatgaggaatccaagaccaagtccgtgcggagaaagagcgcctggaatcgggccaaaaaactggctgatgaggaaaaagagatcttcaacgggcacaaccccccctacggcatcagcttcaacaaggaagagagccggttcgagatcgtggaagaagaagcccaggagatccgggatatcttcgagtccctgaaatacgtgggcgtgtctctgaccatcaaaaaggtgaacgagtacagcaagcggaaatggaccaaccggaacatcaagcacctgctggataccaagtacgtgctgggcagctacatggcccaaagaagagatgaaaacaagaaaaaggtgttcgagagatacatcgagaactactaccctcagatcgtgtcctttgagctttttaacgaggccgtggccagcatgaagaacagagcccacagaaagcactacggcaaccagacagtgggcagcctgaacatcttccggcacagcatcaagtgcagcaactgcaacgccagcatgctgttcgagaaacagaccaatccaaagggcgtggtgtatccttatttccagtgcttcaccagaaaggaactgaagaacggctgcgaccagcctagattcagattcgacctggcctttggcgttttcctggaactggtgaaattcgccacaaccagcagcgagacaatcaaccctaacgactggaacaatgagatcaccagcgtgggatcttttcacaagacactgtttaagctgctgacatctacagagaaggataaggaactggaaaagaagatcagccacgtgaagaatctgctgctggaacagaaaaactaccaggacaacatcaacaaaagcttcgaggcttttaccgacggcatcattcctgccgctttcatcaagaaggcctctgaaaccgaaatcaagatcgaggccctggagcgggaactggccgagctgaacatcgagagcagcaccagaaacgtgtccctgctggtccactcttacaacgacatcatcgatctgtacaagaccgaggctggcagactgaagatcaacagcttcttcaccagcaacaacatcgcctttagcttcagctttgaccaaaaaactagaatgctgaggtgtaaagtgtactacaaggacatccacgtggacgtgatcaacaagaagttccccctgcacaatcctctgaaagagttcggcattgataacctgaatcaatacttcaac

SEQ ID NO: 527 - DNA, 1812 bp, Streptomyces sp.
  source 1..1812: mol_type = genomic DNA
atggacacctacgccggcgcctatgataggcagagcagagagagggagaacagctccgccgcctccccagcaacacagcggagcgccaatgaggacaaggcagccgatctgcagagggaggtggagagggacggcggccggttccggtttgtgggacacttcagcgaggcaccaggcacctccgccttcggcacagcagagaggccagagtttgagagaatcctgaacgagtgcagggcaggccggctgaatatgatcatcgtgtacgacgtgagccggttcagccggctgaaagtgatggatgccatccctatcgtgtccgagctgctggccctgggagtgaccatcgtgtctacacaggagggcgtgtttcggcagggcaacgtgatggacctgatccacctgatcatgagactggatgccagccacaaggagtctagcctgaagtccgccaagatcctggacaccaagaacctgcagagggagctgggaggatacgtgggaggcaaggcaccctacggcttcgagctggtgtccgagaccaaggagatcacacgcaatggccggatggtgaacgtggtcatcaacaagctggcccactctaccacacctctgaccggcccattcgagtttgagcccgatgtgatccggtggtggtggagagagatcaagacacacaagcacctgcccttcaagccaggctctcaggcagcaatccaccctggcagcatcaccggcctgtgcaagaggatggatgcagacgccgtgccaacccgcggcgagacaatcggcaagaagaccgcctcctctgcctgggaccccgcaacagtgatgagaatcctgcgggaccccaggatcgcaggattcgcagccgaagtgatctacaagaagaagcccgacggcacccctaccacaaagatcgagggctacagaatccagagagaccctatcacactgcggccagtggagctggattgtggcccaatcatcgagcccgccgagtggtatgagctgcaggcctggctggacggaagaggcaggggcaagggactgtccagaggccaggccatcctgagcgcaatggacaagctgtactgcgagtgtggcgccgtgatgaccagcaagaggggcgaggagtctatcaaggacagctatcgctgccggagaaggaaggtggtggaccccagcgccccaggacagcacgagggcacatgtaacgtgagcatggccgccctggacaagttcgtggccgagaggatctttaataagatcaggcacgcagagggcgatgaggagaccctggccctgctgtgggaggcagcacgccggtttggcaagctgacagaggcaccagagaagtccggagagagggcaaacctggtggcagagagagccgacgccctgaatgccctggaggagctgtacgaggacagggcagcaggagcctatgatggacctgtgggcaggaagcacttccgcaagcagcaggccgccctgaccctgaggcagcagggagcagaggagcgcctggccgagctggaggccgccgaggcccccaagctgcctctggaccagtggtttcctgaggatgccgacgccgatccaacaggaccaaagtcttggtggggaagggccagcgtggacgataagagagtgttcgtgggcctgtttgtggataagatcgtggtgaccaagtctaccacaggaaggggacagggcacaccaatcgagaagagagccagcatcacctgggccaagccccctacagacgatgacgaggatgacgcccaggacggcaccgaggatgtggcc

SEQ ID NO: 528 - DNA, 1602 bp, Pseudomonas syringae
  source 1..1602: mol_type = genomic DNA
atgaagcaggccatcagctacatccggttcagcagcgccagacaagagggcggaagcagcgtggaaaggcaagagggaatgatcgccaagtggctgctggaccaccaggactacgagctgagcaagctgaactacagcgacctgggcaagagcggctttcacggcgagcatgtgaaagaaggcggcaacttcggcaagctgctgaaggctgtgatggacggctacatcaagcgggacgatgtggtgctggtggaagccatcgatagaaccggcagactgcccgctctgcagatgctgtctgatgtgatcgcccctatcctgagagccggcgtgtccatcatcaccctggacgacaataccacctacaccgaggcctctgttggcggccctcacctgtttatgctggtggccaaaatccaggccgctcacgagtacagcagaaccctgagcagaagagtggaagatagctacaagaagcggcggaaggacgccaaagaaaaaggcgtggcccctaagcggatgacccctgtgtggctgaatagcgacggcaccatccgggaagatgtggccccttggatcaagaccgccttcgagctgtatgtgtccggcgtgggcaagtccacaatcgccaagagactgagagaaagcggcgtggaacggctggccaaagcttctggacctggcgttgaaggctggctgagaaacaaggccgtgatcggaaagtgggagacactgatcggcacccctgagcaccacgtgatcgacgatgtgtaccctctgatcatcgagcccagcctgttctacaaggcccaggtgcacgccgagaagatgaagacccagcggcctatcaagacagccaagcactttctcgtcggcctggtgcactgtggcgagtgcggcaagaactacatcatgcagaacctgcacggcaagccccacagcatgagatgcagaacacggcagagccagaacaactgcagcaacagctacgtggtgcccaagcctgtgctggacgccatctacagatacaccagcgtgaccgccgctctggaagctgttcagctgcaacaactgggcgtgaacgagaaagaaatcgtgaccagagaggccgagctgctgaccatcaccaaacgcgtggaaggctttgtgcaggccgtgaatgaagctggccctatgcctgaactgctgacagccctgaaacaggcccggatcgagagagagtctgccgagaacgccctggttatcctgagggccacagtggttacccctccagccaaccagtggcgcgagatgggaaaagtgtggtccctggaagccgaggacgcccagagactgtctgccatgctgagacaagtgggctacaacatcaccgtcggcaagggcgccatcatcaagtctagccactccgacgtggtgtaccagtacctgggcgtcgacagagtgaaggacatgtacagagtgctggccgacggcgagatgaagctgatccacaagagccaggtggacgactacccctaccacgagccttttcacgaggttgtgggcgaagccaccatggacgagacagacaaagagaacctgctgctgcagtaccagagcagc

SEQ ID NO: 529 - DNA, 1602 bp, Salinispora sp.
  source 1..1602: mol_type = genomic DNA
atgagcaccagcattcccgaggaaagcggccccaacgacctggaacttagaggaacaccagccggcctgcctccatatgctgatctggtggccgccaatcctaacgccatctttgtgggcgcctacagcagaatcagcgacgactggcggaagaacaagagcaagaaggccgctgcctctcgttggagcgctggaaaaggcgtggccaatcagcacagacggaacgacatgaacgccggcagacaccaagtgatcgtggtgcacagatacaccgacaatgacctggtggccagcagactggacgtgttcagacctgacttcgcccagatgctgaaggacctgaagctgggcagaacaaaggacggctacaggctggacggcatcatctgcgtgcaccaggacagactgcagagaaccgacaccgactgggagcactttgtgcatgccctgctggctaagcctggcagactgctgtggacacctagcggctctagcgacctgacagatgagggcgagatcgtgaaaaccggcatcatggccgtgctgaacaaggccgagagcatgaagaagaagcggcggatcagagactggcaccaggatctgattctggacggcctgccacactctggccctagaccttttggctggaacgaggacaagatgaccctgagagccgaggaagccgattatctggcctgggccatcagagagcggatcaagggaaaagccctgagcaccctgtgtgccgaggccaagaagagaggcctgaagggcacaaaaggcggagagatcgcccctaccacactgagccagatgatgacagcccctagagtgtgcggctacagagccaacagaggcacactggccctggacgaaaatggcgcccctatcgttggcaagtgggacaccatctgcaagcccgaggaatgggaagccgtgtgcgccacttttagccctggcagcacctacatgcacagaggacctggtgctcctagagtgaccggcaagcctaagaccgtgaagtacctggccagccagctgctgcggtgtatgaacaaggtggaacgcgacggcgagacacggatctgtaatggcacactgacaggcagccccaccaagagcgccagatctccctacacctacagatgcggcagctgcaacaagaactctatcgctggccccatggtggacagacagatcacaagactgctgctgggcaagctgggagaagcccagatcacatacagaaggcctgagctggcctggcacctggaaagcaccctgaaaaccctggccgatagactggccggcctggaaaacagatggatggccggcgaagtggacgacgagcagttctacagactgagcccaggactgcaggccgaagtgcggaaactgagagctgaaagggccagatgggaactcgagaatgccgccggatctgaggaacccgccgacatcattagaaagtggcggagcggagagctggatctggcccagcgtagaagaatcctgttcgacgtgttcgccgccattcaagtgacccctggccagaagggcagcaagacccctaatcctcaccggctgaagcctatctgggga

SEQ ID NO: 530 - DNA, 1491 bp, unidentified
  source 1..1491: mol_type = unassigned DNA
atgaacgccatctacgccagacagagcgttgacagagccgacagcatcagcatcgagtctcagatcgagttctgccagtacgagatgagaggcgagcagtataaggtgtacaccgaccgtggatatagcggcaagaacaccgatagacctgcttttgccgagatgatgaacgacatcgagaacggcgtgatcggaaaagtggtggtgtacaagctggacagaatctccagaagcatcctggacttcagcaacatgatggaacagttcggcaagcacaaggtggaattcgtgtccaccaccgaaaagttcgacacctcttctccaatgggaagagccatgctgaacatctgcatcgtgttcgcccaactggaacgggaaacaatccagaaaagagtggccgacgcctactacagccggtcccagaaaagcttctacatgggcggacgggtcccctacggcttcagactgattcctacaacaatcgagggaattaaaaccagcatgtacgagatcaacgccgaggaagctgaacaagtgcagctgatctacgagctgtacagcaagcccgagtgcagctacggcgacatcatcagatacttccaggcccacggcattctcaagaacggcaaaccttggggaagaaccagactggccgatatcctgcggaaccccatctacgtgcgggccgatcctagcgtgtatgccttctacagagatcagggcgccatcatggccaatgaccccgccgacttcatcggcaccaacggctgctactactacaagggccaggacagcgccggccgcaagcagatgaatctggaaggcaaccatctggtgctggctcctcacgagggcatcatttctagcgacctgtggctgaagtgcagggtcaagtgtctggaagcccaacaaatcaagccttaccagaaggccaagaatacctggctggccggaaagatcaaatgcggcgcttgtggctacgctctggtggataagcactacagcaccacaagatccagatacctgctgtgcagcaacaagatgaatgctaaagcctgtgaaggtccaggcaccatctacacagatgagttcgagcagattatctacaacgagatgcagaagaagatggcccagtttaagaaactcagacggtgcaagggcaagagagtgaaccctgagctgaccgccctgaatatccagctgacacaggtggaaacagagatcagcagcctgatggacagactgagcgccgccgatgacaccctgttccggtacatctctggcagaatcaaagaactggatggcaagaagcaggagctgatgaagcggatcagcgagagaaagctgcacaaggaagccgactacaccgagatcaacaaccacctgaccatgtgggacgagctgtcttttgacgataagcggcagaccgtggaccagctgattcgggtgatctatgctacatctgatagcatcaagatcgagtggcggatc

SEQ ID NO: 531 - DNA, 1626 bp, Vibrio sp.
  source 1..1626: mol_type = genomic DNA
atgaagaccgcctacagctacatcagattcagcagcagcagacaggctgatggcgacagcatcagaaggcagaccgagctggccagagcatacgccgaggagcacgagctggacctgcaggacgtgtccatctctgatttcggcgtgagcgcttttagaggcagcaacgccaccgaaggcgccctgggcgccttcctgaaactggtcgacgagcagaagatcgatagcgattgctacctgctggtggaatccttcgaccggctgtcccggcaagccgtggaagatgccctggccctgctgctgcaagtggtcaatcggggcatcaccctggtgaccctgagcgacaaccacgtgtacaaaagaggagaacttgatatgaccatgctgatcctgagcattgtgacaatgagcagagctaatgaggaaagcgagatcaagagccagcggacctccgccgcctggtccaaagccagaatcgacgcccttaacggcatcaaggtgaagaacagcagactgcctgactggctgagctggaacgaggataagacagacttcgtggtgaaccagcccgccaaggccacaatccagagaatgttcgagctgagcgccggcggctctggtatcgagatcatcgccaaaacactgaatagcgagggcctgaagacctttaagcagttcaaggaatggcggagcagcggcgtttccaagctgctcagaaaccgcgctatcgtgggagaattccagttttacagaaaggacgccaagggaaatcgcgtggccgtgggcgagcccatcgccggctactaccctgaggtggtgtccaaacagctgttcctgacagtccagcagggcatggacctgagaaaccggagaggcagcggcaacagggccggccagttcacaaacctgttcaccggcctgatccggtgcggagaatgtggctctgctgttgtgacctccagccaaacaaccaagacccctcagggatatctgaagtgcacaatgagatgtgaaagcaaggctagaatgaactacaagtacaccgagccacaggtgctgagcgcccttggatctctgcagagcgtgatcgagaagtaccgaactcccattagcgacgagacagccagcatcgagctggacgtgcgggccctggatgagaaggtgaccactttcgagagcctgctggacaacgcctttaccccaaagctggccgagcggctgcaggaggccgaactccagctggccaacgccaaggccctgctggaatctgagagacagagagtgtctgctgaacagtctcgagaacagatcgtgctgggcctggaacctctggaaagcaccgacgaccggagagcattcaacagcaagctgagaaccgtgatcgacagaatcgagatgatcgagcaccctcatgagaagggcagcgccctcgtgtatttcattaacgactgccctgtgatggaacaacacttcaccaagctgaaaggcgctcacggcacctccagcgaggtgtacgagctgcacagcgatgacgtgcccagccacctgggcagaaccacatctaaaaagcctctggaattcatcatcgaggaatctggagatgtgctgagctttatgcctgcccag

SEQ ID NO: 532 - DNA, 1548 bp, Vibrio harveyi
  source 1..1548: mol_type = genomic DNA
atgcaggacctgcctaagcaggcgtacagctacagaagattcagctttctgacacaaaagttcggcagcagcctgaagagacagaccaagctggcccaagactatgccacccagcacggcctgaccctatcagacacctccttcgaggatctgggcgtgtctgcttatagagccgccaacgccagcgaagatgccggactgggccagttcctgctggctctgcgggaaggcaagatcaccacaccctgcaccctgctggtcgagaacctggacaggctgtcccgggccaagatcaaggtagccatgagacagctgtgggacatcacagatcaggacgtgcatgtggtgaccttggtcgacggcagaatctacaccaaggacatggacttcgaggacatcatgctggccggccttatcatgcagagagcccacgaggaatctgaaacaaaaagcaagcggctgcaggagaagtggcaggagcggcggaccctgggcaagttcatccataagaactgccccttctggctgacacctaacaaggatagaaccgactacgaggtgaacaagtacatcgagactgtgcaccagatctacgcaatggccctgggtggcctcggaagtagggtgatcgcccaggagcttaattccagcggcatcaccgcccctagaggaggcctgtggagcacagccaccatcaccaaggtgctgaacaacagagccgtgctgggcgagtaccagcctaagcagagagtgattgtggacggagtgcggagcgaaaagcctatcggctctcctatccagaattatcctgtgatcctggacagcgattacttcgaccaggtgcagagcgccctgagaggcagacacaagggcaacaaccggaacagcacaaaaacataccgcaatgtgctgaagcacattgcccactgtggctgctgcaacggcacaatccggctgaagcaacaacagcacctgtactacctgcagtgtagcgtggaatgcaaagggagccggcctgtgtccatcagatacctgcacgactggctgaatgaagtgtggatcaccagcgatttcgcctctgtctctctgtctgatgtgccagaggccaaagagctggctaccctggaatctgaactggaaaagctgaccgaggccgttagcggcctggccgctgcttacgccgctaccctggaccccacgatcaacagccagctgctggagacaagcgccaaaaaggtggagacccagaccaagttggacgacctgagaagcgagctgtccccatacaaccagaccaaggccgctcaatttgaacggcagatgctggtggatctggcctttagcgagagaaacgaggtggaaaacttcgtggccagaacaaaactcaccggactgctggcccagctgaaagatttcatcatccacaaaggccagaatgatatcgtgacattcgagatcaagaccgcccagaacgagagcaagacctacaccgccgtgaagaacccctaccacaagaccagaaagctgacgggcaagatttggaactac

SEQ ID NO: 533 - DNA, 1587 bp, Vibrio parahaemolyticus
  source 1..1587: mol_type = genomic DNA
atgagaaaggtgtacagctacatgagattcagcagacccgagcaggccaagggcacctccattgagagacagagcaacttcgccgagcaatacgccctggaacacggactagaactggacaagtccctgaccatgatggacaagggcctgagcgccttccacggcgtccacaagaccaagggtgccttcggccagtttctggccgccgtgaccgccggcaaggtgcctagcggcagcgtgctggtggtggagagcctggatagactgagcagagaggaccccctgatcgcccaggctgccctgaccgacctgatcctgagcggaatcaccgtggtgaccgccgctgataaccaagtttactctcgggaagaaatccagcaaaaccctttcaagctgatcatgagccttgtcgtgatgatcagagctaatgaggaatctgagacaaaacaaagaagatctaacgcctttctgaaaagcgcactgaaccagtaccaggccaacggaaagatcagacggctgggatctgacccatcttggctggacttcaacaaggacaatgacacctactctttcaacgaaagagtggaagtgatccggagaatcctcaacctgtacaacaagggcattggcagcctgaagatcgcccggcagctgacagaggaaagcatcctgaccctgagcggcaagcggagcgcttggggccagaccaccgtggccaacatcgtgaagtcccacgccctgtacggcatgaaacggatcaacgtgcagtccgtcgagtacgacctggaggacttctaccccgccctgattaccaagagcaagtggctggccctgcagcagcaccggaccaagagatcaaagagcatgcacggaagaggcgaggtggtgcatctgatcaccggccacggaaagggcttcaccacatgtggctactgcggcggcggactgggcgctcaaacacagaagcagtataagaggtctggtgagtatagcagaaccgtgaccagactgcactgtctgacacacaaagagacaagcagctgctgcagcagcgtgttcctggagcctatcgagtctgccgtgctgttcgctgcctgcatcggcgtggaaccccagtctctggttccaacagttaacaacgtgaactacagcgccttgatcgacgaggtggagaacaaggtgaagcacatcgttgagcaggtgaccgccggcgctccttgggatctctttaaggatgcccatgataagctgaaactggaaaagcagaagctgatcaaggaacgcgacagccagaaacctgatgtgaatcagagcgacgtgcagaaatgggtcaataagctggtggaactggctgataaggccagagccaacgacaaagaagcccggctgaaatgccggacactgatcaacagcatctgcaagagtatcgtgatcaagctgcggggccacgacctgaaatccgagcctgtggtgacaatcacttttgtgacagatgagcagttcgagttcaaagtgggcaagaatggcgccgtgcagttcgtggacaatggcatggccgtgaacagaagactgaagcctctgctggcc

SEQ ID NO: 534 - DNA, 130 bp, Homo sapiens
  source 1..130: mol_type = genomic DNA
ccccagaaccagccctttgtcttctcccttggcacaggccaggtcatcaagggctgggaccaggggctgctggggtgagtcatgggggaatgggcttgggagtgggggcgtgcatggccaccgcccagga

SEQ ID NO: 535 - DNA, 169 bp, Homo sapiens
  source 1..169: mol_type = genomic DNA
ccttccttatcagtccccaaacatgcaggaggcacacaaagaccttgaggtggtggtggtcacaggaaggtctactggtacacatcaccttctccaatcattcctagcacagcattttgtgcgtcatctcacccagtggtgttcaaattatgctcctgggttttcccag

SEQ ID NO: 536 - protein, 55 aa, Homo sapiens
PSLSVPKHAGGTQRPGGGGHRKVYWYTSPSPIIPSTAFCASSHPVVFKLCSWVFP

SEQ ID NO: 537 - DNA, 121 bp, Homo sapiens
  source 1..121: mol_type = genomic DNA
gagaaggagatgtcatacatatttgggttcttcgtgtccaaagatgatcatgaaatgccttccatagaagtgaggttgaggcatcatgtgatgaggagtgagggtctcttctgtgggggag

SEQ ID NO: 538 - DNA, 176 bp, synthetic construct
  source 1..176: mol_type = other DNA
  misc_difference 139: note = a, c, t, g, unknown or other
  misc_difference 140: note = a, c, t, g, unknown or other
  misc_difference 141: note = a, c, t, g, unknown or other
  misc_difference 142: note = a, c, t, g, unknown or other
  misc_difference 143: note = a, c, t, g, unknown or other
  misc_difference 144: note = a, c, t, g, unknown or other
  misc_difference 145: note = a, c, t, g, unknown or other
  misc_difference 146: note = a, c, t, g, unknown or other
  misc_difference 147: note = a, c, t, g, unknown or other
  misc_difference 148: note = a, c, t, g, unknown or other
  misc_difference 149: note = a, c, t, g, unknown or other
  misc_difference 150: note = a, c, t, g, unknown or other
agcgcagcgagtcagtgagcgaggaagcggaagagcgagcttcgcacgattgcaccttgggagccctaaactcccacagataaacagttaatggtaatgaaataacattaactgtttatctgtgtttaatgcctgccgnnnnnnnnnnnngccgccgaatgcaacctcgatcgagc

SEQ ID NO: 539 - protein, 54 aa, synthetic construct
  VARIANT 42: note = Any amino acid
  VARIANT 43: note = Any amino acid
  VARIANT 44: note = Any amino acid
  VARIANT 45: note = Any amino acid
SAASQARKRKSELRTIAPWEPTPTDKQLMVMKHLFICVCLPXXXXAAECNLDRA

SEQ ID NO: 540 - DNA, 20 bp, synthetic construct
  source 1..20: mol_type = other DNA
tcagtccccaaacatgcagg

SEQ ID NO: 541 - DNA, 20 bp, synthetic construct
  source 1..20: mol_type = other DNA
ggaggcacacaaagaccttg

SEQ ID NO: 542 - DNA, 20 bp, synthetic construct
  source 1..20: mol_type = other DNA
ggcacacaaagaccttgagg

SEQ ID NO: 543 - DNA, 20 bp, synthetic construct
  source 1..20: mol_type = other DNA
acacaaagaccttgaggtgg

SEQ ID NO: 544 - DNA, 20 bp, synthetic construct
  source 1..20: mol_type = other DNA
ttgtgcgtcatctcacccag

SEQ ID NO: 545 - DNA, 20 bp, synthetic construct
  source 1..20: mol_type = other DNA
agcataatttgaacaccact

SEQ ID NO: 546 - DNA, 20 bp, synthetic construct
  source 1..20: mol_type = other DNA
ttatgctcctgggttttccc

SEQ ID NO: 547 - DNA, 20 bp, synthetic construct
  source 1..20: mol_type = other DNA
gacgcacaaaatgctgtgct

SEQ ID NO: 548 - DNA, 20 bp, synthetic construct
  source 1..20: mol_type = other DNA
aatgctgtgctaggaatgat

SEQ ID NO: 549 - DNA, 20 bp, synthetic construct
  source 1..20: mol_type = other DNA
tgtttggggactgataagga

SEQ ID NO: 550 - DNA, 20 bp, synthetic construct
  source 1..20: mol_type = other DNA
tggtgttcaaattatgctcc

SEQ ID NO: 551 - RNA, 20 nt, synthetic construct
  source 1..20: mol_type = other RNA
tcagtccccaaacatgcagg

SEQ ID NO: 552 - RNA, 20 nt, synthetic construct
  source 1..20: mol_type = other RNA
ggaggcacacaaagaccttg

SEQ ID NO: 553 - RNA, 20 nt, synthetic construct
  source 1..20: mol_type = other RNA
ggcacacaaagaccttgagg

SEQ ID NO: 554 - RNA, 20 nt, synthetic construct
  source 1..20: mol_type = other RNA
acacaaagaccttgaggtgg

SEQ ID NO: 555 - RNA, 20 nt, synthetic construct
  source 1..20: mol_type = other RNA
ttgtgcgtcatctcacccag

SEQ ID NO: 556 - RNA, 20 nt, synthetic construct
  source 1..20: mol_type = other RNA
agcataatttgaacaccact

SEQ ID NO: 557 - RNA, 20 nt, synthetic construct
  source 1..20: mol_type = other RNA
ttatgctcctgggttttccc

SEQ ID NO: 558 - RNA, 20 nt, synthetic construct
  source 1..20: mol_type = other RNA
gacgcacaaaatgctgtgct

SEQ ID NO: 559 - RNA, 20 nt, synthetic construct
  source 1..20: mol_type = other RNA
aatgctgtgctaggaatgat

SEQ ID NO: 560 - RNA, 20 nt, synthetic construct
  source 1..20: mol_type = other RNA
tgtttggggactgataagga

SEQ ID NO: 561 - RNA, 20 nt, synthetic construct
  source 1..20: mol_type = other RNA
tggtgttcaaattatgctcc

SEQ ID NO: 562 - protein, 6 aa, synthetic construct
GGSGGS

SEQ ID NO: 563 - protein, 9 aa, synthetic construct
GGSGGSGGS

SEQ ID NO: 564 - protein, 12 aa, synthetic construct
GGSGGSGGSGGS

SEQ ID NO: 565 - protein, 15 aa, synthetic construct
GGSGGSGGSGGSGGS

SEQ ID NO: 566 - protein, 18 aa, synthetic construct
GGSGGSGGSGGSGGSGGS

SEQ ID NO: 567 - protein, 21 aa, synthetic construct
GGSGGSGGSGGSGGSGGSGGS

SEQ ID NO: 568 - protein, 27 aa, synthetic construct
GGSGGSGGSGGSGGSGGSGGSGGSGGS

SEQ ID NO: 569 - protein, 30 aa, synthetic construct
GGSGGSGGSGGSGGSGGSGGSGGSGGSGGS

SEQ ID NO: 570 - protein, 33 aa, synthetic construct
GGSGGSGGSGGSGGSGGSGGSGGSGGSGGSGGS

SEQ ID NO: 571 - protein, 36 aa, synthetic construct
GGSGGSGGSGGSGGSGGSGGSGGSGGSGGSGGSGGS

SEQ ID NO: 572 - protein, 4 aa, synthetic construct
GGGS

SEQ ID NO: 573 - protein, 8 aa, synthetic construct
GGGSGGGS

SEQ ID NO: 574 - protein, 12 aa, synthetic construct
GGGSGGGSGGGS

SEQ ID NO: 575 - protein, 16 aa, synthetic construct
GGGSGGGSGGGSGGGS

SEQ ID NO: 576 - protein, 20 aa, synthetic construct
GGGSGGGSGGGSGGGSGGGS

SEQ ID NO: 577 - protein, 24 aa, synthetic construct
GGGSGGGSGGGSGGGSGGGSGGGS

SEQ ID NO: 578 - protein, 28 aa, synthetic construct
GGGSGGGSGGGSGGGSGGGSGGGSGGGS

SEQ ID NO: 579 - protein, 32 aa, synthetic construct
GGGSGGGSGGGSGGGSGGGSGGGSGGGSGGGS

SEQ ID NO: 580 - protein, 36 aa, synthetic construct
GGGSGGGSGGGSGGGSGGGSGGGSGGGSGGGSGGGS

SEQ ID NO: 581 - protein, 40 aa, synthetic construct
GGGSGGGSGGGSGGGSGGGSGGGSGGGSGGGSGGGSGGGS

SEQ ID NO: 582 - protein, 44 aa, synthetic construct
GGGSGGGSGGGSGGGSGGGSGGGSGGGSGGGSGGGSGGGSGGGS

SEQ ID NO: 583 - protein, 48 aa, synthetic construct
GGGSGGGSGGGSGGGSGGGSGGGSGGGSGGGSGGGSGGGSGGGSGGGS

SEQ ID NO: 584 - protein, 4 aa, synthetic construct
GGSS

SEQ ID NO: 585 - protein, 8 aa, synthetic construct
GGSSGGSS

SEQ ID NO: 586 - protein, 12 aa, synthetic construct
GGSSGGSSGGSS

SEQ ID NO: 587 - protein, 16 aa, synthetic construct
GGSSGGSSGGSSGGSS

SEQ ID NO: 588 - protein, 20 aa, synthetic construct
GGSSGGSSGGSSGGSSGGSS

SEQ ID NO: 589 - protein, 24 aa, synthetic construct
GGSSGGSSGGSSGGSSGGSSGGSS

SEQ ID NO: 590 - protein, 28 aa, synthetic construct
GGSSGGSSGGSSGGSSGGSSGGSSGGSS

SEQ ID NO: 591 - protein, 32 aa, synthetic construct
GGSSGGSSGGSSGGSSGGSSGGSSGGSSGGSS

SEQ ID NO: 592 - protein, 36 aa, synthetic construct
GGSSGGSSGGSSGGSSGGSSGGSSGGSSGGSSGGSS

SEQ ID NO: 593 - protein, 40 aa, synthetic construct
GGSSGGSSGGSSGGSSGGSSGGSSGGSSGGSSGGSSGGSS

SEQ ID NO: 594 - protein, 44 aa, synthetic construct
GGSSGGSSGGSSGGSSGGSSGGSSGGSSGGSSGGSSGGSSGGSS

SEQ ID NO: 595 - protein, 48 aa, synthetic construct
GGSSGGSSGGSSGGSSGGSSGGSSGGSSGGSSGGSSGGSSGGSSGGSS

SEQ ID NO: 596 - protein, 5 aa, synthetic construct
GGGGS

SEQ ID NO: 597 - protein, 15 aa, synthetic construct
GGGGSGGGGSGGGGS

SEQ ID NO: 598 - protein, 30 aa, synthetic construct
GGGGSGGGGSGGGGSGGGGSGGGGSGGGGS

SEQ ID NO: 599 - protein, 45 aa, synthetic construct
GGGGSGGGGSGGGGSGGGGSGGGGSGGGGSGGGGSGGGGSGGGGS

SEQ ID NO: 600 - protein, 60 aa, synthetic construct
GGGGSGGGGSGGGGSGGGGSGGGGSGGGGSGGGGSGGGGSGGGGSGGGGSGGGGSGGGGS

SEQ ID NO: 601 - protein, 10 aa, synthetic construct
GGGGSGGGGS

SEQ ID NO: 602 - protein, 20 aa, synthetic construct
GGGGSGGGGSGGGGSGGGGS

SEQ ID NO: 603 - protein, 25 aa, synthetic construct
GGGGSGGGGSGGGGSGGGGSGGGGS

SEQ ID NO: 604 - protein, 35 aa, synthetic construct
GGGGSGGGGSGGGGSGGGGSGGGGSGGGGSGGGGS

SEQ ID NO: 605 - protein, 40 aa, synthetic construct
GGGGSGGGGSGGGGSGGGGSGGGGSGGGGSGGGGSGGGGS

SEQ ID NO: 606 - protein, 50 aa, synthetic construct
GGGGSGGGGSGGGGSGGGGSGGGGSGGGGSGGGGSGGGGSGGGGSGGGGS

SEQ ID NO: 607 - protein, 55 aa, synthetic construct
GGGGSGGGGSGGGGSGGGGSGGGGSGGGGSGGGGSGGGGSGGGGSGGGGSGGGGS

SEQ ID NO: 608 - protein, 7 aa, synthetic construct
AEAAAKA

SEQ ID NO: 609 - protein, 15 aa, synthetic construct
EAAAKEAAAKEAAAK

SEQ ID NO: 610 - protein, 15 aa, synthetic construct
  VARIANT 1..15: note = This sequence may encompass 1-3 EAAAK repeating units
EAAAKEAAAKEAAAK

SEQ ID NO: 611 - protein, 42 aa, synthetic construct
AEAAKEAAKEAAKEAAKALEAEAAAKEAAAKEAAAKEAAAKA

SEQ ID NO: 612 - protein, 5 aa, synthetic construct
PAPAP

SEQ ID NO: 613 - protein, 12 aa, synthetic construct
AEAAAKEAAAKA

SEQ ID NO: 614 - protein, 68 aa, synthetic construct
  VARIANT 1..68: note = This sequence may encompass 10-34 AP repeating units
APAPAPAPAPAPAPAPAPAPAPAPAPAPAPAPAPAPAPAPAPAPAPAPAPAPAPAPAPAPAPAPAPAP

SEQ ID NO: 615 - protein, 17 aa, synthetic construct
VSQTSKLTRAETVFPDV

SEQ ID NO: 616 - protein, 6 aa, synthetic construct
PLGLWA

SEQ ID NO: 617 - protein, 10 aa, synthetic construct
EDVVCCSMSY

SEQ ID NO: 618 - protein, 7 aa, synthetic construct
GGIERGS

SEQ ID NO: 619 - protein, 10 aa, synthetic construct
TRHRQPRGWE

SEQ ID NO: 620 - protein, 10 aa, synthetic construct
AGNRVRRSVG

SEQ ID NO: 621 - protein, 9 aa, synthetic construct
RRRRRRRRR

SEQ ID NO: 622 - protein, 8 aa, synthetic construct
  VARIANT 2: note = Any amino acid
  VARIANT 4: note = Any amino acid
DXEXNPGP

SEQ ID NO: 623 - protein, 22 aa, synthetic construct
  VARIANT 1: note = May be absent
  VARIANT 2: note = May be absent
  VARIANT 3: note = May be absent
  VARIANT 22: note = May be absent
GSGEGRGSLLTCGDVEENPGPS

SEQ ID NO: 624 - protein, 23 aa, synthetic construct
  VARIANT 1: note = May be absent
  VARIANT 2: note = May be absent
  VARIANT 3: note = May be absent
  VARIANT 23: note = May be absent
GSGATNFSLLKQAGDVEENPGPS

SEQ ID NO: 625 - protein, 24 aa, synthetic construct
  VARIANT 1: note = May be absent
  VARIANT 2: note = May be absent
  VARIANT 3: note = May be absent
  VARIANT 24: note = May be absent
GSGQCTNYALLKLAGDVESNPGPS

SEQ ID NO: 626 - protein, 26 aa, synthetic construct
  VARIANT 1: note = May be absent
  VARIANT 2: note = May be absent
  VARIANT 3: note = May be absent
  VARIANT 26: note = May be absent
GSGVKQTLNFDLLKLAGDVESNPGPS

SEQ ID NO: 627 - protein, 36 aa, synthetic construct; identical to SEQ ID NO: 571
  VARIANT 1..36: note = This sequence may encompass 2-12 GGS repeating units

SEQ ID NO: 628 - protein, 30 aa, synthetic construct; identical to SEQ ID NO: 598

SEQ ID NO: 629 - protein, 48 aa, synthetic construct; identical to SEQ ID NO: 595
  VARIANT 1..48: note = This sequence may encompass 2-12 GGSS repeating units

SEQ ID NO: 630 - protein, 60 aa, synthetic construct; identical to SEQ ID NO: 600
  VARIANT 1..60: note = This sequence may encompass 2-12 GGGGS repeating units

SEQ ID NO: 631 - protein, 6 aa, synthetic construct
RVLAEA